(19) United States
(12) Patent Application Publication
(10) Pub. No.: US 2026/0152484 A1
(43) Pub. Date: Jun. 4, 2026

(54) HETEROCYCLIC COMPOUNDS AS TRIGGERING RECEPTOR EXPRESSED ON MYELOID CELLS 2 AGONISTS

(71) Applicant: Vigil Neuroscience, Inc., Watertown, MA (US)

(72) Inventors: Jonathan B. HOUZE, Watertown, MA (US); Bhaumik PANDYA, Watertown, MA (US); Alan P. KAPLAN, Watertown, MA (US)

(73) Assignee: Vigil Neuroscience, Inc., Watertown, MA (US)

(21) Appl. No.: 18/707,462

(22) PCT Filed: Nov. 9, 2022
(86) PCT No.: PCT/US2022/079515

§ 371 Date: May 3, 2024

Related U.S. Application Data
(60) Provisional application No. 63/263,812, filed on Nov. 9, 2021.

(51) Int. Cl. C07D 401/14; A61K 31/4375; A61K 31/4725; A61K 31/4985; A61K 31/502; A61K 31/5025; A61K 31/5377; C07D 237/32; C07D 401/04; C07D 401/10; C07D 403/10; C07D 403/12; C07D 405/04; C07D 405/14; C07D 471/04; C07D 491/056; C07D 513/04

(52) U.S. Cl. CPC C07D 401/14; A61K 31/4375; A61K 31/4725; A61K 31/4985; A61K 31/502; A61K 31/5025; A61K 31/5377; C07D 237/32; C07D 401/04; C07D 401/10; C07D 403/10; C07D 403/12; C07D 405/04; C07D 405/14; C07D 471/04; C07D 491/056; C07D 513/04

(57) ABSTRACT

The present disclosure provides compounds of Formula I, useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

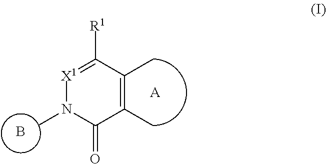

### CROSS REFERENCE TO RELATED APPLICATION

This application claims the benefit of U.S. Provisional Application No. 63/263,812, filed Nov. 9, 2021, the entirety of which is incorporated herein by reference.

### FIELD

The present disclosure provides compounds useful for the activation of Triggering Receptor Expressed on Myeloid Cells 2 (“TREM2”). This disclosure also provides pharmaceutical compositions comprising the compounds, uses of the compounds, and compositions for treatment of, for example, a neurodegenerative disorder. Further, the disclosure provides intermediates useful in the synthesis of compounds of Formula I.

### BACKGROUND

Microglia are resident innate immune cells in the brain and are important for the maintenance of homeostatic conditions in the central nervous system (Hickman et al. Nat Neurosci 2018, Li and Barres, Nat Rev Immunol., 2018). These resident macrophages express a variety of receptors that allow them to sense changes in their microenvironment and alter their phenotypes to mediate responses to invading pathogens, proteotoxic stress, cellular injury, and other infarcts that can occur in health and disease. Id. Microglia reside in the parenchyma of the brain and spinal cord where they interact with neuronal cell bodies (Cserep et al. Science, 2019), neuronal processes (Paolicelli et al. Science, 2011, Ikegami et al. Neruopathology, 2019) in addition to other types of glial cells (Domingues et al. Front Cell Dev Biol, 2016; Liddelow et al. Nature, 2017, Shinozaki et al. Cell Rep., 2017), playing roles in a multitude of physiological processes. With the ability to rapidly proliferate in response to stimuli, microglia characteristically exhibit myeloid cell functions such as phagocytosis, cytokine/chemokine release, antigen presentation, and migration (Colonna and Butovsky, Annu Rev Immunol, 2017). More specialized functions of microglia include the ability to prune synapses from neurons and directly communicate with their highly arborized cellular processes that survey the area surrounding the neuronal cell bodies (Hong et al. Curr Opin Neurobiol, 2016; Sellgren et al. Nat Neurosci, 2019).

The plasticity of microglia and their diverse states as described through single-cells RNASeq profiling are thought to arise through the integration of signaling from a diverse array of cell surface receptors (Hickman et al. Nat Neurosci 2013). Collectively known as the microglial “sensome,” these receptors are responsible for transducing activating or activation-suppressing intracellular signaling and include protein families such as Sialic acid-binding immunoglobulin-type lectins (“SIGLEC”), Toll-like receptors (“TLR”), Fc receptors, nucleotide-binding oligomerization domain (“NOD”) and purinergic G protein-coupled receptors. Doens and Fernandez 2014, Madry and Attwell 2015, Hickman and El Khoury 2019. Similar to other cells of the myeloid lineage, the composition of microglial sensomes is dynamically regulated and acts to recognize molecular pattern that direct phenotypic responses to homeostatic changes in the central nervous system (“CNS”). Id. One of the receptors selectively expressed by brain microglia is TREM2, composed of a single-pass transmembrane domain, an extracellular stalk region, and extracellular immunoglobulin variable (“IgV”)-like domain responsible for ligand interaction (Kleinberger et al. Sci Transl Med, 2014). As TREM2 does not possess intracellular signal transduction-mediating domains, biochemical analysis has illustrated that interaction with adaptor proteins DAP10 and DAP12 mediate downstream signal transduction following ligand recognition (Peng et al. Sci Signal 2010; Jay et al. Mol Neurodegener, 2017). TREM2/DAP12 complexes in particular act as a signaling unit that can be characterized as pro-activation on microglial phenotypes in addition to peripheral macrophages and osteoclasts (Otero et al. J Immunol, 2012; Kobayashi et al. J Neurosci, 2016; Jaitin et al., Cell, 2019. In the CNS, signaling through TREM2 has been studied in the context of ligands such as phospholipids, cellular debris, apolipoproteins, and myelin (Wang et al. Cell, 2015; Kober and Brett, J Mol Biol, 2017; Shirotani et al., Sci Rep, 2019). In mice lacking functional TREM2 expression or expressing a mutated form of the receptor, a core observation is blunted microglial responses to insults such as oligodendrocyte demyelination, stroke-induced tissue damage in the brain, and proteotoxic inclusions in vivo (Cantoni et al., Acta Neuropathol, 2015, Wu et al., Mol Brain, 2017).

Coding variants in the TREM12 locus has been associated with late onset Alzheimer's disease (“LOAD”) in human genome-wide association studies, linking a loss-of-receptor function to a gain in disease risk (Jonsson et al. N Engl J Med 2013, Sims et al. Nat Genet 2017). Genetic variation of other genes selectively expressed by microglia in the CNS, for example, CD33, PLCg2 and MS4A4A/6A have reached genome-wide significance for their association with LOAD risk (Hollingworth et al. Nat Genet 2011, Sims et al. Nat Genet 2017, Deming et al. Sci Transl Med 2019). Together, these genetic findings link together in a putative biochemical circuit that highlights the importance of microglial innate immune function in LOAD. Additionally, increase or elevation in the soluble form of TREM2 (“sTREM2”) in the cerebrospinal fluid (CSF) of human subjects is associated with disease progression and emergence of pathological hallmarks of LOAD including phosphorylated Tau (Suarez-Calvet et al. Mol Neurodegener 2019). Furthermore, natural history and human biology studies indicate that baseline sTREM2 levels in the CSF can stratify the rate of temporal lobe volume loss and episodic memory decline in longitudinally monitored cohorts (Ewers et al. Sci Transl Med 2019).

In addition to human genetic evidence supporting a role of TREM2 in LOAD, homozygous loss-of-function mutations in TREM12 are causal for an early onset dementia syndrome known as Polycystic lipomembranous osteodysplasia with sclerosing leukoencephalopathy (“PLOSL”) or Nasu-Hakola disease (“NHD”) (Golde et al. Alzheimers Res Ther 2013, Dardiotis et al. Neurobiol Aging 2017). This progressive neurodegenerative disease typically manifests in the 3^{rd} decade of life and is pathologically characterized by loss of myelin in the brain concomitant with gliosis, unresolved neuroinflammation, and cerebral atrophy. Typical neuropsychiatric presentations are often preceded by osseous abnormalities, such as bone cysts and loss of peripheral bone density (Bianchin et al. Cell Mol Neurobiol 2004; Madry et al. Clin Orthop Relat Res 2007, Bianchin et al. Nat Rev Neurol 2010). Given that osteoclasts of the myeloid lineage are also known to express TREM2, the PLOSL-related symptoms of wrist and ankle pain, swelling, and fractures indicate that TREM2 may act to regulate bone homeostasis through defined signaling pathways that parallel the microglia in the CNS (Paloneva et al. J Exp Med 2003, Otero et al. J Immunol 2012). The link between TREM2 function and PLOSL has illustrated the importance of the receptor in sustaining key physiological aspects of myeloid cell function in the human body.

Efforts have been made to model the biology of TREM2 in mice prompting the creation of TREM2 knock out (“KO”) mice in addition to the LOAD-relevant TREM2 R47H loss-of-function mutant transgenic mice (Ulland et al. Cell, 2017, Kang et al. Hum Mol Genet 2018). Although unable to recapitulate the neurological manifestations of PLOSL, TREM12 KO mice show abnormalities in bone ultrastructure (Otero et al. J Immunol 2012). When the TREM2 KO or mutant mice have been crossed onto familial Alzheimer's disease transgenic mouse background such as the 5×FAD amyloidogenic mutation lines, marked phenotypes have been observed (Ulrich et al. Neuron, 2017). These in vivo phenotypes of TREM2 loss-of-function in the CNS include elevated the plaque burden and lower levels of secreted microglial factors SPP1 and Osteopontin that are characteristic of the microglial response to amyloid pathology (Ulland et al. Cell, 2017). Other rodent studies have demonstrated that loss of TREM2 leads to decreased microglial clustering around plaques and emergence of less compact plaque morphology in familial AD amyloid models (Parhizkar et al. Nat Neurosci 2019). With regards to the Tau protein pathology that is observed in LOAD, familial tauopathy models in mice demonstrated an enhanced spreading of pathological human Tau aggregates from point of injection into mouse brain in TREM2 KO mice (Leyns et al. Nat Neurosci 2019). Furthermore, single-cell RNASeq studies with the TREM2 KO mice in aged scenarios, 5×FAD familial Alzheimer's disease model mice, and Amyotrophic Lateral Sclerosis SOD1 mutant mouse backgrounds indicate that TREM2 receptor function is critical for a conserved set of phenotypic transformations within microglial populations in response to CNS pathology (Keren-Shaul et al. Cell 2017).

In rodent models where TREM2 expression levels are elevated, brain amyloid pathology in the 5×FAD transgenic mice displayed reduced plaque volume and altered morphology (Lee et al. Neuron, 2018). The changes in immunohistological markers relating to brain amyloid pathology were also accompanied by an attenuated presence of dystrophic neurites when TREM12 was overexpressed. Id. Therefore, the pharmacological activation of TREM2 is a target of interest for treating or preventing neurological, neurodegenerative and other diseases. Despite many attempts to alter disease progression by targeting the pathological hallmarks of LOAD through anti-amyloid and anti-Tau therapeutics, there is a need for activators of TREM2 to address the genetics-implicated neuroimmune aspects of, for example, LOAD. Such TREM2 activators may be suitable for use as therapeutic agents and remain in view of the significant continuing societal burden that remains unmitigated for diseases, such as Alzheimer's disease.

### SUMMARY

Provided herein is a compound of Formula I

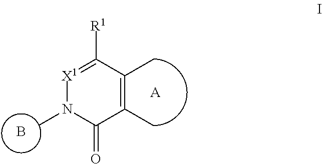

or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CH or N;
Ring A together with the 6-membered ring system to which it is fused forms a bicyclic ring system of formula

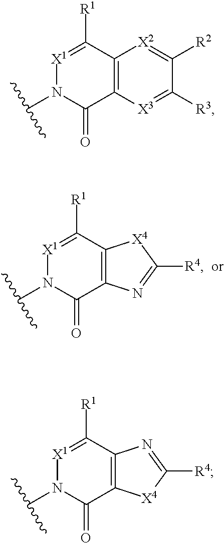

R² and R are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
provided that at least one of R² and R³ is not hydrogen;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X² is CH, CR¹⁴, or N;
X³ is CH, CR¹⁵, or N;
R⁴ is selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁴ is NR, O or S;
Ring B is

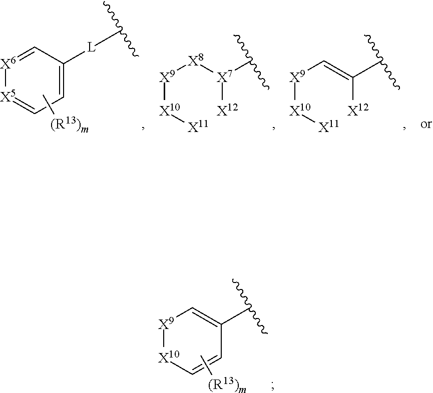

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
each R¹³ is independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
R¹⁴ and R¹⁵ are each independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

Also provided herein is a pharmaceutical composition comprising a compound of Formula I, or a tautomer thereof, or a pharmaceutically acceptable salt of said compound or said tautomer, and a pharmaceutically acceptable excipient.

Also provided herein is a compound of Formula I, or a tautomer thereof, or a pharmaceutically acceptable salt of said compound or said tautomer, or a pharmaceutical composition as described hereinabove, for use in treating or preventing a condition associated with a loss of function of human TREM2.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein is a compound of Formula I

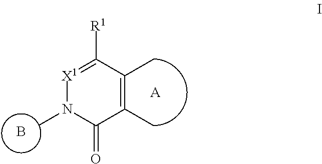

or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CH, CR¹⁶, or N;
Ring A together with the 6-membered ring system to which it is fused forms a bicyclic ring system of formula

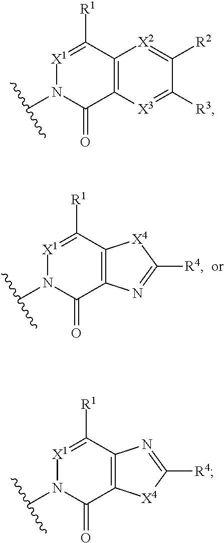

R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
provided that at least one of R² and R³ is not hydrogen;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X² is CH, CR¹⁴, or N;
X³ is CH, CR¹⁵, or N;
R⁴ is selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁴ is NR, O or S;
Ring B is

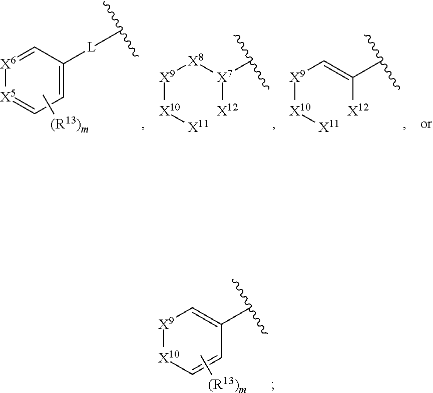

L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁴ and R¹⁵ are each independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
R¹⁴ and R¹⁵ are each independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

In some embodiments, when L is a direct bond, R⁵, R⁶ and R¹³ are not halogen. In some embodiments, when L is a direct bond, R⁵, R⁶ and R¹³ are not haloalkyl.

In some embodiments, the compound is not a compound selected from:

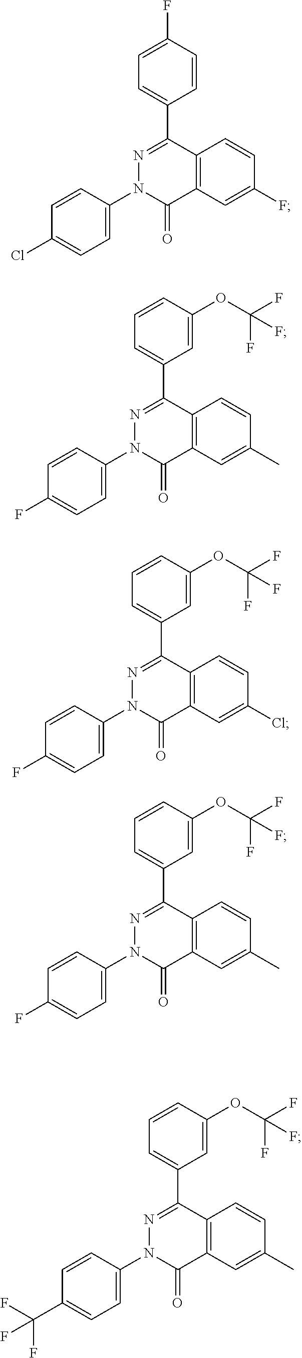

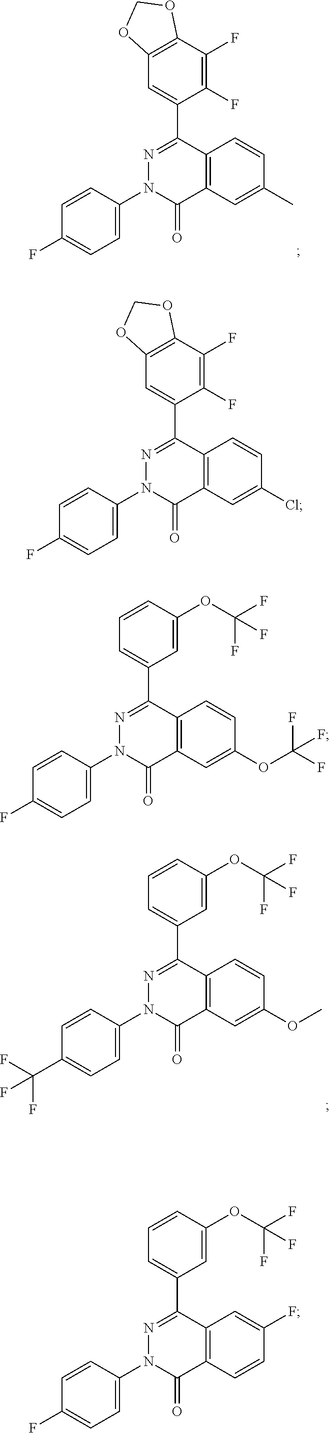 [SMILES: Cc1ccc2c(-c3cc4c(c(F)c3F)OCO4)nn(-c3ccc(F)cc3)c(=O)c2c1 ; O=c1c2cc(Cl)ccc2c(-c2cc3c(c(F)c2F)OCO3)nn1-c1ccc(F)cc1 ; O=c1c2cc(OC(F)(F)F)ccc2c(-c2cccc(OC(F)(F)F)c2)nn1-c1ccc(F)cc1 ; COc1ccc2c(-c3cccc(OC(F)(F)F)c3)nn(-c3ccc(C(F)(F)F)cc3)c(=O)c2c1 ; O=c1c2ccc(F)cc2c(-c2cccc(OC(F)(F)F)c2)nn1-c1ccc(F)cc1]

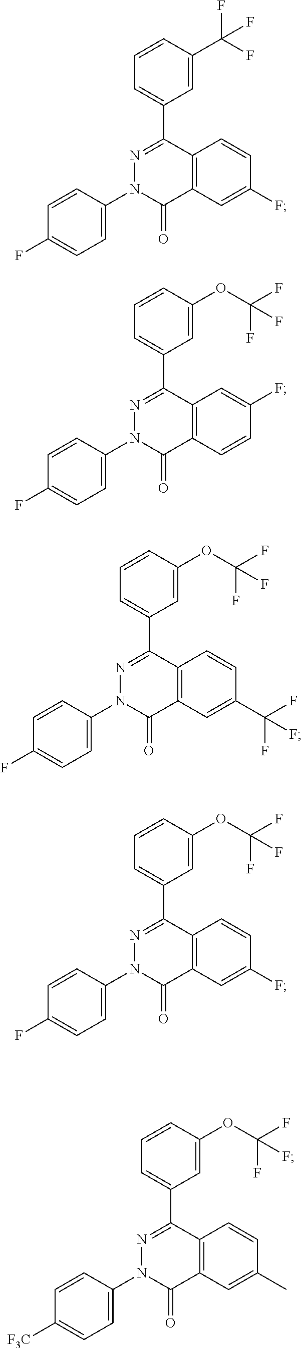 [SMILES: O=c1c2cc(F)ccc2c(-c2cccc(C(F)(F)F)c2)nn1-c1ccc(F)cc1 ; O=c1c2ccc(F)cc2c(-c2cccc(OC(F)(F)F)c2)nn1-c1ccc(F)cc1 ; O=c1c2cc(C(F)(F)F)ccc2c(-c2cccc(OC(F)(F)F)c2)nn1-c1ccc(F)cc1 ; O=c1c2cc(F)ccc2c(-c2cccc(OC(F)(F)F)c2)nn1-c1ccc(F)cc1 ; Cc1ccc2c(-c3cccc(OC(F)(F)F)c3)nn(-c3ccc(C(F)(F)F)cc3)c(=O)c2c1]

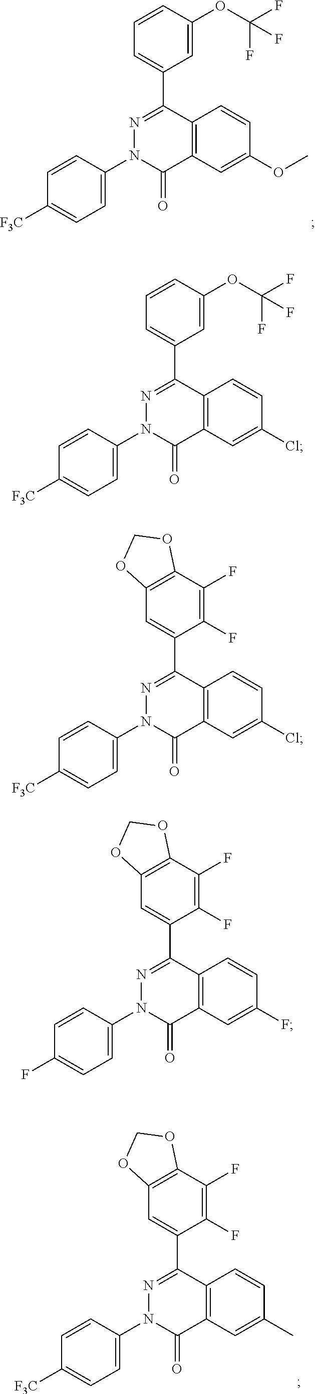 [SMILES: COc1ccc2c(-c3cccc(OC(F)(F)F)c3)nn(-c3ccc(C(F)(F)F)cc3)c(=O)c2c1 ; O=c1c2cc(Cl)ccc2c(-c2cccc(OC(F)(F)F)c2)nn1-c1ccc(C(F)(F)F)cc1 ; O=c1c2cc(Cl)ccc2c(-c2cc3c(c(F)c2F)OCO3)nn1-c1ccc(C(F)(F)F)cc1 ; O=c1c2cc(F)ccc2c(-c2cc3c(c(F)c2F)OCO3)nn1-c1ccc(F)cc1 ; Cc1ccc2c(-c3cc4c(c(F)c3F)OCO4)nn(-c3ccc(C(F)(F)F)cc3)c(=O)c2c1]

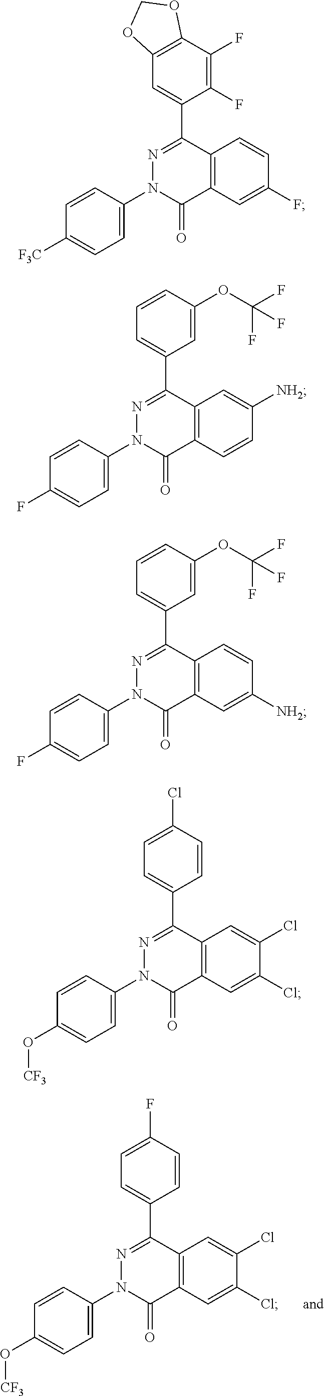 [SMILES: O=c1c2cc(F)ccc2c(-c2cc3c(c(F)c2F)OCO3)nn1-c1ccc(C(F)(F)F)cc1 ; Nc1ccc2c(=O)n(-c3ccc(F)cc3)nc(-c3cccc(OC(F)(F)F)c3)c2c1 ; Nc1ccc2c(-c3cccc(OC(F)(F)F)c3)nn(-c3ccc(F)cc3)c(=O)c2c1 ; O=c1c2cc(Cl)c(Cl)cc2c(-c2ccc(Cl)cc2)nn1-c1ccc(OC(F)(F)F)cc1 ; O=c1c2cc(Cl)c(Cl)cc2c(-c2ccc(F)cc2)nn1-c1ccc(OC(F)(F)F)cc1 ; "and"]

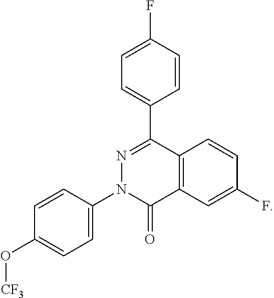 [SMILES: O=c1c2cc(F)ccc2c(-c2ccc(F)cc2)nn1-c1ccc(OC(F)(F)F)cc1]

In some embodiments, the compound is a compound of Formula II:

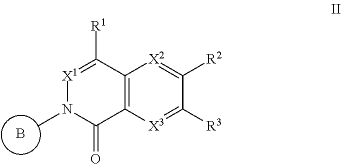

or a tautomer thereof, or a pharmaceutically acceptable salt of said compound or said tautomer, wherein each variable is as defined above and described in embodiments herein both singly and in combination.

In some embodiments, the compound is a compound of Formula IIa:

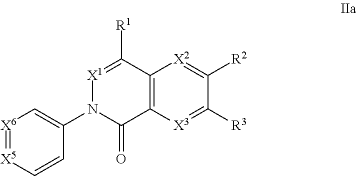

or a tautomer thereof, or a pharmaceutically acceptable salt of said compound or said tautomer, wherein each variable is as defined above and described in embodiments herein both singly and in combination.

In some embodiments, the compound is a compound of Formula IIa*:

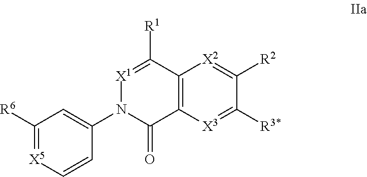

or a tautomer thereof, or a pharmaceutically acceptable salt of said compound or said tautomer, wherein each variable is as defined above and described in embodiments herein both singly and in combination.

In some embodiments, the compound is a compound of Formula IIa**:

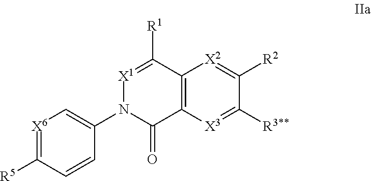

or a tautomer thereof, or a pharmaceutically acceptable salt of said compound or said tautomer, wherein each variable is as defined above and described in embodiments herein both singly and in combination.

In some embodiments, the compound is a compound of Formula IIb:

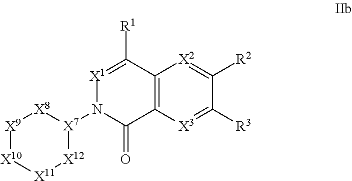

or a tautomer thereof, or a pharmaceutically acceptable salt of said compound or said tautomer, wherein each variable is as defined above and described in embodiments herein both singly and in combination.

In some embodiments, the compound is a compound of Formula IIb*:

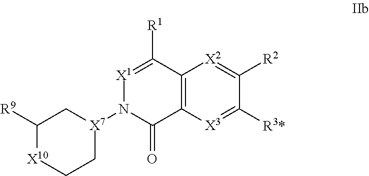

or a tautomer thereof, or a pharmaceutically acceptable salt of said compound or said tautomer, wherein each variable is as defined above and described in embodiments herein both singly and in combination.

In some embodiments, the compound is a compound of Formula IIb**:

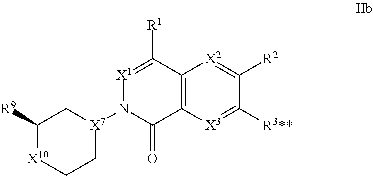

or a tautomer thereof, or a pharmaceutically acceptable salt of said compound or said tautomer, wherein each variable is as defined above and described in embodiments herein both singly and in combination.

In some embodiments, the compound is a compound of Formula IIb***:

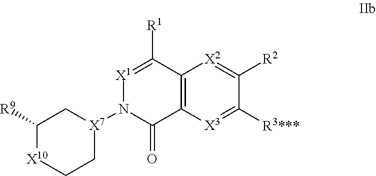

or a tautomer thereof, or a pharmaceutically acceptable salt of said compound or said tautomer, wherein each variable is as defined above and described in embodiments herein both singly and in combination.

In some embodiments, the compound is a compound of Formula IIb′:

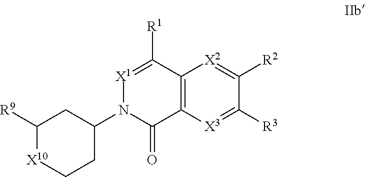

or a tautomer thereof, or a pharmaceutically acceptable salt of said compound or said tautomer, wherein each variable is as defined above and described in embodiments herein both singly and in combination.

In some embodiments, the compound is a compound of Formula IIb″:

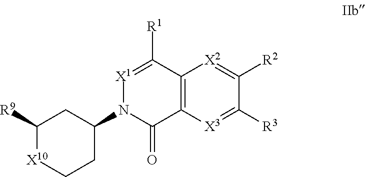

or a tautomer thereof, or a pharmaceutically acceptable salt of said compound or said tautomer, wherein each variable is as defined above and described in embodiments herein both singly and in combination.

In some embodiments, the compound is a compound of Formula IIb′″:

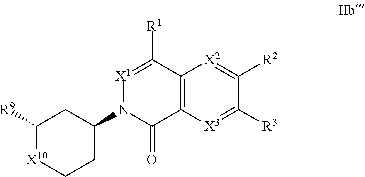

or a tautomer thereof, or a pharmaceutically acceptable salt of said compound or said tautomer, wherein each variable is as defined above and described in embodiments herein both singly and in combination.

In some embodiments, the compound is a compound of Formula IIb″″:

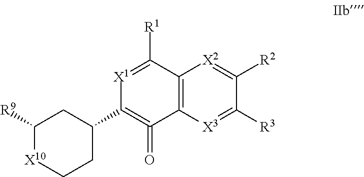

or a tautomer thereof, or a pharmaceutically acceptable salt of said compound or said tautomer, wherein each variable is as defined above and described in embodiments herein both singly and in combination.

In some embodiments, the compound is a compound of Formula IIb′″″:

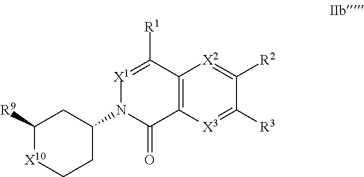

or a tautomer thereof, or a pharmaceutically acceptable salt of said compound or said tautomer, wherein each variable is as defined above and described in embodiments herein both singly and in combination.

In some embodiments, the compound is a compound of Formula III:

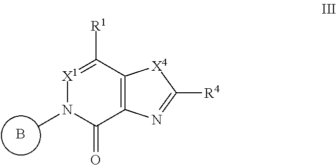

or a tautomer thereof, or a pharmaceutically acceptable salt of said compound or said tautomer, wherein each variable is as defined above and described in embodiments herein both singly and in combination.

In some embodiments, the compound is a compound of Formula IIIa:

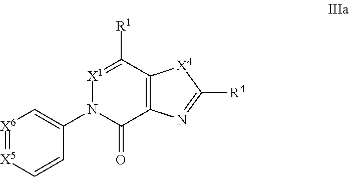

or a tautomer thereof, or a pharmaceutically acceptable salt of said compound or said tautomer, wherein each variable is as defined above and described in embodiments herein both singly and in combination.

In some embodiments, the compound is a compound of Formula IIIb:

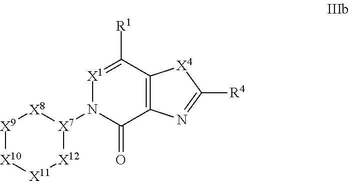

or a tautomer thereof, or a pharmaceutically acceptable salt of said compound or said tautomer, wherein each variable is as defined above and described in embodiments herein both singly and in combination.

In some embodiments, the compound is a compound of Formula IIIc:

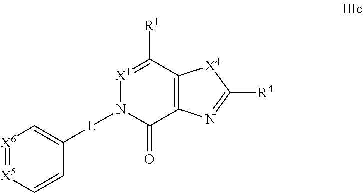

or a tautomer thereof, or a pharmaceutically acceptable salt of said compound or said tautomer, wherein each variable is as defined above and described in embodiments herein both singly and in combination.

In some embodiments, the compound is a compound of Formula IV:

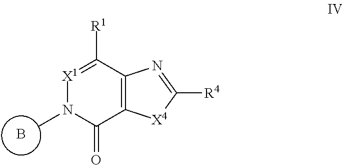

or a tautomer thereof, or a pharmaceutically acceptable salt of said compound or said tautomer, wherein each variable is as defined above and described in embodiments herein both singly and in combination.

In some embodiments, the compound is a compound of Formula IVa:

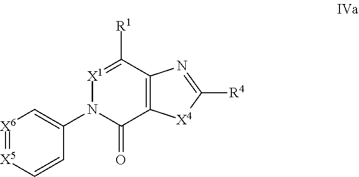

or a tautomer thereof, or a pharmaceutically acceptable salt of said compound or said tautomer, wherein each variable is as defined above and described in embodiments herein both singly and in combination.

In some embodiments, the compound is a compound of Formula IVb:

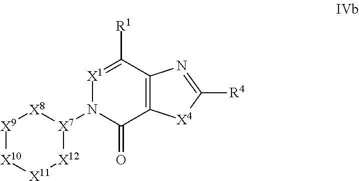

or a tautomer thereof, or a pharmaceutically acceptable salt of said compound or said tautomer, wherein each variable is as defined above and described in embodiments herein both singly and in combination.

In some embodiments, the compound is a compound of Formula IVc:

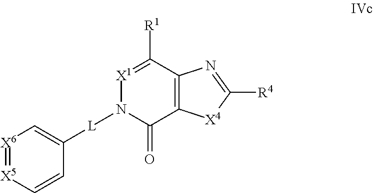

or a tautomer thereof, or a pharmaceutically acceptable salt of said compound or said tautomer, wherein each variable is as defined above and described in embodiments herein both singly and in combination.

As defined generally above, R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹ is an optionally substituted C₁₋₆ aliphatic group. In some embodiments, R¹ is —OR. In some embodiments, R¹ is —NR₂. In some embodiments, R¹ is —C(═O)R. In some embodiments, R¹ is —C(═O)OR. In some embodiments, R¹ is —C(═O)NR₂. In some embodiments, R² is —SO₂R. In some embodiments, R¹ is —SO₂NR₂. In some embodiments, R¹ is C₁₋₆haloalkyl. In some embodiments, R¹ is an optionally substituted OCH₂—(C₃₋₆cycloalkyl). In some embodiments, R¹ is an optionally substituted 3-8 membered saturated or partially unsaturated monocyclic carbocyclic ring. In some embodiments, R¹ is an optionally substituted 5-12 membered saturated or partially unsaturated bridged carbocyclic ring. In some embodiments, R¹ is an optionally substituted 7-12 membered saturated or partially unsaturated bicyclic carbocyclic ring. In some embodiments, R¹ is an optionally substituted phenyl. In some embodiments, R¹ is an optionally substituted 8-10 membered bicyclic aromatic carbocyclic ring. In some embodiments, R¹ is an optionally substituted 3-8 membered saturated or partially unsaturated monocyclic heterocyclic ring (having 1-2 heteroatoms independently selected from nitrogen, oxygen, and sulfur). In some embodiments, R¹ is an optionally substituted 6-12 membered saturated or partially unsaturated bridged heterocyclic ring (having 1-4 heteroatoms independently selected from nitrogen, oxygen, and sulfur). In some embodiments, R¹ is an optionally substituted 7-12 membered saturated or partially unsaturated bicyclic heterocyclic ring (having 1-4 heteroatoms independently selected from nitrogen, oxygen, and sulfur). In some embodiments, R¹ is an optionally substituted 5-6 membered monocyclic heteroaromatic ring (having 1-4 heteroatoms independently selected from nitrogen, oxygen, and sulfur). In some embodiments, R¹ is an optionally substituted 8-10 membered bicyclic heteroaromatic ring (having 1-5 heteroatoms independently selected from nitrogen, oxygen, and sulfur).

In some embodiments, R¹ is a cyclic group selected from a 3-8 membered saturated or partially unsaturated monocyclic carbocyclic ring, a 6-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R¹ is phenyl, optionally substituted with 1-3 substituents independently selected from halogen, C₁₋₆ aliphatic, —OR^{∘}, or C₁₋₆haloalkyl. In some embodiments, R¹ is phenyl, optionally substituted with 1-3 halogen. In some embodiments, R¹ is a 5-12 membered saturated or partially unsaturated bridged carbocyclic ring, optionally substituted with 1-3 substituents independently selected from halogen, C₁₋₆ aliphatic, —OR^{∘}, or C₁₋₆haloalkyl. In some embodiments, R¹ is a C₅₋₈tricycloalkyl ring, optionally substituted with 1-3 substituents independently selected from halogen, C₁₋₆ aliphatic, —OR^{∘}, or C₁₋₆haloalkyl. In some embodiments, R¹ is 5-6 membered monocyclic heteroaromatic ring (having 1-4 heteroatoms independently selected from nitrogen, oxygen, and sulfur), optionally substituted with 1-3 substituents independently selected from halogen, C₁₋₆ aliphatic, —OR^{∘}, or C₁₋₆haloalkyl. In some embodiments, R¹ is 5-6 membered monocyclic heteroaromatic ring (having 1-4 heteroatoms independently selected from nitrogen, oxygen, and sulfur), optionally substituted with 1-3 halogen. In some embodiments, the compound is a compound of any one of Formulas II, IIa, IIa*, IIa**, IIb, IIb*, IIb**, IIb***, IIb′, IIb″, IIb′″, IIb″″, IIb′″″, III, IIIa, IIIb, IIIc, IV, IVa, IVb, or IVc, wherein R1 is optionally substituted phenyl.

In some embodiments, R¹ is optionally substituted C₃₋₆cycloalkyl, optionally substituted spiro[3.3]heptanyl, optionally substituted spiro[5.2]octanyl, optionally substituted

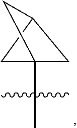 [SMILES: *C12CC(C1)C2]

optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted piperidine-1-yl, or optionally substituted —OCH₂—(C₃₋₄cycloalkyl). In some embodiments, R¹ is optionally substituted C₃₋₆cycloalkyl. In some embodiments, R¹ is optionally substituted spiro[3.3]heptanyl. In some embodiments, R¹ is optionally substituted spiro[5.2]octanyl. In some embodiments, R¹ is optionally substituted

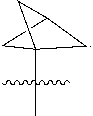 [SMILES: *C12CC(C1)C2]

In some embodiments, R¹ is optionally substituted cyclopent-1-en-1-yl. In some embodiments, R¹ is optionally substituted cyclohex-1-en-1-yl. In some embodiments, R¹ is optionally substituted phenyl. In some embodiments, R¹ is optionally substituted pyridinyl. In some embodiments, R¹ is optionally substituted aziridine-1-yl. In some embodiments, R¹ is optionally substituted pyrrolidine-1-yl. In some embodiments, R¹ is optionally substituted azabicyclo[3.1.0]hexan-3-yl. In some embodiments, R¹ is optionally substituted piperidine-1-yl. In some embodiments, R¹ is optionally substituted —OCH₂—(C₃₋₄cycloalkyl).

In some embodiments, R¹ is optionally substituted with 1-3 groups that are independently halogen; —(CH₂)₀₋₆R^{∘}; —(CH₂)₀₋₆OR^{∘}; —O(CH₂)₀₋₆R^{∘}, —O—(CH₂)₀₋₆—C(O)OR^{∘}; —(CH₂)₀₋₆CH(OR^{∘})₂; —(CH₂)₀₋₆—SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR^{∘}₂; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆—C(O)OR^{∘}; —(CH₂)₀₋₆C(O)SR^{∘}; —(CH₂)₀₋₆C(O)OSiR^{∘}₃; —(CH₂)₀₋₆OC(O)R^{∘}; —OC(O)(CH₂)₀₋₆—SR^{∘}, —(CH₂)₀₋₆—SC(O)R^{∘}; —(CH₂)₀₋₆C(O)NR^{∘}₂; —C(S)NR^{∘}₂; —C(S)SR^{∘}; —SC(S)SR^{∘}, —(CH₂)₀₋₆OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₆SSR^{∘}; —(CH₂)₀₋₆S(O)₂R^{∘}; —(CH₂)₀₋₆—S(O)₂OR^{∘}; —(CH₂)₀₋₆—OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined elsewhere herein and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur). In some embodiments, R¹ is optionally substituted with one or more —SF₅ groups.

In some embodiments, R¹ is —CH₂CH₂CF₃,

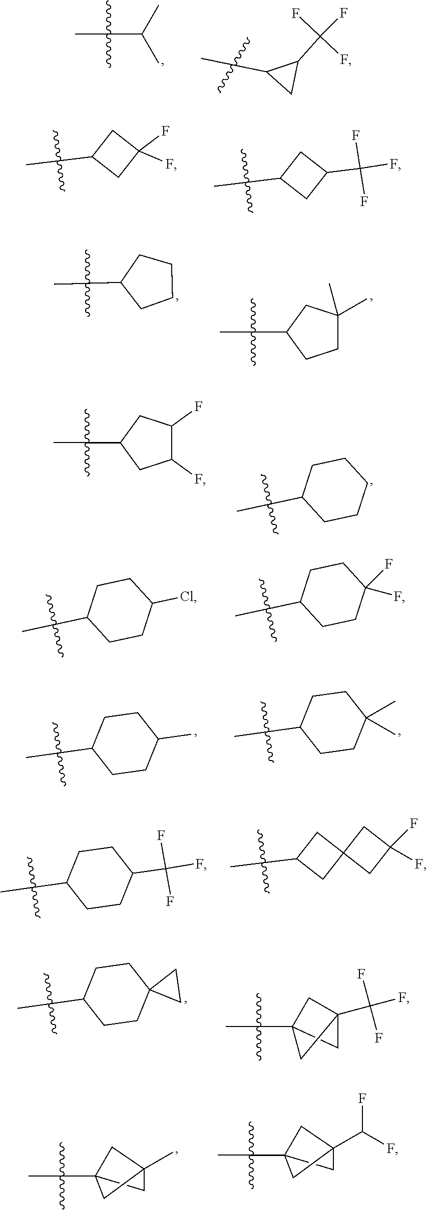

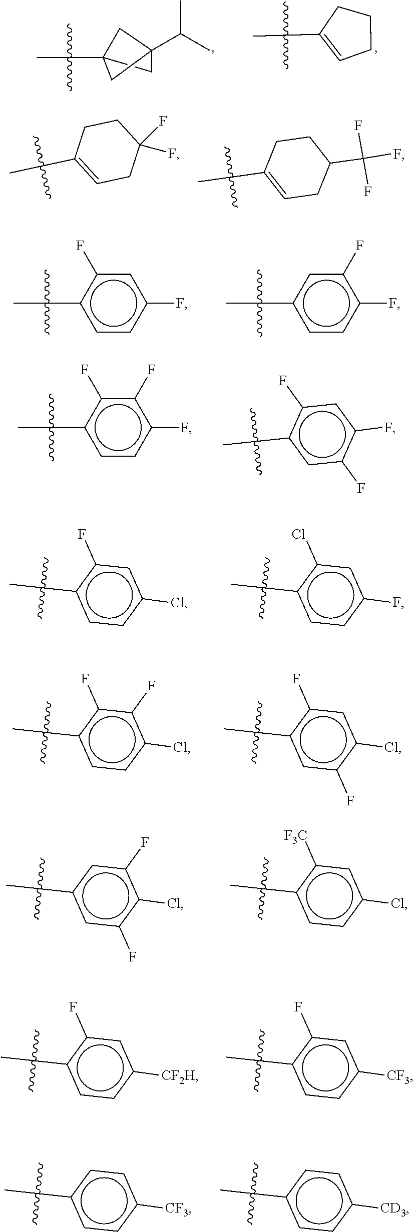

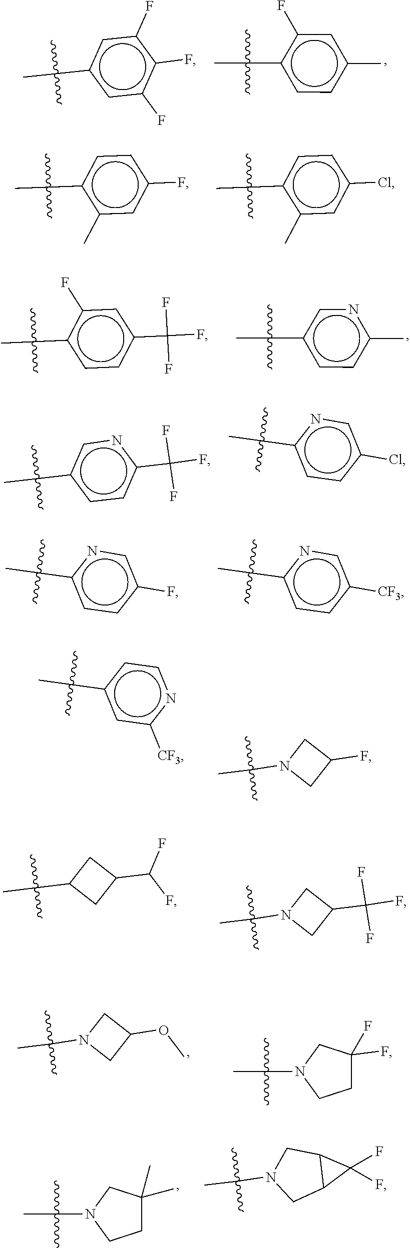

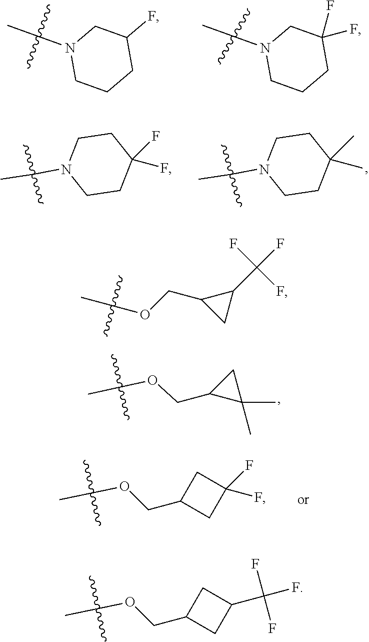

In some embodiments, R¹ is

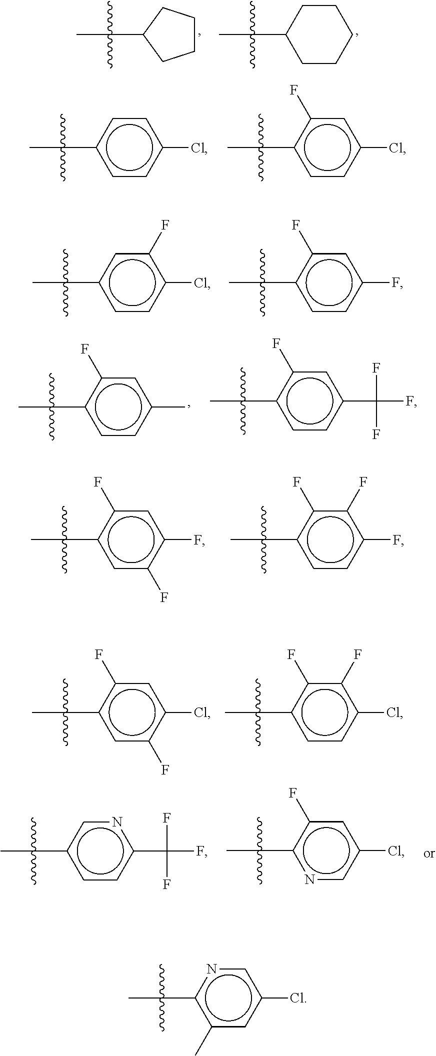

In some embodiments, R¹ is a substituent selected from those shown below:

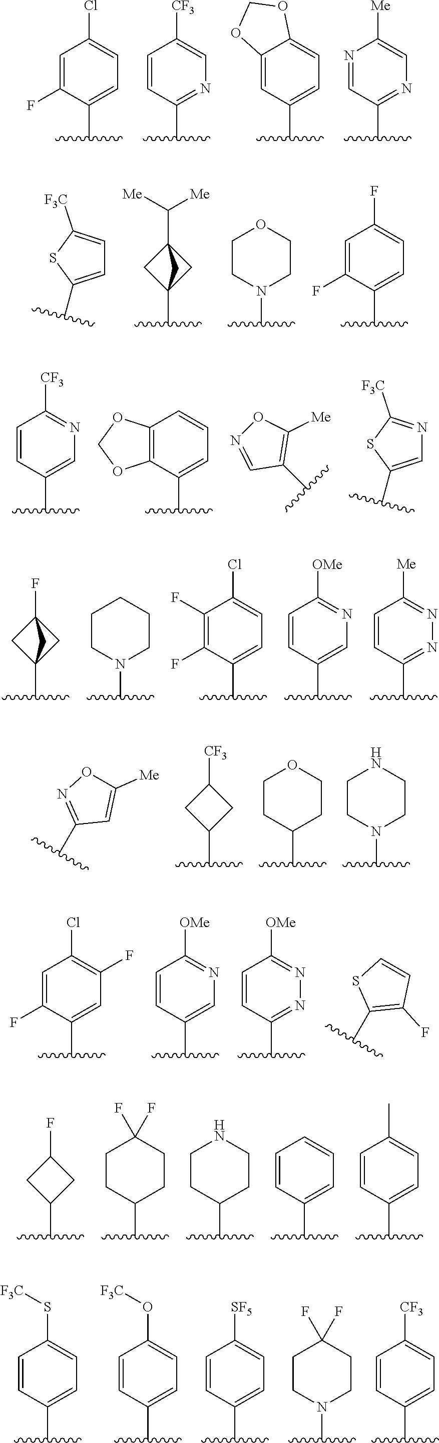

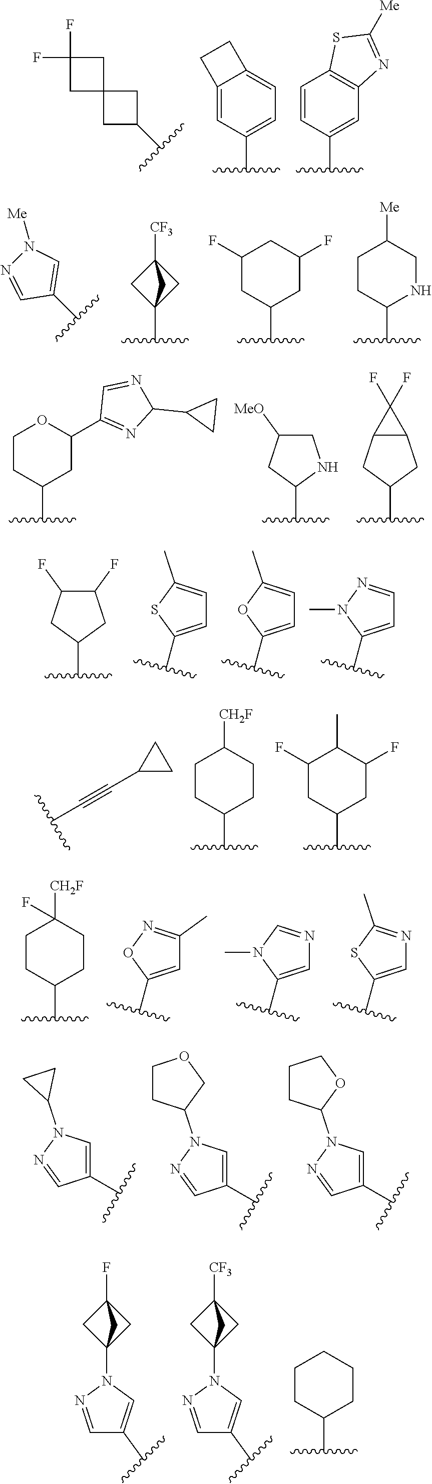

In some embodiments, R¹ is

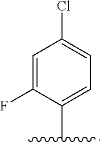 [SMILES: *c1ccc(Cl)cc1F]

In some embodiments, R¹ is

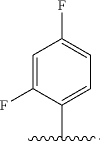 [SMILES: *c1ccc(F)cc1F]

In some embodiments, R¹ is

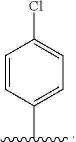 [SMILES: *c1ccc(Cl)cc1]

In some embodiments, R¹ is

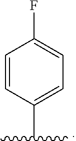 [SMILES: *c1ccc(F)cc1]

In some embodiments, R¹ is selected from those depicted in Table A below.

As defined generally above, X¹ is CH, CR¹⁶, or N. In some embodiments, X¹ is CH. In some embodiments, X¹ is CH or N. In some embodiments, X¹ is CR¹⁶. In some embodiments, X¹ is N. In some embodiments, X¹ is CCH₃. In some embodiments, X¹ is selected from those depicted in Table A below.

As defined generally above, R¹⁶ is an optionally substituted C₁₋₆ aliphatic group. In some embodiments, R¹⁶ is a C₁₋₆ aliphatic group. In some embodiments, R¹⁶ is a C₁₋₃ aliphatic group. In some embodiments, R¹⁶ is a methyl group.

As defined generally above, Ring A together with the 6-membered ring system to which it is fused forms a bicyclic ring system of formula

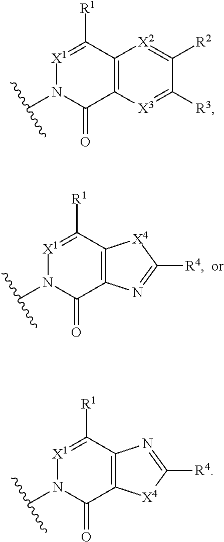

In some embodiments, Ring A forms a bicyclic system of formula

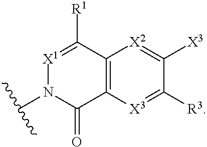

In some embodiments, Ring A forms a bicyclic system of formula

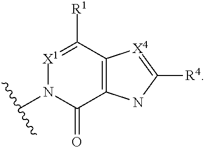

In some embodiments, Ring A forms a bicyclic system of formula

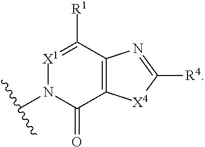

In some embodiments, Ring A forms a bicyclic system of formula

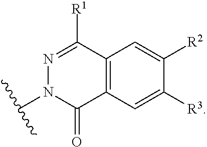

In some embodiments, Ring A forms a bicyclic system of formula

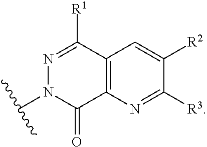

In some embodiments, Ring A forms a bicyclic system of formula

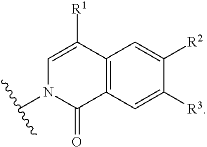

In some embodiments, Ring A forms a bicyclic system of formula

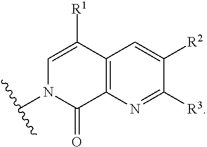

In some embodiments, Ring A forms a bicyclic system of formula

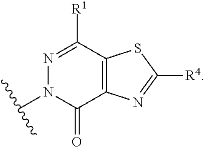

In some embodiments, Ring A forms a bicyclic system of formula

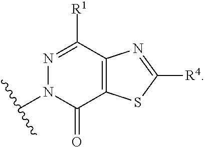

In some embodiments, Ring A is selected from those depicted in Table A below.

As defined generally above, R² and R³ are each independently hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted, provided that at least one of R² and R³ is not hydrogen; or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R² is an optionally substituted C₁₋₆ aliphatic group. In some embodiments, R² is halogen. In some embodiments, R² is —OR. In some embodiments, R² is —NR₂. In some embodiments, R² is —C(═O)R. In some embodiments, R² is —C(═O)OR. In some embodiments, R² is —C(═O)NR₂. In some embodiments, R² is —SO₂R. In some embodiments, R² is —SO₂NR₂. In some embodiments, R² is C₁₋₆haloalkyl. In some embodiments, R² is C₁₋₆haloalkoxy. In some embodiments, R² is an optionally substituted 3-8 membered saturated or partially unsaturated monocyclic carbocyclic ring. In some embodiments, R² is an optionally substituted 6-12 membered saturated or partially unsaturated bridged carbocyclic ring. In some embodiments, R² is an optionally substituted 7-12 membered saturated or partially unsaturated bicyclic carbocyclic ring. In some embodiments, R² is an optionally substituted phenyl. In some embodiments, R² is an optionally substituted 8-10 membered bicyclic aromatic carbocyclic ring. In some embodiments, R² is an optionally substituted 3-8 membered saturated or partially unsaturated monocyclic heterocyclic ring (having 1-2 heteroatoms independently selected from nitrogen, oxygen, and sulfur). In some embodiments, R² is an optionally substituted 6-12 membered saturated or partially unsaturated bridged heterocyclic ring (having 1-4 heteroatoms independently selected from nitrogen, oxygen, and sulfur). In some embodiments, R² is an optionally substituted 7-12 membered saturated or partially unsaturated bicyclic heterocyclic ring (having 1-4 heteroatoms independently selected from nitrogen, oxygen, and sulfur). In some embodiments, R² is an optionally substituted 5-6 membered monocyclic heteroaromatic ring (having 1-4 heteroatoms independently selected from nitrogen, oxygen, and sulfur). In some embodiments, R² is an optionally substituted 8-10 membered bicyclic heteroaromatic ring (having 1-5 heteroatoms independently selected from nitrogen, oxygen, and sulfur). In some embodiments, R² is selected from those depicted in Table A below.

In some embodiments, R³ is an optionally substituted C₁₋₆ aliphatic group. In some embodiments, R³ is halogen. In some embodiments, R³ is —OR. In some embodiments, R³ is —NR₂. In some embodiments, R³ is —C(═O)R. In some embodiments, R³ is —C(═O)OR. In some embodiments, R³ is —C(═O)NR₂. In some embodiments, R³ is —SO₂R. In some embodiments, R³ is —SO₂NR₂. In some embodiments, R³ is C₁₋₆haloalkyl. In some embodiments, R³ is C₁₋₆haloalkoxy. In some embodiments, R³ is an optionally substituted 3-8 membered saturated or partially unsaturated monocyclic carbocyclic ring. In some embodiments, R³ is an optionally substituted 6-12 membered saturated or partially unsaturated bridged carbocyclic ring. In some embodiments, R³ is an optionally substituted 7-12 membered saturated or partially unsaturated bicyclic carbocyclic ring. In some embodiments, R³ is an optionally substituted phenyl. In some embodiments, R³ is an optionally substituted 8-10 membered bicyclic aromatic carbocyclic ring. In some embodiments, R³ is an optionally substituted 3-8 membered saturated or partially unsaturated monocyclic heterocyclic ring (having 1-2 heteroatoms independently selected from nitrogen, oxygen, and sulfur). In some embodiments, R³ is an optionally substituted 6-12 membered saturated or partially unsaturated bridged heterocyclic ring (having 1-4 heteroatoms independently selected from nitrogen, oxygen, and sulfur). In some embodiments, R³ is an optionally substituted 7-12 membered saturated or partially unsaturated bicyclic heterocyclic ring (having 1-4 heteroatoms independently selected from nitrogen, oxygen, and sulfur). In some embodiments, R³ is an optionally substituted 5-6 membered monocyclic heteroaromatic ring (having 1-4 heteroatoms independently selected from nitrogen, oxygen, and sulfur). In some embodiments, R³ is an optionally substituted 8-10 membered bicyclic heteroaromatic ring (having 1-5 heteroatoms independently selected from nitrogen, oxygen, and sulfur). In some embodiments, R³ is selected from those depicted in Table A below.

In some embodiments, R² is hydrogen. In some embodiments, R² is methyl. In some embodiments, R² is Cl. In some embodiments, R² is a C₁₋₃ haloalkyl. In some embodiments, R² is 3-8 membered saturated monocyclic heterocyclic ring (having 1-2 heteroatoms independently selected from nitrogen, oxygen, and sulfur). In some embodiments, R² is an azetidinyl group. In some embodiments, R² is optionally substituted ethyl. In some embodiments, R² is methoxy. In some embodiments, R² is —CH₂F. In some embodiments, R² is —OCH₂F. In some embodiments, R² is —CD₃.

In some embodiments, R³ is hydrogen. In some embodiments, R³ is methyl. In some embodiments, R³ is Cl. In some embodiments, R³ is —CD₃.

In some embodiments, R² is H and R³ is methyl. In some embodiments, R² is methyl and R³ is methyl. In some embodiments, R² is Cl and R³ is Cl.

In some embodiments, R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R² and R³ are taken together with their intervening atoms to form an optionally substituted 3-8 membered saturated or partially unsaturated monocyclic carbocyclic ring. In some embodiments, R² and R³ are taken together with their intervening atoms to form an optionally substituted 6-12 membered saturated or partially unsaturated bridged carbocyclic ring. In some embodiments, R² and R³ are taken together with their intervening atoms to form an optionally substituted 7-12 membered saturated or partially unsaturated bicyclic carbocyclic ring. In some embodiments, R² and R are taken together with their intervening atoms to form an optionally substituted phenyl. In some embodiments, R² and R³ are taken together with their intervening atoms to form an optionally substituted 8-10 membered bicyclic aromatic carbocyclic ring. In some embodiments, R² and R are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R² and R³ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R² and R³ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R² and R are taken together with their intervening atoms to form an optionally substituted 5-6 membered monocyclic heteroaromatic ring (having 1-4 heteroatoms independently selected from nitrogen, oxygen, and sulfur). In some embodiments, R² and R³ are taken together with their intervening atoms to form an optionally substituted 8-10 membered bicyclic heteroaromatic ring (having 1-5 heteroatoms independently selected from nitrogen, oxygen, and sulfur).

In some embodiments, R² and R³ are taken together with their intervening atoms to form a dioxole ring.

As defined generally above, X² is CH, CR¹⁴, or N. In some embodiments, X² is CH. In some embodiments, X² is CR¹⁴. In some embodiments, X² is N. In some embodiments, X² is selected from those depicted in Table A below.

As defined generally above, X³ is CH, CR¹⁵, or N. In some embodiments, X³ is CH. In some embodiments, X³ is CR¹⁵. In some embodiments, X³ is N. In some embodiments, X³ is selected from those depicted in Table A below.

As defined generally above, R¹⁴ and R¹⁵ are each independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy.

In some embodiments, R¹⁴ is an optionally substituted C₁₋₆ aliphatic group. In some embodiments, R¹⁴ is halogen. In some embodiments, R¹⁴ is —OR. In some embodiments, R¹⁴ is —CN. In some embodiments, R¹⁴ is —NR₂. In some embodiments, R¹⁴ is —C(═O)R. In some embodiments, R¹⁴ is —C(═O)OR. In some embodiments, R¹⁴ is —C(═O)NR₂. In some embodiments, R¹⁴ is —SO₂R. In some embodiments, R¹⁴ is —SO₂NR₂. In some embodiments, R¹⁴ is C₁₋₆haloalkyl. In some embodiments, R¹⁴ is C₁₋₆haloalkoxy. In some embodiments, R¹⁴ is —CD₃. In some embodiments, R¹⁴ is selected from those depicted in Table A below.

In some embodiments, R¹⁵ is an optionally substituted C₁₋₆ aliphatic group. In some embodiments, R¹⁵ is halogen. In some embodiments, R¹⁵ is —OR. In some embodiments, R¹⁵ is —CN. In some embodiments, R¹⁵ is —NR₂. In some embodiments, R¹⁵ is —C(═O)R. In some embodiments, R¹⁵ is —C(═O)OR. In some embodiments, R¹⁵ is —C(═O)NR₂. In some embodiments, R¹⁵ is —SO₂R. In some embodiments, R¹⁵ is —SO₂NR₂. In some embodiments, R¹⁵ is C₁₋₆haloalkyl. In some embodiments, R¹⁵ is C₁₋₆haloalkoxy. In some embodiments, R¹⁵ is —CD₃. In some embodiments, R¹⁵ is selected from those depicted in Table A below.

As defined generally above, R⁴ is selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁴ is hydrogen. In some embodiments, R⁴ is an optionally substituted C₁₋₆ aliphatic group. In some embodiments, R⁴ is halogen. In some embodiments, R⁴ is —OR. In some embodiments, R⁴ is —CN. In some embodiments, R⁴ is —NR₂. In some embodiments, R⁴ is —C(═O)R. In some embodiments, R⁴ is —C(═O)OR. In some embodiments, R⁴ is —C(═O)NR₂. In some embodiments, R⁴ is —SO₂R. In some embodiments, R⁴ is —SO₂NR₂. In some embodiments, R⁴ is C₁₋₆haloalkyl. In some embodiments, R⁴ is C₁₋₆haloalkoxy. In some embodiments, R⁴ is an optionally substituted 3-8 membered saturated or partially unsaturated monocyclic carbocyclic ring. In some embodiments, R⁴ is an optionally substituted 7-12 membered saturated or partially unsaturated bicyclic carbocyclic ring, phenyl. In some embodiments, R⁴ is an optionally substituted 8-10 membered bicyclic aromatic carbocyclic ring. In some embodiments, R⁴ is an optionally substituted 3-8 membered saturated or partially unsaturated monocyclic heterocyclic ring (having 1-2 heteroatoms independently selected from nitrogen, oxygen, and sulfur). In some embodiments, R⁴ is an optionally substituted 7-12 membered saturated or partially unsaturated bicyclic heterocyclic ring (having 1-4 heteroatoms independently selected from nitrogen, oxygen, and sulfur). In some embodiments, R⁴ is an optionally substituted 5-6 membered monocyclic heteroaromatic ring (having 1-4 heteroatoms independently selected from nitrogen, oxygen, and sulfur). In some embodiments, R⁴ is an optionally substituted 8-10 membered bicyclic heteroaromatic ring (having 1-5 heteroatoms independently selected from nitrogen, oxygen, and sulfur).

In some embodiments, R⁴ is methyl. In some embodiments, R⁴ is —NH₂. In some embodiments, R⁴ is —CF₃. In some embodiments, R⁴ is —NHR. In some embodiments, R⁴ is selected from those depicted in Table A below.

As defined generally above, X⁴ is NR, O or S. As defined generally above, X⁴ is NH, NMe, O or S. In some embodiments, X⁴ is NR. In some embodiments, X⁴ is NH. In some embodiments, X⁴ is NMe. In some embodiments, X⁴ is O. In some embodiments, X⁴ is S. In some embodiments, X⁴ is selected from those depicted in Table A below.

As defined generally above, Ring B is

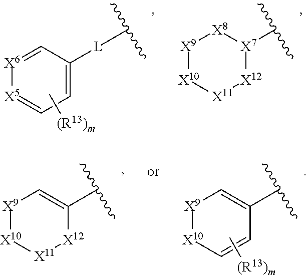

In some embodiments, Ring B is

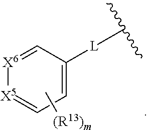

In some embodiments, Ring B is

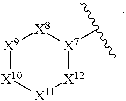

In some embodiments, Ring B is

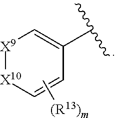

In some embodiments, Ring B is

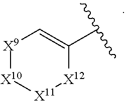

As defined generally above, L is a bond or an optionally substituted straight chain or branched C₁₋₆ alkylene. In some embodiments, L is a bond. In some embodiments, L is an optionally substituted straight chain or branched C₁₋₆ alkylene. In some embodiments, L is optionally substituted ethylene. In some embodiments, L is optionally substituted methylene. In some embodiments, L is selected from those depicted in Table A below.

As defined generally above, X⁵ is CH, N or CR⁵. In some embodiments, X⁵ is CH. In some embodiments, X⁵ is N. In some embodiments, X⁵ is CR⁵. In some embodiments, X⁵ is selected from those depicted in Table A below.

As defined generally above, X⁶ is CH, N or CR⁶. In some embodiments, X⁶ is CH. In some embodiments, X⁶ is N. In some embodiments, X⁶ is CR⁶. In some embodiments, X⁶ is selected from those depicted in Table A below.

In some embodiments, X⁵ is N and X⁶ is CH. In some embodiments, X⁵ is N and X⁶ is CR⁶. In some embodiments, X⁵ is CH and X⁶ is N. In some embodiments, X⁵ is CR⁵ and X⁶ is N. In some embodiments, X⁵ is CH and X⁶ is CH. In some embodiments, X⁵ is CH and X⁶ is CR⁶. In some embodiments, X⁵ is CR⁵ and X⁶ is CH.

As defined generally above, each R¹³ is independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R¹³ is an optionally substituted C₁₋₆ aliphatic group. In some embodiments, R¹³ is halogen. In some embodiments, R³ is —OR. In some embodiments, R³ is —CN. In some embodiments, R¹³ is —NR₂. In some embodiments, R³ is —C(═O)R. In some embodiments, R³ is —C(═O)OR. In some embodiments, R¹³ is —C(═O)NR₂. In some embodiments, R³ is —SO₂R. In some embodiments, R¹³ is —SO₂NR₂. In some embodiments, R¹³ is C₁₋₆haloalkyl. In some embodiments, R¹³ is C₁₋₆haloalkoxy. In some embodiments, R¹³ is —CD₃. In some embodiments, R¹³ is selected from those depicted in Table A below.

As defined generally above, m is 0, 1 or 2. In some embodiments, m is 0. In some embodiments, m is 1. In some embodiments, m is 2.

In some embodiments, Ring B is

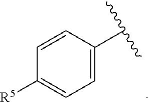

In some embodiments, Ring B is

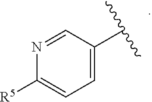

In some embodiments, Ring B is

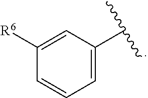

In some embodiments, Ring B is

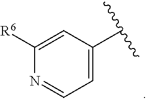

In some embodiments, Ring B is

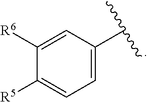

As defined generally above, R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁵ is an optionally substituted C₁₋₆ aliphatic group. In some embodiments, R⁵ is —OR. In some embodiments, R⁵ is —NR₂. In some embodiments, R⁵ is —C(═O)R. In some embodiments, R⁵ is —C(═O)OR. In some embodiments, R⁵ is —C(═O)NR₂. In some embodiments, R⁵ is —SO₂R. In some embodiments, R⁵ is —SO₂NR₂. In some embodiments, R⁵ is C₁₋₆haloalkyl. In some embodiments, R⁵ is C₁₋₆haloalkoxy. In some embodiments, R⁵ is an optionally substituted 3-8 membered saturated or partially unsaturated monocyclic carbocyclic ring. In some embodiments, R⁵ is an optionally substituted 6-12 membered saturated or partially unsaturated bridged carbocyclic ring. In some embodiments, R⁵ is an optionally substituted 7-12 membered saturated or partially unsaturated bicyclic carbocyclic ring. In some embodiments, R⁵ is an optionally substituted phenyl. In some embodiments, R⁵ is an optionally substituted 8-10 membered bicyclic aromatic carbocyclic ring. In some embodiments, R⁵ is an optionally substituted 3-8 membered saturated or partially unsaturated monocyclic heterocyclic ring (having 1-2 heteroatoms independently selected from nitrogen, oxygen, and sulfur). In some embodiments, R⁵ is an optionally substituted 6-12 membered saturated or partially unsaturated bridged heterocyclic ring (having 1-4 heteroatoms independently selected from nitrogen, oxygen, and sulfur). In some embodiments, R⁵ is an optionally substituted 7-12 membered saturated or partially unsaturated bicyclic heterocyclic ring (having 1-4 heteroatoms independently selected from nitrogen, oxygen, and sulfur). In some embodiments, R⁵ is an optionally substituted 5-6 membered monocyclic heteroaromatic ring (having 1-4 heteroatoms independently selected from nitrogen, oxygen, and sulfur). In some embodiments, R⁵ is an optionally substituted 8-10 membered bicyclic heteroaromatic ring (having 1-5 heteroatoms independently selected from nitrogen, oxygen, and sulfur).

In some embodiments, R⁵ is F. In some embodiments, R⁵ is Cl. In some embodiments, R is —OCF₃. In some embodiments, R⁵ is cyclopropyl. In some embodiments, R⁵ is selected from those depicted in Table A below.

In some embodiments, R⁶ is an optionally substituted C₁₋₆ aliphatic group. In some embodiments, R⁶ is —OR. In some embodiments, R⁶ is —NR₂. In some embodiments, R⁶ is —C(═O)R. In some embodiments, R⁶ is —C(═O)OR. In some embodiments, R⁶ is —C(═O)NR₂. In some embodiments, R⁶ is —SO₂R. In some embodiments, R⁶ is —SO₂NR₂. In some embodiments, R⁶ is C₁₋₆haloalkyl. In some embodiments, R⁶ is C₁₋₆haloalkoxy. In some embodiments, R⁶ is an optionally substituted 3-8 membered saturated or partially unsaturated monocyclic carbocyclic ring. In some embodiments, R⁶ is an optionally substituted 6-12 membered saturated or partially unsaturated bridged carbocyclic ring. In some embodiments, R⁶ is an optionally substituted 7-12 membered saturated or partially unsaturated bicyclic carbocyclic ring. In some embodiments, R⁶ is an optionally substituted phenyl. In some embodiments, R⁶ is an optionally substituted 8-10 membered bicyclic aromatic carbocyclic ring. In some embodiments, R⁶ is an optionally substituted 3-8 membered saturated or partially unsaturated monocyclic heterocyclic ring (having 1-2 heteroatoms independently selected from nitrogen, oxygen, and sulfur). In some embodiments, R⁶ is an optionally substituted 6-12 membered saturated or partially unsaturated bridged heterocyclic ring (having 1-4 heteroatoms independently selected from nitrogen, oxygen, and sulfur). In some embodiments, R⁶ is an optionally substituted 7-12 membered saturated or partially unsaturated bicyclic heterocyclic ring (having 1-4 heteroatoms independently selected from nitrogen, oxygen, and sulfur). In some embodiments, R⁶ is an optionally substituted 5-6 membered monocyclic heteroaromatic ring (having 1-4 heteroatoms independently selected from nitrogen, oxygen, and sulfur). In some embodiments, R⁶ is an optionally substituted 8-10 membered bicyclic heteroaromatic ring (having 1-5 heteroatoms independently selected from nitrogen, oxygen, and sulfur).

In some embodiments, R⁶ is F. In some embodiments, R⁶ is Cl. In some embodiments, R⁶ is —OCF₃. In some embodiments, R⁶ is cyclopropyl. In some embodiments, R⁶ is cyclobutyl. In some embodiments, R⁶ is optionally substituted pyrazolyl. In some embodiments, R⁶ is optionally substituted pyridinyl. In some embodiments, R⁶ is optionally substituted pyrimidinyl. In some embodiments, R⁶ is optionally substituted pyridazinyl. In some embodiments, R⁶ is optionally substituted imidazolyl. In some embodiments, R⁶ is optionally substituted triazolyl. In some embodiments, R⁶ is optionally substituted oxazolyl. In some embodiments, R⁶ is optionally substituted thiazolyl. In some embodiments, R⁶ is optionally substituted oxadiazolyl. In some embodiments, R⁶ is optionally substituted thiadiazolyl. In some embodiments, R⁶ is optionally substituted oxetanyl. In some embodiments, R⁶ is optionally substituted azetidinyl. In some embodiments, R⁶ is optionally substituted piperidinyl. In some embodiments, R⁶ is optionally substituted piperazinyl. In some embodiments, R⁶ is selected from those depicted in Table A below.

In some embodiments, R⁵ and R⁶ are independently a substituent selected from hydrogen and those shown below:

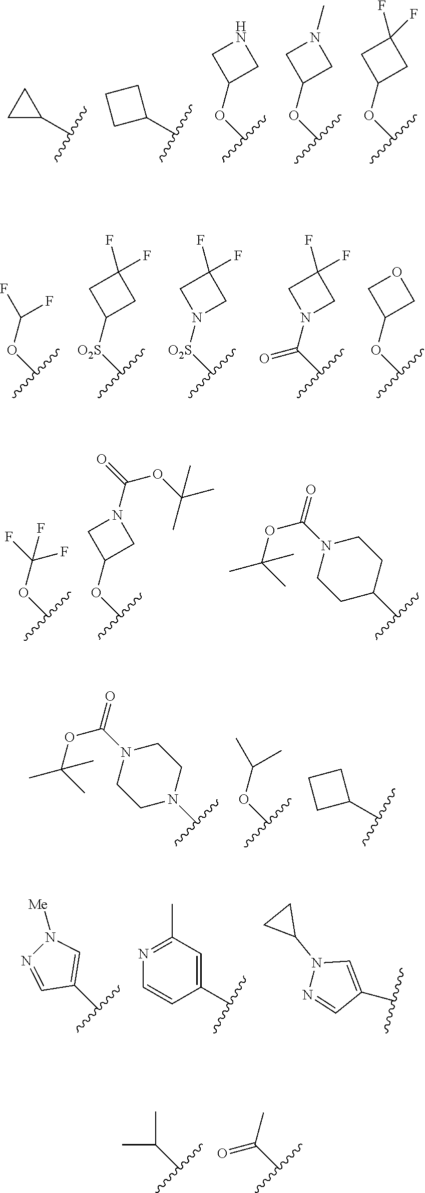

In some embodiments, W and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁵ and R⁶ are taken together with their intervening atoms to form an optionally substituted 3-8 membered saturated or partially unsaturated monocyclic carbocyclic ring. In some embodiments, R⁵ and R⁶ are taken together with their intervening atoms to form an optionally substituted 6-12 membered saturated or partially unsaturated bridged carbocyclic ring. In some embodiments, R⁵ and R⁶ are taken together with their intervening atoms to form an optionally substituted 7-12 membered saturated or partially unsaturated bicyclic carbocyclic ring. In some embodiments, R and R are taken together with their intervening atoms to form an optionally substituted phenyl. In some embodiments, R⁵ and R⁶ are taken together with their intervening atoms to form an optionally substituted 8-10 membered bicyclic aromatic carbocyclic ring. In some embodiments, R⁵ and R⁶ are taken together with their intervening atoms to form an optionally substituted 3-8 membered saturated or partially unsaturated monocyclic heterocyclic ring (having 1-2 heteroatoms independently selected from nitrogen, oxygen, and sulfur). In some embodiments, R⁵ and R⁶ are taken together with their intervening atoms to form an optionally substituted 6-12 membered saturated or partially unsaturated bridged heterocyclic ring (having 1-4 heteroatoms independently selected from nitrogen, oxygen, and sulfur). In some embodiments, R⁵ and R⁶ are taken together with their intervening atoms to form an optionally substituted 7-12 membered saturated or partially unsaturated bicyclic heterocyclic ring (having 1-4 heteroatoms independently selected from nitrogen, oxygen, and sulfur). In some embodiments, R⁵ and R⁶ are taken together with their intervening atoms to form an optionally substituted 5-6 membered monocyclic heteroaromatic ring (having 1-4 heteroatoms independently selected from nitrogen, oxygen, and sulfur). In some embodiments, R⁵ and R⁶ are taken together with their intervening atoms to form an optionally substituted 8-10 membered bicyclic heteroaromatic ring (having 1-5 heteroatoms independently selected from nitrogen, oxygen, and sulfur).

In some embodiments, R⁵ and R⁶ are taken together with their intervening atoms to form a dioxole ring.

As defined generally above, X⁷ is N, CH, or CR⁷. In some embodiments, X⁷ is N. In some embodiments, X⁷ is CH. In some embodiments, X⁷ is CR⁷. In some embodiments, X⁷ is CCH₃. In some embodiments, X⁷ is COH. In some embodiments, X⁷ is CF. In some embodiments, X⁷ is selected from those depicted in Table A below.

As defined generally above, X⁸ is O, NR⁸, C(R⁸)₂, CHR⁸, SO₂, or C═O. In some embodiments, X⁸ is O. In some embodiments, X⁸ is NR. In some embodiments, X⁸ is C(R)₂. In some embodiments, X⁸ is CHR⁸. In some embodiments, X⁸ is SO₂. In some embodiments, X⁸ is CH₂. In some embodiments, X⁸ is C═O. In some embodiments, X⁸ is selected from those depicted in Table A below.

As defined generally above, X⁹ is O, NR⁹, C(R⁹)₂, CHR⁹, SO₂, or C═O. In some embodiments, X⁹ is O. In some embodiments, X⁹ is NR⁹. In some embodiments, X⁹ is C(R⁹)₂. In some embodiments, X⁹ is CHR⁹. In some embodiments, X⁹ is SO₂. In some embodiments, X⁹ is CH₂. In some embodiments, X⁹ is C═O. In some embodiments, X⁹ is selected from those depicted in Table A below.

As defined generally above, X¹⁰ is O, NR¹⁰, C(R¹⁰)₂, CHR¹⁰, SO₂, or C═O. In some embodiments, X¹⁰ is O. In some embodiments, X¹⁰ is NR¹⁰. In some embodiments, X¹⁰ is C(R¹⁰)₂. In some embodiments, X¹⁰ is CHR¹⁰. In some embodiments, X¹⁰ is SO₂. In some embodiments, X¹⁰ is C═O. In some embodiments, X¹⁰ is CH₂, CF₂, or O. In some embodiments, X¹⁰ is CH₂. In some embodiments, X¹⁰ is NR¹⁰, or O. In some embodiments, X¹⁰ is NMe, NH, or O. In some embodiments, X¹⁰ is selected from those depicted in Table A below.

As defined generally above, X¹¹ is O, NR¹¹, C(R¹¹)₂, CHR¹¹, SO₂, or C═O. In some embodiments, X¹¹ is O. In some embodiments, X¹¹ is NR¹¹. In some embodiments, X¹¹ is C(R¹¹)₂. In some embodiments, X¹¹ is CHR¹¹. In some embodiments, X¹¹ is SO₂. In some embodiments, X¹¹ is CH₂. In some embodiments, X¹¹ is C═O. In some embodiments, X¹¹ is selected from those depicted in Table A below.

As defined generally above, X¹² is a direct bond, O, NR¹², C(R¹²)₂, CHR¹², —CH₂CH₂—, —OCH₂—, SO₂, or C═O. In some embodiments, X¹² is O. In some embodiments, X¹² is NR¹². In some embodiments, X¹² is C(R¹²)₂. In some embodiments, X¹² is CHR¹². In some embodiments, X¹² is CH₂. In some embodiments, X¹² is SO₂. In some embodiments, X¹² is C═O. In some embodiments, X¹² is —CH₂CH₂—. In some embodiments, X¹² is —OCH₂—. In some embodiments, X¹² is a direct bond. In some embodiments, X¹² is selected from those depicted in Table A below.

In some embodiments, when any of X⁷, X⁸, X⁹, X¹⁰, X¹¹, or X¹² is N, O or SO₂, then neither of the neighboring positions in Ring B are N, O or SO₂.

In some embodiments, when any one of X⁸, X⁹, X¹⁰, X¹¹, or X¹² is C═O, then neither of the neighboring positions in Ring B are C═O or SO₂.

As defined generally above, R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy. In some embodiments, R⁷ is an optionally substituted aliphatic group. In some embodiments, R⁷ is halogen. In some embodiments, R⁷ is —OR. In some embodiments, R⁷ is —NR₂. In some embodiments, R⁷ is —C(═O)R. In some embodiments, R⁷ is —C(═O)OR. In some embodiments, R⁷ is —C(═O)NR₂. In some embodiments, R⁷ is —SO₂R. In some embodiments, R⁷ is —SO₂NR₂. In some embodiments, R⁷ is C₁₋₆haloalkyl. In some embodiments, R⁷ is C₁₋₆haloalkoxy. In some embodiments, R⁷ is methyl. In some embodiments, R⁷ is OH. In some embodiments, R⁷ is F. In some embodiments, R⁷ is selected from those depicted in Table A below.

As defined generally above, each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted; or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted.

In some embodiments, R⁸ is hydrogen. In some embodiments, R⁸ is an optionally substituted C₁₋₆ aliphatic group. In some embodiments, R⁸ —OR. In some embodiments, R⁸ is —NR₂. In some embodiments, R⁸ is —C(═O)R. In some embodiments, R⁸ is —C(═O)OR. In some embodiments, R⁸ is —C(═O)NR₂. In some embodiments, R⁸ is —SO₂R. In some embodiments, R⁸ is —SO₂NR₂. In some embodiments, R⁸ is C₁₋₆haloalkyl. In some embodiments, R⁸ is C₁₋₆haloalkoxy. In some embodiments, R⁸ is an optionally substituted 3-8 membered saturated or partially unsaturated monocyclic carbocyclic ring. In some embodiments, R⁸ is an optionally substituted 7-12 membered saturated or partially unsaturated bicyclic carbocyclic ring. In some embodiments, R⁸ is an optionally substituted phenyl. In some embodiments, R⁸ is an optionally substituted 8-10 membered bicyclic aromatic carbocyclic ring. In some embodiments, R⁸ is an optionally substituted 3-8 membered saturated or partially unsaturated monocyclic heterocyclic ring (having 1-2 heteroatoms independently selected from nitrogen, oxygen, and sulfur). In some embodiments, R⁸ is an optionally substituted 7-12 membered saturated or partially unsaturated bicyclic heterocyclic ring (having 1-4 heteroatoms independently selected from nitrogen, oxygen, and sulfur). In some embodiments, R⁸ is an optionally substituted 5-6 membered monocyclic heteroaromatic ring (having 1-4 heteroatoms independently selected from nitrogen, oxygen, and sulfur). In some embodiments, R⁸ is an optionally substituted 8-10 membered bicyclic heteroaromatic ring (having 1-5 heteroatoms independently selected from nitrogen, oxygen, and sulfur). In some embodiments, R⁸ is methyl. In some embodiments, R⁸ is —OH. In some embodiments, R⁸ is F. In some embodiments, R⁸ is methoxy. In some embodiments, R⁸ is —CH₂OH. In some embodiments, wherein X⁸ is C(R)₂, each R⁸ is independently selected from any of the aforementioned substituents. In some embodiments, wherein X⁸ is C(R⁸)₂, both R⁸ are the same. In some embodiments, R⁸ is selected from those depicted in Table A below.

In some embodiments, R⁹ is hydrogen. In some embodiments, R⁹ is an optionally substituted C₁₋₆ aliphatic group. In some embodiments, R⁹ —OR. In some embodiments, R⁹ is —NR₂. In some embodiments, R⁹ is —C(═O)R. In some embodiments, R⁹ is —C(═O)OR. In some embodiments, R⁹ is —C(═O)NR₂. In some embodiments, R⁹ is —SO₂R. In some embodiments, R⁹ is —SO₂NR₂. In some embodiments, R⁹ is C₁₋₆haloalkyl. In some embodiments, R⁹ is C₁₋₆haloalkoxy. In some embodiments, R⁹ is an optionally substituted 3-8 membered saturated or partially unsaturated monocyclic carbocyclic ring. In some embodiments, R⁹ is an optionally substituted 7-12 membered saturated or partially unsaturated bicyclic carbocyclic ring. In some embodiments, R⁹ is an optionally substituted phenyl. In some embodiments, R⁹ is an optionally substituted 8-10 membered bicyclic aromatic carbocyclic ring. In some embodiments, R⁹ is an optionally substituted 3-8 membered saturated or partially unsaturated monocyclic heterocyclic ring (having 1-2 heteroatoms independently selected from nitrogen, oxygen, and sulfur). In some embodiments, R⁹ is an optionally substituted 7-12 membered saturated or partially unsaturated bicyclic heterocyclic ring (having 1-4 heteroatoms independently selected from nitrogen, oxygen, and sulfur). In some embodiments, R⁹ is an optionally substituted 5-6 membered monocyclic heteroaromatic ring (having 1-4 heteroatoms independently selected from nitrogen, oxygen, and sulfur). In some embodiments, R⁹ is an optionally substituted 8-10 membered bicyclic heteroaromatic ring (having 1-5 heteroatoms independently selected from nitrogen, oxygen, and sulfur). In some embodiments, R⁹ is methyl. In some embodiments, R⁹ is —OH. In some embodiments, R⁹ is F. In some embodiments, R⁹ is methoxy. In some embodiments, R⁹ is —CH₂OH. In some embodiments, wherein X⁹ is C(R⁹)₂, each R⁹ is independently selected from any of the aforementioned substituents. In some embodiments, wherein X⁹ is C(R⁹)₂, both R⁹ are the same. In some embodiments, R⁹ is selected from those depicted in Table A below.

In some embodiments, R⁹ is optionally substituted pyrazolyl. In some embodiments, R⁹ is optionally substituted pyridinyl. In some embodiments, R⁹ is optionally substituted pyrimidinyl. In some embodiments, R⁹ is optionally substituted pyridazinyl. In some embodiments, R⁹ is optionally substituted imidazolyl. In some embodiments, R⁹ is optionally substituted triazolyl. In some embodiments, R⁹ is optionally substituted oxazolyl. In some embodiments, R⁹ is optionally substituted thiazolyl. In some embodiments, R⁹ is optionally substituted oxadiazolyl. In some embodiments, R⁹ is optionally substituted thiadiazolyl. In some embodiments, R⁹ is optionally substituted oxetanyl. In some embodiments, R⁹ is optionally substituted azetidinyl. In some embodiments, R⁹ is optionally substituted piperidinyl. In some embodiments, R⁹ is optionally substituted piperazinyl.

In some embodiments, R⁹ is substituted with an optionally substituted 3-6 membered saturated or partially unsaturated monocyclic carbocyclic ring. In some embodiments, R⁹ is substituted with an optionally substituted 5-8 membered saturated or partially unsaturated bicyclic carbocyclic ring. In some embodiments, R⁹ is substituted with an optionally substituted 3-6 membered saturated or partially unsaturated monocyclic heterocyclic ring. In some embodiments, R⁹ is substituted with an optionally substituted C₁₋₆ aliphatic group. In some embodiments, R⁹ is substituted with a methyl group. In some embodiments, R⁹ is substituted with a —CD₃ group. In some embodiments, R⁹ is substituted with a methoxy group. In some embodiments, R⁹ is substituted with a cyclopropyl group. In some embodiments, R⁹ is substituted with an optionally substituted

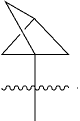 [SMILES: *C12CC(C1)C2]

In some embodiments, R⁹ is —OR, wherein R is an optionally substituted 5-6 membered heteroaryl ring (having 1-4 heteroatoms independently selected from nitrogen, oxygen, and sulfur). In some embodiments, R⁹ is —NHR, wherein R is an optionally substituted 5-6 membered heteroaryl ring (having 1-4 heteroatoms independently selected from nitrogen, oxygen, and sulfur). In some embodiments, R⁹ is —N(CH₃)R, wherein R is an optionally substituted 5-6 membered heteroaryl ring (having 1-4 heteroatoms independently selected from nitrogen, oxygen, and sulfur). In some embodiments, R⁹ is —C(═O)N(CH₃)R, wherein R is an optionally substituted 5-6 membered heteroaryl ring (having 1-4 heteroatoms independently selected from nitrogen, oxygen, and sulfur). In some embodiments, R⁹ is —C(═O)NHR, wherein R is an optionally substituted 5-6 membered heteroaryl ring (having 1-4 heteroatoms independently selected from nitrogen, oxygen, and sulfur).

In some embodiments, R⁹ is a substituent selected from those shown below:

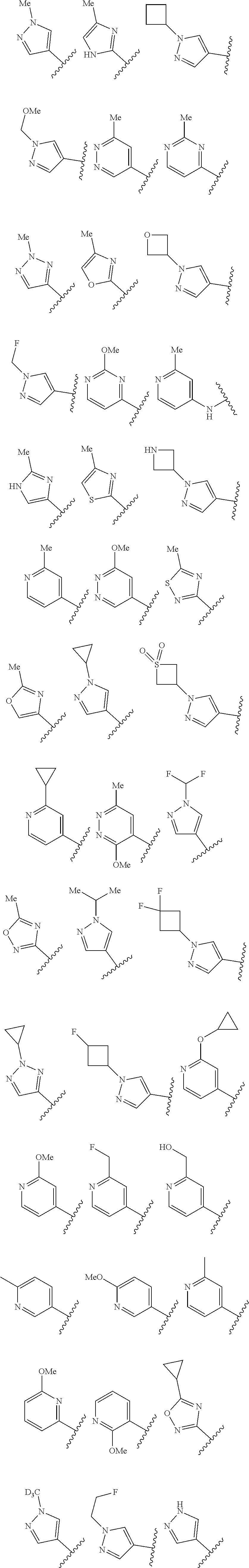

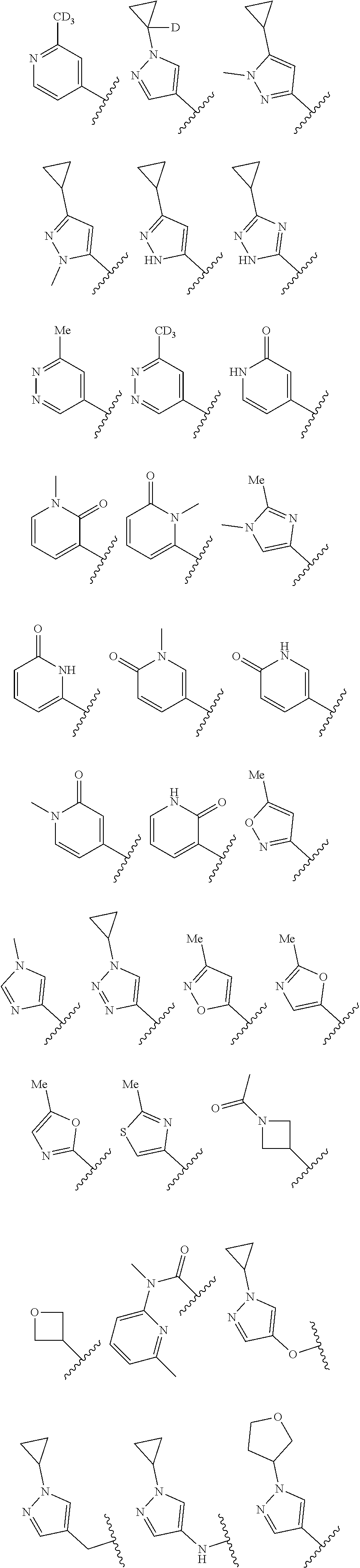

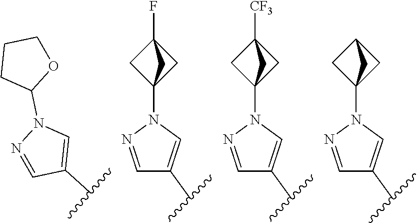

In some embodiments, R⁹ is methyl, tetrahydrofuran-3-yl,

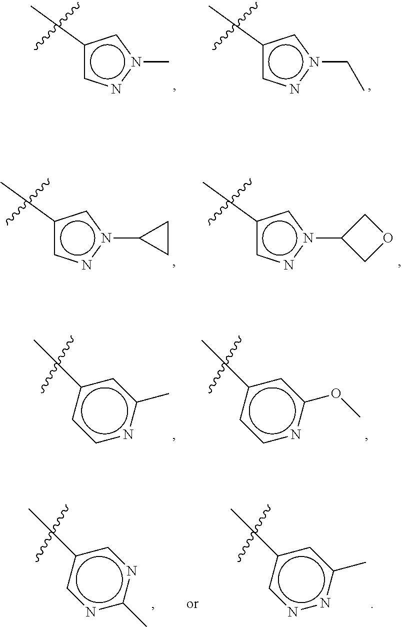

In some embodiments, R⁹ is methyl, tetrahydrofuran-3-yl,

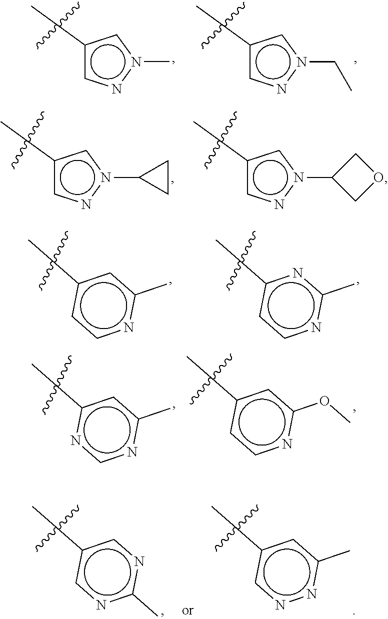

In some embodiments, R⁹ is

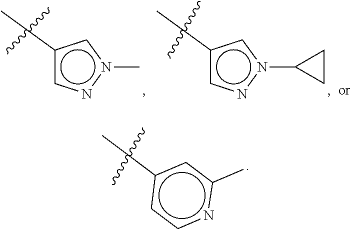

In some embodiments, R⁹ is

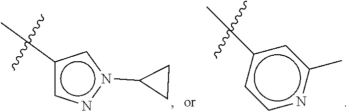

In some embodiments, R⁹ is

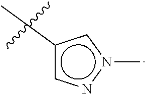

In some embodiments, R⁹ is

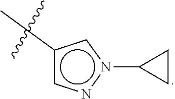

In some embodiments, R⁹ is

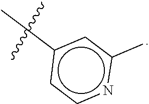

In some embodiments, R¹⁰ is hydrogen. In some embodiments, R¹⁰ is an optionally substituted C₁₋₆ aliphatic group. In some embodiments, R¹⁰—OR. In some embodiments, R¹⁰ is —NR₂. In some embodiments, R¹⁰ is —C(═O)R. In some embodiments, R¹⁰ is —C(═O)OR. In some embodiments, R¹⁰ is —C(═O)NR₂. In some embodiments, R¹⁰ is —SO₂R. In some embodiments, R¹⁰ is —SO₂NR₂. In some embodiments, R¹⁰ is C₁₋₆haloalkyl. In some embodiments, R¹⁰ is C₁₋₆haloalkoxy. In some embodiments, R¹⁰ is an optionally substituted 3-8 membered saturated or partially unsaturated monocyclic carbocyclic ring. In some embodiments, R¹⁰ is an optionally substituted 7-12 membered saturated or partially unsaturated bicyclic carbocyclic ring. In some embodiments, R¹⁰ is an optionally substituted phenyl. In some embodiments, R¹⁰ is an optionally substituted 8-10 membered bicyclic aromatic carbocyclic ring. In some embodiments, R¹⁰ is an optionally substituted 3-8 membered saturated or partially unsaturated monocyclic heterocyclic ring (having 1-2 heteroatoms independently selected from nitrogen, oxygen, and sulfur). In some embodiments, R¹⁰ is an optionally substituted 7-12 membered saturated or partially unsaturated bicyclic heterocyclic ring (having 1-4 heteroatoms independently selected from nitrogen, oxygen, and sulfur). In some embodiments, R¹⁰ is an optionally substituted 5-6 membered monocyclic heteroaromatic ring (having 1-4 heteroatoms independently selected from nitrogen, oxygen, and sulfur). In some embodiments, R¹⁰ is an optionally substituted 8-10 membered bicyclic heteroaromatic ring (having 1-5 heteroatoms independently selected from nitrogen, oxygen, and sulfur). In some embodiments, R¹⁰ is methyl. In some embodiments, R¹⁰ is —OH. In some embodiments, R¹⁰ is F. In some embodiments, R¹⁰ is methoxy. In some embodiments, R¹⁰ is —CH₂OH. In some embodiments, wherein X¹⁰ is C(R¹⁰)₂, each R¹⁰ is independently selected from any of the aforementioned substituents. In some embodiments, wherein X¹⁰ is C(R¹⁰)₂, both R¹⁰ are the same. In some embodiments, R¹⁰ is selected from those depicted in Table A below.

In some embodiments, R¹¹ is hydrogen. In some embodiments, R¹¹ is an optionally substituted C₁₋₆ aliphatic group. In some embodiments, R¹¹ —OR. In some embodiments, R¹¹ is —NR₂. In some embodiments, R¹¹ is —C(═O)R. In some embodiments, R¹¹ is —C(═O)OR. In some embodiments, R¹¹ is —C(═O)NR₂. In some embodiments, R¹¹ is —SO₂R. In some embodiments, R¹¹ is —SO₂NR₂. In some embodiments, R¹¹ is C₁₋₆haloalkyl. In some embodiments, R¹¹ is C₁₋₆haloalkoxy. In some embodiments, R¹¹ is an optionally substituted 3-8 membered saturated or partially unsaturated monocyclic carbocyclic ring. In some embodiments, R¹¹ is an optionally substituted 7-12 membered saturated or partially unsaturated bicyclic carbocyclic ring. In some embodiments, R¹¹ is an optionally substituted phenyl. In some embodiments, R¹¹ is an optionally substituted 8-10 membered bicyclic aromatic carbocyclic ring. In some embodiments, R¹¹ is an optionally substituted 3-8 membered saturated or partially unsaturated monocyclic heterocyclic ring (having 1-2 heteroatoms independently selected from nitrogen, oxygen, and sulfur). In some embodiments, R¹¹ is an optionally substituted 7-12 membered saturated or partially unsaturated bicyclic heterocyclic ring (having 1-4 heteroatoms independently selected from nitrogen, oxygen, and sulfur). In some embodiments, R¹¹ is an optionally substituted 5-6 membered monocyclic heteroaromatic ring (having 1-4 heteroatoms independently selected from nitrogen, oxygen, and sulfur). In some embodiments, R¹¹ is an optionally substituted 8-10 membered bicyclic heteroaromatic ring (having 1-5 heteroatoms independently selected from nitrogen, oxygen, and sulfur). In some embodiments, R¹¹ is methyl. In some embodiments, R¹¹ is —OH. In some embodiments, R¹¹ is F. In some embodiments, R¹¹ is methoxy. In some embodiments, R¹¹ is —CH₂OH. In some embodiments, wherein X¹¹ is C(R¹¹)₂, each R¹¹ is independently selected from any of the aforementioned substituents. In some embodiments, wherein X¹¹ is C(R¹¹)₂, both R¹¹ are the same. In some embodiments, R¹¹ is selected from those depicted in Table A below.

In some embodiments, R¹² is hydrogen. In some embodiments, R¹² is an optionally substituted C₁₋₆ aliphatic group. In some embodiments, R¹² —OR. In some embodiments, R¹² is —NR₂. In some embodiments, R¹² is —C(═O)R. In some embodiments, R¹² is —C(═O)OR. In some embodiments, R¹² is —C(═O)NR₂. In some embodiments, R¹² is —SO₂R. In some embodiments, R¹² is —SO₂NR₂. In some embodiments, R¹² is C₁₋₆haloalkyl. In some embodiments, R¹² is C₁₋₆haloalkoxy. In some embodiments, R¹² is an optionally substituted 3-8 membered saturated or partially unsaturated monocyclic carbocyclic ring. In some embodiments, R¹² is an optionally substituted 7-12 membered saturated or partially unsaturated bicyclic carbocyclic ring. In some embodiments, R¹² is an optionally substituted phenyl. In some embodiments, R¹² is an optionally substituted 8-10 membered bicyclic aromatic carbocyclic ring. In some embodiments, R¹² is an optionally substituted 3-8 membered saturated or partially unsaturated monocyclic heterocyclic ring (having 1-2 heteroatoms independently selected from nitrogen, oxygen, and sulfur). In some embodiments, R¹² is an optionally substituted 7-12 membered saturated or partially unsaturated bicyclic heterocyclic ring (having 1-4 heteroatoms independently selected from nitrogen, oxygen, and sulfur). In some embodiments, R¹² is an optionally substituted 5-6 membered monocyclic heteroaromatic ring (having 1-4 heteroatoms independently selected from nitrogen, oxygen, and sulfur). In some embodiments, R¹² is an optionally substituted 8-10 membered bicyclic heteroaromatic ring (having 1-5 heteroatoms independently selected from nitrogen, oxygen, and sulfur). In some embodiments, R¹² is methyl. In some embodiments, R¹² is —OH. In some embodiments, R¹² is F. In some embodiments, R¹² is methoxy. In some embodiments, R¹² is —CH₂OH. In some embodiments, wherein X¹² is C(R¹²)₂, each R¹² is independently selected from any of the aforementioned substituents. In some embodiments, wherein X¹² is C(R¹²)₂, both R¹² are the same. In some embodiments, R¹² is selected from those depicted in Table A below.

In some embodiments, Ring B is a substituent selected from those shown below:

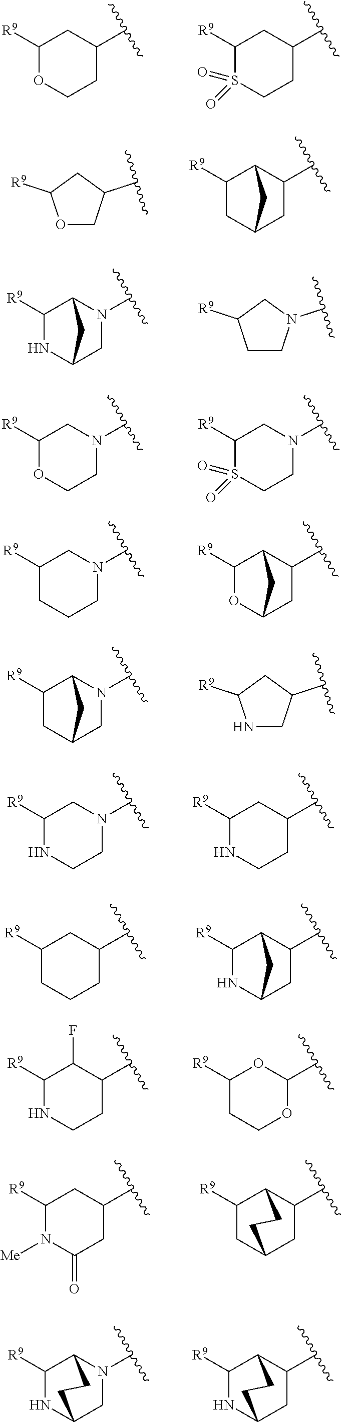

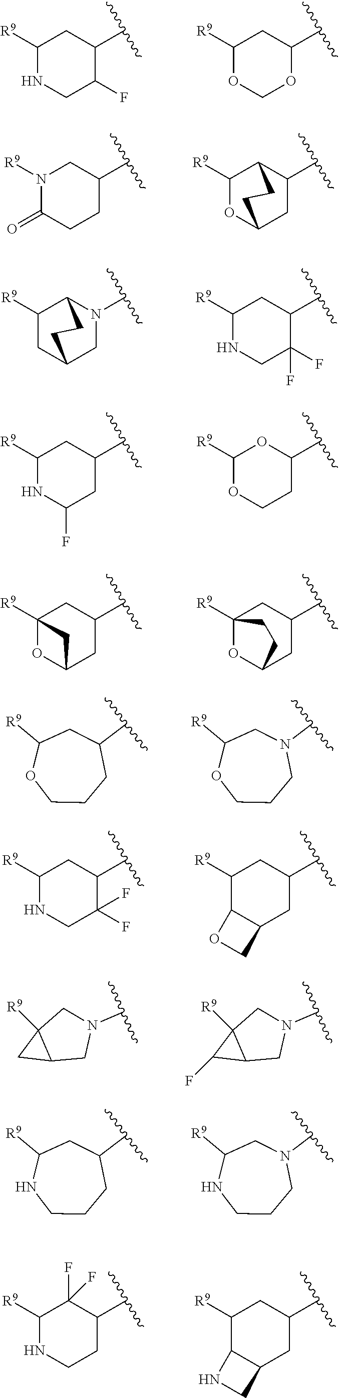

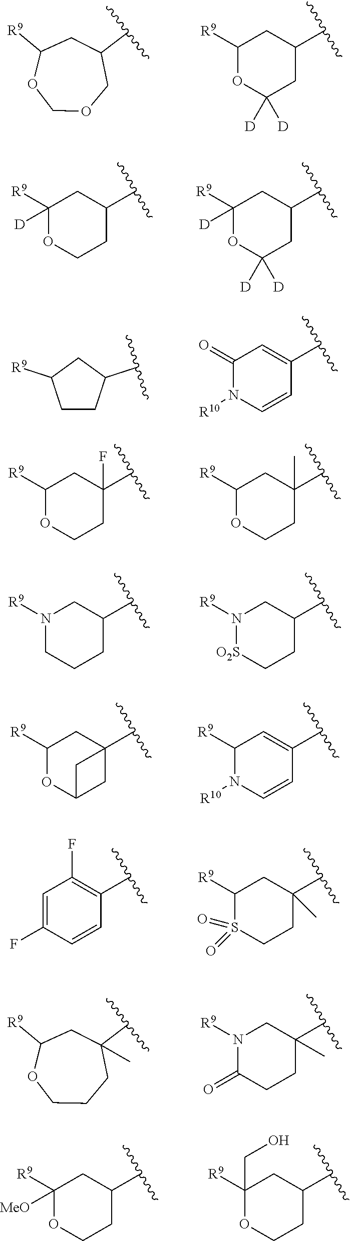

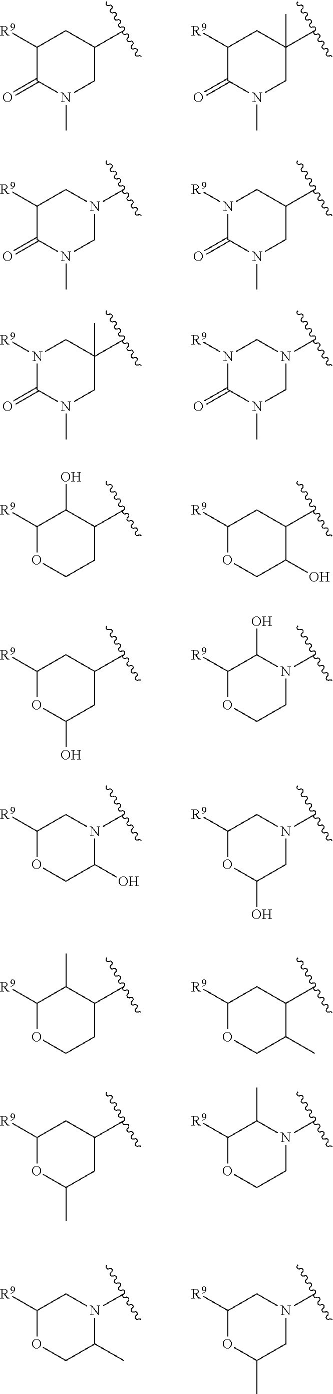

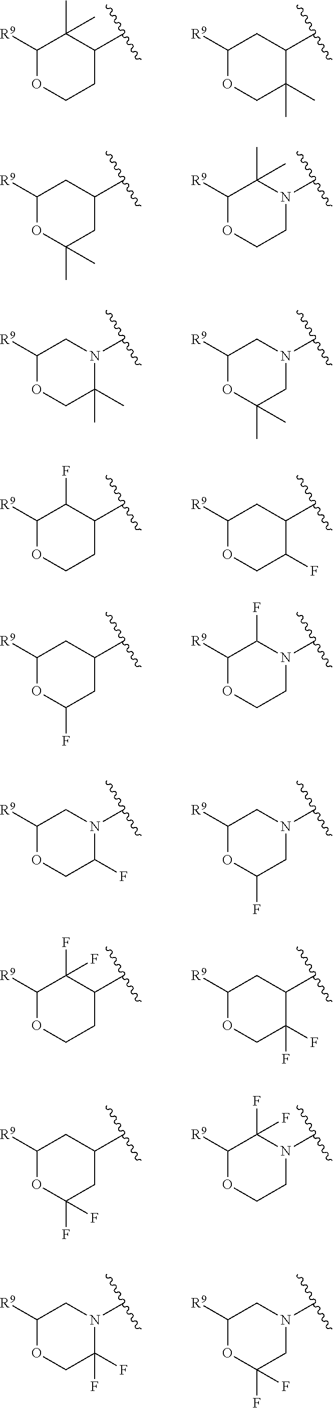

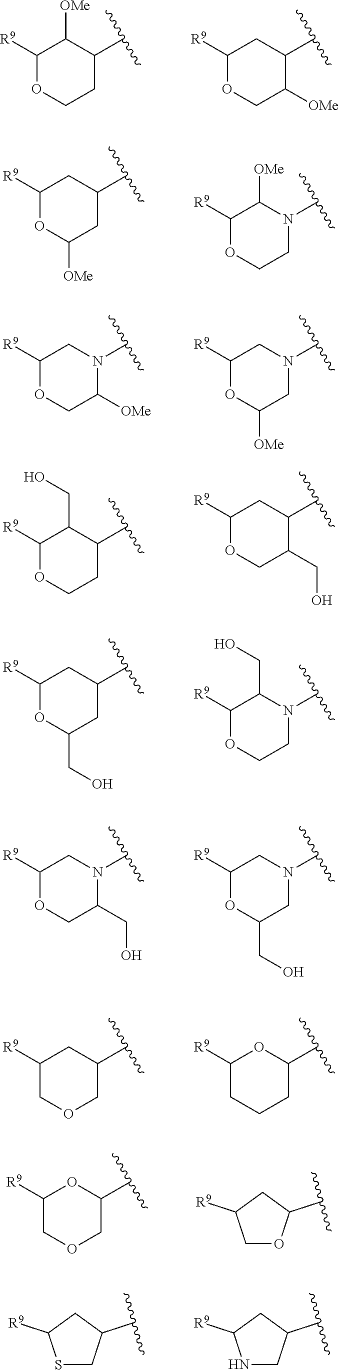

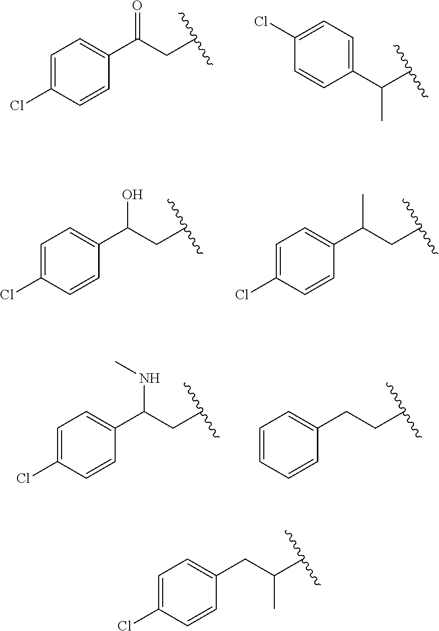

In some embodiments, Ring B is

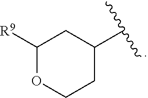

In some embodiments, Ring B is

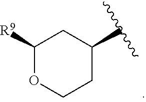

In some embodiments, Ring B is

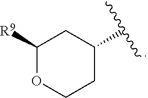

In some embodiments, Ring B is

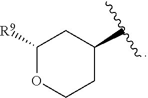

In some embodiments, Ring B is

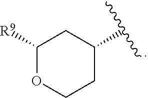

In some embodiments, Ring B is

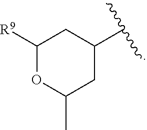

In some embodiments, Ring B is

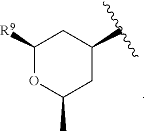

In some embodiments, Ring B is

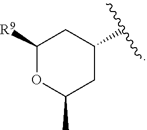

In some embodiments, Ring B is

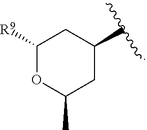

In some embodiments, Ring B is

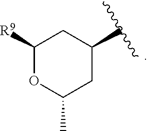

In some embodiments, Ring B is

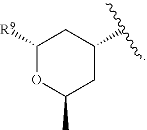

In some embodiments, Ring B is

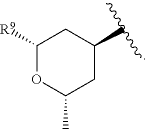

In some embodiments, Ring B is

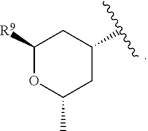

In some embodiments, Ring B is

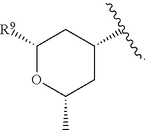

In some embodiments, Ring B is

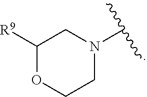

In some embodiments, Ring B is

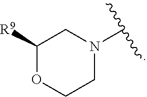

In some embodiments, Ring B is

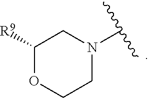

In some embodiments, Ring B is

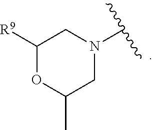

In some embodiments, Ring B is

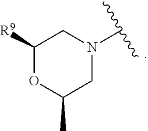

In some embodiments, Ring B is

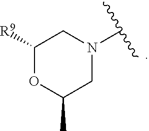

In some embodiments, Ring B is

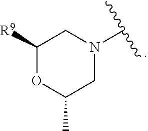

In some embodiments, Ring B is

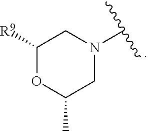

In some embodiments, Ring B is

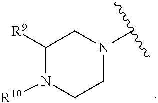

In some embodiments, Ring B is

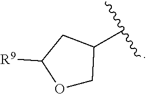

In some embodiments, Ring B is

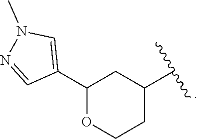 [SMILES: *C1CCOC(c2cnn(C)c2)C1]

In some embodiments, Ring B is

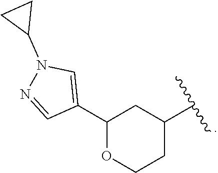 [SMILES: *C1CCOC(c2cnn(C3CC3)c2)C1]

In some embodiments, Ring B is

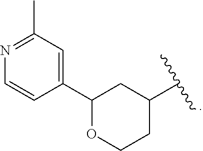 [SMILES: *C1CCOC(c2ccnc(C)c2)C1]

In some embodiments, Ring B is

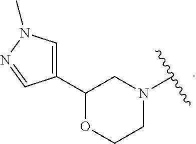 [SMILES: *N1CCOC(c2cnn(C)c2)C1]

In some embodiments, Ring B is

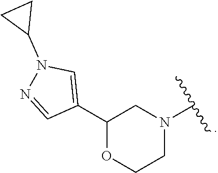 [SMILES: *N1CCOC(c2cnn(C3CC3)c2)C1]

In some embodiments, Ring B is

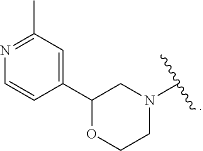 [SMILES: *N1CCOC(c2ccnc(C)c2)C1]

In some embodiments, Ring B is

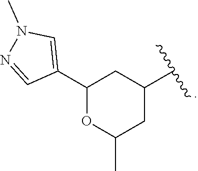 [SMILES: *C1CC(C)OC(c2cnn(C)c2)C1]

In some embodiments, Ring B is

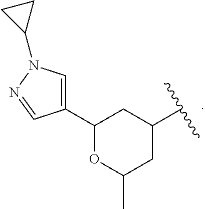 [SMILES: *C1CC(C)OC(c2cnn(C3CC3)c2)C1]

In some embodiments, Ring B is

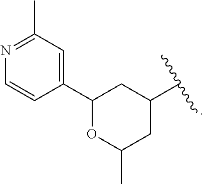 [SMILES: *C1CC(C)OC(c2ccnc(C)c2)C1]

In some embodiments, Ring B is

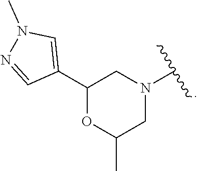 [SMILES: *N1CC(C)OC(c2cnn(C)c2)C1]

In some embodiments, Ring B is

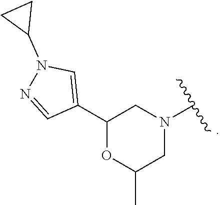 [SMILES: *N1CC(C)OC(c2cnn(C3CC3)c2)C1]

In some embodiments, Ring B is

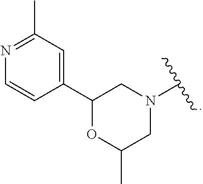 [SMILES: *N1CC(C)OC(c2ccnc(C)c2)C1]

In some embodiments, Ring B is

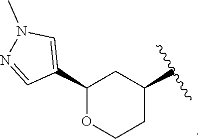 [SMILES: *[C@H]1CCO[C@@H](c2cnn(C)c2)C1]

In some embodiments, Ring B is

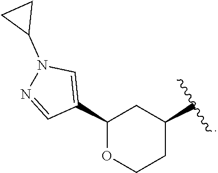 [SMILES: *[C@H]1CCO[C@@H](c2cnn(C3CC3)c2)C1]

In some embodiments, Ring B is

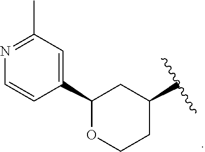 [SMILES: *[C@H]1CCO[C@@H](c2ccnc(C)c2)C1]

In some embodiments, Ring B is

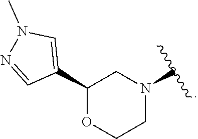

In some embodiments, Ring B is

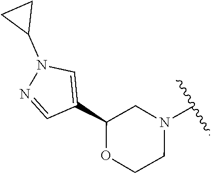

In some embodiments, Ring B is

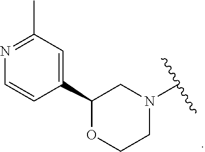 [SMILES: *N1CCO[C@@H](c2ccnc(C)c2)C1]

In some embodiments, Ring B is

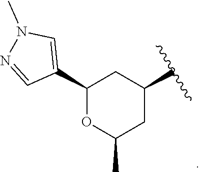 [SMILES: *[C@H]1C[C@@H](C)O[C@@H](c2cnn(C)c2)C1]

In some embodiments, Ring B is

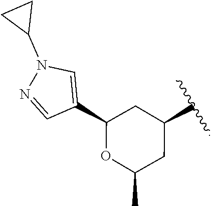

In some embodiments, Ring B is

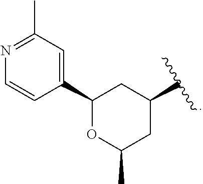 [SMILES: *[C@H]1C[C@@H](C)O[C@@H](c2ccnc(C)c2)C1]

In some embodiments, Ring B is

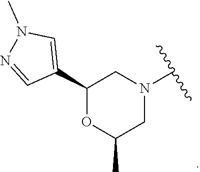 [SMILES: *N1C[C@@H](C)O[C@@H](c2cnn(C)c2)C1]

In some embodiments, Ring B is

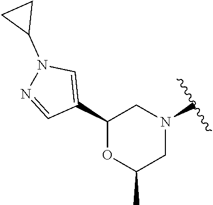

In some embodiments, Ring B is

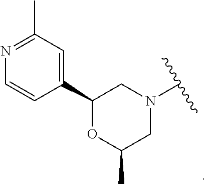 [SMILES: *N1C[C@@H](C)O[C@@H](c2ccnc(C)c2)C1]

In some embodiments, at least one hydrogen atom of the compound is a deuterium atom. In some embodiments, at least one C₁-C₆ aliphatic group of the compound is substituted with at least one deuterium atom. In some embodiments, at least one C₁-C₆alkyl group of the compound is substituted with at least one deuterium atom. In some embodiments, R² is —CD₃. In some embodiments, R³ is —CD₃. In some embodiments, R² and R³ are both —CD₃. In some embodiments, R⁴ is —CD₃.

Exemplary compounds of the invention are set forth in Table A, below. In some embodiments, the compound is a compound set forth in Table A, or a pharmaceutically acceptable salt thereof.

TABLE A
Exemplary Compounds

| I-# | Structure |
|---|---|
| I-1 | 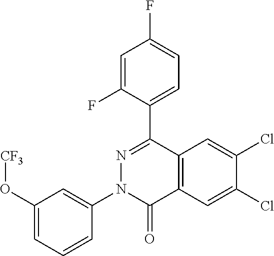 [SMILES: O=c1c2cc(Cl)c(Cl)cc2c(-c2ccc(F)cc2F)nn1-c1cccc(OC(F)(F)F)c1] |
| I-2 | 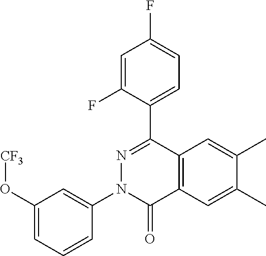 [SMILES: Cc1cc2c(-c3ccc(F)cc3F)nn(-c3cccc(OC(F)(F)F)c3)c(=O)c2cc1C] |
| I-3 | 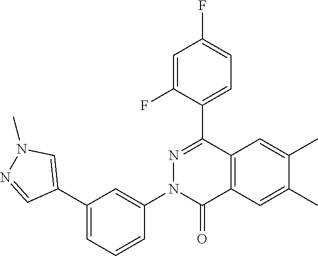 [SMILES: Cc1cc2c(-c3ccc(F)cc3F)nn(-c3cccc(-c4cnn(C)c4)c3)c(=O)c2cc1C] |
| I-4 | 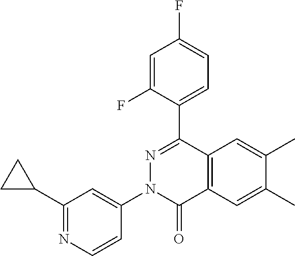 [SMILES: Cc1cc2c(-c3ccc(F)cc3F)nn(-c3ccnc(C4CC4)c3)c(=O)c2cc1C] |
| I-5 | 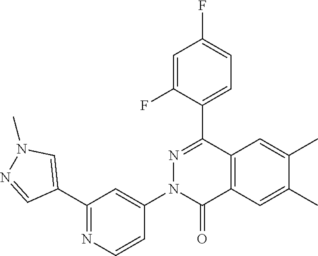 [SMILES: Cc1cc2c(-c3ccc(F)cc3F)nn(-c3ccnc(-c4cnn(C)c4)c3)c(=O)c2cc1C] |
| I-6 | 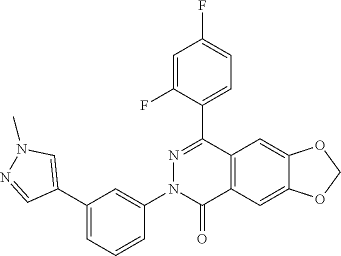 [SMILES: Cn1cc(-c2cccc(-n3nc(-c4ccc(F)cc4F)c4cc5c(cc4c3=O)OCO5)c2)cn1] |
| I-7 | 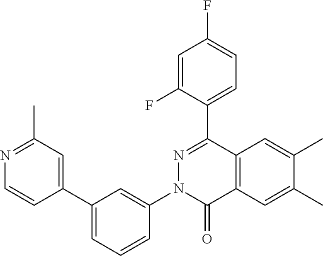 [SMILES: Cc1cc(-c2cccc(-n3nc(-c4ccc(F)cc4F)c4cc(C)c(C)cc4c3=O)c2)ccn1] |
| I-8 | 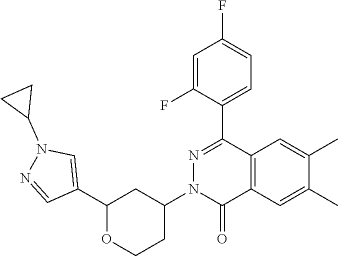 [SMILES: Cc1cc2c(-c3ccc(F)cc3F)nn(C3CCOC(c4cnn(C5CC5)c4)C3)c(=O)c2cc1C] |
| I-9 | 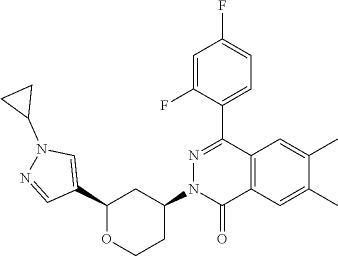 [SMILES: Cc1cc2c(-c3ccc(F)cc3F)nn([C@H]3CCO[C@@H](c4cnn(C5CC5)c4)C3)c(=O)c2cc1C] |
| I-10 | 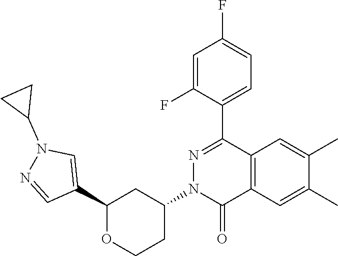 [SMILES: Cc1cc2c(-c3ccc(F)cc3F)nn([C@@H]3CCO[C@@H](c4cnn(C5CC5)c4)C3)c(=O)c2cc1C] |
| I-11 | 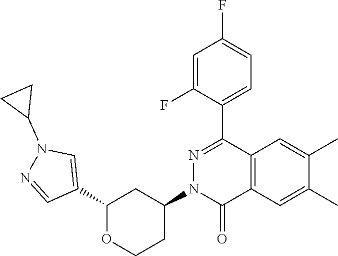 [SMILES: Cc1cc2c(-c3ccc(F)cc3F)nn([C@H]3CCO[C@H](c4cnn(C5CC5)c4)C3)c(=O)c2cc1C] |
| I-12 | 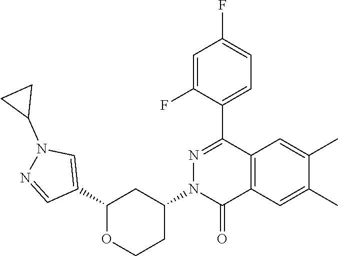 [SMILES: Cc1cc2c(-c3ccc(F)cc3F)nn([C@@H]3CCO[C@H](c4cnn(C5CC5)c4)C3)c(=O)c2cc1C] |
| I-13 | 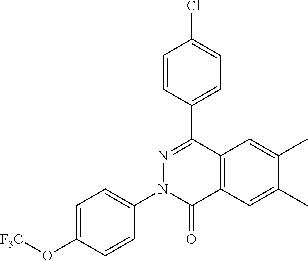 [SMILES: Cc1cc2c(-c3ccc(Cl)cc3)nn(-c3ccc(OC(F)(F)F)cc3)c(=O)c2cc1C] |
| I-14 | 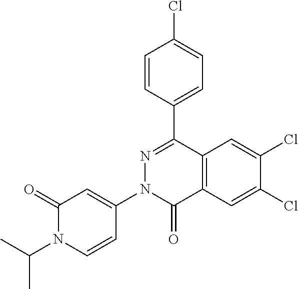 [SMILES: CC(C)n1ccc(-n2nc(-c3ccc(Cl)cc3)c3cc(Cl)c(Cl)cc3c2=O)cc1=O] |
| I-15 | 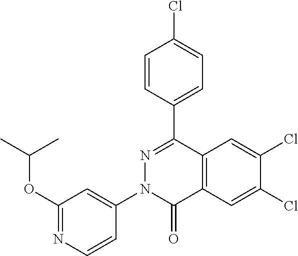 [SMILES: CC(C)Oc1cc(-n2nc(-c3ccc(Cl)cc3)c3cc(Cl)c(Cl)cc3c2=O)ccn1] |
| I-16 | 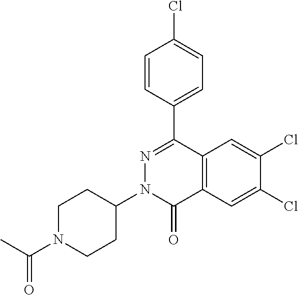 [SMILES: CC(=O)N1CCC(n2nc(-c3ccc(Cl)cc3)c3cc(Cl)c(Cl)cc3c2=O)CC1] |
| I-17 | 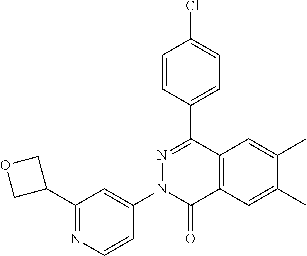 [SMILES: Cc1cc2c(-c3ccc(Cl)cc3)nn(-c3ccnc(C4COC4)c3)c(=O)c2cc1C] |
| I-18 | 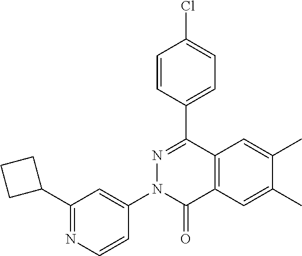 [SMILES: Cc1cc2c(-c3ccc(Cl)cc3)nn(-c3ccnc(C4CCC4)c3)c(=O)c2cc1C] |
| I-19 | 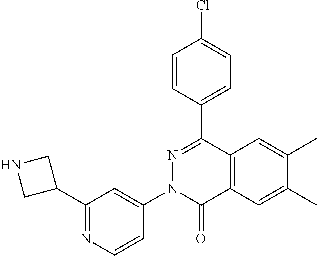 [SMILES: Cc1cc2c(-c3ccc(Cl)cc3)nn(-c3ccnc(C4CNC4)c3)c(=O)c2cc1C] |
| I-20 | 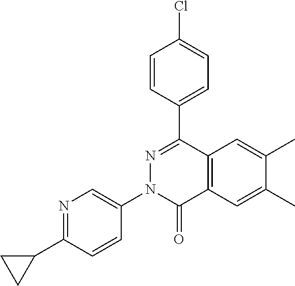 [SMILES: Cc1cc2c(-c3ccc(Cl)cc3)nn(-c3ccc(C4CC4)nc3)c(=O)c2cc1C] |
| I-21 | 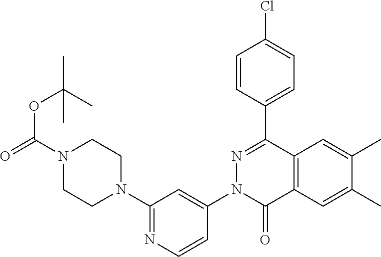 [SMILES: Cc1cc2c(-c3ccc(Cl)cc3)nn(-c3ccnc(N4CCN(C(=O)OC(C)(C)C)CC4)c3)c(=O)c2cc1C] |
| I-22 | 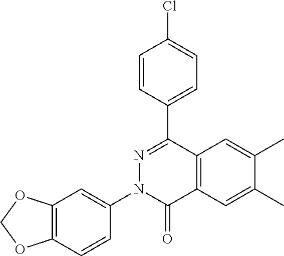 [SMILES: Cc1cc2c(-c3ccc(Cl)cc3)nn(-c3ccc4c(c3)OCO4)c(=O)c2cc1C] |
| I-23 | 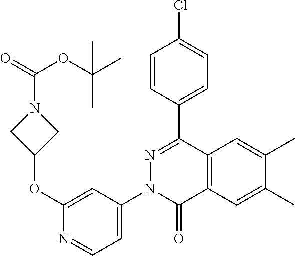 [SMILES: Cc1cc2c(-c3ccc(Cl)cc3)nn(-c3ccnc(OC4CN(C(=O)OC(C)(C)C)C4)c3)c(=O)c2cc1C] |
| I-24 | 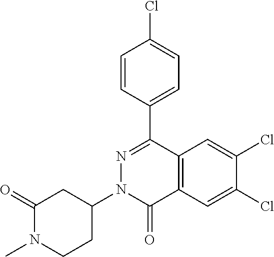 [SMILES: CN1CCC(n2nc(-c3ccc(Cl)cc3)c3cc(Cl)c(Cl)cc3c2=O)CC1=O] |
| I-25 | 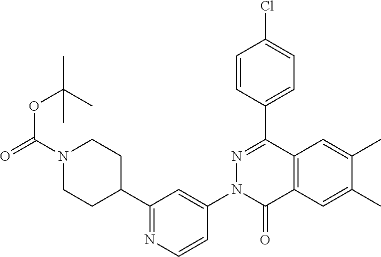 [SMILES: Cc1cc2c(-c3ccc(Cl)cc3)nn(-c3ccnc(C4CCN(C(=O)OC(C)(C)C)CC4)c3)c(=O)c2cc1C] |
| I-26 | 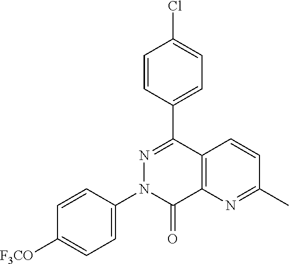 [SMILES: Cc1ccc2c(-c3ccc(Cl)cc3)nn(-c3ccc(OC(F)(F)F)cc3)c(=O)c2n1] |
| I-27 | 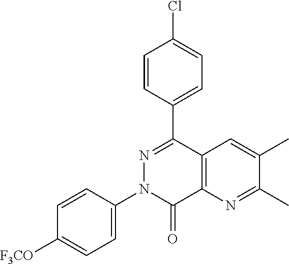 [SMILES: Cc1cc2c(-c3ccc(Cl)cc3)nn(-c3ccc(OC(F)(F)F)cc3)c(=O)c2nc1C] |
| I-28 | 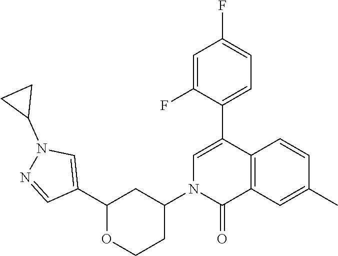 [SMILES: Cc1ccc2c(-c3ccc(F)cc3F)cn(C3CCOC(c4cnn(C5CC5)c4)C3)c(=O)c2c1] |
| I-29 | 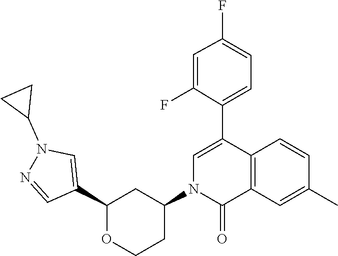 [SMILES: Cc1ccc2c(-c3ccc(F)cc3F)cn([C@H]3CCO[C@@H](c4cnn(C5CC5)c4)C3)c(=O)c2c1] |
| I-30 | 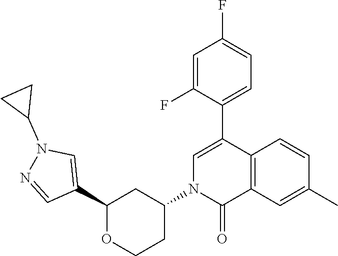 [SMILES: Cc1ccc2c(-c3ccc(F)cc3F)cn([C@@H]3CCO[C@@H](c4cnn(C5CC5)c4)C3)c(=O)c2c1] |
| I-31 | 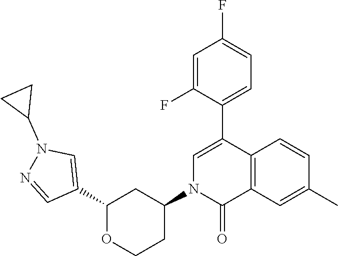 [SMILES: Cc1ccc2c(-c3ccc(F)cc3F)cn([C@H]3CCO[C@H](c4cnn(C5CC5)c4)C3)c(=O)c2c1] |
| I-32 | 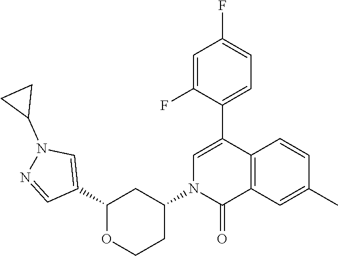 [SMILES: Cc1ccc2c(-c3ccc(F)cc3F)cn([C@@H]3CCO[C@H](c4cnn(C5CC5)c4)C3)c(=O)c2c1] |
| I-33 | 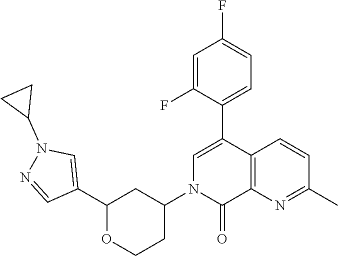 [SMILES: Cc1ccc2c(-c3ccc(F)cc3F)cn(C3CCOC(c4cnn(C5CC5)c4)C3)c(=O)c2n1] |
| I-34 | 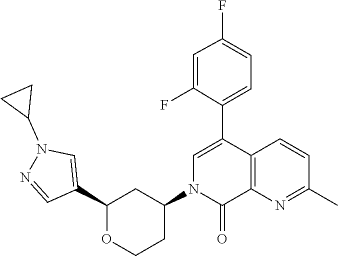 [SMILES: Cc1ccc2c(-c3ccc(F)cc3F)cn([C@H]3CCO[C@@H](c4cnn(C5CC5)c4)C3)c(=O)c2n1] |
| I-35 | 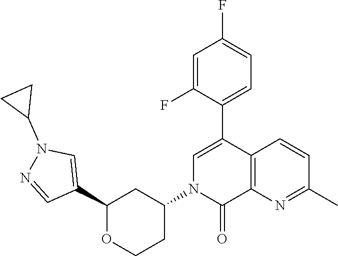 [SMILES: Cc1ccc2c(-c3ccc(F)cc3F)cn([C@@H]3CCO[C@@H](c4cnn(C5CC5)c4)C3)c(=O)c2n1] |
| I-36 | 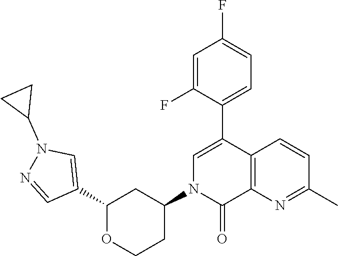 [SMILES: Cc1ccc2c(-c3ccc(F)cc3F)cn([C@H]3CCO[C@H](c4cnn(C5CC5)c4)C3)c(=O)c2n1] |
| I-37 | 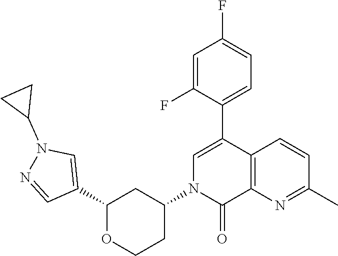 [SMILES: Cc1ccc2c(-c3ccc(F)cc3F)cn([C@@H]3CCO[C@H](c4cnn(C5CC5)c4)C3)c(=O)c2n1] |
| I-38 | 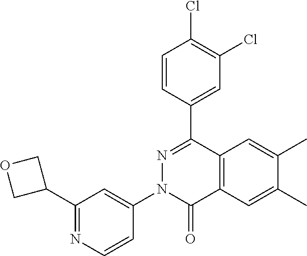 [SMILES: Cc1cc2c(-c3ccc(Cl)c(Cl)c3)nn(-c3ccnc(C4COC4)c3)c(=O)c2cc1C] |
| I-39 | 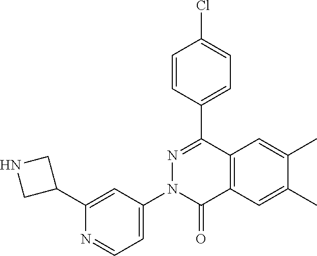 [SMILES: Cc1cc2c(-c3ccc(Cl)cc3)nn(-c3ccnc(C4CNC4)c3)c(=O)c2cc1C] |
| I-40 | 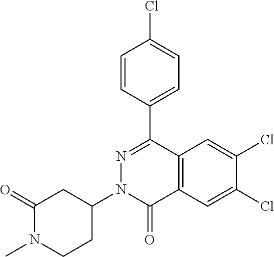 [SMILES: CN1CCC(n2nc(-c3ccc(Cl)cc3)c3cc(Cl)c(Cl)cc3c2=O)CC1=O] |
| I-41 | 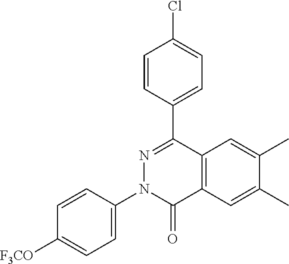 [SMILES: Cc1cc2c(-c3ccc(Cl)cc3)nn(-c3ccc(OC(F)(F)F)cc3)c(=O)c2cc1C] |
| I-42 | 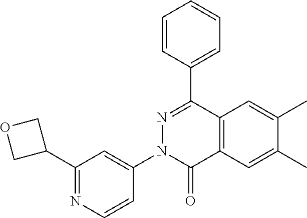 [SMILES: Cc1cc2c(-c3ccccc3)nn(-c3ccnc(C4COC4)c3)c(=O)c2cc1C] |
| I-43 | 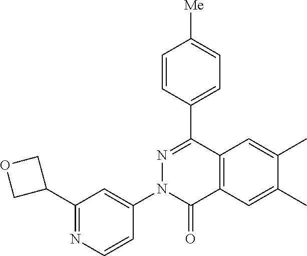 [SMILES: Cc1ccc(-c2nn(-c3ccnc(C4COC4)c3)c(=O)c3cc(C)c(C)cc23)cc1] |
| I-44 | 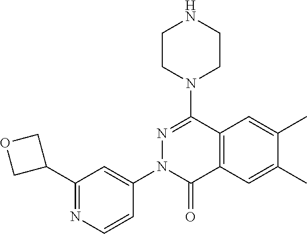 [SMILES: Cc1cc2c(N3CCNCC3)nn(-c3ccnc(C4COC4)c3)c(=O)c2cc1C] |
| I-45 | 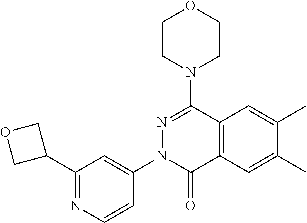 [SMILES: Cc1cc2c(N3CCOCC3)nn(-c3ccnc(C4COC4)c3)c(=O)c2cc1C] |
| I-46 | 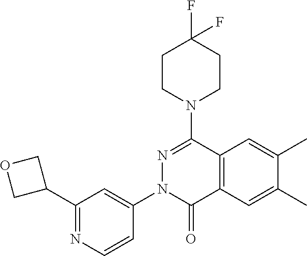 [SMILES: Cc1cc2c(N3CCC(F)(F)CC3)nn(-c3ccnc(C4COC4)c3)c(=O)c2cc1C] |
| I-47 | 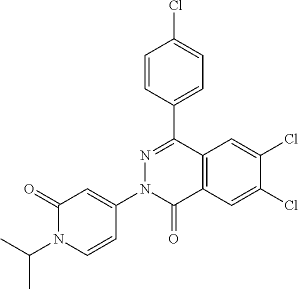 [SMILES: CC(C)n1ccc(-n2nc(-c3ccc(Cl)cc3)c3cc(Cl)c(Cl)cc3c2=O)cc1=O] |
| I-48 | 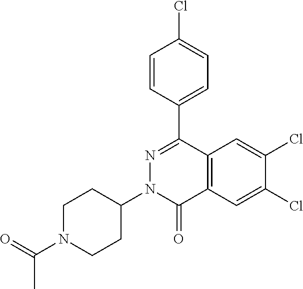 [SMILES: CC(=O)N1CCC(n2nc(-c3ccc(Cl)cc3)c3cc(Cl)c(Cl)cc3c2=O)CC1] |
| I-49 | 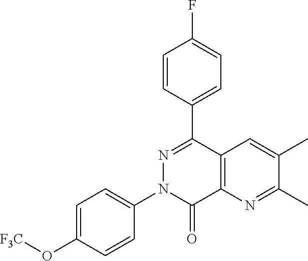 [SMILES: Cc1cc2c(-c3ccc(F)cc3)nn(-c3ccc(OC(F)(F)F)cc3)c(=O)c2nc1C] |
| I-50 | 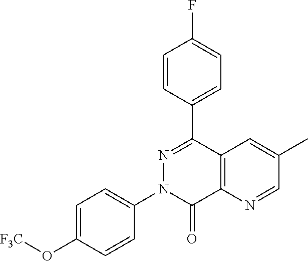 [SMILES: Cc1cnc2c(=O)n(-c3ccc(OC(F)(F)F)cc3)nc(-c3ccc(F)cc3)c2c1] |
| I-51 | 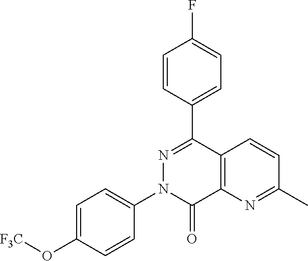 [SMILES: Cc1ccc2c(-c3ccc(F)cc3)nn(-c3ccc(OC(F)(F)F)cc3)c(=O)c2n1] |
| I-52 | 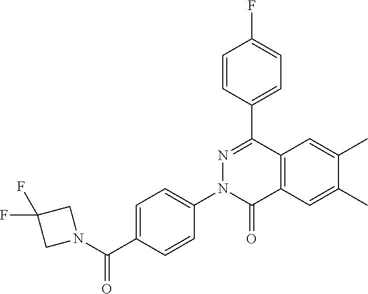 [SMILES: Cc1cc2c(-c3ccc(F)cc3)nn(-c3ccc(C(=O)N4CC(F)(F)C4)cc3)c(=O)c2cc1C] |
| I-53 | 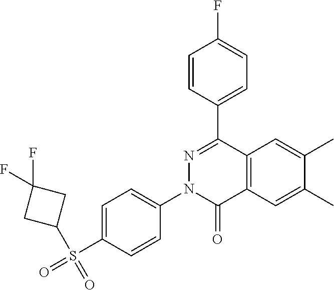 [SMILES: Cc1cc2c(-c3ccc(F)cc3)nn(-c3ccc(S(=O)(=O)C4CC(F)(F)C4)cc3)c(=O)c2cc1C] |
| I-54 | 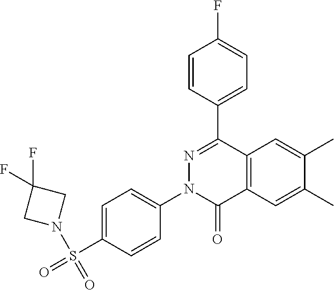 [SMILES: Cc1cc2c(-c3ccc(F)cc3)nn(-c3ccc(S(=O)(=O)N4CC(F)(F)C4)cc3)c(=O)c2cc1C] |
| I-55 | 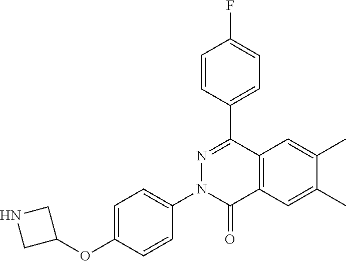 [SMILES: Cc1cc2c(-c3ccc(F)cc3)nn(-c3ccc(OC4CNC4)cc3)c(=O)c2cc1C] |
| I-56 | 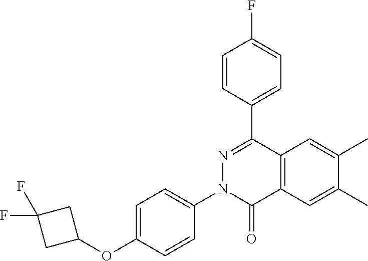 [SMILES: Cc1cc2c(-c3ccc(F)cc3)nn(-c3ccc(OC4CC(F)(F)C4)cc3)c(=O)c2cc1C] |
| I-57 | 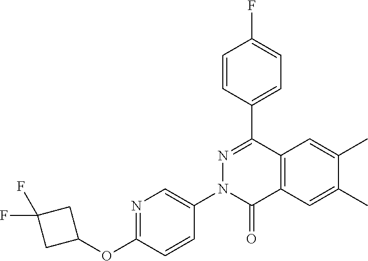 [SMILES: Cc1cc2c(-c3ccc(F)cc3)nn(-c3ccc(OC4CC(F)(F)C4)nc3)c(=O)c2cc1C] |
| I-58 | 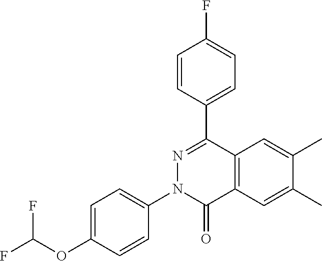 [SMILES: Cc1cc2c(-c3ccc(F)cc3)nn(-c3ccc(OC(F)F)cc3)c(=O)c2cc1C] |
| I-59 | 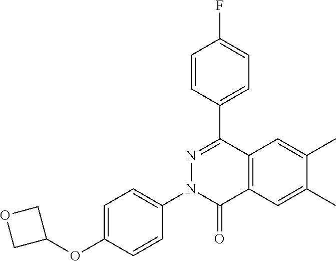 [SMILES: Cc1cc2c(-c3ccc(F)cc3)nn(-c3ccc(OC4COC4)cc3)c(=O)c2cc1C] |
| I-60 | 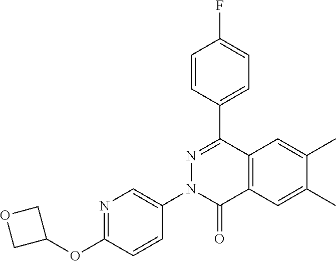 [SMILES: Cc1cc2c(-c3ccc(F)cc3)nn(-c3ccc(OC4COC4)nc3)c(=O)c2cc1C] |
| I-61 | 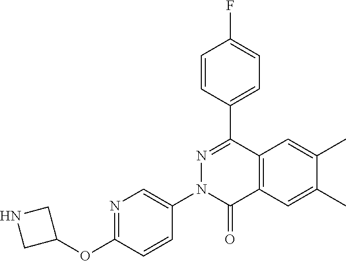 [SMILES: Cc1cc2c(-c3ccc(F)cc3)nn(-c3ccc(OC4CNC4)nc3)c(=O)c2cc1C] |
| I-62 | 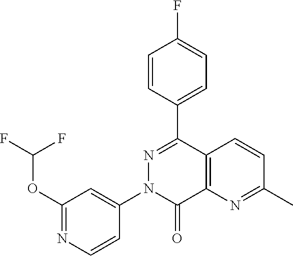 [SMILES: Cc1ccc2c(-c3ccc(F)cc3)nn(-c3ccnc(OC(F)F)c3)c(=O)c2n1] |
| I-63 | 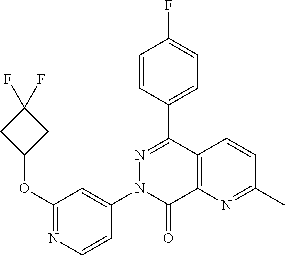 [SMILES: Cc1ccc2c(-c3ccc(F)cc3)nn(-c3ccnc(OC4CC(F)(F)C4)c3)c(=O)c2n1] |
| I-64 | 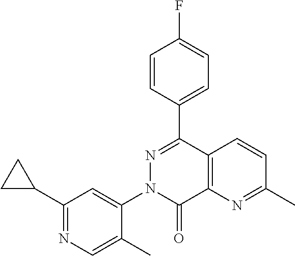 [SMILES: Cc1ccc2c(-c3ccc(F)cc3)nn(-c3cc(C4CC4)ncc3C)c(=O)c2n1] |
| I-65 | 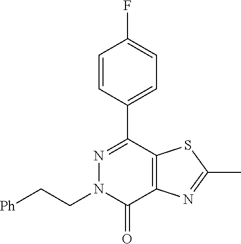 [SMILES: Cc1nc2c(=O)n(CCc3ccccc3)nc(-c3ccc(F)cc3)c2s1] |
| I-66 | 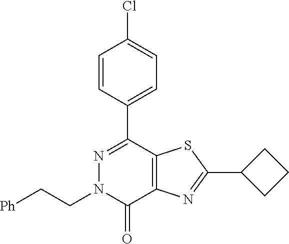 [SMILES: O=c1c2nc(C3CCC3)sc2c(-c2ccc(Cl)cc2)nn1CCc1ccccc1] |
| I-67 | 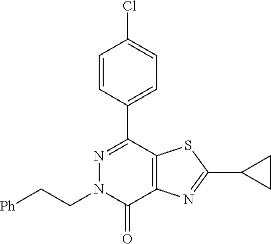 [SMILES: O=c1c2nc(C3CC3)sc2c(-c2ccc(Cl)cc2)nn1CCc1ccccc1] |
| I-68 | 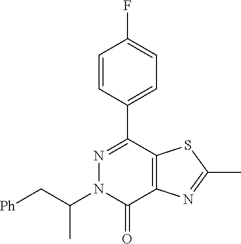 [SMILES: Cc1nc2c(=O)n(C(C)Cc3ccccc3)nc(-c3ccc(F)cc3)c2s1] |
| I-69 | 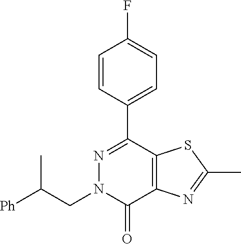 [SMILES: Cc1nc2c(=O)n(CC(C)c3ccccc3)nc(-c3ccc(F)cc3)c2s1] |
| I-70 | 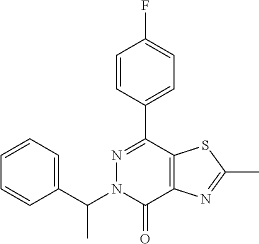 [SMILES: Cc1nc2c(=O)n(C(C)c3ccccc3)nc(-c3ccc(F)cc3)c2s1] |
| I-71 | 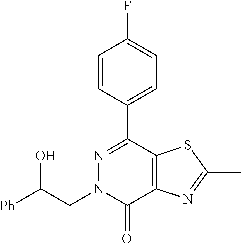 [SMILES: Cc1nc2c(=O)n(CC(O)c3ccccc3)nc(-c3ccc(F)cc3)c2s1] |
| I-72 | 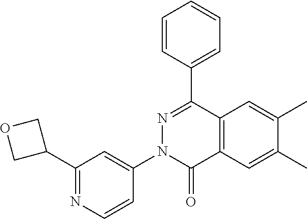 [SMILES: Cc1cc2c(-c3ccccc3)nn(-c3ccnc(C4COC4)c3)c(=O)c2cc1C] |
| I-73 | 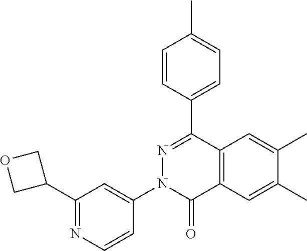 [SMILES: Cc1ccc(-c2nn(-c3ccnc(C4COC4)c3)c(=O)c3cc(C)c(C)cc23)cc1] |
| I-74 | 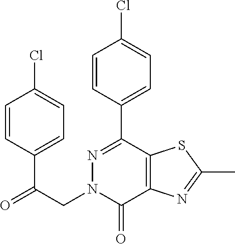 [SMILES: Cc1nc2c(=O)n(CC(=O)c3ccc(Cl)cc3)nc(-c3ccc(Cl)cc3)c2s1] |
| I-75 | 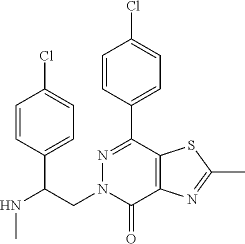 [SMILES: CNC(Cn1nc(-c2ccc(Cl)cc2)c2sc(C)nc2c1=O)c1ccc(Cl)cc1] |
| I-76 | 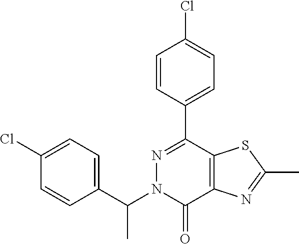 [SMILES: Cc1nc2c(=O)n(C(C)c3ccc(Cl)cc3)nc(-c3ccc(Cl)cc3)c2s1] |
| I-77 | 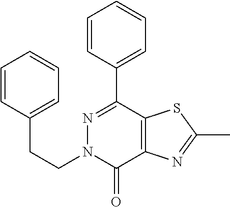 [SMILES: Cc1nc2c(=O)n(CCc3ccccc3)nc(-c3ccccc3)c2s1] |
| I-78 | 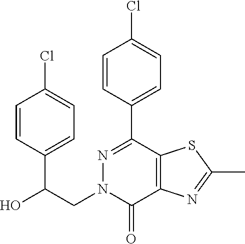 [SMILES: Cc1nc2c(=O)n(CC(O)c3ccc(Cl)cc3)nc(-c3ccc(Cl)cc3)c2s1] |
| I-79 | 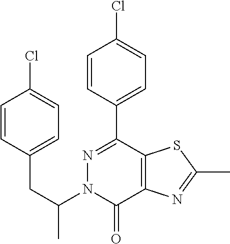 [SMILES: Cc1nc2c(=O)n(C(C)Cc3ccc(Cl)cc3)nc(-c3ccc(Cl)cc3)c2s1] |
| I-80 | 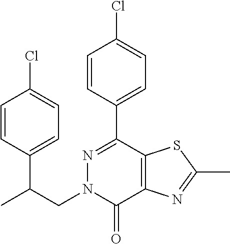 [SMILES: Cc1nc2c(=O)n(CC(C)c3ccc(Cl)cc3)nc(-c3ccc(Cl)cc3)c2s1] |
| I-81 | 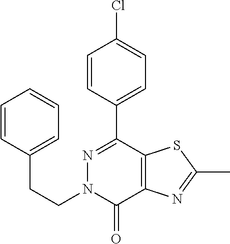 [SMILES: Cc1nc2c(=O)n(CCc3ccccc3)nc(-c3ccc(Cl)cc3)c2s1] |
| I-82 | 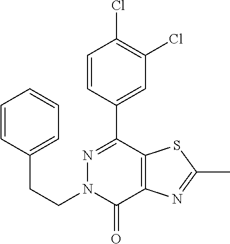 [SMILES: Cc1nc2c(=O)n(CCc3ccccc3)nc(-c3ccc(Cl)c(Cl)c3)c2s1] |
| I-83 | 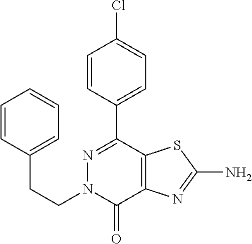 [SMILES: Nc1nc2c(=O)n(CCc3ccccc3)nc(-c3ccc(Cl)cc3)c2s1] |
| I-84 | 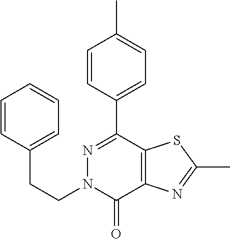 [SMILES: Cc1ccc(-c2nn(CCc3ccccc3)c(=O)c3nc(C)sc23)cc1] |
| I-85 | 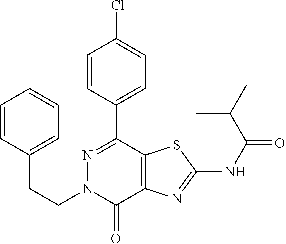 [SMILES: CC(C)C(=O)Nc1nc2c(=O)n(CCc3ccccc3)nc(-c3ccc(Cl)cc3)c2s1] |
| I-86 | 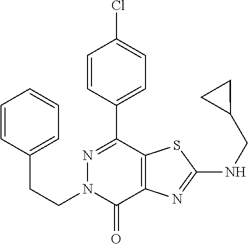 [SMILES: O=c1c2nc(NCC3CC3)sc2c(-c2ccc(Cl)cc2)nn1CCc1ccccc1] |
| I-87 | 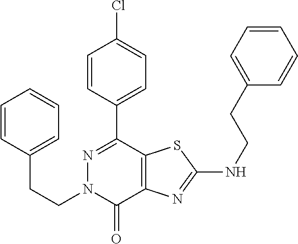 [SMILES: O=c1c2nc(NCCc3ccccc3)sc2c(-c2ccc(Cl)cc2)nn1CCc1ccccc1] |
| I-88 | 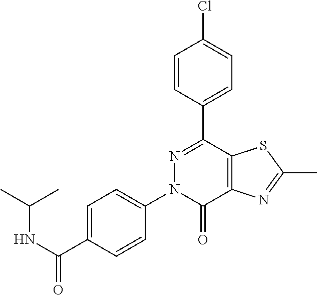 [SMILES: Cc1nc2c(=O)n(-c3ccc(C(=O)NC(C)C)cc3)nc(-c3ccc(Cl)cc3)c2s1] |
| I-89 | 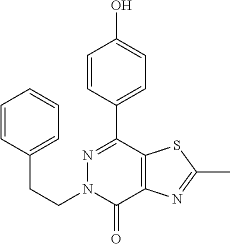 [SMILES: Cc1nc2c(=O)n(CCc3ccccc3)nc(-c3ccc(O)cc3)c2s1] |
| I-90 | 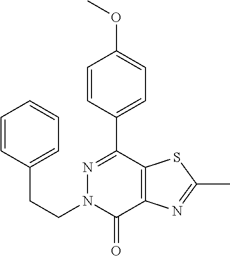 [SMILES: COc1ccc(-c2nn(CCc3ccccc3)c(=O)c3nc(C)sc23)cc1] |
| I-91 | 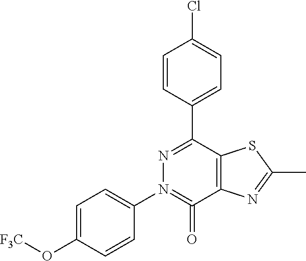 |
| I-92 | 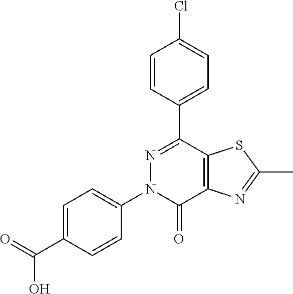 [SMILES: Cc1nc2c(=O)n(-c3ccc(C(=O)O)cc3)nc(-c3ccc(Cl)cc3)c2s1] |
| I-93 | 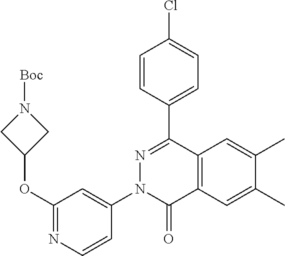 [SMILES: Cc1cc2c(-c3ccc(Cl)cc3)nn(-c3ccnc(OC4CN(C(=O)OC(C)(C)C)C4)c3)c(=O)c2cc1C] |
| I-94 | 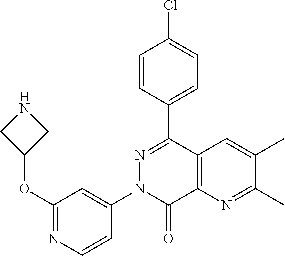 [SMILES: Cc1cc2c(-c3ccc(Cl)cc3)nn(-c3ccnc(OC4CNC4)c3)c(=O)c2nc1C] |
| I-95 | 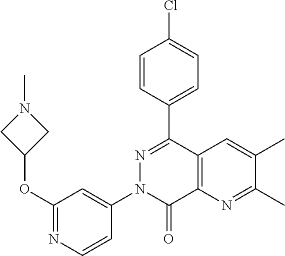 [SMILES: Cc1cc2c(-c3ccc(Cl)cc3)nn(-c3ccnc(OC4CN(C)C4)c3)c(=O)c2nc1C] |

Exemplary compounds of the invention are set forth in Table A-2, below. In some embodiments, the compound is a compound set forth in Table A-2, or a pharmaceutically acceptable salt thereof.

TABLE A-2

| I# | Structure |
|---|---|
| I-96 | 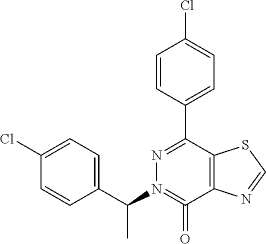 |
| I-97 | 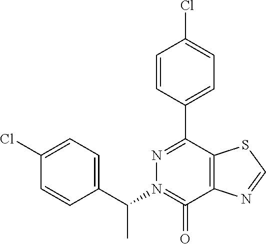 |
| I-98 | 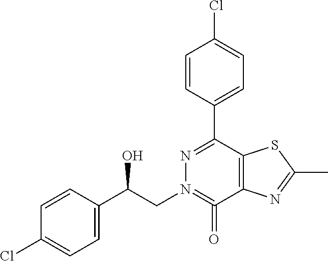 |
| I-99 | 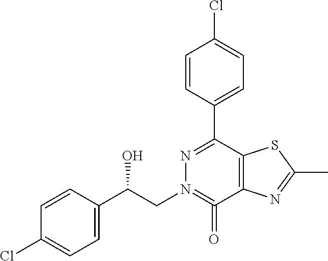 |
| I-100 | 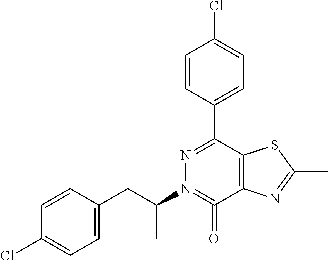 |
| I-101 | 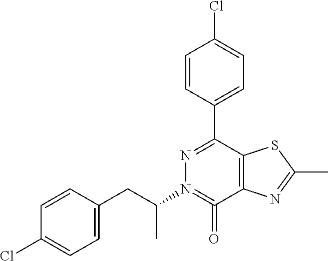 |
| I-102 | 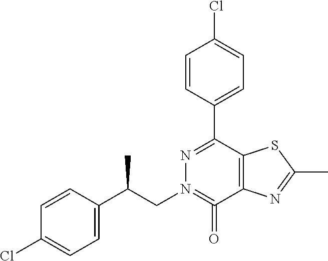 |
| I-103 | 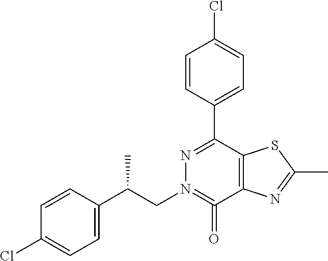 |
| I-104 | 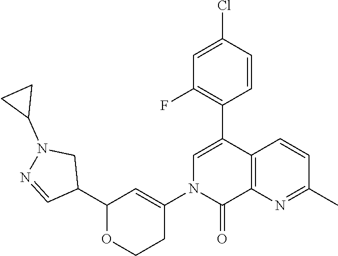 [SMILES: Cc1ccc2c(-c3ccc(Cl)cc3F)cn(C3=CC(C4C=NN(C5CC5)C4)OCC3)c(=O)c2n1] |
| I-105 | 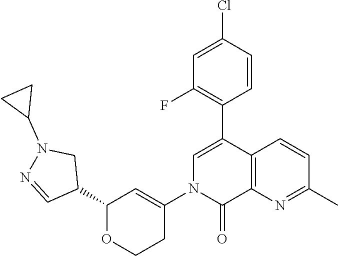 [SMILES: Cc1ccc2c(-c3ccc(Cl)cc3F)cn(C3=C[C@@H](C4C=NN(C5CC5)C4)OCC3)c(=O)c2n1] |
| I-106 | 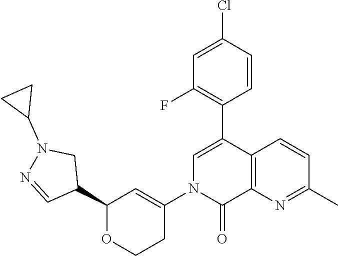 [SMILES: Cc1ccc2c(-c3ccc(Cl)cc3F)cn(C3=C[C@H](C4C=NN(C5CC5)C4)OCC3)c(=O)c2n1] |
| I-107 | 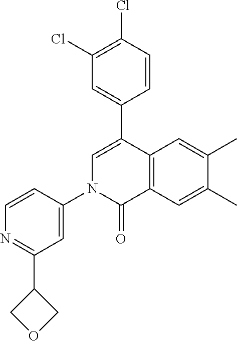 [SMILES: Cc1cc2c(-c3ccc(Cl)c(Cl)c3)cn(-c3ccnc(C4COC4)c3)c(=O)c2cc1C] |
| I-108 | 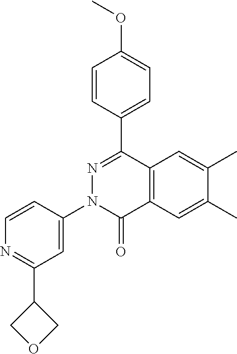 [SMILES: COc1ccc(-c2nn(-c3ccnc(C4COC4)c3)c(=O)c3cc(C)c(C)cc23)cc1] |
| I-109 | 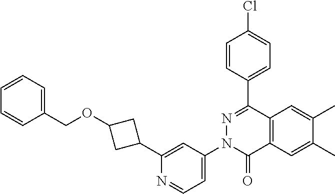 [SMILES: Cc1cc2c(-c3ccc(Cl)cc3)nn(-c3ccnc(C4CC(OCc5ccccc5)C4)c3)c(=O)c2cc1C] |
| I-110 | 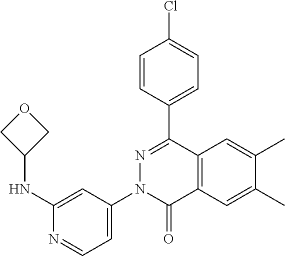 [SMILES: Cc1cc2c(-c3ccc(Cl)cc3)nn(-c3ccnc(NC4COC4)c3)c(=O)c2cc1C] |
| I-111 | 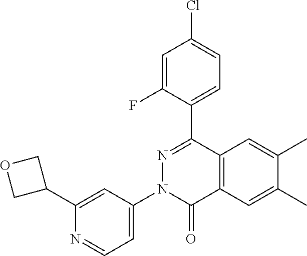 [SMILES: Cc1cc2c(-c3ccc(Cl)cc3F)nn(-c3ccnc(C4COC4)c3)c(=O)c2cc1C] |
| I-112 | 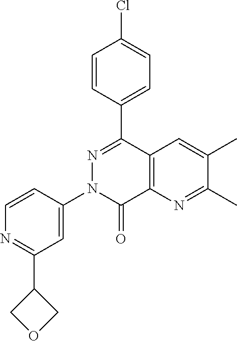 [SMILES: Cc1cc2c(-c3ccc(Cl)cc3)nn(-c3ccnc(C4COC4)c3)c(=O)c2nc1C] |
| I-113 | 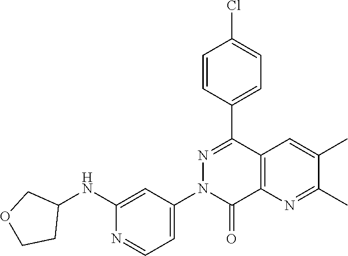 [SMILES: Cc1cc2c(-c3ccc(Cl)cc3)nn(-c3ccnc(NC4CCOC4)c3)c(=O)c2nc1C] |
| I-114 | 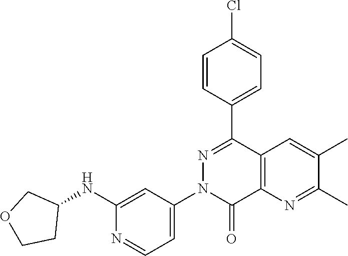 [SMILES: Cc1cc2c(-c3ccc(Cl)cc3)nn(-c3ccnc(N[C@@H]4CCOC4)c3)c(=O)c2nc1C] |
| I-115 | 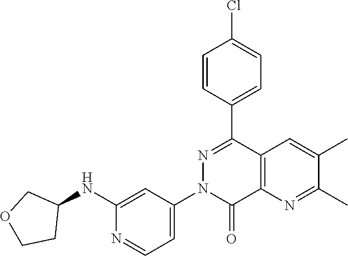 [SMILES: Cc1cc2c(-c3ccc(Cl)cc3)nn(-c3ccnc(N[C@H]4CCOC4)c3)c(=O)c2nc1C] |
| I-116 | 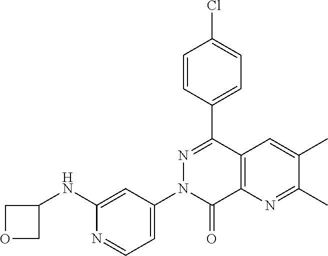 [SMILES: Cc1cc2c(-c3ccc(Cl)cc3)nn(-c3ccnc(NC4COC4)c3)c(=O)c2nc1C] |
| I-117 | 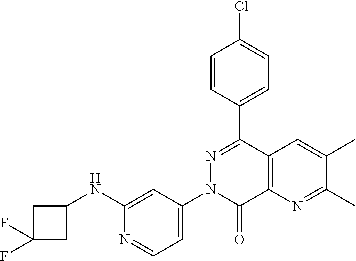 [SMILES: Cc1cc2c(-c3ccc(Cl)cc3)nn(-c3ccnc(NC4CC(F)(F)C4)c3)c(=O)c2nc1C] |
| I-118 | 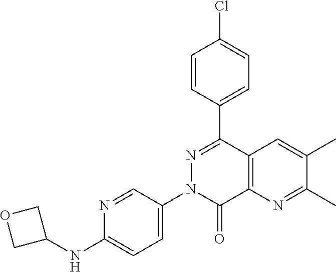 [SMILES: Cc1cc2c(-c3ccc(Cl)cc3)nn(-c3ccc(NC4COC4)nc3)c(=O)c2nc1C] |
| I-119 | 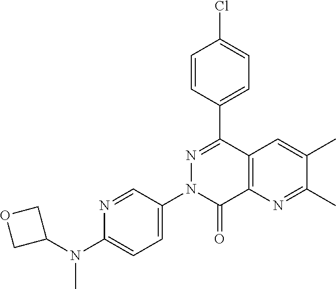 [SMILES: Cc1cc2c(-c3ccc(Cl)cc3)nn(-c3ccc(N(C)C4COC4)nc3)c(=O)c2nc1C] |
| I-120 | 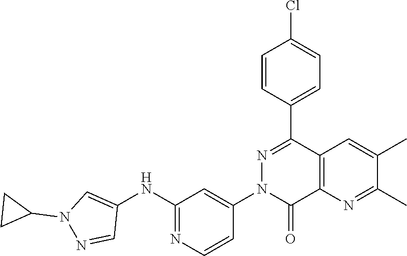 [SMILES: Cc1cc2c(-c3ccc(Cl)cc3)nn(-c3ccnc(Nc4cnn(C5CC5)c4)c3)c(=O)c2nc1C] |
| I-121 | 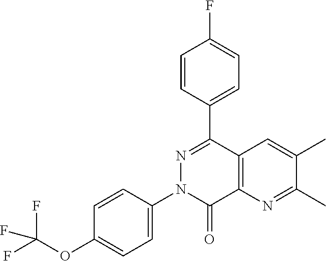 [SMILES: Cc1cc2c(-c3ccc(F)cc3)nn(-c3ccc(OC(F)(F)F)cc3)c(=O)c2nc1C] |
| I-122 | 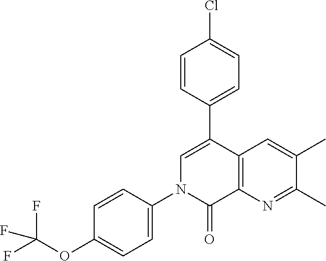 [SMILES: Cc1cc2c(-c3ccc(Cl)cc3)cn(-c3ccc(OC(F)(F)F)cc3)c(=O)c2nc1C] |
| I-123 | 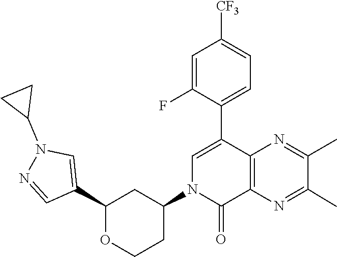 [SMILES: Cc1nc2c(-c3ccc(C(F)(F)F)cc3F)cn([C@H]3CCO[C@@H](c4cnn(C5CC5)c4)C3)c(=O)c2nc1C] |
| I-124 | 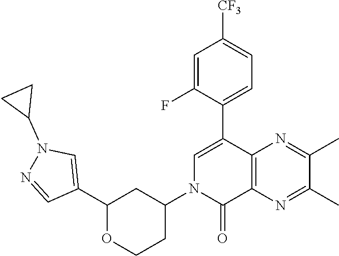 [SMILES: Cc1nc2c(-c3ccc(C(F)(F)F)cc3F)cn(C3CCOC(c4cnn(C5CC5)c4)C3)c(=O)c2nc1C] |
| I-125 | 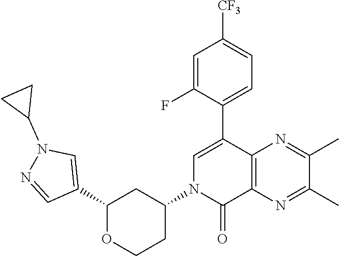 [SMILES: Cc1nc2c(-c3ccc(C(F)(F)F)cc3F)cn([C@@H]3CCO[C@H](c4cnn(C5CC5)c4)C3)c(=O)c2nc1C] |
| I-126 | 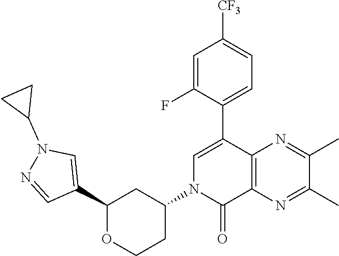 [SMILES: Cc1nc2c(-c3ccc(C(F)(F)F)cc3F)cn([C@@H]3CCO[C@@H](c4cnn(C5CC5)c4)C3)c(=O)c2nc1C] |
| I-127 | 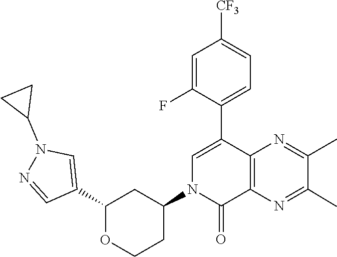 [SMILES: Cc1nc2c(-c3ccc(C(F)(F)F)cc3F)cn([C@H]3CCO[C@H](c4cnn(C5CC5)c4)C3)c(=O)c2nc1C] |

The foregoing merely summarizes certain aspects of this disclosure and is not intended, nor should it be construed, as limiting the disclosure in any way.

### Formulation and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

In one aspect, the invention provides a pharmaceutical composition comprising a compound of the present disclosure, or a tautomer thereof, or a pharmaceutically acceptable salt of said compound or said tautomer, and a pharmaceutically acceptable excipient.

In another aspect, the invention provides a compound of the present disclosure, or a tautomer thereof, or a pharmaceutically acceptable salt of said compound or said tautomer, or a pharmaceutical composition comprising said compound, or said tautomer, or said salt, for use as a medicament.

### Pharmaceutically Acceptable Compositions

According to some embodiments, the present disclosure provides a composition comprising a compound of this disclosure or a pharmaceutically acceptable derivative thereof and a pharmaceutically acceptable carrier, adjuvant, or vehicle. The amount of compound in compositions of this disclosure is such that it is effective to measurably activate a TREM2 protein, or a mutant thereof, in a biological sample or in a patient. In certain embodiments, the amount of compound in compositions of this disclosure is such that it is effective to measurably activate a TREM2 protein, or a mutant thereof, in a biological sample or in a patient. In certain embodiments, a composition of this disclosure is formulated for administration to a patient in need of such composition. In some embodiments, a composition of this disclosure is formulated for oral administration to a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

Without wishing to be bound by any particular theory, the following is noted: TREM2 has been implicated in several myeloid cell processes, including phagocytosis, proliferation, survival, and regulation of inflammatory cytokine production. Ulrich and Holtzman 2016. In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes. Guerreiro et al. 2013. More recently, variants in the TREM2 gene have been linked to increased risk for Alzheimer's disease (AD) and other forms of dementia including frontotemporal dementia. Jonsson et al. 2013, Guerreiro, Lohmann et al. 2013, and Jay, Miller et al. 2015. In particular, the R47H variant has been identified in genome-wide studies as being associated with increased risk for late-onset AD with an overall adjusted odds ratio (for populations of all ages) of 2.3, second only to the strong genetic association of ApoE to Alzheimer's. The R47H mutation resides on the extracellular lg V-set domain of the TREM2 protein and has been shown to impact lipid binding and uptake of apoptotic cells and Abeta (Wang et al. 2015; Yeh et al. 2016), suggestive of a loss-of-function linked to disease. Further, postmortem comparison of AD patients' brains with and without the R47H mutation are supportive of a novel loss-of-microglial barrier function for the carriers of the mutation, with the R47H carrier microglia putatively demonstrating a reduced ability to compact plaques and limit their spread. Yuan et al. 2016. Impairment in microgliosis has been reported in animal models of prion disease, multiple sclerosis, and stroke, suggesting that TREM2 may play an important role in supporting microgliosis in response to pathology or damage in the central nervous system. Ulrich and Holtzman 2016. In addition, knockdown of TREM2 has been shown to aggravate a-syn-induced inflammatory responses in vitro and exacerbate dopaminergic neuron loss in response to AAV-SYN in vivo (a model of Parkinson's disease), suggesting that impaired microglial TREM2 signaling exacerbates neurodegeneration by modulating microglial activation states. Guo et. al. 2019. A variety of animal models also suggest that Toll-Like Receptor (TLR) signaling is important in the pathogenesis of Rheumatoid Arthritis (RA) via persistent expression of pro-inflammatory cytokines by macrophages. Signaling through TREM2/DAP12 inhibits TLR responses by reducing MAPK (Erk1/2) activation, suggesting that TREM2 activation may act as a negative regulator of TLR driven RA pathogenesis. Huang and Pope 2009.

In view of the data indicating that deficits in TREM2 activity affect macrophage and microglia function, the compounds disclosed herein are of particular use in disorders, such as those described above and in the embodiments that follow and in neurodegenerative disorders more generally.

In one aspect, the invention provides a compound of the present disclosure, or a tautomer thereof, or a pharmaceutically acceptable salt of said compound or said tautomer, or a pharmaceutical composition thereof for use in treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in treating or preventing Parkinson's disease, rheumatoid arthritis, Alzheimer's disease, Nasu-Hakola disease, frontotemporal dementia, multiple sclerosis, prion disease, or stroke.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with a loss of function of human TREM2.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing Parkinson's disease, rheumatoid arthritis, Alzheimer's disease, Nasu-Hakola disease, frontotemporal dementia, multiple sclerosis, prion disease, or stroke.

In another aspect, the invention provides a method of treating or preventing a condition associated with a loss of function of human TREM2 in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

In another aspect, the invention provides a method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound of the present disclosure, or a tautomer thereof, or a pharmaceutically acceptable salt of said compound or said tautomer, or a pharmaceutical composition thereof.

### CSF1R

CSF1R is a cell-surface receptor primarily for the cytokine colony stimulating factor 1 (CSF-1), also known until recently as macrophage colony-stimulating factor (M-CSF), which regulates the survival, proliferation, differentiation and function of mononuclear phagocytic cells, including microglia of the central nervous system. CSF1R is composed of a highly glycosylated extracellular ligand-binding domain, a trans-membrane domain and an intracellular tyrosine-kinase domain. Binding of CSF-1 to CSF1R results in the formation of receptor homodimers and subsequent auto-phosphorylation of several tyrosine residues in the cytoplasmic domain, notably Syk. In the brain, CSF1R is predominantly expressed in microglial cells. It has been found that microglia in CSF1R+/− patients are depleted and show increased apoptosis (Oosterhof et al., 2018).

The present invention relates to the unexpected discovery that administration of a TREM2 agonist can rescue the loss of microglia in cells having mutations in CSF1R. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in CSF1R signaling caused by a decrease in the concentration of its ligand. In a 5×FAD murine Alzheimer's disease model of amyloid pathology, doses of a CSF1R inhibitor that almost completely eliminate microglia in the brains of wild-type animals show surviving microglia clustered around the amyloid plaques (Spangenberg et al, Nature Communications 2019). Plaque amyloid has been demonstrated in the past to be a ligand for TREM2, and it has been shown that microglial engagement with amyloid is dependent on TREM2 (Condello et al, Nat Comm., 2015). The present invention relates to the unexpected discovery that it is activation of TREM2 that rescued the microglia in the presence of the CSF1R inhibitor, and that this effect is also observed in patients suffering from loss of microglia due to CSF1R mutation. This discovery has not been previously taught or suggested in the available art.

Adult-onset leukoencephalopathy with axonal spheroids and pigmented glia (ALSP), previously recognized as hereditary diffuse leukoencephalopathy with axonal spheroids (HDLS) or pigmentary orthochromatic leukodystrophy (POLD), is an autosomal-dominant central nervous system disease that manifests in the form of variable behavioral, cognitive and motor function changes in patients suffering from the disease. ALSP is characterized by patchy cerebral white matter abnormalities visible by magnetic resonance imaging. However, the clinical symptoms and MRI changes are not specific to ALSP and are common for other neurological conditions, including Nasu-Hakola disease (NHD) and AD, making diagnosis and treatment of ALSP very difficult.

Recent studies have discovered that ALSP is a Mendelian disorder in which patients carry a heterozygous loss of function mutation in the kinase domain of CSF1R, suggesting a reduced level of signaling on the macrophage colony-stimulating factor (M-CSF)/CSF1R axis (Rademakers et al, Nat Genet 2012; Konno et al, Neurology 2018). In one aspect, the present invention relates to the surprising discovery that activation of the TREM2 pathway can rescue the loss of microglia in CSF1R+/− ALSP patients, preventing microglia apoptosis, thereby treating the ALSP condition.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of Colony stimulating factor 1 receptor (CSF1R, also known as macrophage colony-stimulating factor receptor/M-CSFR, or cluster of differentiation 115/CD115).

In one aspect, the invention provides a compound of the present disclosure, or a tautomer thereof, or a pharmaceutically acceptable salt of said compound or said tautomer, or a pharmaceutical composition thereof for use in treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of CSF1R.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

In another aspect, the invention provides a method of treating or preventing a disease or disorder associated with dysfunction of CSF1R in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the subject is selected for treatment based on a diagnosis that includes the presence of a mutation in a CSF1R gene affecting the function of CSF1R. In some embodiments, the mutation in the CSF1R gene is a mutation that causes a decrease in CSF1R activity or a cessation of CSF1R activity. In some embodiments, the disease or disorder is caused by a heterozygous CSF1R mutation. In some embodiments, the disease or disorder is caused by a homozygous CSF1R mutation. In some embodiments, the disease or disorder is caused by a splice mutation in the csf1r gene. In some embodiments, the disease or disorder is caused by a missense mutation in the csf1r gene. In some embodiments, the disease or disorder is caused by a mutation in the catalytic kinase domain of CSF1R. In some embodiments, the disease or disorder is caused by a mutation in an immunoglobulin domain of CSF1R. In some embodiments, the disease or disorder is caused by a mutation in the ectodomain of CSF1R. In some embodiments, the disease or disorder is a disease or disorder resulting from a change (e.g. increase, decrease or cessation) in the activity of CSF1R. In some embodiments, the disease or disorder is a disease or disorder resulting from a decrease or cessation in the activity of CSF1R. CSF1R related activities that are changed in the disease or disorder include, but are not limited to: decrease or loss of microglia function; increased microglia apoptosis; decrease in Src signaling; decrease in Syk signaling; decreased microglial proliferation; decreased microglial response to cellular debris; decreased phagocytosis; and decreased release of cytokines in response to stimuli. In some embodiments, the disease or disorder is caused by a loss-of-function mutation in CSF1R. In some embodiments, the loss-of-function mutation results in a complete cessation of CSF1R function. In some embodiments, the loss-of-function mutation results in a partial loss of CSF1R function, or a decrease in CSF1R activity.

In another aspect, the invention provides a method of treating or preventing adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats or prevents ALSP, which is an encompassing and superseding name for both HDLS and POLD. In some embodiments, the disease or disorder is a homozygous mutation in CSF1R. In some embodiments, the method treats or prevents pediatric-onset leukoencephalopathy. In some embodiments, the method treats or prevents congenital absence of microglia. In some embodiments, the method treats or prevents brain abnormalities neurodegeneration and dysosteosclerosis (BANDDOS).

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSF1R dysfunction, or having a mutation in a gene affecting the function of CSF1R, the method comprising administering to the subject a therapeutically effective amount of a compound of the present disclosure, or a tautomer thereof, or a pharmaceutically acceptable salt of said compound or said tautomer, or a pharmaceutical composition thereof.

### ABCD1

The ABCD1 gene provides instructions for producing the adrenoleukodystrophy protein (ALDP). ABCD1 (ALDP) maps to Xq28. ABCD1 is a member of the ATP-binding cassette (ABC) transporter superfamily. The superfamily contains membrane proteins that translocate a wide variety of substrates across extra- and intracellular membranes, including metabolic products, lipids and sterols, and drugs. ALDP is located in the membranes of cell structures called peroxisomes. Peroxisomes are small sacs within cells that process many types of molecules. ALDP brings a group of fats called very long-chain fatty acids (VLCFAs) into peroxisomes, where they are broken down. As ABCD1 is highly expressed in microglia, it is possible that microglial dysfunction and their close interaction with other cell types actively participates in neurodegenerative processes (Gong et al., Annals of Neurology. 2017; 82(5):813-827). It has been shown that severe microglia loss and damage is an early feature in patients with cerebral form of x-linked ALD (cALD) carrying ABCD1 mutations (Bergner et al., Glia. 2019; 67: 1196-1209). It has also been shown that ABCD1-deficiency leads to an impaired plasticity of myeloid lineage cells that is reflected in incomplete establishment of anti-inflammatory responses, thus possibly contributing to the devastating rapidly progressive demyelination in cerebral adrenoleukodystrophy (Weinhor et al., BRAIN 2018: 141; 2329-2342). These findings emphasize microglia/monocytes/macrophages as crucial therapeutic targets for preventing or stopping myelin destruction in patients with X-linked adrenoleukodystrophy.

The present invention relates to the unexpected discovery that administration of a TREM2 agonist can rescue the loss of microglia in cells having mutations in the ABCD1 gene. It has been previously shown that TREM2 agonist antibody 4D9 increases ATP luminescence (a measure of cell number and activity) in a dose dependent manner when the levels of M-CSF in media are reduced to 5 ng/mL (Schlepckow et al, EMBO Mol Med., 2020) and that TREM2 agonist AL002c increases ATP luminescence when M-CSF is completely removed from the media (Wang et al, J. Exp. Med.; 2020, 217(9): e20200785). This finding suggests that TREM2 agonism can compensate for deficiency in ABCD1 function leading to sustained activation, proliferation, chemotaxis of microglia, maintenance of anti-inflammatory environment and reduced astrocytosis caused by a decrease in ABCD1 and accumulation of VLCFAs. The present invention relates to the unexpected discovery that activation of TREM2 can rescue the microglia in the presence of the ABCD1 mutation and an increase in VLCFA, and that this effect may be also observed in patients suffering from loss of microglia due to ABCD1 mutation. This discovery has not been previously taught or suggested in the available art.

In one aspect, the invention provides a compound of the present disclosure, or a tautomer thereof, or a pharmaceutically acceptable salt of said compound or said tautomer, or a pharmaceutical composition thereof for use in treating or preventing a condition associated with dysfunction of ATP-binding cassette transporter 1 (ABCD1).

In one aspect, the invention provides a compound of the present disclosure, or a tautomer thereof, or a pharmaceutically acceptable salt of said compound or said tautomer, or a pharmaceutical composition thereof for use in treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing a condition associated with dysfunction of ABCD1.

In one aspect, the invention provides a compound of the present disclosure, or a tautomer thereof, or a pharmaceutically acceptable salt of said compound or said tautomer, or a pharmaceutical composition thereof for use in the preparation of a medicament for treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

In yet another aspect, the invention provides a method of treating or preventing a disease or disorder associated with dysfunction of ABCD1 in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the patient is selected for treatment based on a diagnosis that includes the presence of a mutation in an ABCD1 gene affecting the function of ABCD1. In some embodiments, the mutation in the ABCD1 gene is a mutation that causes a decrease in ABCD1 activity or a cessation of ABCD1 activity. In some embodiments, the disease or disorder is caused by a heterozygous ABCD1 mutation. In some embodiments, the disease or disorder is caused by a homozygous ABCD1 mutation. In some embodiments, the disease or disorder is caused by a splice mutation in the ABCD1 gene. In some embodiments, the disease or disorder is caused by a missense mutation in the ABCD1 gene. In some embodiments, the disease or disorder is a disease or disorder resulting from a change (e.g. increase, decrease or cessation) in the activity of ABCD1. In some embodiments, the disease or disorder is a disease or disorder resulting from a decrease or cessation in the activity of ABCD1. ABCD1 related activities that are changed in the disease or disorder include, but are not limited to peroxisomal import of fatty acids and/or fatty acyl-CoAs and production of adrenoleukodystrophy protein (ALDP). In some embodiments, the disease or disorder is caused by a loss-of-function mutation in ABCD1. In some embodiments, the loss-of-function mutation results in a complete cessation of ABCD1 function. In some embodiments, the loss-of-function mutation results in a partial loss of ABCD1 function, or a decrease in ABCD1 activity. In some embodiments, the disease or disorder is caused by a homozygous mutation in ABCD1. In some embodiments, the disease or disorder is a neurodegenerative disorder. In some embodiments, the disease or disorder is a neurodegenerative disorder caused by and/or associated with an ABCD1 dysfunction. In some embodiments, the disease or disorder is an immunological disorder. In some embodiments, the disease or disorder is an immunological disorder caused by and/or associated with an ABCD1 dysfunction.

In yet another aspect, the invention provides a method of treating or preventing X-linked adrenoleukodystrophy (x-ALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX) in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, any of the aforementioned diseases are present in a patient exhibiting ABCD1 dysfunction or having a mutation in a gene affecting the function of ABCD1. In some embodiments, the method treats or prevents X-linked adrenoleukodystrophy (x-ALD). In some embodiments, the x-ALD is a cerebral form of x-linked ALD (cALD). In some embodiments, the method treats or prevents Addison disease wherein the patient has been found to have a mutation in one or more ABCD1 genes affecting ABCD1 function. In some embodiments, the method treats or prevents Addison disease, wherein the patient has a loss-of-function mutation in ABCD1.

In yet another aspect, the invention provides a method of treating or preventing Nasu-Hakola disease, Alzheimer's disease, frontotemporal dementia, multiple sclerosis, Guillain-Barre syndrome, amyotrophic lateral sclerosis (ALS), or Parkinson's disease, wherein any of the aforementioned diseases or disorders are present in a patient exhibiting ABCD1 dysfunction, or having a mutation in a gene affecting the function of ABCD1, the method comprising administering to the subject a therapeutically effective amount of a compound of the present disclosure, or a tautomer thereof, or a pharmaceutically acceptable salt of said compound or said tautomer, or a pharmaceutical composition thereof.

### Autism Spectrum Disorders

It has been found that TREM2 deficient mice exhibit symptoms reminiscent of autism spectrum disorders (ASDs) (Filipello et al., Immunity, 2018, 48, 979-991). It has also been found that microglia depletion of the autophagy Aatg7 gene results in defective synaptic pruning and results in increased dendritic spine density, and abnormal social interaction and repetitive behaviors indicative of ASDs (Kim, et al., Molecular Psychiatry, 2017, 22, 1576-1584). Further studies have shown that increased dendritic spin density detected in post-mortem ASD brains, likely caused by defective synaptic pruning, results in circuit hypoconnectivity and behavioral defects and are a potential origin of a number of neurodevelopmental diseases (Tang, et al., Neuron, 2014, 83, 1131-1143). Without intending to be limited to any particular theory, these findings suggest that TREM2 activation can reverse microglia depletion, and therefore correct the defective synaptic pruning that is central to neurodevelopmental diseases such as ASDs. The present invention relates to the unexpected discovery that activation of TREM2, using a compound of the present invention, can rescue microglia in subjects suffering from an ASD. This discovery has not been previously taught or suggested in the available art.

In another aspect, the present invention provides a compound of the present disclosure, or a tautomer thereof, or a pharmaceutically acceptable salt of said compound or said tautomer, or a pharmaceutical composition thereof for use in treating autism or autism spectrum disorders.

In yet another aspect, the present invention provides a compound of the present disclosure, or a tautomer thereof, or a pharmaceutically acceptable salt of said compound or said tautomer, or a pharmaceutical composition thereof for use in the preparation of a medicament for treating autism or autism spectrum disorders.

In yet another aspect, the present invention provides a method of treating autism or autism spectrum disorders in a subject in need thereof, the method comprising administering to the subject a therapeutically effective amount of a compound of the present disclosure, or a tautomer thereof, or a pharmaceutically acceptable salt of said compound or said tautomer, or a pharmaceutical composition thereof. In some embodiments, the method treats autism. In some embodiments, the method treats Asperger syndrome.

In some embodiments, the disclosure provides a method of increasing the activity of TREM2, the method comprising contacting a compound of the present disclosure, or a pharmaceutically acceptable salt thereof with the TREM2. In some embodiments, the contacting takes place in vitro. In some embodiments, the contacting takes place in vivo. In some embodiments, the TREM2 is human TREM2.

### Combination Therapies

Depending upon the particular condition, or disease, to be treated, additional therapeutic agents, which are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present disclosure provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this disclosure may also be combined with include, without limitation: treatments for Parkinson's disease, rheumatoid arthritis, Alzheimer's disease, Nasu-Hakola disease, frontotemporal dementia, multiple sclerosis, prion disease, or stroke.

As used herein, the term “combination,” “combined,” and related terms refers to the simultaneous or sequential administration of therapeutic agents in accordance with this disclosure. For example, a combination of the present disclosure may be administered with another therapeutic agent simultaneously or sequentially in separate unit dosage forms or together in a single unit dosage form.

The amount of additional therapeutic agent present in the compositions of this disclosure will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the present disclosure, as part of a multiple dosage regimen. Alternatively, one or more other therapeutic agents may be part of a single dosage form, mixed together with a compound of this disclosure in a single composition. If administered as a multiple dosage regime, one or more other therapeutic agent and a compound or composition of the present disclosure may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the present disclosure are administered as a multiple dosage regimen within greater than 24 hours a parts.

In one embodiment, the present disclosure provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

### Definitions

The following definitions are provided to assist in understanding the scope of this disclosure.

Unless otherwise indicated, all numbers expressing quantities of ingredients, reaction conditions, and so forth used in the specification or claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated. For example, (1R)-1-methyl-2-(trifluoromethyl)cyclohexane is meant to encompass (1R,2R)-1-methyl-2-(trifluoromethyl)cyclohexane and (1R,2S)-1-methyl-2-(trifluoromethyl)cyclohexane. A bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of the other enantiomer and diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labelled forms thereof or a pharmaceutically acceptable salt of any of the foregoing may exist in solvated or unsolvated forms.

The term “solvate” as used herein refers to a molecular complex comprising a compound or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labelled forms thereof or a pharmaceutically acceptable salt of any of the foregoing.

### Miscellaneous Definitions

This section will define additional terms used to describe the scope of the compounds, compositions and uses disclosed herein.

Compounds of this present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 101^{st} Ed. Additionally, general principles of organic chemistry are described in “Organic Chemistry”, Thomas Sorrell, University Science Books, Sausalito: 2005, and “March's Advanced Organic Chemistry: Reactions Mechanisms and Structure”, 8^{th} Ed., Ed.: Smith, M. B., John Wiley & Sons, New York: 2019, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1 to 6 aliphatic carbon atoms. In some embodiments, aliphatic groups contain 1 to 5 aliphatic carbon atoms. In other embodiments, aliphatic groups contain 1 to 4 aliphatic carbon atoms. In still other embodiments, aliphatic groups contain 1 to 3 aliphatic carbon atoms, and in yet other embodiments, aliphatic groups contain 1 to 2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphonates and phosphates), boron, etc. In some embodiments, a bicyclic group has 7-12 ring members and 0-4 heteroatoms independently selected from nitrogen, oxygen, and sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

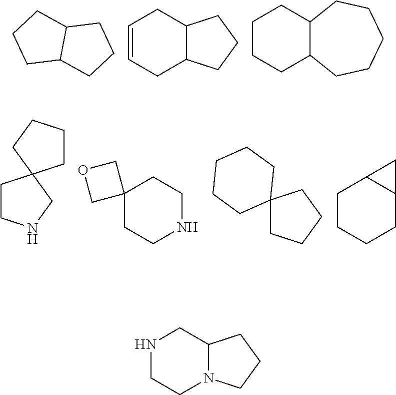

Exemplary bridged bicyclics include:

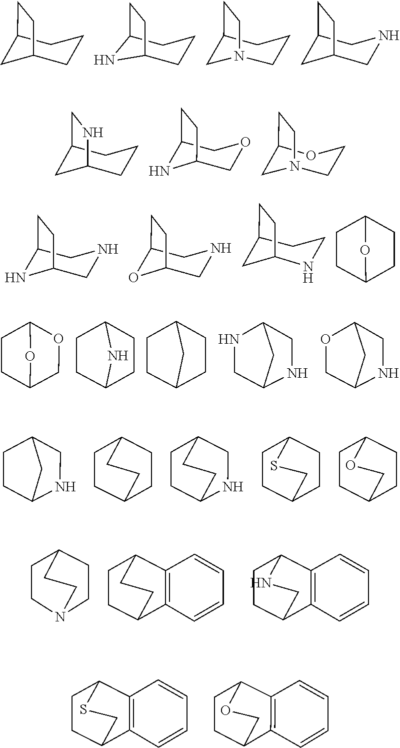

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen; or an oxygen, sulfur, nitrogen, phosphorus, or silicon atom in a heterocyclic ring.

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7 to 10-membered bicyclic heterocyclic moiety that is either saturated or partially unsaturated, and having, in addition to carbon atoms, one or more, preferably 1 to 4, heteroatoms, as defined above. When used in reference to a ring atom of a heterocycle, the term “nitrogen” includes a substituted nitrogen. As an example, in a saturated or partially unsaturated ring (having 0 to 3 heteroatoms selected from oxygen, sulfur and nitrogen.

A heterocyclic ring can be attached to a provided compound at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic or bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₆R^{∘}; —(CH₂)₀₋₆OR^{∘}; —O(CH₂)₀₋₆R^{∘}, —O—(CH₂)₀₋₆C(O)OR^{∘}; —(CH₂)₀₋₆CH(OR^{∘})₂; —(CH₂)₀₋₆SR^{∘}; —(CH₂)₀₋₆Ph, which Ph may be substituted with R^{∘}; —(CH₂)₀₋₄₆O(CH₂)₀₋₁Ph which Ph may be substituted with R^{∘}; —CH═CHPh, which Ph may be substituted with R^{∘}; —(CH₂)₀₋₆O(CH₂)₀₋₁-pyridyl which pyridyl may be substituted with R^{∘}; —NO₂; —CN; —N₃; —(CH₂)₀₋₆N(R^{∘})₂; —(CH₂)₀₋₆N(R^{∘})C(O)R^{∘}; —N(R^{∘})C(S)R^{∘}; —(CH₂)₀₋₆N(R^{∘})C(O)NR⁰²; —N(R^{∘})C(S)NR^{∘}₂; —(CH₂)₀₋₆N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₆C(O)R^{∘}; —C(S)R^{∘}; —(CH₂)₀₋₆C(O)OR^{∘}; —(CH₂)₀₋₆C(O)SR^{∘}; —(CH₂)₀₋₆C(O)OSiR^{∘}₃; —(CH₂)₀₋₆OC(O)R^{∘}; —OC(O)(CH₂)₀₋₆—SR^{∘}, —(CH₂)₀₋₆SC(O)R^{∘}; —(CH₂)₀₋₆C(O)NR^{∘}₂; —C(S)NR^{∘}₂; —C(S)SR^{∘}; —SC(S)SR^{∘}, —(CH₂)₀₋₆OC(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₆—SSR^{∘}; —(CH₂)₀₋₆S(O)₂R^{∘}; —(CH₂)₀₋₆—S(O)₂OR^{∘}; —(CH₂)₀₋₆—OS(O)₂R^{∘}; —S(O)₂NR^{∘}₂; —(CH₂)₀₋₆S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})₂; —OP(O)(R^{∘})OR^{∘}; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring (having 0 to 4 heteroatoms independently selected from nitrogen, oxygen, and sulfur), or, notwithstanding the definition above, two independent occurrences of R^{∘}, taken together with their intervening atom(s), form a 3- to 12-membered saturated, partially unsaturated, or aryl mono- or bicyclic ring (having 0 to 4 heteroatoms independently selected from nitrogen, oxygen, and sulfur), which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur). Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*2)₂₋₃O—, or —S(C(R*₂))₂₋₃S—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur). Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring (having 0 to 4 heteroatoms independently selected from nitrogen, oxygen, and sulfur).

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(RR^{†})S(O)₂RR^{†}; wherein each RR^{†} is independently hydrogen, C₁₋₆ aliphatic which may be substituted as defined below, unsubstituted —OPh, or an unsubstituted 5 to 6-membered saturated, partially unsaturated, or aryl ring (having 0 to 4 heteroatoms independently selected from nitrogen, oxygen, and sulfur), or, notwithstanding the definition above, two independent occurrences of R^{†}, taken together with their intervening atom(s) form an unsubstituted 3 to 12-membered saturated, partially unsaturated, or aryl mono- or bicyclic ring (having 0 to 4 heteroatoms independently selected from nitrogen, oxygen, and sulfur).

Suitable substituents on the aliphatic group of RR^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5 to 6-membered saturated, partially unsaturated, or aryl ring (having 0 to 4 heteroatoms independently selected from nitrogen, oxygen, and sulfur).

As used herein, the term “provided compound” or “compound of the present disclosure” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure.

The terms “patient” and “subject” as used herein refer to humans and mammals, including, but not limited to, primates, cows, sheep, goats, horses, dogs, cats, rabbits, rats, and mice. In one embodiment the subject is a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily or degratorily active metabolite or residue thereof.

The terms “C₁₋₃alkyl,” “C₁₋₅alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₅alky, or C₁₋₆alkyl include, but are not limited to, methyl, ethyl, n-propyl, iso-propyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆cycloalkyl” as used herein refers to a saturated carbocyclic molecule wherein the cyclic framework has 3 to 6 carbon atoms. Representative examples of C₃₋₅cycloalkyl include, but are not limited to, cyclopropyl, cyclobutyl, cyclopentyl, and cyclohexyl.

The terms “diC₁₋₃alkylamino” as used herein refer to —NR*R**, wherein R* and R** independently represent a C₁₋₃alkyl as defined herein. Representative examples of diC₁₋₃alkylamino include, but are not limited to, —N(CH₃)₂, —N(CH₂CH₃)₂, —N(CH₃)(CH₂CH₃), —N(CH₂CH₂CH₃)₂, and —N(CH(CH₃)₂)₂.

The term “C₁₋₃alkoxy” and “C₁₋₆alkoxy” as used herein refer to —OR^{#}, wherein R^{#} represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy.

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈spiroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.0^{1,3}]pentanyl,

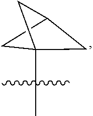 [SMILES: *C12CC(C1)C2]

tricyclo[2.1.1.0^{1,4}]hexanyl, tricyclo[3.1.1.0^{1,5}]hexanyl, and tricyclo[3.2.1.0^{1,5}]octanyl.

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following scheme are the variables as defined for Formula I, unless otherwise noted. All starting materials are either commercially available, for example, from Merck Sigma-Aldrich Inc. and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein. As used below, Z is a leaving group, which can include but is not limited to, halogens (e.g. fluoride, chloride, bromide, iodide), sulfonates (e.g. mesylate, tosylate, benzenesulfonate, brosylate, nosylate, triflate), diazonium, and the like. As used below, in certain embodiments Y is an organometal coupling reagent group, which can include but are not limited to, boronic acids and esters, organotin and organozinc reagents.

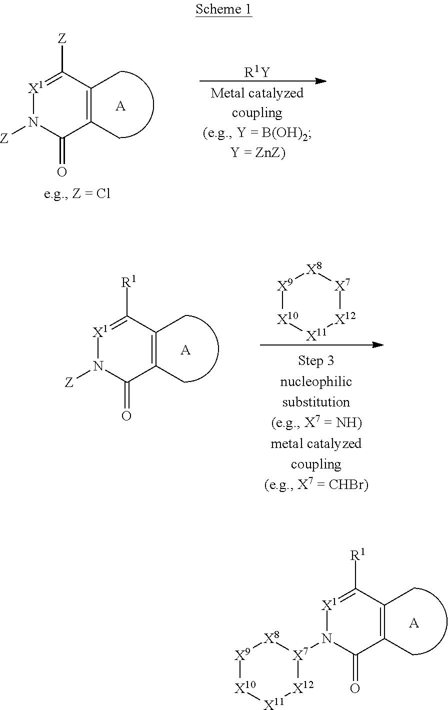

As can be appreciated by the skilled artisan, the above synthetic scheme and representative examples are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds.

Purification methods for the compounds described herein are known in the art and include, for example, crystallization, chromatography (for example, liquid and gas phase), extraction, distillation, trituration, and reverse phase HPLC.

The disclosure further encompasses “intermediate” compounds, including structures produced from the synthetic procedures described, whether isolated or generated in-situ and not isolated, prior to obtaining the finally desired compound. These intermediates are included in the scope of this disclosure. Exemplary embodiments of such intermediate compounds are set forth in the Examples below.

### EXAMPLES

This section provides specific examples of compounds of Formula I and methods of making the same.

List of Abbreviations

| | |
|---|---|
| aq or aq. | aqueous |
| DCM | dichloromethane |
| DMAP | 4-dimethylaminopyridine |
| DMF | N,N-dimethylformamide |
| DMSO | dimethyl sulfoxide |
| Dppf, DPPF or dppf | 1,1′-bis(diphenylphosphino)ferrocene |
| eq or eq. or equiv. | equivalent |
| ESI or ES | electrospray ionization |
| Et | ethyl |
| EtOAc or EA | ethyl acetate |
| g | gram(s) |
| h or hr | hour(s) |
| HPLC | high pressure liquid chromatography |
| iPr | isopropyl |
| iPr₂NEt or DIPEA | N-ethyl diisopropylamine (Hunig's base) |
| LC MS, LCMS, LC-MS or | liquid chromatography mass spectroscopy |
| LC/MS | |
| m/z | mass divided by charge |
| Me | methyl |
| CH₃CN | acetonitrile |
| MeOH | methanol |
| mg | milligrams |
| min | minutes |
| mL | milliliters |
| MS | mass spectra |
| n-BuLi | n-butyllithium |
| NMR | nuclear magnetic resonance |
| PE | Petroleum ether |
| Ph | phenyl |
| RT or rt or r.t. | room temperature |
| RuPhos Pd G3 | (2-Dicyclohexylphosphino-2′,6′-diisopropoxy-1,1′- |
| | biphenyl)[2-(2′-amino-1,1′-biphenyl)]palladium(II) |
| | methanesulfonate |
| sat. | saturated |
| SFC | supercritical fluid chromatography |
| TEA or Et₃N | triethylamine |
| THF | tetrahydrofuran |
| Xantphos Pd G3 | [(4,5-Bis(diphenylphosphino)-9,9-dimethylxanthene)-2-(2′- |
| | amino-1,1′-biphenyl)]palladium(II) methanesulfonate |
| PE | Petroleum ether |

### General Analytical and Purification Methods

Provided in this section are descriptions of the general analytical and purification methods used to prepare the specific compounds provided herein.

### Chromatography:

Unless otherwise indicated, crude product-containing residues were purified by passing the crude material or concentrate through either a Biotage brand silica gel column pre-packed with flash silica (SiO₂) or reverse phase flash silica (C18) and eluting the product off the column with a solvent gradient as indicated. For example, a description of silica gel (0-40% EtOAc/hexane) means the product was obtained by elution from the column packed with silica using a solvent gradient of 0% to 40% EtOAc in hexanes.

### Preparative HPLC Method:

Where so indicated, the compounds described herein were purified via reverse phase HPLC using Waters Fractionlynx semi-preparative HPLC-MS system utilizing one of the following two HPLC columns: (a) Phenominex Gemini column (5 micron, C18, 150×30 mm) or (b) Waters X-select CSH column (5 micron, C18, 100×30 mm).

A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

### Analytical HPLC Method:

Where so indicated, the compounds described herein were analyzed using an Aglilent 1100 series instrument with DAD detector.

### Flash Chromatography Method:

Where so indicated, flash chromatography was performed on Teledyne Isco instruments using pre-packaged disposable SiO₂ stationary phase columns with eluent flow rate range of 15 to 200 mL/min, UV detection (254 and 220 nm).

### Preparative Chiral Supercritical Fluid Chromatography (SFC) Method:

Where so indicated, the compounds described herein were purified via chiral SFC using one of the two following chiral SFC columns: (a) Chiralpak IG 2×25 cm, 5 μm or (b) Chiralpak AD-H 2×15 cm, 5 μm.

Some CP Analytical-SFC experiments were run on SFC Method Station (Thar, Waters) with the following conditions: Column temperature: 40° C., Mobile phase: CO₂/Methanol (0.2% Methanol Ammonia)=Flow: 4.0 ml/min, Back Pressure: 120 Bar, Detection wavelength: 214 nm.

Some CP Analytical-SFC experiments were run on SFC-80 (Thar, Waters) with the following conditions: Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method: Acidic reversed phase MPLC: Instrument type: Reveleris™ prep MPLC; Column: Phenomenex LUNA C18 (3) (150×25 mm, 10); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### Proton NMR Spectra:

Unless otherwise indicated, all ¹H NMR spectra were collected on a Bruker NMR Instrument at 300, 400 or 500 Mhz or a Varian NMR Instrument at 400 Mhz. Where so characterized, all observed protons are reported as parts-per-million (ppm) downfield from tetramethylsilane (TMS) using the internal solvent peak as reference. All NMR were collected at about 25° C.

### Mass Spectra (MS)

Unless otherwise indicated, all mass spectral data for starting materials, intermediates and/or exemplary compounds are reported as mass/charge (m/z), having an [M+H]⁺ molecular ion. The molecular ion reported was obtained by electrospray detection method (commonly referred to as an ESI MS) utilizing a Waters Acquity UPLC/MS system or a Gemini-NX UPLC/MS system. Compounds having an isotopic atom, such as bromine and the like, are generally reported according to the detected isotopic pattern, as appreciated by those skilled in the art.

### Compound Names

The compounds disclosed and described herein have been named using the IUPAC naming function of ChemDraw Professional 17.0.

### Specific Examples

Provided in this section are the procedures to synthesize specific examples of the compounds provided herein. All starting materials are either commercially available from Sigma-Aldrich Inc., unless otherwise noted, or known in the art and may be synthesized by employing known procedures using ordinary skill.

### Example A1: Synthesis of Intermediates

### Method Int-1

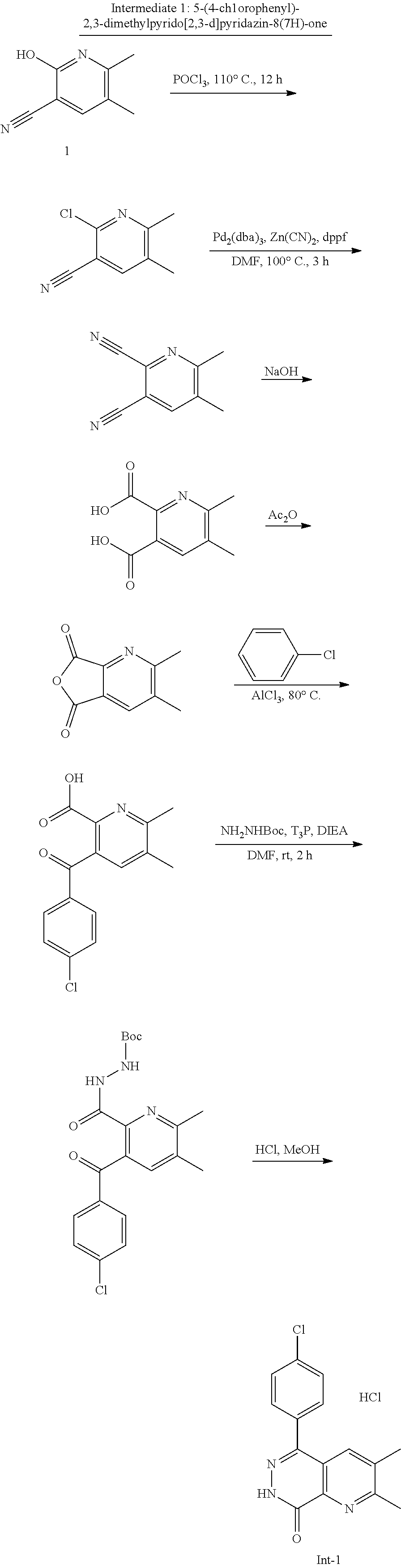

Step 1: A solution of 2-hydroxy-5,6-dimethylnicotinonitrile (1.00 eq, 4500 mg, 30.4 mmol) in POCl₃ (9.51 eq, 27 mL, 289 mmol) was prepared and the mixture was stirred at 20° C. for 1 h. The reaction mixture was heated to 110° C. for 12 h, at which point TLC indicated that the starting material was consumed completely and one new spot formed (PE/EtOAc=0/1, starting material R_{f}=0.5; PE/EtOAc=10/1, new spot R_{f}=0.5). The reaction mixture was poured into saturated aqueous NaHCO₃ (100 mL) and adjusted to pH>7 at 0° C. Then the mixture was extracted with EtOAc (50 mL×3). The organic layer was washed with brine and dried with Na₂SO₄. The solution was concentrated to give a crude residue. The residue was purified by column chromatography (SiO₂, PE/EtOAc=20/1 to 10/1) to afford 2-chloro-5,6-dimethylnicotinonitrile (5000 mg, 30.0 mmol, 98.81% yield) as a white solid. MS: 167.2=[M+H]+, ESI+. ¹H NMR (400 MHz, CDCl₃) δ=7.70 (s, 1H), 2.56 (s, 3H), 2.32 (s, 3H).

Step 2: To a mixture of 1,1′-Bis(diphenylphosphino)ferrocene (0.1000 eq, 1531 mg, 2.76 mmol), Zn(CN)₂ (1.00 eq, 3242 mg, 27.6 mmol) and 2-chloro-5,6-dimethyl-pyridine-3-carbonitrile (1.00 eq, 4600 mg, 27.6 mmol) in DMF (60 mL) was added Pd₂(dba)₃ (0.1000 eq, 1588 mg, 2.76 mmol) at 25° C. under N₂. The mixture was heated to 100° C. under N₂ for 3 h. LCMS showed that the starting material was consumed completely and mass of the desired product was detected (m/z=158.1=[M+H]+, ESI+). The reaction mixture was filtered and quenched by addition of 1 N HCl (100 mL) at 0° C., and then extracted with ethyl acetate (80 mL×2). The combined organic layers were washed with brine (100 mL), dried over Na₂SO₄, filtered and concentrated under reduced pressure to give a residue. The residue was purified by column chromatography (SiO₂, Petroleum ether/Ethyl acetate=1/0 to 3/1; Petroleum ether/Ethyl acetate=3/1, Rf=0.4) to afford 5,6-dimethylpyridine-2,3-dicarbonitrile (4000 mg, 21.9 mmol, 79.27% yield) as a brown solid. MS: 158.2=[M+H]+, ESI+. ¹H NMR (400 MHz, DMSO-d6) δ=8.35 (s, 1H), 2.57 (s, 3H), 2.39 (s, 3H).

Step 3: 5,6-dimethylpyridine-2,3-dicarbonitrile (1.00 eq, 3700 mg, 23.5 mmol) was added to a solution of NaOH (15.0 eq, 14125 mg, 353 mmol) in ethanol (50 mL) and water (50 mL). The mixture was stirred at 100° C. for 16 h. LCMS showed that the starting material was consumed completely and the mass of the desired product was detected (93%, MS: 196.1=[M+H]+, ESI+). The reaction mixture was diluted by addition H₂O (80 mL) at 0° C. and extracted with Ethyl acetate (50 mL×2). The combined aqueous layers were adjusted pH>7 with 1 N HCl (500 mL). The combined aqueous phase was concentrated under vacuum to give a yellow solid. The solid was dissolved in MeOH (50 mL) and filtered. The filtrated was concentrated under vacuum to give crude 5,6-dimethylpyridine-2,3-dicarboxylic acid (6500 mg, 33.3 mmol, 141.47% yield) as yellow solid. MS: 178.0=[M−H₂O+H]+, ESI+. ¹H NMR (400 MHz, DMSO-d₆) δ=7.98 (s, 1H), 2.49 (br s, 3H), 2.32 (s, 3H)

Step 4: 5,6-dimethylpyridine-2,3-dicarboxylic acid (1.00 eq, 6000 mg, 30.7 mmol) was added to Ac₂O (50 mL) to form a solution. The mixture was stirred at 100° C. for 16 h. LCMS of a sample of the reaction mixture quenched by MeOH showed that the starting material was consumed completely and the mass of the desired ester product was detected (58%, MS: 210.1=[M+MeOH]+, ESI+). The mixture was concentrated under reduced pressure to give a residue. The residue was purified by column chromatography (SiO₂, Petroleum ether/Ethyl acetate=1/0 to 1/1; Petroleum ether/Ethyl acetate=1/1, the desired product R_{f}=0.5) to afford 2,3-dimethylfuro[3,4-b]pyridine-5,7-dione (2700 mg, 15.2 mmol, 49.57% yield) as yellow solid. MS: 210.2=[M+MeOH+H]+, ESI+. ¹H NMR (400 MHz, CDCl3) δ=8.00 (d, J=18.7 Hz, 1H), 2.52 (d, J=11.4 Hz, 3H).

Step 5: To a mixture of 2,3-dimethylfuro[3,4-b]pyridine-5,7-dione (1.00 eq, 2700 mg, 15.2 mmol) in PhCl (32.3 eq, 50 mL, 492 mmol) was added AlCl₃ (32.3 eq, 50 mL, 492 mmol) under N₂. Then the reaction mixture was heated to 80° C. for 3 h. LCMS showed that the starting material was consumed completely, the mass of the desired product was detected (20%, MS: 290.0=[M+H]+, ESI+). The reaction mixture was quenched by MeOH (100 mL) at 0° C. and concentrated under reduced pressure to give a residue. The residue was purified by flash silica gel chromatography (ISCO; 120 g SepaFlash Silica Flash Column, Eluent of 0˜10% DCM/MeOH; flow rate: 80 mL/min) to afford 3-(4-chlorobenzoyl)-5,6-dimethylpicolinic acid (1900 mg, 6.56 mmol, 43.03% yield) as a yellow solid. MS: 290.1=[M+H]+, ESI+. ¹H NMR (400 MHz, DMSO-d₆) δ=7.74 (s, 1H), 7.67-7.63 (m, 2H), 7.60-7.55 (m, 2H), 2.56 (s, 3H), 2.36 (s, 3H)

Step 6: To a mixture of 3-(4-chlorobenzoyl)-5,6-dimethyl-pyridine-2-carboxylic acid (1.00 eq, 1700 mg, 5.87 mmol), tert-butyl N-aminocarbamate (2.00 eq, 1551 mg, 11.7 mmol) and DIPEA (5.00 eq, 5.1 mL, 29.3 mmol) in DMF (20 mL) was added 2,4,6-tripropyl-1,3,5,2,4,6-trioxatriphosphinane 2,4,6-trioxide (3.00 eq, 11198 mg, 17.6 mmol). The mixture was stirred at 25° C. for 2 h. LCMS showed the starting material was consumed completely and a major peak with the mass of the desired product was detected (68%, MS: 404.0=[M+H]+, ESI+). The mixture was diluted with water (80 mL) and extracted with ethyl acetate (80 mL×2). The combined organic layers were washed with an aqueous solution, then with brine (100 mL) and then dried over Na₂SO₄. The solvent was filtered and the filtrate was concentrated under reduced pressure. The crude product was purified by column chromatography on silica gel and eluted with petroleum ether/ethyl acetate=1/0 to 0/1 (petroleum ether/ethyl acetate=1/1, the desired product R_{f}=0.5) to give tert-butyl N-[[3-(4-chlorobenzoyl)-5,6-dimethyl-pyridine-2-carbonyl]amino]carbamate (1770 mg, 3.81 mmol, 64.98% yield) as a yellow solid. MS: 403.9=[M+H]+, ESI+. ¹H NMR (400 MHz, CDCl₃) δ=7.74-7.63 (m, 1H), 7.46-7.33 (m, 4H), 2.62 (d, J=19.2 Hz, 3H), 2.37 (d, J=18.5 Hz, 3H), 1.62 (d, J=3.7 Hz, 5H), 1.50-1.47 (m, 9H).

Step 7: Tert-butyl N-[[3-(4-chlorobenzoyl)-5,6-dimethyl-pyridine-2-carbonyl]amino]carbamate (1.00 eq, 1770 mg, 4.38 mmol) was added to a solution of HCl/MeOH (18.3 eq, 20 mL, 80.0 mmol). The mixture was stirred at 25° C. for 2 h. LCMS showed the starting material was consumed completely and a major peak with the mass of the desired product was detected (88%, MS: 286.2=[M+H]+, ESI+). The reaction mixture was concentrated under reduced pressure to give 5-(4-chlorophenyl)-2,3-dimethyl-7H-pyrido[2,3-d]pyridazin-8-one hydrochloride (1450 mg, 4.50 mmol, 102.69% yield) as yellow solid. MS: 285.7=[M+H]+, ESI+. ¹H NMR (400 MHz, DMSO-d₆) δ=13.08 (s, 1H), 7.83 (s, 1H), 7.61 (s, 4H), 2.67 (s, 3H), 2.41 (s, 3H).

### Method Int-2

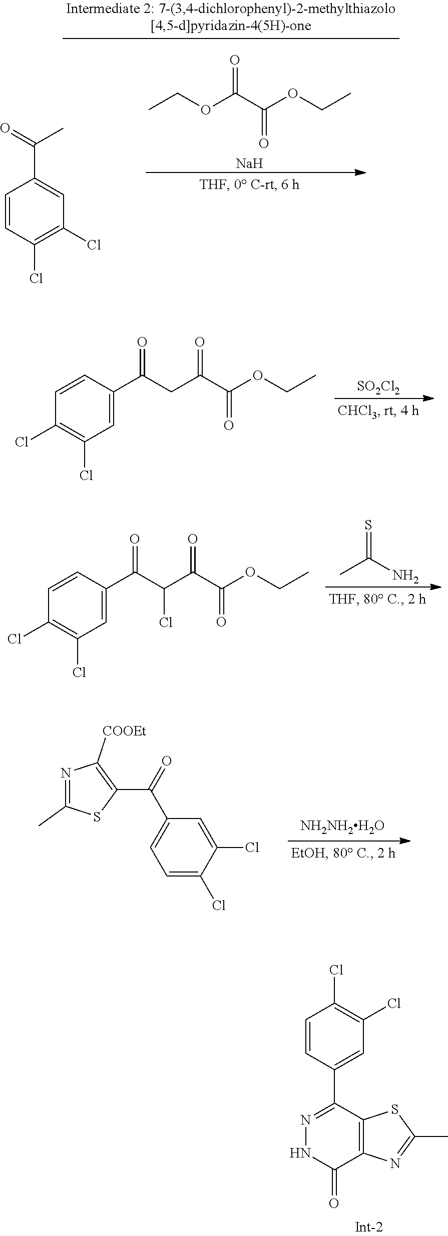

Step 1: To a solution of 1-(3,4-dichlorophenyl)ethanone (5.0 g, 26.5 mmol, 1 eq) in THF (25 mL) was added NaH (1.27 g, 52.9 mmol, 2 eq) in portions at 0° C. After the mixture was stirred for 30 minutes, diethyl oxalate (5.80 g, 39.7 mmol, 1.5 eq) was added dropwise at 0° C. The reaction mixture was warmed up to 25° C. and stirred for 6 hours. The reaction was quenched with HCl (1N, 100 mL) and extracted with EtOAc (100 mL×3). The combined organic phase was washed with brine (100 mL×2), dried over anhydrous sodium sulfate, filtered and concentrated under reduced pressure to give a residue, which was purified by SGC (Petroleum ether/Ethyl acetate=90/10) to afford ethyl 4-(3,4-dichlorophenyl)-2,4-dioxobutanoate (3.6 g, 42.4%) as a yellow oil. LCMS: (M−H)⁺=286.8, Retention time=1.377 min.

Step 2: To a solution of ethyl 4-(3,4-dichlorophenyl)-2,4-dioxo-butanoate (3.6 g, 12.5 mmol, 1 eq) in chloroform (30 mL) at 25° C. under N₂ was added SO₂Cl₂ (8.4 g, 62.3 mmol, 5 eq) dropwise. The reaction mixture was stirred at 25° C. for 4 hours. The mixture was quenched with H₂O (50 mL) and extracted with DCM (50 mL×3). The combined organic phase was washed with brine (50 mL×2), dried over anhydrous sodium sulfate, filtered and concentrated under reduced pressure to give a residue, which was purified by SGC (Petroleum ether/Ethyl acetate=93/7) to afford ethyl 3-chloro-4-(3,4-dichlorophenyl)-2,4-dioxobutanoate (2.5 g, 59.2%) as a yellow oil. LCMS: (M−H)⁺=321.0, Retention time=1.387 min.

Step 3: To a solution of ethyl 3-chloro-4-(3,4-dichlorophenyl)-2,4-dioxo-butanoate (2.2 g, 6.80 mmol, 1 eq) in THF (30 mL) was added ethanethioamide (613 mg, 8.16 mmol, 1.2 eq). The mixture was stirred at room temperature for 2 hours and then heated to 80° C. for 2 hours. After the reaction was completed, the reaction mixture was diluted with H₂O (50 mL) and extracted with EtOAc (50 mL×3). The combined organic phase was washed with brine (50 mL×2), dried over anhydrous sodium sulfate, filtered and concentrated under reduced pressure to give a residue, which was purified by SGC (Petroleum ether/Ethyl acetate=88/12) to afford ethyl 5-(3,4-dichlorobenzoyl)-2-methylthiazole-4-carboxylate (1.5 g, 60.9%) as a white solid. LCMS: (M+H)⁺=344.0, Retention time=1.471 min.

Step 4: To a solution of ethyl 5-(3,4-dichlorobenzoyl)-2-methyl-thiazole-4-carboxylate (500 mg, 1.45 mmol, 1 eq) in ethanol (5 mL) was added hydrazine hydrate (109 mg, 2.18 mmol, 1.5 eq). The mixture was stirred at 80° C. for 2 hours. After reaction was completed, the mixture was filtrated and the filtrate cake was washed with EtOAc. The product was dried under vacuum to afford 7-(3,4-dichlorophenyl)-2-methylthiazolo[4,5-d]pyridazin-4 (5H)-one (Int-2) (350 mg, 73.3%) as a light yellow solid. LCMS: (M+H)⁺=312.1, Retention time=1.231 min. ¹H NMR (400 MHz, DMSO) δ 13.43 (s, 1H), 8.00-8.00 (d, J=2, 1H), 7.85-7.83 (d, J=8.4, 1H), 7.78-7.76 (dd, J=2.4, J=8.4, 1H), 2.88 (s, 3H).

TABLE 1
Int-3 was prepared following the procedure described in Method Int-2, as follows:

| Int # | Structure & Name | Reaction modifications | Characterization Data |
|---|---|---|---|
| 3 | 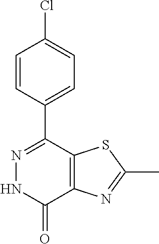 [SMILES: Cc1nc2c(=O)[nH]nc(-c3ccc(Cl)cc3)c2s1] | Using 1-(4- chlorophenyl)ethan-1- one in place of 1-(3,4- dichlorophenyl)ethanone | 1H NMR (400 MHz, DMSO) δ 13.35 (s, 1H), 7.84-7.81 (m, 2H), 7.66-7.62 (m, 2H), 2.88 (s, 3H). LCMS: (M + H)+ = 278 |
| | 7-(4-chlorophenyl)-2- | | |
| | methylthiazolo[4,5- | | |
| | d]pyridazin-4(5H)-one | | |
| 4 | 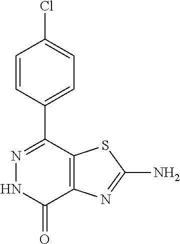 [SMILES: Nc1nc2c(=O)[nH]nc(-c3ccc(Cl)cc3)c2s1] | Using thiourea in place of ethanethioamide | ¹H NMR (400 MHz, DMSO) δ 8.26 (s, 2H), 7.68 − 7.64 (m, 2H), 7.62 − 7.57 (m, 2H), 7.32 − 7.20 (m, 5H), 4.42 (t, J = 7.4 Hz, 2H), 3.09 (t, J = 7.6 Hz, 2H). LCMS (M + H) ⁺ = 383.10 |
| | 2-amino-7-(4- | | |
| | chlorophenyl)thiazolo[4,5- | | |
| | d]pyridazin-4(5H)-one | | |

### Example A2: Synthesis of Exemplary Compounds

### Method 1

### Example 1: 5-(4-chlorophenyl)-2,3-dimethyl-7-(4-(trifluoromethoxy)phenyl)pyrido[2,3-d]pyridazin-8 (7H)-one

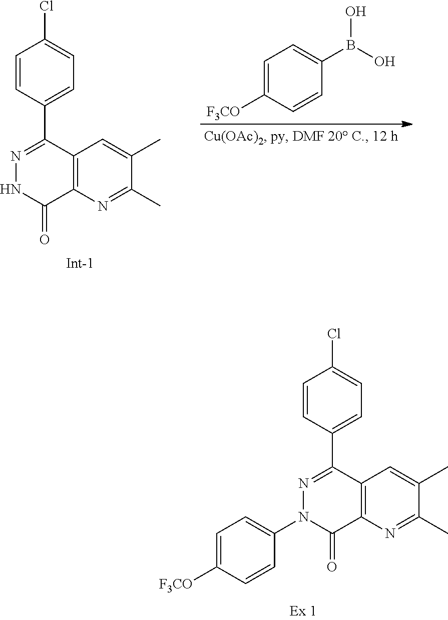 [SMILES: Cc1cc2c(-c3ccc(Cl)cc3)n[nH]c(=O)c2nc1C ; OB(O)c1ccc(OC(F)(F)F)cc1 ; Cc1cc2c(-c3ccc(Cl)cc3)nn(-c3ccc(OC(F)(F)F)cc3)c(=O)c2nc1C]

To a mixture of [4-(trifluoromethoxy)phenyl]boronic acid (2.00 eq, 72 mg, 0.350 mmol) and 5-(4-chlorophenyl)-2,3-dimethyl-7H-pyrido[2,3-d]pyridazin-8-one (1.00 eq, 50 mg, 0.175 mmol) in DMF (1 mL) was added pyridine (3.00 eq, 0.042 mL, 0.525 mmol) and Cu(OAc)₂ (1.10 eq, 35 mg, 0.192 mmol). The mixture was degassed with O₂ 3 times. The mixture was stirred at 25° C. under O₂ for 12 h. LCMS of a sample of the reaction mixture showed starting material was consumed completely and a peak with the mass of the desired product was detected (70%, MS: 445.9=[M+H]+, ESI+). The mixture was filtered and the filtrate was purified by prep-HPLC (FA in water:ACN=100% to 0%, 220 & 254 nm) and lyophilized to give 5-(4-chlorophenyl)-2,3-dimethyl-7-[4-(trifluoromethoxy)phenyl]pyrido[2,3-d]pyridazin-8-one (28 mg, 0.0635 mmol, 36.28% yield) as an off-white solid. MS: 445.8=[M+H]+, ESI+. ¹HNMR (400 MHz, CDCl₃) δ=7.87-7.81 (m, 2H), 7.75 (s, 1H), 7.55 (d, J=1.2 Hz, 3H), 7.33 (d, J=8.8 Hz, 2H), 2.82 (s, 3H), 2.48 (s, 3H).

### Methods 2A, 2B and 2C

### Examples 24, 25 and 26: 7-(4-chlorophenyl)-5-(2-(4-chlorophenyl)-2-oxoethyl)-2-methylthiazolo[4,5-d]pyridazin-4 (5H)-one; 7-(4-chlorophenyl)-5-(2-(4-chlorophenyl)-2-(methylamino)ethyl)-2-methylthiazolo[4,5-d]pyridazin-4 (5H)-one; and 5-(4-chlorophenyl)-2,3-dimethyl-7-[4-(trifluoromethoxy)phenyl]pyrido[2,3-d]pyridazin-8-one

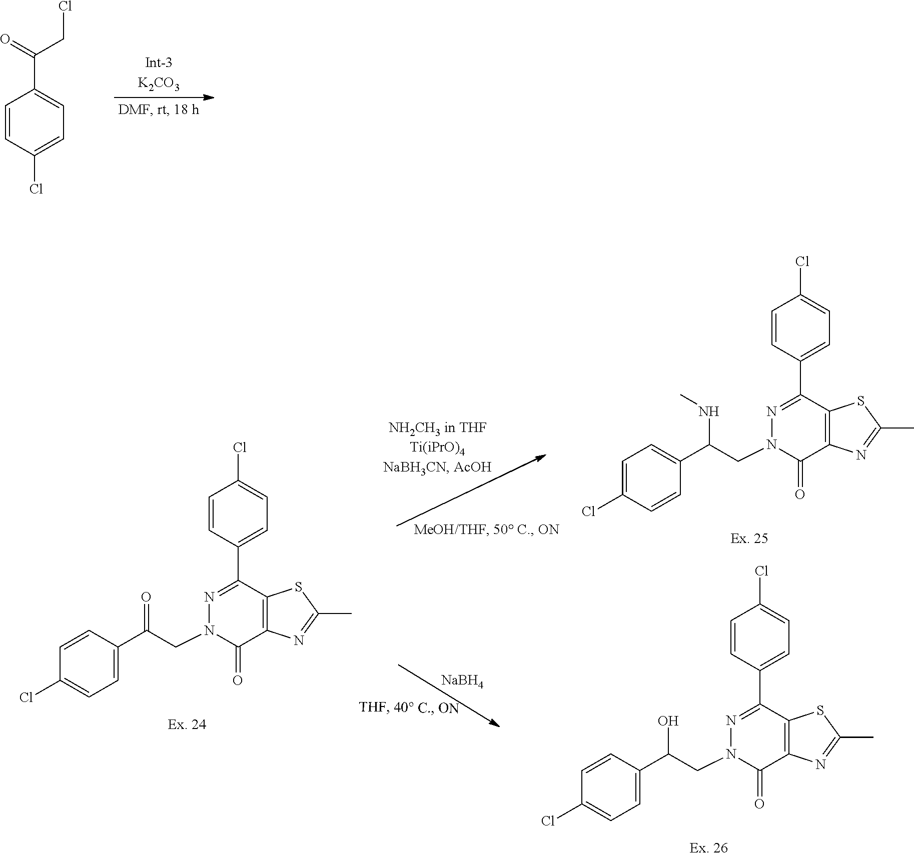 [SMILES: O=C(CCl)c1ccc(Cl)cc1 ; Cc1nc2c(=O)n(CC(=O)c3ccc(Cl)cc3)nc(-c3ccc(Cl)cc3)c2s1 ; "NaBH4" ; Cc1nc2c(=O)n(CC(O)c3ccc(Cl)cc3)nc(-c3ccc(Cl)cc3)c2s1 ; CNC(Cn1nc(-c2ccc(Cl)cc2)c2sc(C)nc2c1=O)c1ccc(Cl)cc1]

Method 2A—Synthesis of Example 24: To a mixture of 7-(4-chlorophenyl)-2-methyl-5H-thiazolo[4,5-d]pyridazin-4-one (Int-3) (200 mg, 7.2 mmol, 1 eq) in DMF (3 mL) were added K₂CO₃ (299 mg, 2.2 mmol, 3 eq) and 2-chloro-1-(4-chlorophenyl)ethan-1-one (204 mg, 1.1 mmol, 1.5 eq). The mixture was stirred at 25° C. under N₂ atmosphere overnight. After the reaction was completed, the mixture was diluted with H₂O (30 mL) and extracted with EtOAc (30 mL×3). The organic layer was washed with brine (50 mL), dried over Na₂SO₄, and concentrated in vacuo to give the crude product. The crude product was purified by silica gel flash chromatographer (Petroleum ether:EtOAc=60:40) to give 7-(4-chlorophenyl)-5-(2-(4-chlorophenyl)-2-oxoethyl)-2-methylthiazolo[4,5-d]pyridazin-4 (5H)-one (Ex. 24) as a light yellow solid (200 mg, 61.3%). LCMS: (M+H)⁺=430.1, Retention time=1.542 min. HPLC: purity=100% (254 nm); purity=99.74% (214 nm); Retention time=3.934 min. ¹H NMR (400 MHz, CDCl3) δ 7.98-7.96 (m, 2H), 7.75-7.72 (m, 2H), 7.51-7.45 (m, 4H), 5.82 (s, 2H), 2.96 (s, 3H).

Method 2B—Synthesis of Example 25: To a mixture of 7-(4-chlorophenyl)-5-[2-(4-chlorophenyl)-2-oxo-ethyl]-2-methyl-thiazolo[4,5-d]pyridazin-4-one (Ex. 24) (200 mg, 0.47 mmol, 1 eq) in THF (5 mL) was added NaCNBH₃ (146 mg, 2.32 mmol, 5 eq). The mixture was stirred at 60° C. under N₂ atmosphere overnight. The mixture was diluted with DCM (50 mL) and water (50 mL), and then extracted with DCM (20 mL×3). The organic layer was combined, washed with brine (20 mL), dried over Na₂SO₄, and concentrated in vacuo to give the crude product. The crude product was purified by prep-HPLC to give 7-(4-chlorophenyl)-5-(2-(4-chlorophenyl)-2-(methylamino)ethyl)-2-methylthiazolo[4,5-d]pyridazin-4 (5H)-one (Ex. 25) as a white solid (44.2 mg, 21.9%). LCMS: (M+H)⁺=432.1, Retention time=1.464 min. HPLC: purity=100% (254 nm); purity=99.68% (214 nm); Retention time=3.688 min. ¹H NMR (400 MHz, DMSO) δ 7.72-7.70 (m, 2H), 7.64-7.62 (m, 2H), 7.39 (s, 4H), 5.72 (s, 1H), 5.14-5.10 (m, 1H), 4.52-4.46 (m, 1H), 4.32-4.27 (m, 1H), 2.88 (s, 3H)

Method 2C—Synthesis of Example 26: To a mixture of [4-(trifluoromethoxy)phenyl]boronic acid (2.00 eq, 72 mg, 0.350 mmol) and 5-(4-chlorophenyl)-2,3-dimethyl-7H-pyrido[2,3-d]pyridazin-8-one (1.00 eq, 50 mg, 0.175 mmol) in DMF (1 mL) was added pyridine (3.00 eq, 0.042 mL, 0.525 mmol) and Cu(OAc)₂ (1.10 eq, 35 mg, 0.192 mmol). The mixture was degassed with O₂ 3 times. The mixture was stirred at 25° C. under 02 for 12 h. LCMS of a sample of the reaction mixture showed starting material was consumed completely and a peak with the mass of the desired product was detected (70%, MS: 445.9=[M+H]+, ESI+). The mixture was filtered and the filtrate was purified by prep-HPLC (FA in water:ACN=100% to 0%, 220 & 254 nm) and lyophilized to give 5-(4-chlorophenyl)-2,3-dimethyl-7-[4-(trifluoromethoxy)phenyl]pyrido[2,3-d]pyridazin-8-one (Ex. 26, 28 mg, 0.0635 mmol, 36.28% yield) as an off-white solid. MS: 445.8=[M+H]+, ESI+. ¹HNMR (400 MHz, CDCl₃) δ=7.87-7.81 (m, 2H), 7.75 (s, 1H), 7.55 (d, J=1.2 Hz, 3H), 7.33 (d, J=8.8 Hz, 2H), 2.82 (s, 3H), 2.48 (s, 3H).

### Methods 3A, 3B and 3C

### Examples 28, 29 and 34: 7-(4-chlorophenyl)-2-methyl-5-phenethylthiazolo[4,5-d]pyridazin-4 (5H)-one; 2-methyl-5-phenethyl-7-phenylthiazolo[4,5-d]pyridazin-4 (5H)-one; and 2-methyl-5-phenethyl-7-(p-tolyl)thiazolo[4,5-d]pyridazin-4 (5H)-one

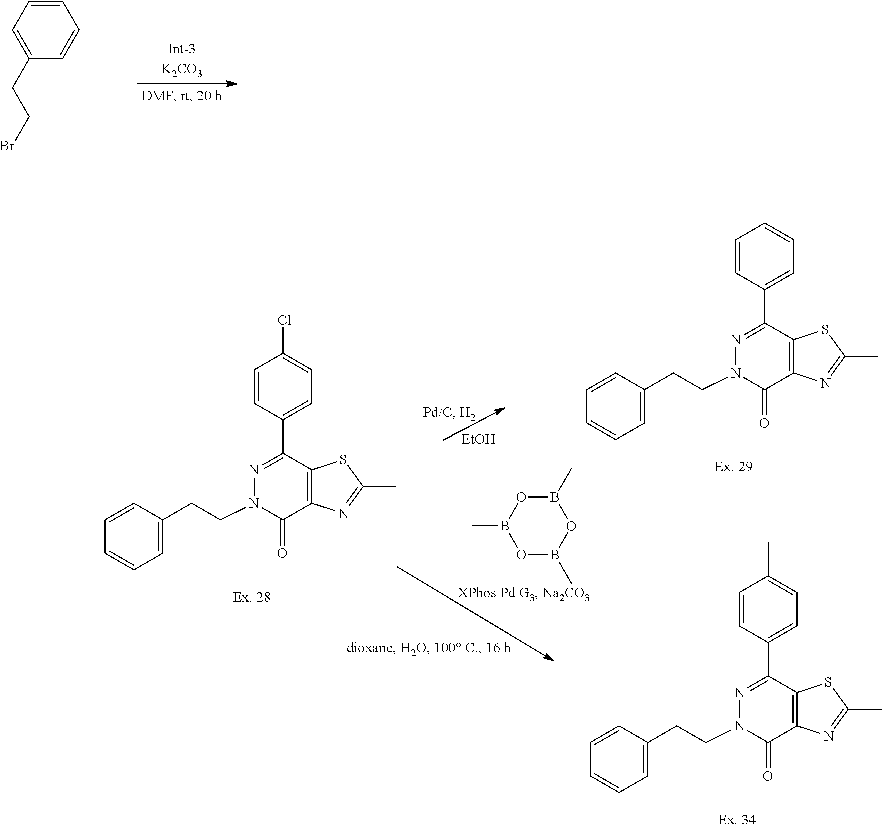

Method 3A—Synthesis of Example 28: To a solution of Int-3 (80 mg, 0.29 mmol, 1.0 eq) in DMF (3.0 mL) was added K₂CO₃ (119 mg, 0.86 mmol, 3.0 eq) and (2-bromoethyl)benzene (266 mg, 1.44 mmol, 5.0 eq). The reaction mixture was stirred at 25° C. for 16 hours. The reaction mixture was diluted with water (20 mL) and extracted with EtOAc (20 mL×2). The organic layer was dried over Na₂SO₄ and evaporated to give the crude product. The residue was purified by SGC (50% ethyl acetate in petroleum ether) to give the desired product 7-(4-chlorophenyl)-2-methyl-5-phenethylthiazolo[4,5-d]pyridazin-4 (5H)-one (Ex. 28) (100 mg, 91%) as a white solid. LCMS: (M+H)⁺=382, Retention time=1.582 min. HPLC: purity=99.23% (254 nm); purity=100% (214 nm); Retention time=3.728 min. ¹H NMR (400 MHz, DMSO) δ 7.72-7.70 (m, 2H), 7.63-7.61 (m, 2H), 7.29-7.18 (m, 5H), 4.52-4.49 (t, J=7.2, 2H), 3.15-3.14 (t, J=7.2, 2H), 2.88 (s, 3H).

Method 3B—Synthesis of Example 29: To a mixture of 7-(4-chlorophenyl)-2-methyl-5-phenethylthiazolo[4,5-d]pyridazin-4 (5H)-one (Ex. 28) (200 mg, 0.52 mmol, 1.00 eq) in ethanol (2 mL) were added Pd/C (111 mg, 1.05 mmol, 2 eq) and Et₃N (5 mg, 0.05 mmol, 0.1 eq). The reaction flask was evacuated and refilled with H₂ three times. Then the reaction mixture was stirred at room temperature for 4 hours. The mixture was filtered and evaporated. The residue was purified with LC8AP prep-HPLC to afford 2-methyl-5-phenethyl-7-phenylthiazolo[4,5-d]pyridazin-4 (5H)-one (Ex. 29) (45 mg, 24.7%) as a white solid. LCMS: (M+H)⁺=348.1, Retention time=1.461 min. HPLC: purity=98.927% (254 nm); purity=99.004% (214 nm); Retention time=4.108 min. ¹H NMR (400 MHz, DMSO) δ 7.73-7.63 (m, 2H), 7.55-7.53 (m, 3H), 7.30-7.20 (m, 5H), 4.51 (t, J=7.3 Hz, 2H), 3.14 (t, J=7.3 Hz, 2H), 2.87 (s, 3H).

Method 3C—Synthesis of Example 34: To a solution of Ex. 28 (200 mg, 0.52 mmol, 1.0 eq) in dioxane/H₂O (=10:1) (5.0 mL) were added Na₂CO₃ (166 mg, 1.57 mmol, 3.0 eq), 2,4,6-trimethyl-1,3,5,2,4,6-trioxatriborinane (328 mg, 2.62 mmol, 5.0 eq), and Xphos-Pd-G3 (44 mg, 0.05 mmol, 0.1 eq). The mixture was stirred at 100° C. for 16 hours. The reaction was diluted with water (20 ml) and extracted with EtOAc (20 ml×3). The combined organic layer was washed with brine, dried and concentrated under reduced pressure. The residue was purified with prep-HPLC to obtain 2-methyl-5-phenethyl-7-(p-tolyl)thiazolo[4,5-d]pyridazin-4 (5H)-one (Ex. 34) (80 mg, 41.4%) as a yellow solid. Method 4

### Example 30: 7-(4-chlorophenyl)-5-(1-(4-chlorophenyl)propan-2-yl)-2-methylthiazolo[4,5-d]pyridazin-4 (5H)-one

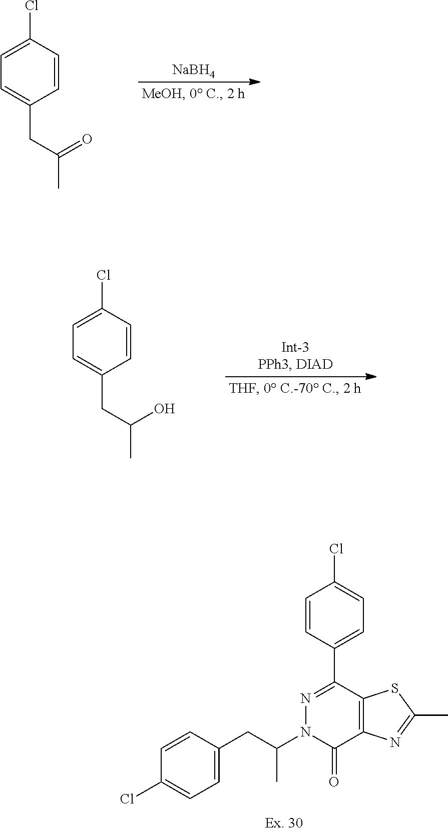 [SMILES: CC(=O)Cc1ccc(Cl)cc1 ; "NaBH4" ; CC(O)Cc1ccc(Cl)cc1 ; Cc1nc2c(=O)n(C(C)Cc3ccc(Cl)cc3)nc(-c3ccc(Cl)cc3)c2s1]

Step 1: To a mixture of 1-(4-chlorophenyl)propan-2-one (1 g, 5.9 mmol, 1.0 eq) in MeOH (10 mL) was added NaBH₄ (0.33 g, 8.9 mmol, 1.5 eq) portionwise at 0° C. under N₂. The mixture was stirred for 2.0 hours. After the reaction was completed, the mixture was quenched with NaHCO₃ (aq) (20 mL), extracted with DCM (20 mL×3), dried over Na₂SO₄, and concentrated in vacuo to give 1-(4-chlorophenyl)propan-2-ol (1.02 g, 95.8%) as a colorless oil.

Step 2: To a mixture of Int-3 (200 mg, 0.72 mmol, 1.0 eq), 1-(4-chlorophenyl)propan-2-ol (135 mg, 0.79 mmol, 1.1 eq), triphenylphosphine (283 mg, 1.08 mmol, 1.5 eq) in THF (5 mL) was added DIAD (218 mg, 1.08 mmol, 1.5 eq) in THF (3 mL) dropwise at 0° C. under N₂. The resulting mixture was stirred at 70° C. for 2 hours. The reaction mixture was diluted with water (20 mL) and extracted with EtOAc (20 mL×2). The organic layer was dried over sodium sulfate and evaporated. The product was purified by prep-HPLC to give 7-(4-chlorophenyl)-5-(1-(4-chlorophenyl)propan-2-yl)-2-methylthiazolo[4,5-d]pyridazin-4 (5H)-one (Ex. 30) (170 mg, 52.1%) as a white solid. LCMS: (M+H)⁺=431, Retention time=1.478 min. HPLC: purity=98.47% (254 nm); purity=95.28% (214 nm); Retention time=5.094 min. ¹H NMR (400 MHz, DMSO) δ 7.89 (d, J=8.6 Hz, 2H), 7.68 (d, J=8.6 Hz, 2H), 7.25 (d, J=8.4 Hz, 2H), 7.16 (d, J=8.4 Hz, 2H), 5.64-5.48 (m, 1H), 3.21-3.05 (m, 2H), 2.85 (s, 3H), 1.42 (d, J=6.6 Hz, 3H).

### Methods 5A and 5B

### Examples 35 and 36: N-[7-(4-chlorophenyl)-4-oxo-5-(2-phenylethyl)thiazolo[4,5-d]pyridazin-2-yl]-2-methyl-propanamide; and 7-(4-chlorophenyl)-2-(cyclopropylmethylamino)-5-(2-phenylethyl)thiazolo[4,5-d]pyridazin-4-one

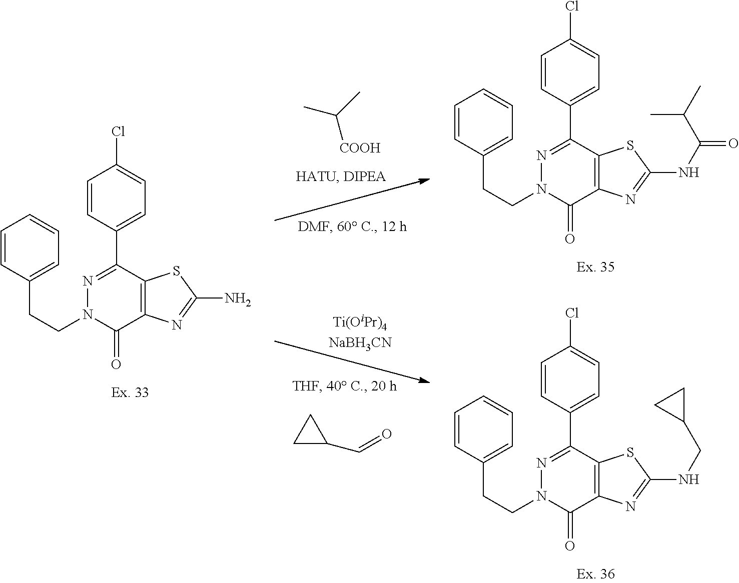

Method 5A—Synthesis of Example 35: To a solution of 2-methylpropanoic acid (17 mg, 0.20 mmol, 1.50 eq) in DMF (2.5 mL) were added HATU (74 mg, 0.20 mmol, 1.50 eq), 2-amino-7-(4-chlorophenyl)-5-(2-phenylethyl)thiazolo[4,5-d]pyridazin-4-one (50 mg, 0.13 mmol, 1.0 eq) and DIEA (0.032 mL, 0.20 mmol, 1.5 eq). The resulting mixture was stirred at 60° C. for 20 hours. After the reaction was cooled to room temperature, the mixture was diluted with water and extracted with ethyl acetate (20 mL×3). The combined organic phase was washed with brine (10 mL×2), dried over anhydrous sodium sulfate, filtrated and concentrated under reduced pressure to give a residue, which was purified by flash chromatography (Petroleum ether/Ethyl acetate=75/25) to give N-[7-(4-chlorophenyl)-4-oxo-5-(2-phenylethyl) thiazolo [4,5-d]pyridazin-2-yl]-2-methyl-propanamide (Ex. 35) as a white solid (25 mg, 41.4%). LCMS (M+H)⁺=453.00, Retention time=1.290 min. HPLC: purity=98.34% (254 nm); purity=97.29% (214 nm); Retention time=4.965 min. ¹H NMR (400 MHz, CDCl₃) δ 11.30 (s, 1H), 7.68 (d, J=8.4 Hz, 2H), 7.47 (d, J=8.4 Hz, 2H), 7.33-7.18 (m, 5H), 4.66 (t, J=7.4 Hz, 2H), 3.24 (t, J=7.4 Hz, 2H), 3.07-2.91 (m, 1H), 1.35 (d, J=5.9 Hz, 6H).

Method 3B—Synthesis of Example 36: To a solution of 2-amino-7-(4-chlorophenyl)-5-(2-phenylethyl) thiazolo [4,5-d]pyridazin-4-one (72 mg, 0.19 mmol, 1.0 eq) in THF (5 mL) were added cyclopropanecarboxaldehyde (132 mg, 1.88 mmol, 10 eq) and Ti(OⁱPr)₄ (160 mg, 0.56 mmol, 3.0 eq). The reaction mixture was stirred at 40° C. for 12 hours. Then NaBH₃CN (47 mg, 0.75 mmol, 4.0 eq) was added and the reaction was stirred at 40° C. for and additional 20 hours. The mixture was diluted with water and extracted with DCM (3×20 mL). The combined organic layer was washed with brine, dried over Na₂SO₄, and concentrated under reduced pressure. The crude product was purified by flash chromatography (DCM) and then further purified by recrystallization in DCM (10 mL) at −15° C. The precipitate was filtered and triturated in MeOH/EtOH (3 mL/3 mL) to give 7-(4-chlorophenyl)-2-(cyclopropylmethylamino)-5-(2-phenylethyl)thiazolo[4,5-d]pyridazin-4-one (Ex. 29) as a white solid. LCMS (M+H)⁺=437.20, Retention time=1.416 min. HPLC: purity=96.46% (254 nm); purity=84.62% (214 nm); Retention time=3.909 min. ¹H NMR (400 MHz, DMSO) δ 8.92 (s, 1H), 7.64 (d, J=8.4 Hz, 2H), 7.59 (d, J=8.4 Hz, 2H), 7.28-7.19 (m, 5H), 4.43 (t, J=7.3 Hz, 2H), 3.28 (t, J=6.0 Hz, 2H), 3.09 (t, J=7.3 Hz, 2H), 1.19-1.02 (m, 1H), 0.63-0.42 (m, 2H), 0.28 (q, J=4.5 Hz, 2H).

### Method 6

### Example 37: 7-(4-chlorophenyl)-5-(2-phenylethyl)-2-(2-phenylethylamino)thiazolo[4,5-d]pyridazin-4-one

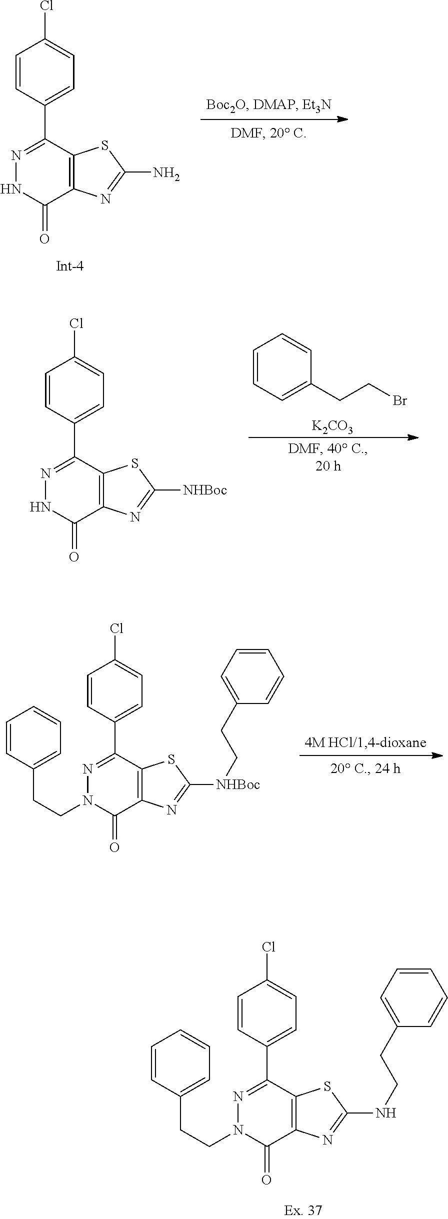

Step 1: To a mixture of 2-amino-7-(4-chlorophenyl)-5H-thiazolo[4,5-d]pyridazin-4-one (Int-4) [115 mg, 0.41 mmol, 1.0 eq] and Boc₂O (108 mg, 0.50 mmol, 1.20 eq) in DMF [5.0 mL] were added DMAP (5.0 mg, 0.041 mmol, 0.1 eq) and Et₃N (63 mg, 0.62 mmol, 1.5 eq). The resulting mixture was stirred at 20° C. for 16 hours. Then the reaction was quenched with saturated NaHCO₃ and extracted with DCM (10 mL×3). The combined organic phase was washed with brine (10 mL×3), dried over anhydrous sodium sulfate, filtrated and concentrated under reduced pressure. The residue was purified by flash chromatography with Petroleum ether/Ethyl acetate (=25/75) to give the tert-butyl N-[7-(4-chlorophenyl)-4-oxo-5H-thiazolo[4,5-d]pyridazin-2-yl]carbamate. (145 mg, 91.8%). LCMS (M+H)⁺=379.13, Retention time=1.199 min

Step 2: To a mixture of tert-butyl N-[7-(4-chlorophenyl)-4-oxo-5H-thiazolo[4,5-d]pyridazin-2-yl]carbamate [145 mg, 0.38 mmol, 1.0 eq] and K₂CO₃ [158 mg, 1.15 mmol, 3.0 eq] in DMF [3 mL] was added (2-bromoethyl) benzene [212 mg, 1.15 mmol, 3.0 eq] dropwise. The resulting mixture was stirred at 40° C. for 12 hours. The reaction mixture was diluted water and extracted with DCM (10 mL×3). The combined organic phase was washed with brine (10 mL×3), dried over anhydrous sodium sulfate, filtrated and concentrated under reduced pressure to give a residue, which was purified by flash chromatography with Petroleum ether/Ethyl acetate (=60/40) to give the tert-butyl N-[7-(4-chlorophenyl)-4-oxo-5-(2-phenylethyl)thiazolo[4,5-d]pyridazin-2-yl]carbamate (78 mg, 34.8%). LCMS [M+H]+: 587.31, Retention time=1.977 min.

Step 3: To a solution of tert-butyl N-[7-(4-chlorophenyl)-4-oxo-5-(2-phenylethyl) thiazolo [4,5-d]pyridazin-2-yl]-N-(2-phenylethyl) carbamate (23 mg, 0.04 mmol, 1.0 eq) in 1,4-dioxane (0.5 mL) was added 4 M HCl in 1,4-dioxane (2.0 mL) dropwise at 0° C. The reaction mixture was allowed to warm to room temperature and stirred for 24 hours. The mixture was concentrated at 45° C. under reduced pressure. The desired product was obtained by trituration in ethyl acetate (5 mL) as a white solid. (5.23 mg, 23.3%). LCMS (M+H)⁺=487.24, Retention time=1.698 min. HPLC: purity=95.75% (254 nm); purity=84.88% (214 nm); Retention time=4.451 min. ¹H NMR (400 MHz, CDCl₃) δ 7.57-7.48 (m, 2H), 7.46 (t, J=6.8 Hz, 2H), 7.35-7.18 (m, 10H), 4.71-4.54 (m, 2H), 3.80-3.56 (m, 2H), 3.24-3.20 (m, 2H), 3.09-3.05 (m, 2H).

### Example 38: 4-(4-chlorophenyl)-2-(6-cyclopropyl-3-pyridyl)-6,7-dimethyl-phthalazin-1-one I-20

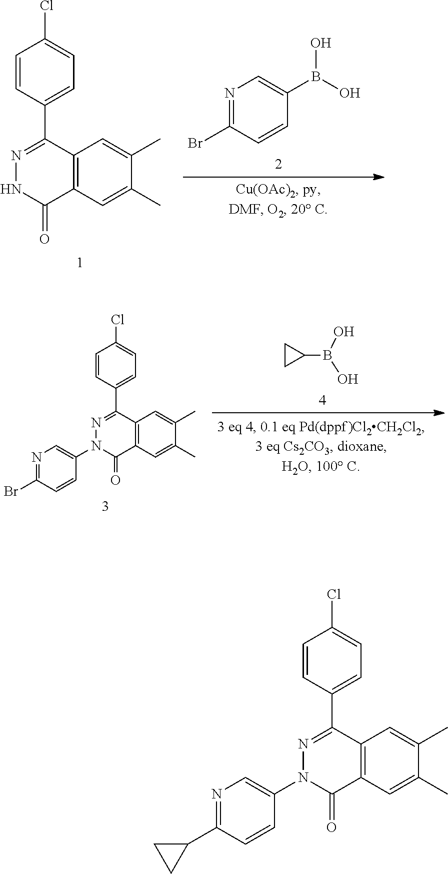

Step 1: A mixture of 4-(4-chlorophenyl)-6,7-dimethyl-2H-phthalazin-1-one (1.00 eq, 200 mg, 0.702 mmol) and (6-bromo-3-pyridyl)boronic acid (2.00 eq, 284 mg, 1.40 mmol) in DMF (8 mL) was added pyridine (3.00 eq, 0.17 mL, 2.11 mmol) and Cu(OAc)₂ (1.10 eq, 140 mg, 0.773 mmol). The mixture was degassed with O₂ for 3 times. The mixture was stirred at 25° C. under O₂ (15 psi) for 12 h. LCMS showed 8% starting material remained and a peak with desired MS (29.9%, MS: 442 [M+H]+, ESI pos). The mixture was added to 40 mL water and extracted with EtOAc (50 mL×3). The organic phase was concentrated under vacuum to give a crude. The crude was added into MeOH (10 mL) and stirred for 30 min. The mixture was filtered and the filter cake was concentrated under vacuum to give 2-(6-bromo-3-pyridyl)-4-(4-chlorophenyl)-6,7-dimethyl-phthalazin-1-one (150 mg, 0.340 mmol, 48.46% yield) as white solid. LCMS: (M+H)⁺=442.1. ¹H NMR (400 MHz, CHLOROFORM-d) δ=8.48-8.40 (m, 1H), 8.37-8.31 (m, 1H), 8.20-8.14 (m, 1H), 8.00-7.94 (m, 1H), 7.61-7.51 (m, 4H), 7.47-7.42 (m, 1H), 2.53-2.48 (m, 3H), 2.44-2.40 (m, 3H).

Step 2: To a mixture of Pd(dppf)Cl₂·CH₂Cl₂ (0.100 eq, 5.5 mg, 0.00681 mmol) and 2-(6-bromo-3-pyridyl)-4-(4-chlorophenyl)-6,7-dimethyl-phthalazin-1-one (1.00 eq, 30 mg, 0.0681 mmol) in 1,4-Dioxane (0.5000 mL) and Water (0.1000 mL) was added Cs₂CO₃ (3.00 eq, 66 mg, 0.204 mmol), cyclopropylboronic acid (3.00 eq, 18 mg, 0.204 mmol). The mixture was degassed with N₂ for 1 min. The mixture was stirred at 100° C. under N₂ for 4 h. LCMS showed no starting material remained and 32.7% desired MS (25%, MS: 401.9 [M+H]+, ESI pos) found. The reaction mixture was diluted with water 5 mL and extracted with EtOAc 30 mL (10 mL*3). The combined organic layers were washed with aq. sat. NaCl (5 mL), dried over [Drying Na₂SO₄], filtered and concentrated under reduced pressure to give a residue. The residue was purified by prep-TLC (SiO₂, PE:EtOAc=2:1, R_{f}=0.25) to give a crude product. The crude product was purified by prep-HPLC (Phenomenex Synergi C18 150*25 mm*10 um, water (FA)-ACN) and lyophilized to give 4-(4-chlorophenyl)-2-(6-cyclopropyl-3-pyridyl)-6,7-dimethyl-phthalazin-1-one (2.5 mg, 0.00622 mmol, 9.14% yield) as white solid. LCMS: (M+H)⁺=401.9; Rt: 0.930 min; 100% purity at 220 nm. H NMR (400 MHz, CHLOROFORM-d) δ=8.54 (d, J=5.8 Hz, 1H), 8.36 (s, 1H), 7.81 (br s, 1H), 7.72 (br d, J=4.3 Hz, 1H), 7.61-7.51 (m, 4H), 7.45 (s, 1H), 2.51 (s, 3H), 2.43 (s, 3H), 2.28-2.16 (m, 1H), 1.14-1.07 (m, 4H).

### Example 39: tert-butyl 4-[4-[4-(4-chlorophenyl)-6,7-dimethyl-1-oxo-phthalazin-2-yl]-2-pyridyl]piperazine-1-carboxylate I-21

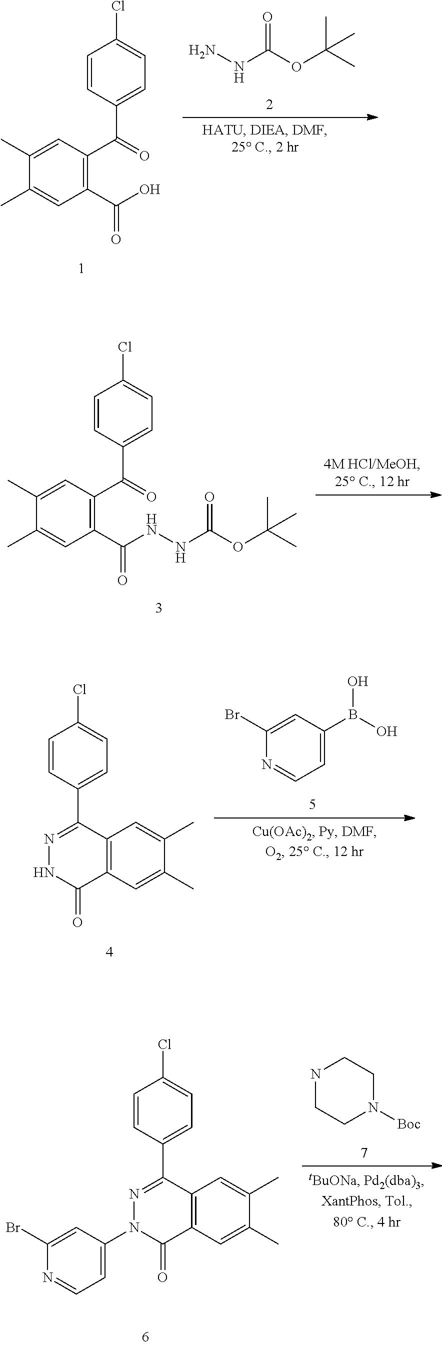

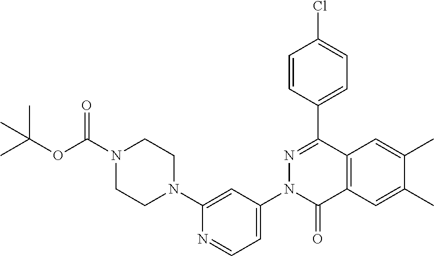 [SMILES: Cc1cc2c(-c3ccc(Cl)cc3)nn(-c3ccnc(N4CCN(C(=O)OC(C)(C)C)CC4)c3)c(=O)c2cc1C]

Step 1: A mixture of 2-(4-chlorobenzoyl)-4,5-dimethyl-benzoic acid (1.00 eq, 1350 mg, 4.68 mmol), tert-butyl N-aminocarbamate (1.10 eq, 680 mg, 5.14 mmol) and DIEA (5.00 eq, 3.9 mL, 23.4 mmol) in DMF (20 mL) was added HATU (2.00 eq, 3556 mg, 9.35 mmol). The mixture was stirred at 25° C. for 2 hours. LCMS (5-95AB/1.5 min): RT=0.706 min, showed 77.9% of new peak. The reaction was diluted with water (100 mL) and then extracted with ethyl acetate (100 mL*3). The combined organic layers were dried over Na₂SO₄, filtered and concentrated under reduced pressure to give a residue. The residue was purified by flash chromatography on silica gel eluting with PE/EtOAc (3:1˜1:1) (TLC, PE EtOAc=1:2, R_{f}=0.40) to afford tert-butyl N-[[2-(4-chlorobenzoyl)-4,5-dimethyl-benzoyl]amino]carbamate (1.90 g, 4.52 mmol, 96.73% yield) as white solid. LCMS: Rt: 0.959 min; [M+H]⁺=424.9; 95.9% purity at 220 nm.

Step 2: A mixture of tert-butyl N-[[2-(4-chlorobenzoyl)-4,5-dimethyl-benzoyl]amino]carbamate (1.00 eq, 1900 mg, 4.52 mmol) in Methanol (20 mL) was added HCl/MeOH (15.0 eq, 17 mL, 67.8 mmol). The mixture was stirred at 25° C. for 12 hours. LCMS (5-95AB/1.5 min): RT=0.840 min, [M+H]⁺ 285.1 showed 91.6% of desired product. The reaction mixture was concentrated under vacuum to give a crude product. Then the crude product was slurried in MeOH and stirred for 15 min. After that, the suspension was filtered by a filter. The filtrate was concentrated under reduced pressure to afford 4-(4-chlorophenyl)-6,7-dimethyl-2H-phthalazin-1-one (970 mg, 3.37 mmol, 74.57% yield) as white solid, LCMS: Rt: 0.663 min; [M+H]⁺=285.0; 99% purity at 220 nm and ¹H NMR (400 MHz, DMSO-d6) δ ppm 2.36 (s, 3H) 2.44 (s, 3H) 7.42 (s, 1H) 7.61 (s, 4H) 8.10 (s, 1H) 12.74 (s, 1H).

Step 3: A mixture of 4-(4-chlorophenyl)-6,7-dimethyl-2H-phthalazin-1-one (1.00 eq, 300 mg, 1.05 mmol) and (2-bromo-4-pyridyl)boronic acid (2.00 eq, 425 mg, 2.11 mmol) in DMF (10 mL) was added pyridine (3.00 eq, 0.26 mL, 3.16 mmol) and Cu(OAc)₂ (1.10 eq, 210 mg, 1.16 mmol). The mixture was degassed with 02 for 3 times. The mixture was stirred at 25° C. under 02 for 12 hours. LCMS (5-95AB/1.5 min): RT=0.825 min, [M+H]⁺ 442.0 showed 17.7% of desired product. The mixture was added to 150 mL water and extracted with EtOAc (150 mL×3). The organic phase was concentrated under vacuum to give a crude. The crude was added MeOH and a lot of solid remained. The mixture was stirred for 0.5 h and filtered. The filter cake was concentrated under vacuum to give 2-(2-bromo-4-pyridyl)-4-(4-chlorophenyl)-6,7-dimethyl-phthalazin-1-one (370 mg, 0.817 mmol, 77.53% yield) as white solid. LCMS: Rt: 1.008 min; [M+H]⁺=442.0; 97.3% purity at 220 nm.

Step 4: To a solution of 2-(2-bromo-4-pyridyl)-4-(4-chlorophenyl)-6,7-dimethyl-phthalazin-1-one (1.00 eq, 100 mg, 0.227 mmol) in toluene (3 mL) was added tert-butyl piperazine-1-carboxylate (1.20 eq, 51 mg, 0.272 mmol), tBuONa (1.50 eq, 33 mg, 0.340 mmol), Pd₂(dba)₃ (0.200 eq, 26 mg, 0.0454 mmol) and XantPhos (0.0700 eq, 9.2 mg, 0.0159 mmol) at 25° C. The reaction mixture was degassed with N₂ for 3 times. The mixture was stirred at 80° C. under N₂ for 12 hours. LCMS (5-95AB/1.5 min): RT=0.711 min, 546.3=[M+H]+, ESI+, showed 64.6% of desired product. The reaction mixture was concentrated under reduced pressure to afford a residue. The residue was purified by prep-TLC (PE:EtOAc=2:1, R_{f}=0.4) to afford crude product as yellow solid, which confirmed by LCMS (5-95AB/1.5 min): RT=0.709 min, 546.3=[M+H]+, ESI+, showed 88.1% of product. The crude product was further purified by prep-HPLC (Column, [Phenomenex luna C18 150*25 mm*10 um]; mobile phase: [ACN] and [H₂O](conditions: [water (0.225% FA)-ACN], B %: 55%-85%; Detector, UV 254 nm. RT: [10 min]) to afford tert-butyl 4-[4-[4-(4-chlorophenyl)-6,7-dimethyl-1-oxo-phthalazin-2-yl]-2-pyndyl]piperazine-1-carboxylate (59 mg, 0.104 mmol, 45.78% yield) as gray solid. LCMS: Rt: 0.905 min; [M+H]⁺=546.0; 96.8% purity at 220 nm and ¹H NMR (400 MHz, DMSO-d6) δ ppm 1.42 (s, 9H) 2.40 (s, 3H) 2.47 (s, 3H) 3.42 (br s, 4H) 3.48-3.53 (m, 4H) 7.04 (d, J=5.50 Hz, 1H) 7.20 (s, 1H) 7.49 (s, 1H) 7.61-7.74 (m, 4H) 8.20-8.24 (m, 2H).

### Example 40: 2-(1,3-benzodioxol-5-yl)-4-(4-chlorophenyl)-6,7-dimethyl-phthalazin-1-one I-22

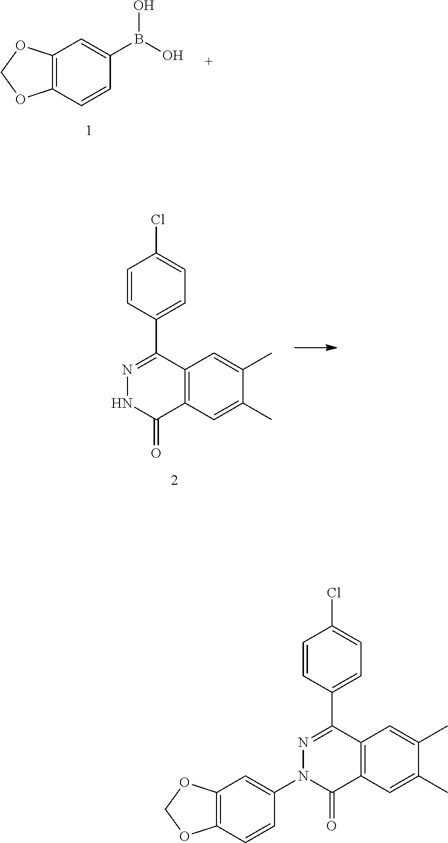 [SMILES: OB(O)c1ccc2c(c1)OCO2 ; Cc1cc2c(-c3ccc(Cl)cc3)n[nH]c(=O)c2cc1C ; Cc1cc2c(-c3ccc(Cl)cc3)nn(-c3ccc4c(c3)OCO4)c(=O)c2cc1C]

Step 1: A mixture of 4-(4-chlorophenyl)-6,7-dimethyl-2H-phthalazin-1-one (1.00 eq, 80 mg, 0.281 mmol) and 1,3-benzodioxol-5-ylboronic acid (2.00 eq, 93 mg, 0.562 mmol) in DMF (2 mL) was added pyridine (3.00 eq, 0.068 mL, 0.843 mmol) and Cu(OAc)₂ (1.10 eq, 56 mg, 0.309 mmol). The mixture was degassed with O₂ for 3 times. The mixture was stirred at 80° C. for 12 h under O₂ (15 psi). TLC (PE/EA=2/1) showed raw material was consumed and two new spots formed. LCMS showed the raw material was consumed and the major peak with desired MS (404.8 [M+H]+; ESI+), The reaction mixture was poured into water (20 mL) and then extracted with DCM (15 mL*2) and the organics washed with 10 mL saturated brine solution. The organics were then separated and dried (Na₂SO₄) before concentration to dryness. The crude was then purified by prep-TLC (DCM, R_{f}=0.4) and freeze-drying to give 2-(1,3-benzodioxol-5-yl)-4-(4-chlorophenyl)-6,7-dimethyl-phthalazin-1-one (16 mg, 0.0405 mmol, 14.42% yield) as white solid which was confirmed by LCMS, H NMR. LCMS: (M+H)+=404.8; purity=100% (UV=220 nm); Retention time=1.038 min). 1H NMR (400 MHz, CHLOROFORM-d) δ=8.37-8.31 (m, 1H), 7.61-7.49 (m, 4H), 7.46-7.42 (m, 1H), 7.19-7.12 (m, 2H), 6.92-6.87 (m, 1H), 6.04-6.01 (m, 2H), 2.52-2.48 (m, 3H), 2.44-2.40 (m, 3H).

### Example 41: tert-butyl 3-[[4-[4-(4-chlorophenyl)-6,7-dimethyl-1-oxo-phthalazin-2-yl]-2-pyridyl]oxy]azetidine-1-carboxylate I-23

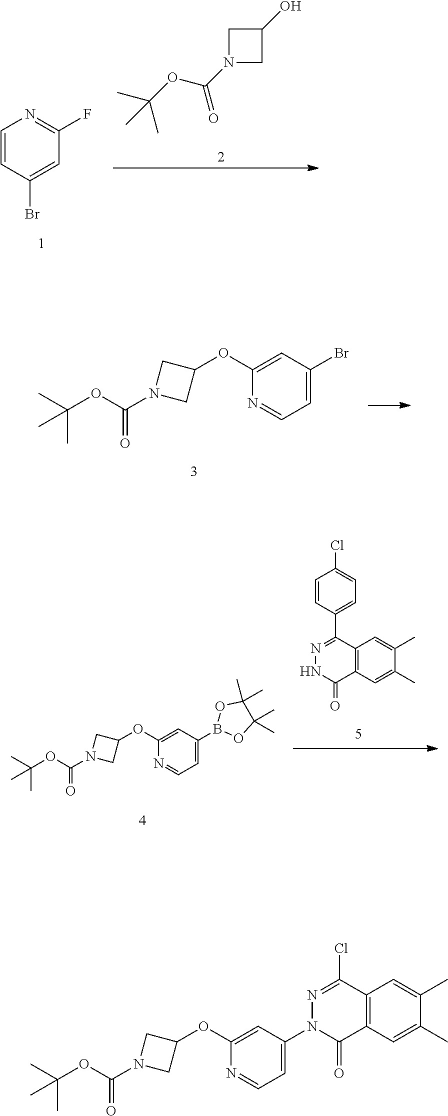

Step 1: To a solution of tert-butyl 3-hydroxyazetidine-1-carboxylate (1.00 eq, 0.98 g, 5.68 mmol) in DMF (10 mL) was added NaH (1.00 eq, 0.23 g, 5.68 mmol) at 0° C. and stirred for 0.5 h and then 4-bromo-2-fluoro-pyridine (1.00 eq, 1.00 g, 5.68 mmol) was added to the mixture and stirred for 16 h at 25° C. LCMS showed the raw material was consumed completely and the major peak showed desired MS (274.7 [M−C₄H₈⁺²]+; ESI+). The mixture was added water (2 mL) slowly and suspension turned to be clear solution. Then the mixture was poured into water (30 mL) and extracted with EtOAc (20 mL*2) and the organics washed with 10 mL saturated brine solution. The organics were then separated and dried (Na₂SO₄) before concentration to dryness. The crude was then purified by silica gel column (PE/EA=10/1, R_{f}=0.6) to give tert-butyl 3-[(4-bromo-2-pyridyl)oxy]azetidine-1-carboxylate (1.10 g, 3.34 mmol, 58.81% yield) as colorless oil which was confirmed by H NMR: ¹H NMR (400 MHz, CHLOROFORM-d) δ=7.95-7.91 (m, 1H), 7.08-7.04 (m, 1H), 7.01-6.98 (m, 1H), 5.34-5.26 (m, 1H), 4.35-4.28 (m, 2H), 3.99-3.92 (m, 2H), 1.47-1.44 (m, 10H).

Step 2: To a solution of tert-butyl 3-[(4-bromo-2-pyridyl)oxy]azetidine-1-carboxylate (1.00 eq, 1.00 g, 3.04 mmol) in 1,4-Dioxane (20 mL) was added 4,4,5,5-tetramethyl-2-(4,4,5,5-tetramethyl-1,3,2-dioxaborolan-2-yl)-1,3,2-dioxaborolane (1.50 eq, 1.16 g, 4.56 mmol) and KOAc (2.50 eq, 745 mg, 7.59 mmol), then Pd(dppf)Cl₂·CH₂Cl₂ (0.100 eq, 246 mg, 0.304 mmol) was added to the mixture under N₂. The reaction was stirred for 3 h at 100° C. under N₂. TLC (PE/EA=3/1) showed raw material was consumed (uv, R_{f}=0.5) and the new blue spot (Cerium Ammonium Molybdate, R_{f}=0.2) formed. LCMS showed the raw material was consumed completely and the major peak formed. The reaction was pour into water (30 mL) and then extracted with EtOAc (20 mL*2) and the organics washed with 10 mL saturated brine solution. The organics were then separated and dried (Na₂SO₄) before concentration to dryness. The crude purified by silica gel column (PE/EA=1/0-3/1) to give tert-butyl 3-[[4-(4,4,5,5-tetramethyl-1,3,2-dioxaborolan-2-yl)-2-pyridyl]oxy]azetidine-1-carboxylate (1.10 g, 2.92 mmol, 96.24% yield) as white solid which was confirmed by 1H NMR (400 MHz, CHLOROFORM-d) δ=8.14-8.09 (m, 1H), 7.23-7.20 (m, 1H), 7.18-7.14 (m, 1H), 5.36-5.28 (m, 1H), 4.36-4.28 (m, 2H), 4.00-3.92 (m, 2H), 1.36-1.33 (m, 12H).

Step 3: To a solution of 4-(4-chlorophenyl)-6,7-dimethyl-2H-phthalazin-1-one (1.00 eq, 60 mg, 0.211 mmol) and tert-butyl 3-[[4-(4,4,5,5-tetramethyl-1,3,2-dioxaborolan-2-yl)-2-pyridyl]oxy]azetidine-1-carboxylate (2.00 eq, 159 mg, 0.421 mmol) in DMF (4 mL) was added pyridine (3.00 eq, 0.051 mL, 0.632 mmol) and Cu(OAc)₂ (1.10 eq, 42 mg, 0.232 mmol) and then the mixture was degassed with O₂ for three times and then stirred for 16 h at 80° C. under O₂ (15 psi). LCMS showed the raw material was consumed most and the major peak showed desired MS (533.0 [M+H]+; ESI+). The reaction was pour into water (20 mL) and extracted with EtOAc (15 mL*2) and the organics washed with 10 mL saturated brine solution. The organics were then separated and dried (Na₂SO₄) before concentration to dryness. The crude was then purified by prep-TLC (PE/EA=2/1, R_{f}=0.5) twice and freeze-drying to give tert-butyl 3-[[4-[4-(4-chlorophenyl)-6,7-dimethyl-1-oxo-phthalazin-2-yl]-2-pyridyl]oxy]azetidine-1-carboxylate (14 mg, 0.0262 mmol, 12.44% yield) as white solid which was confirmed by LCMS. LCMS: (M+H)⁺=533.0; purity=99.084% (UV=220 nm); Retention time=1.157 min. 1H NMR (400 MHz, CHLOROFORM-d) δ=8.38-8.34 (m, 1H), 8.20-8.15 (m, 1H), 7.61-7.51 (m, 5H), 7.47-7.44 (m, 1H), 7.39-7.36 (m, 1H), 5.39-5.33 (m, 1H), 4.38-4.31 (m, 2H), 4.04-3.97 (m, 2H), 2.52-2.49 (m, 3H), 2.44-2.41 (m, 3H), 1.47-1.44 (m, 9H).

### Example 41: 6,7-dichloro-4-(4-chlorophenyl)-2-(1-methyl-2-oxopiperidin-4-yl)phthalazin-1 (2H)-one I-24

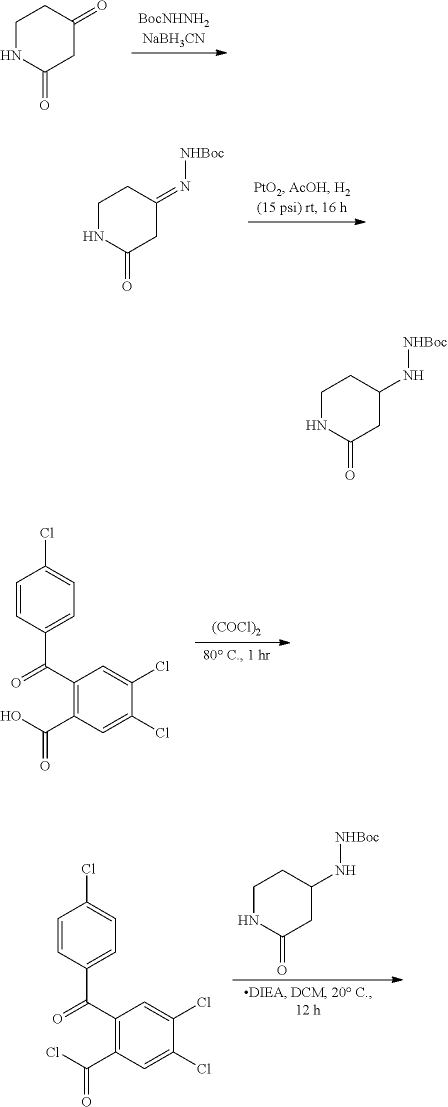

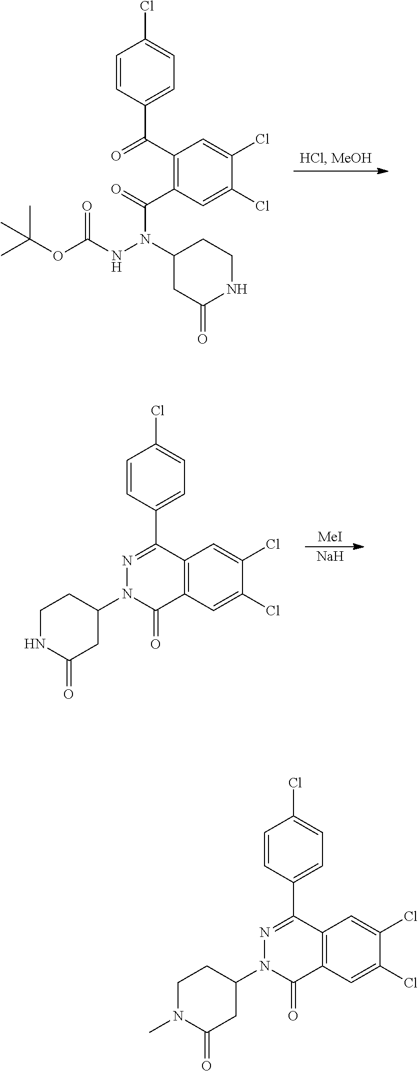

Step 1: A mixture of piperidine-2,4-dione (1.00 eq, 500 mg, 4.42 mmol) and tert-butyl hydrazinecarboxylate (1.00 eq, 584 mg, 4.42 mmol) in MeOH (10 mL) was stirred at 25° C. for 12 h. then NaBH₃CN (10.0 eq, 2786 mg, 44.2 mmol) was added and the mixture was stirred at 25° C. for 2 h. LCMS showed that the desired mass was detected. The mixture was concentrated under reduced pressure to give a residue. The residue was purified by column chromatography (SiO₂, PE/EtOAc=1/0 to 0/1 (0.1% NH₃·H₂O), PE/EtOAc=0/1, R_{f}=0.6) to afford tert-butyl (E)-2-(2-oxopiperidin-4-ylidene)hydrazine-1-carboxylate (860 mg, 3.78 mmol, 85.61% yield) as yellow solid, checked by H NMR: HW-2021-01-015-P1A. (M+H)⁺=228.1; purity=99% (220 nm); Retention time=0.373 min. ¹H NMR (400 MHz, DMSO-d₆) δ=8.83 (br s, 1H), 8.10 (s, 1H), 6.51 (br s, 1H), 4.45 (d, J=1.0 Hz, 1H), 3.15 (dt, J=2.2, 6.8 Hz, 2H), 2.21 (t, J=6.8 Hz, 2H), 1.44-1.39 (m, 9H)

Step 2: To a solution of tert-butyl (E)-2-(2-oxopiperidin-4-ylidene)hydrazine-1-carboxylate (1.00 eq, 500 mg, 2.20 mmol) in Ethanol (10 mL) and acetic acid (36.1 eq, 4.5 mL, 79.4 mmol) was added PtO₂ (0.200 eq, 100 mg, 0.441 mmol) under N₂. The suspension was degassed under vacuum and purged with H₂ three times. The mixture was stirred under H₂ (15 psi) at 25° C. for 16 hours. LCMS showed that the starting material was consumed completely and the desired mass was detected (98%, MS: 459.2 [2M+H]⁺, ESI pos). The mixture was filtered and concentrated under reduced pressure to give a residue. The residue was purified by column chromatography (SiO₂, DCM/MeOH=1/0 to 10/1; DCM:MeOH=10/1, the desired product R_{f}=0.25) to afford tert-butyl 2-(2-oxopiperidin-4-yl)hydrazine-1-carboxylate (320 mg, 1.34 mmol, 60.90% yield) as white solid, checked by LCMS and H NMR: (2M+H)⁺=459.1; purity=96% (220 nm); Retention time=0.740 min. ¹H NMR (400 MHz, DMSO-d6) δ=8.25 (br s, 1H), 7.46-7.27 (m, 1H), 4.52 (br s, 1H), 3.27-3.18 (m, 2H), 3.02-2.91 (m, 1H), 2.23 (dd, J=5.1, 17.4 Hz, 1H), 2.00-1.87 (m, 1H), 1.76-1.66 (m, 1H), 1.55-1.44 (m, 1H), 1.38 (s, 9H)

Step 3: A solution of 4,5-dichloro-2-(4-chlorobenzoyl)benzoic acid (1.00 eq, 100 mg, 0.303 mmol) in oxalyl dichloride (1.00 eq, 39 mg, 0.303 mmol) and was heated to 80° C. for 1 h. LCMS showed that the starting material was consumed completely and detected the desired mass (78%, MS: 344.9 [M−Cl+MeOH+2]⁺, ESI pos). The mixture was concentrated under reduced pressure to give 4,5-dichloro-2-(4-chlorobenzoyl)benzoyl chloride (105 mg, 0.302 mmol, 99.43% yield) as white solid, which was used directly for the next step. [M−Cl+MeOH+2]⁺=344.9; purity=78% (220 nm); Retention time=0.771 min.

Step 4: To a mixture of pyridine (3.00 eq, 0.073 mL, 0.905 mmol) and tert-butyl 2-(2-oxopiperidin-4-yl)hydrazine-1-carboxylate (2.00 eq, 138 mg, 0.603 mmol) in DCM (1 mL) was added a solution of 4,5-dichloro-2-(4-chlorobenzoyl)benzoyl chloride (1.00 eq, 105 mg, 0.302 mmol) in DCM (1 mL) and DMF (2 mL). The mixture was stirred at 25° C. for 2 h. LCMS: HW-2021-01-052-P1A (a drop of the mixture was quenched by MeOH) showed that the starting material was remained. The mixture was stirred at 25° C. for 12 h. LCMS: HW-2021-01-052-P1A1 (a drop of the mixture was quenched by MeOH) showed the starting material was consumed completely and the desired mass was detected (47%, MS: 485.9 [M−55]⁺, ESI pos). The residue was purified by column chromatography (SiO₂, PE/EtOAc=1/0 to 0/1; PE/EtOAc=1/1, R_{f}=0.3) to give tert-butyl 2-(4,5-dichloro-2-(4-chlorobenzoyl)benzoyl)-2-(2-oxopiperidin-4-yl)hydrazine-1-carboxylate (110 mg, 0.203 mmol, 67.41% yield) as yellow solid, checked by LCMS and H NMR [M−tBu+H+2]⁺=487.7; purity=97% (220 nm); Retention time=0.973 min. n¹H NMR (400 MHz, CDCl₃) δ=7.80-7.73 (m, 2H), 7.70-7.65 (m, 1H), 7.58-7.48 (m, 3H), 7.45-7.40 (m, 1H), 4.93-4.79 (m, 1H), 3.53-3.32 (m, 2H), 2.64 (br d, J=9.5 Hz, 2H), 2.30-2.20 (m, 2H), 1.30-1.25 (m, 9H).

Step 5: A solution of tert-butyl 2-(4,5-dichloro-2-(4-chlorobenzoyl)benzoyl)-2-(2-oxopiperidin-4-yl)hydrazine-1-carboxylate (1.00 eq, 110 mg, 0.203 mmol) in HCl/MeOH (216 eq, 11 mL, 44.0 mmol) was stirred at 25° C. for 2 h. LCMS showed the starting material was consumed completely and a major peak with desired MS (70%, MS: 421.9 [M+H]+, ESI pos) was detected. The mixture was concentrated under reduced pressure to give a residue. The residue was purified by prep-TLC (SiO₂, DCM/MeOH=10/1) to afford 6,7-dichloro-4-(4-chlorophenyl)-2-(2-oxopiperidin-4-yl)phthalazin-1 (2H)-one (50 mg, 0.117 mmol, 57.63% yield) as white solid, checked by LCMS: [M+H]⁺=422.0; purity=94% (220 nm); Retention time=0.903 min.

Step 6: To a solution of 6,7-dichloro-4-(4-chlorophenyl)-2-(2-oxopiperidin-4-yl)phthalazin-1 (2H)-one (1.00 eq, 40 mg, 0.0946 mmol) in THF (1 mL) was added NaH (2.00 eq, 7.6 mg, 0.189 mmol) at 0° C. Then Mel (3.00 eq, 0.018 mL, 0.284 mmol) was added and stirred at 25° C. for 12 h. LCMS showed that a little starting material remained and two peaks with the desired mass (65%, MS: 436.1 [M+H]⁺, ESI pos). The reaction mixture was quenched by MeOH (10 mL) and concentrated under reduced pressure to give a residue. The residue was purified by prep-TLC (SiO₂, PE/EtOAc=1/1; the desired product R_{f}=0.45, the starting material R_{f}=0.4) to afforded 6,7-dichloro-4-(4-chlorophenyl)-2-(1-methyl-2-oxopiperidin-4-yl)phthalazin-1 (2H)-one (13 mg, 0.0286 mmol, 30.27% yield) as white solid. [M+H]⁺=437.7; purity=95.5% (220 nm); Retention time=0.978 min. ¹H NMR (400 MHz, CDCl₃) δ=8.60 (s, 1H), 7.83 (s, 1H), 7.56-7.52 (m, 2H), 7.51-7.47 (m, 2H), 5.52 (ddt, J=3.5, 5.8, 9.6 Hz, 1H), 3.52-3.44 (m, 1H), 3.35 (td, J=5.0, 12.4 Hz, 1H), 2.99-2.92 (m, 4H), 2.82-2.74 (m, 1H), 2.40-2.30 (m, 1H), 2.23-2.15 (m, 1H).

### Example 42: tert-butyl 4-[4-[4-(4-chlorophenyl)-6,7-dimethyl-1-oxo-phthalazin-2-yl]-2-pyridyl]piperidine-1-carboxylate I-25

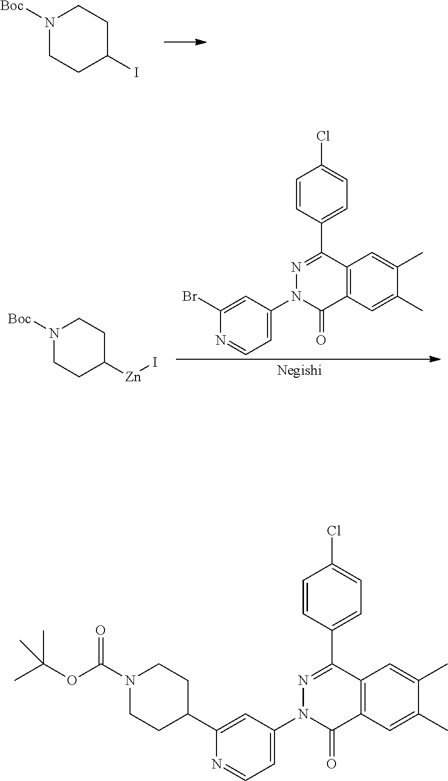

Step 1: Zinc (3 eq, 126 mg, 1.93 mmol) was suspended in LiCl (0.5 M in THF) (1.00 eq, 1.5 mL, 0.643 mmol). 1,2-Dibromoethane (0.0500 eq, 0.0028 mL, 0.0321 mmol) was added and the suspension was stirred at 55° C. for 20 min. Cooled down, then TMSCl (0.0500 eq, 0.0041 mL, 0.0321 mmol) was introduced and the mixture was stirred at 55° C. for 20 min. Cooled down then iodine (0.0200 eq, 3.3 mg, 0.0129 mmol) in THF (0.5 mL) was introduced and the reaction was stirred at 55° C. for additional 20 min. Tert-butyl 4-iodopiperidine-1-carboxylate (1.00 eq, 200 mg, 0.643 mmol) in THF (1.5 mL) (99.5%, Extra Dry over Molecular Sieve, Stabilized, Acros) was then added to the warm (55° C.) suspension of activated zinc. The mixture was stirred at 55° C. overnight. The mixture solution (3 mL) was used for next step directly without further purification.

Step 2: To a solution of 2-(2-bromo-4-pyridyl)-4-(4-chlorophenyl)-6,7-dimethyl-phthalazin-1-one (1.00 eq, 90 mg, 0.204 mmol) and C-phos (0.100 eq, 8.9 mg, 0.0204 mmol) in THF (2.5 mL) (99.5%, Extra Dry over Molecular Sieve, Stabilized, Acros) was added Pd(OAc)₂ (0.0500 eq, 2.3 mg, 0.0102 mmol). The mixture was degassed with N₂ for 3 times and (1-tert-butoxycarbonyl-4-piperidyl)-iodo-zinc (1.06 eq, 2.5 mL, 0.217 mmol) was added, then the mixture was stirred at 55° C. for 2 h. LCMS showed that ˜39% desired MS (39%, MS: 545.1 [M+H]+, ESI pos) was detected. The mixture was cooled to rt, diluted with EtOAc (20 mL) and water (30 mL). The phase were separated and extracted by EtOAc (30 mL×2). The organic phase was washed with brine and dried over Na₂SO₄, filtered and concentrated in vacuo. The residue was purified by prep-TLC (PE:EtOAc=1:1, UV, R_{f}=0.3) to give a residue. The residue was purified by reversed phase-HPLC (FA condition; 0.05% FA in water:CAN=0% to 100%, 220&254 nm) and lyophilized to give tert-butyl 4-[4-[4-(4-chlorophenyl)-6,7-dimethyl-1-oxo-phthalazin-2-yl]-2-pyridyl]piperidine-1-carboxylate (33 mg, 0.0580 mmol, 28.40% yield) as off-white solid. LCMS: (M+H)+=545.1; purity=95.8% (UV 220 nm); Retention time=0.723 min. ¹H NMR (400 MHz, DMSO-d6): δ=8.65-8.58 (m, 1H), 8.27-8.21 (m, 1H), 7.75-7.70 (m, 3H), 7.69-7.62 (m, 3H), 7.52-7.47 (m, 1H), 4.15-3.99 (m, 2H), 2.99-2.91 (m, 1H), 2.90-2.77 (m, 2H), 2.48-2.47 (m, 3H), 2.41 (s, 3H), 1.89-1.81 (m, 2H), 1.67-1.55 (m, 2H), 1.45-1.37 (m, 9H)

### Example 43: 5-(4-chlorophenyl)-2,3-dimethyl-7-(4-(trifluoromethoxy)phenyl)pyrido[2,3-d]pyridazin-8 (7H)-one 1-27

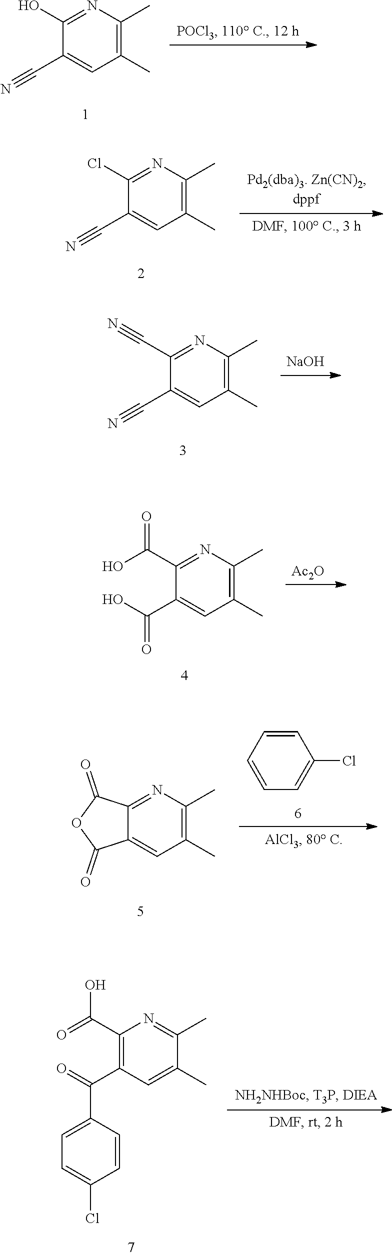

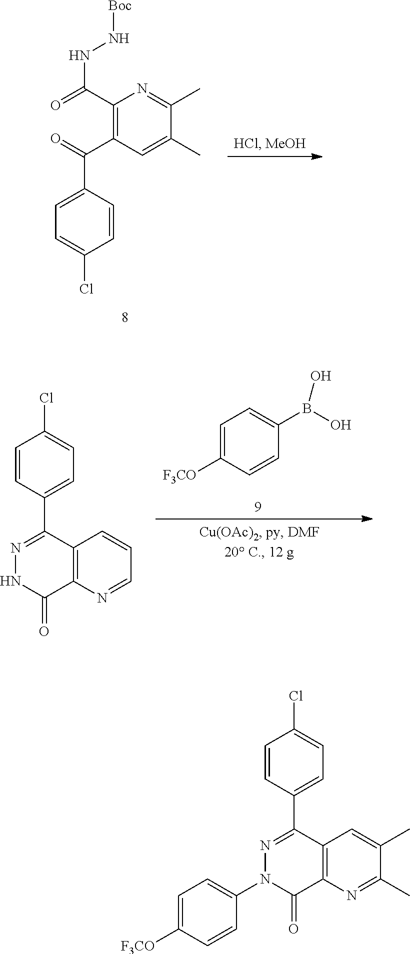 [SMILES: Cc1cc(C(=O)c2ccc(Cl)cc2)c(C(=O)NNC(=O)OC(C)(C)C)nc1C ; O=c1[nH]nc(-c2ccc(Cl)cc2)c2cccnc12 ; OB(O)c1ccc(OC(F)(F)F)cc1 ; Cc1cc2c(-c3ccc(Cl)cc3)nn(-c3ccc(OC(F)(F)F)cc3)c(=O)c2nc1C]

Step 1: To a solution of 2-hydroxy-5,6-dimethylnicotinonitrile (1.00 eq, 4500 mg, 30.4 mmol) in POCl₃ (9.51 eq, 27 mL, 289 mmol). The mixture was stirred at 20° C. for 1 h. Then the reaction mixture was warmed to 110° C. for 12 h. TLC indicated the starting material was consumed completely and one new spot formed (PE/EtOAc=0/1, starting material R_{f}=0.5; PE/EtOAc=10/1, new spot R_{f}=0.5). The reaction mixture was poured into saturated aqueous NaHCO₃ (100 mL) and adjusted PH>7 at 0° C. Then the mixture was extracted with EtOAc (50 mL×3). The organic layer was washed with brine, dried by Na₂SO₄. The solution was concentrated to give the residue. The residue was purified by column chromatography (SiO₂, PE/EtOAc=20/1 to 10/1) to afford 2-chloro-5,6-dimethylnicotinonitrile (5000 mg, 30.0 mmol, 98.81% yield) was obtained as white solid. Confirmed by LCMS. [M+H]⁺=167.2; purity=99.8% (220 nm); Retention time=0.723 min. ¹H NMR (400 MHz, CDCl₃) δ=7.70 (s, 1H), 2.56 (s, 3H), 2.32 (s, 3H).

Step 2: To a mixture of 1,1′-Bis(diphenylphosphino)ferrocene (0.1000 eq, 1531 mg, 2.76 mmol), Zn(CN)₂ (1.00 eq, 3242 mg, 27.6 mmol) and 2-chloro-5,6-dimethyl-pyridine-3-carbonitrile (1.00 eq, 4600 mg, 27.6 mmol) in DMF (60 mL) was added Pd₂(dba)₃ (0.1000 eq, 1588 mg, 2.76 mmol) at 25° C. under N₂. The mixture was heated to 100° C. under N₂ for 3 h. LCMS showed that the starting material was consumed completely and detected the desired mass (68%, Rt: 0.426 min; [M+H]+=158.1 at 220 nm). The reaction mixture was filtered and quenched by addition 1 N HCl (100 mL) at 0° C., and then extracted with EtOAc (80 mL×2). The combined organic layers were washed with brine (100 mL), dried over Na₂SO₄, filtered and concentrated under reduced pressure to give a residue. The residue was purified by column chromatography (SiO₂, PE/EtOAc=1/0 to 3/1; PE/EtOAc=3/1, R_{f}=0.4) to afford 5,6-dimethylpyridine-2,3-dicarbonitrile (4000 mg, 21.9 mmol, 79.27% yield) as brown solid, checked by LCMS: [M+H]⁺=158.2; purity=86.7% (220 nm); Retention time=0.589 min.

Step 3: To a solution of 5,6-dimethylpyridine-2,3-dicarbonitrile (1.00 eq, 3700 mg, 23.5 mmol) and NaOH (15.0 eq, 14125 mg, 353 mmol) in ethanol (50 mL), water (50 mL). The mixture was stirred at 100° C. for 16 h. LCMS showed that the starting material was consumed completely and desired mass was detected (76%, Rt: 0.124 min; [M+H]+=196.1 at 220 nm. The reaction mixture was diluted by addition H₂O (80 mL) at 0° C. and extracted with EtOAc (50 mL×2). The combined aqueous layers were adjusted pH<7 with 1 N HCl (500 mL). The combined aqueous phase was concentrated under vacuum to give yellow solid. The solid was dissolved in MeOH (50 mL) and filtered. The filtrated was concentrated under vacuum to give crude 5,6-dimethylpyridine-2,3-dicarboxylic acid (6500 mg, 33.3 mmol, 141.47% yield) as yellow solid, checked by LCMS: [M−H₂O+H]⁺=178.0; purity=93.9% (220 nm); Retention time=0.132 min. H NMR (400 MHz, DMSO-d₆) δ=7.98 (s, 1H), 2.49 (br s, 3H), 2.32 (s, 3H).

Step 4: To a solution of 5,6-dimethylpyridine-2,3-dicarboxylic acid (1.00 eq, 6000 mg, 30.7 mmol) in Ac₂O (50 mL). The mixture was stirred at 100° C. for 16 h., a drop of reaction mixture quenched by MeOH) showed that the starting material was consumed completely and the desired ester mass was detected (58%, Rt: 0.469 min; [M+MeOH]+=210.1 at 220 nm). The mixture was concentrated under reduced pressure to give a residue. The residue was purified by column chromatography (SiO₂, PE/EtOAc=1/0 to 1/1; PE/EtOAc=1/1, the desired product R_{f}=0.5) to afford 2,3-dimethylfuro[3,4-b]pyridine-5,7-dione (2700 mg, 15.2 mmol, 49.57% yield) as yellow solid, checked by LCMS: [M+MeOH]⁺=210.2; purity=83% (220 nm); Retention time=0.595 min. H NMR (400 MHz, CDCl₃) δ=8.00 (d, J=18.7 Hz, 1H), 2.52 (d, J=11.4 Hz, 3H).

Step 5: A mixture of 2,3-dimethylfuro[3,4-b]pyridine-5,7-dione (1.00 eq, 2700 mg, 15.2 mmol) in PhCl (32.3 eq, 50 mL, 492 mmol) was added AlCl₃ (6.00 eq, 12193 mg, 91.4 mmol) under N₂. Then the reaction mixture was heated to 80° C. for 3 h. LCMS (HW-2021-01-071-P1A1) showed that the starting material was consumed completely, the desired mass was detected (20%, Rt: 0.487 min; [M+H]⁺=290.0 at 220 nm). The reaction mixture was quenched by MeOH (100 mL) at 0° C. and concentrated under reduced pressure to give a residue. The residue was purified by flash silica gel chromatography (ISCO; 120 g SepaFlash Silica Flash Column, Eluent of 0˜10% DCM/MeOH; flow rate: 80 mL/min) to afford 3-(4-chlorobenzoyl)-5,6-dimethylpicolinic acid (1900 mg, 6.56 mmol, 43.03% yield) as yellow solid, checked by LCMS [M+H]⁺=290.1; purity=67% (220 nm); Retention time=0.769 min. H NMR (400 MHz, DMSO-d₆) δ=7.74 (s, 1H), 7.67-7.63 (m, 2H), 7.60-7.55 (m, 2H), 2.56 (s, 3H), 2.36 (s, 3H)

Step 6: A mixture of 3-(4-chlorobenzoyl)-5,6-dimethyl-pyridine-2-carboxylic acid (1.00 eq, 1700 mg, 5.87 mmol), tert-butyl N-aminocarbamate (2.00 eq, 1551 mg, 11.7 mmol) and DIPEA (5.00 eq, 5.1 mL, 29.3 mmol) in DMF (20 mL) was added T₃P (3.00 eq, 11198 mg, 17.6 mmol). The mixture was stirred at 25° C. for 2 h. LCMS showed the starting material was consumed completely and a major peak with desired MS (68%, Rt: 0.645 min; [M+H]+=404.1 at 220 nm). The mixture was diluted with water (80 mL) and extracted with EtOAc (80 mL×2). The combined organic layers were washed with an aqueous solution with brine (100 mL) and dried over Na₂SO₄. The solvent was filtered and the filtrate was concentrated under reduced pressure. The crude product was purified by column chromatography on silica gel eluted with PE/EtOAc=1/0 to 0/1 (PE/EtOAc=1/1, the desired product R_{f}=0.5) to give tert-butyl N-[[3-(4-chlorobenzoyl)-5,6-dimethyl-pyridine-2-carbonyl]amino]carbamate (1770 mg, 3.81 mmol, 64.98% yield) as yellow solid, checked by LCMS: [M+H]+=403.9; purity=87% (220 nm); Retention time=0.900 min H NMR (400 MHz, CDCl₃) δ=7.74-7.63 (m, 1H), 7.46-7.33 (m, 4H), 2.62 (d, J=19.2 Hz, 3H), 2.37 (d, J=18.5 Hz, 3H), 1.62 (d, J=3.7 Hz, 5H), 1.50-1.47 (m, 9H)

Step 7: To a solution of tert-butyl N-[[3-(4-chlorobenzoyl)-5,6-dimethyl-pyridine-2-carbonyl]amino]carbamate (1.00 eq, 1770 mg, 4.38 mmol) in HCl/MeOH (18.3 eq, 20 mL, 80.0 mmol). The mixture was stirred at 25° C. for 2 h. LCMS (YT-2021-01-012-P1A) showed the starting material was consumed completely and a major peak with desired MS (88%, Rt: 0.839 min; [M+H]⁺=286.2 at 220 nm). The reaction mixture was concentrated under reduced pressure to give 5-(4-chlorophenyl)-2,3-dimethyl-7H-pyrido[2,3-d]pyridazin-8-one; hydrochloride (1450 mg, 4.50 mmol, 102.69% yield) as yellow solid, checked by LCMS: YT-2021-01-12-1 (P1): [M+H]⁺=285.7; purity=92.3% (220 nm); Retention time=0.877 min MS: 285.7=[M+H]+, ESI+1H NMR (400 MHz, DMSO-d₆) δ=13.08 (s, 1H), 7.83 (s, 1H), 7.61 (s, 4H), 2.67 (s, 3H), 2.41 (s, 3H)

Step 8: A mixture of [4-(trifluoromethoxy)phenyl]boronic acid (2.00 eq, 72 mg, 0.350 mmol) and 5-(4-chlorophenyl)-2,3-dimethyl-7H-pyrido[2,3-d]pyridazin-8-one (1.00 eq, 50 mg, 0.175 mmol) in DMF (1 mL) was added pyridine (3.00 eq, 0.042 mL, 0.525 mmol) and Cu(OAc)₂ (1.10 eq, 35 mg, 0.192 mmol). The mixture was degassed with O₂ for 3 times. The mixture was stirred at 25° C. under O₂ for 12 h. LCMS showed starting material was consumed completely and a peak with desired MS (70%, Rt: 1.034 min; [M+H]+=445.9 at 220 nm) was detected. The mixture was filtered and the filtrate was purified by prep-HPLC (FA in water:MeCN=100% to 0%, 220 & 254 nm) and lyophilized to give 5-(4-chlorophenyl)-2,3-dimethyl-7-[4-(trifluoromethoxy)phenyl]pyrido[2,3-d]pyridazin-8-one (28 mg, 0.0635 mmol, 36.28% yield) as off-white solid, checked by H NMR: [M+H]⁺=445.8; purity=100% (220 nm); Retention time=1.029 min 1H NMR (400 MHz, CDCl₃) δ=7.87-7.81 (m, 2H), 7.75 (s, 1H), 7.55 (d, J=1.2 Hz, 3H), 7.33 (d, J=8.8 Hz, 2H), 2.82 (s, 3H), 2.48 (s, 3H).

### Example 44: 5-(4-chlorophenyl)-2-methyl-7-[4-(trifluoromethoxy)phenyl]pyrido[2,3-d]pyridazin-8-one I-26

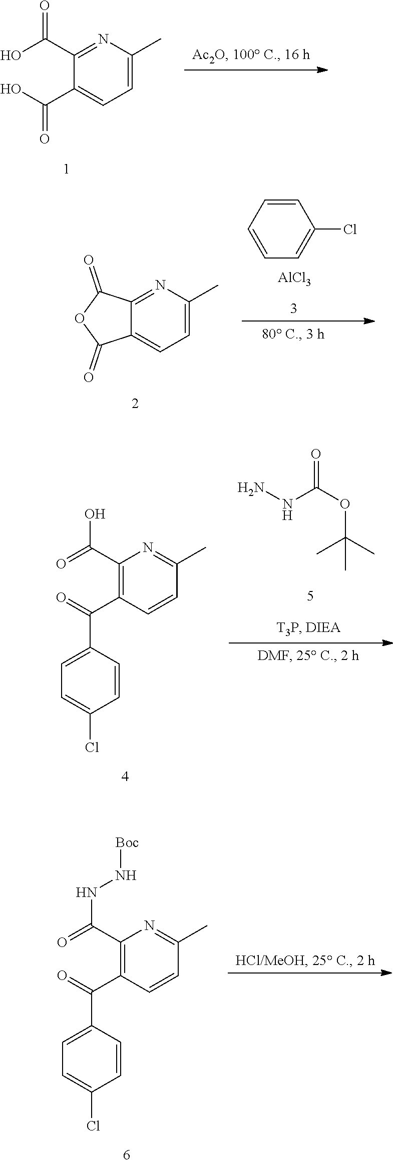

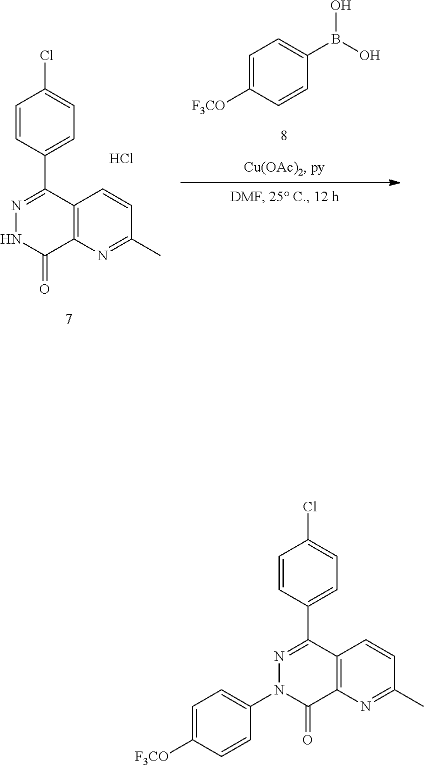 [SMILES: Cc1ccc2c(-c3ccc(Cl)cc3)n[nH]c(=O)c2n1 ; Cl ; OB(O)c1ccc(OC(F)(F)F)cc1 ; Cc1ccc2c(-c3ccc(Cl)cc3)nn(-c3ccc(OC(F)(F)F)cc3)c(=O)c2n1]

Step 1: To a solution of 6-methylpyridine-2,3-dicarboxylic acid (1.00 eq, 5000 mg, 27.6 mmol) in Acetic anhydride (50 mL). The mixture was stirred at 100° C. for 16 h. LCMS showed that the starting material was consumed completely and the desired mass was detected (64%, MS: 196.2 [M+H]⁺, ESI pos). The mixture was concentrated under reduced pressure to give a residue. The residue was purified by column chromatography (SiO₂, PE/EtOAc=1/0 to 1/1; PE/EtOAc=1/1, the desired product R_{f}=0.5) to afford 2-methylfuro[3,4-b]pyridine-5,7-dione (2700 mg, 16.6 mmol, 59.97% yield) as yellow solid. (M+H)⁺=196.2; purity=80% (220 nm); Retention time=0.530 min. ¹H NMR (400 MHz, CDCl₃) δ=8.23 (d, J=8.0 Hz, 1H), 7.66 (d, J=8.0 Hz, 1H), 2.86 (s, 3H)

Step 2: A mixture of AlCl₃ (6.00 eq, 10789 mg, 80.9 mmol) in PhCl (29.2 eq, 40 mL, 393 mmol) was added 2-methylfuro[3,4-b]pyridine-5,7-dione (1.00 eq, 2200 mg, 13.5 mmol) under N₂. Then the reaction mixture was heated to 80° C. for 3 h. LCMS showed that the starting material was consumed completely, two peaks with desired mass were detected (4% and 29%, MS: 275.9 [M+H]⁺, ESI pos). The reaction mixture was quenched by addition MeOH (100 mL) at 0° C. and concentrated under reduced pressure to give a residue. The crude product was purified by column chromatography on silica gel eluted with DCM/MeOH=1/0 to 10/1 (DCM/MeOH=10/1, the desired product R_{f}=0.4) to give 3-(4-chlorobenzoyl)-6-methyl-pyridine-2-carboxylic acid (4000 mg, 11.8 mmol, 87.14% yield) as yellow solid. n(M+H)⁺=275.8; purity=99% (220 nm); Retention time=0.928 min. n¹H NMR (400 MHz, DMSO-d6) δ=7.84 (br d, J=8.0 Hz, 1H), 7.60-7.54 (m, 3H), 7.49 (br d, J=8.0 Hz, 2H), 2.54 (s, 2H)

Step 3: A mixture of 3-(4-chlorobenzoyl)-6-methyl-pyridine-2-carboxylic acid (1.00 eq, 400 mg, 1.45 mmol), tert-butyl N-aminocarbamate (2.00 eq, 384 mg, 2.90 mmol) and DIPEA (5.00 eq, 1.3 mL, 7.25 mmol) in DMF (3 mL) was added 2,4,6-tripropyl-1,3,5,2,4,6-trioxatriphosphinane 2,4,6-trioxide (3.00 eq, 2769 mg, 4.35 mmol). The mixture was stirred at 25° C. for 2 h. LCMS showed the starting material was consumed completely and a major peak with desired MS (87%, MS: 334.3 [M+23]⁺, ESI pos). The mixture was diluted with water (40 mL) and extracted with ethyl acetate (40 mL) twice. The combined organic layers were washed with an aqueous solution with brine (50 mL) and dried over Na₂SO₄. The solvent was filtered and concentrated under reduced pressure. The crude product was purified by column chromatography on silica gel eluted with PE/EtOAc=1/0 to 0/1 (PE/EtOAc=1/1, the desired product R_{f}=0.5) to give tert-butyl N-[[3-(4-chlorobenzoyl)-6-methyl-pyridine-2-carbonyl]amino]carbamate (430 mg, 1.10 mmol, 76.02% yield) as yellow solid. (M+23)⁺=333.7; purity=99% (220 nm); Retention time=0.883 min. ¹H NMR (400 MHz, DMSO-d6) δ=9.30-8.87 (m, 1H), 7.71-7.60 (m, 1H), 7.48-7.28 (m, 6H), 2.59 (br s, 3H), 1.41-1.26 (m, 9H).

Step 4: A solution of tert-butyl N-[[3-(4-chlorobenzoyl)-6-methyl-pyridine-2-carbonyl]amino]carbamate (1.00 eq, 430 mg, 1.10 mmol) in HCl/MeOH (53.8 eq, 15 mL, 59.4 mmol) was stirred at 25° C. for 2 h. LCMS showed the starting material was consumed completely and a major peak with desired MS (84%, MS: 272.0 [M+H]⁺, ESI pos). The reaction mixture was concentrated under reduced pressure to give a residue. The crude product was triturated with ethyl acetate (10 mL) for 2 min. The solution was filtered to give 5-(4-chlorophenyl)-2-methyl-7H-pyrido[2,3-d]pyridazin-8-one (290 mg, 1.07 mmol, 96.76% yield) as white solid. (M+H)⁺=282.1; purity=99% (220 nm); Retention time=0.721 min. ¹H NMR (400 MHz, DMSO-d6) δ=13.11 (s, 1H), 8.00 (d, J=8.4 Hz, 1H), 7.77 (d, J=8.4 Hz, 1H), 7.61 (s, 4H), 2.72-2.69 (m, 3H)

Step 5: A mixture of [4-(trifluoromethoxy)phenyl]boronic acid (2.00 eq, 120 mg, 0.584 mmol) and 5-(4-chlorophenyl)-2-methyl-7H-pyrido[2,3-d]pyridazin-8-one; hydrochloride (1.00 eq, 90 mg, 0.292 mmol) in DMF (3 mL) was added pyridine (3.00 eq, 0.071 mL, 0.876 mmol) and Cu(OAc)₂ (1.10 eq, 58 mg, 0.321 mmol). The mixture was degassed with 02 for 3 times. The mixture was stirred at 25° C. under 02 (15 psi) for 12 h. LCMS showed most of starting material was still remained. The mixture was added pyridine (5.00 eq, 0.12 mL, 1.46 mmol) and [4-(trifluoromethoxy)phenyl]boronic acid (2.00 eq, 120 mg, 0.584 mmol) and Cu(OAc)₂ (1.10 eq, 58 mg, 0.321 mmol). The mixture was degassed with 02 for 3 times. The mixture was stirred at 25° C. under 02 (15 psi) for 12 h. LCMS showed the starting material was consumed completely and a major peak with desired MS (53%, MS: 431.8[M+H]+, ESI pos). The reaction mixture was filtered and the filtrate was purified by prep-HPLC (Phenomenex C18 75×30 mm×3 um; mobile phase: [water (0.1% FA)-ACN]; B %: 50%-80%, 7 min) to give 5-(4-chlorophenyl)-2-methyl-7-[4-(trifluoromethoxy)phenyl]pyrido[2,3-d]pyridazin-8-one (36 mg, 0.0799 mmol, 27.36% yield) as light yellow solid. (M+H)⁺=432.1; purity=99% (220 nm); Retention time=0.922 min. ¹H NMR (400 MHz, DMSO-d6) δ=8.07 (d, J=8.4 Hz, 1H), 7.87-7.79 (m, 3H), 7.73-7.62 (m, 4H), 7.54 (d, J=8.7 Hz, 2H), 2.75 (s, 3H)

### Example 45: Synthesis of 7-(4-chlorophenyl)-5-(2-(4-chlorophenyl)-2-(methylamino)ethyl)-2-methylthiazolo[4,5-d]pyridazin-4 (5H)-one I-75; 7-(4-chlorophenyl)-5-(2-(4-chlorophenyl)-2-oxoethyl)-2-methylthiazolo[4,5-d]pyridazin-4 (5H)-one I-74; and 7-(4-chlorophenyl)-5-(2-(4-chlorophenyl)-2-hydroxyethyl)-2-methylthiazolo[4,5-d]pyridazin-4 (5H)-one I-78

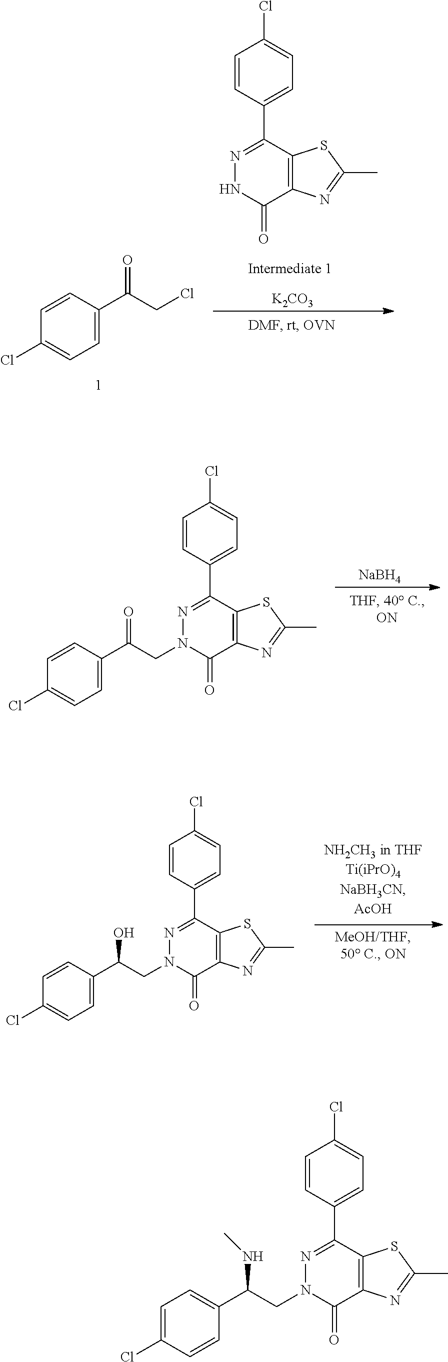

Step 1: To a mixture of 7-(4-chlorophenyl)-2-methyl-5H-thiazolo[4,5-d]pyridazin-4-one (intermediate 1) (200 mg, 7.2 mmol, 1 eq) in DMF (3 mL) were added K₂CO₃ (299 mg, 2.2 mmol, 3 eq) and compound 1 (204 mg, 1.1 mmol, 1.5 eq). The mixture was stirred at 25° C. under N₂ atmosphere overnight. After the reaction was completed, the mixture was diluted with H₂O (30 mL) and extracted with EtOAc (30 mL×3). The organic layer was washed with brine (50 mL), dried over Na₂SO₄, and concentrated in vacuo to give the crude product. The crude product was purified by silica gel flash chromatographer (Petroleum ether:EtOAc=60:40) to give the desired product as a light yellow solid (200 mg, 61.3%). LCMS: (M+H)⁺=430.1, Retention time=1.542 min HPLC: purity=100% (254 nm); purity=99.74% (214 nm); Retention time=3.934 min 1H NMR (400 MHz, CDCl3) δ 7.98-7.96 (m, 2H), 7.75-7.72 (m, 2H), 7.51-7.45 (m, 4H), 5.82 (s, 2H), 2.96 (s, 3H)

Step 2: To a mixture of 7-(4-chlorophenyl)-5-[2-(4-chlorophenyl)-2-oxo-ethyl]-2-methyl-thiazolo[4,5-d]pyridazin-4-one (200 mg, 0.47 mmol, 1 eq) in THF (5 mL) was added NaCNBH₃ (146 mg, 2.32 mmol, 5 eq). The mixture was stirred at 60° C. under N₂ atmosphere overnight. The mixture was diluted with DCM (50 mL) and water (50 mL), and then extracted with DCM (20 mL×3). The organic layer was combined, washed with brine (20 mL), dried over Na₂SO₄, and concentrated in vacuo to give the crude product. The crude product was purified by prep-HPLC to give the desired product as a white solid (44.2 mg, 21.9%). LCMS: (M+H)⁺=432.1, Retention time=1.464 min. HPLC: purity=100% (254 nm); purity=99.68% (214 nm); Retention time=3.688 min. 1H NMR (400 MHz, DMSO) δ 7.72-7.70 (m, 2H), 7.64-7.62 (m, 2H), 7.39 (s, 4H), 5.72 (s, 1H), 5.14-5.10 (m, 1H), 4.52-4.46 (m, 1H), 4.32-4.27 (m, 1H), 2.88 (s, 3H)

Step 3: To a mixture of 7-(4-chlorophenyl)-5-[2-(4-chlorophenyl)-2-oxo-ethyl]-2-methyl-thiazolo[4,5-d]pyridazin-4-one (300 mg, 0.69 mmol, 1 eq), titanium tetraisopropanolate (396 mg, 1.39 mmol, 3 eq) and acetic acid (42 mg, 0.69 mmol, 1 eq) in THF/MeOH (2 mL/2 mL) was added NH₂CH₃ in THF (1 mL, 2.09 mmol, 2 mol/L, 3 eq). The mixture was stirred for 0.5 h under N₂ and then NaBH₃CN (66 mg, 1.04 mmol, 1.5 eq) was added in portions. The resulting mixture was stirred at 50° C. overnight. The mixture was diluted with NH₄Cl(aq) (10 mL), extracted with EtOAc (20 mL×3), dried over Na₂SO₄, and concentrated in vacuo to give the crude product. The crude product was purified by prep-HPLC to give the desired product as a white solid (39.2 mg, 12.5%). LCMS: (M+H)⁺=445.1, Retention time=1.229 min. HPLC: purity=100% (254 nm); purity=100% (214 nm); Retention time=3.639 min. 1H NMR (400 MHz, DMSO) δ 7.66-7.60 (m, 4H), 7.39-7.33 (m, 4H), 4.57-4.52 (m, 1H), 4.29-4.24 (m, 1H), 4.11-4.07 (t, J=6.4 Hz, 1H), 2.87 (s, 3H), 2.12 (s, 3H).

### Example 46: Synthesis of 7-(4-chlorophenyl)-5-(1-(4-chlorophenyl)ethyl)-2-methylthiazolo[4,5-d]pyridazin-4 (5H)-one I-76

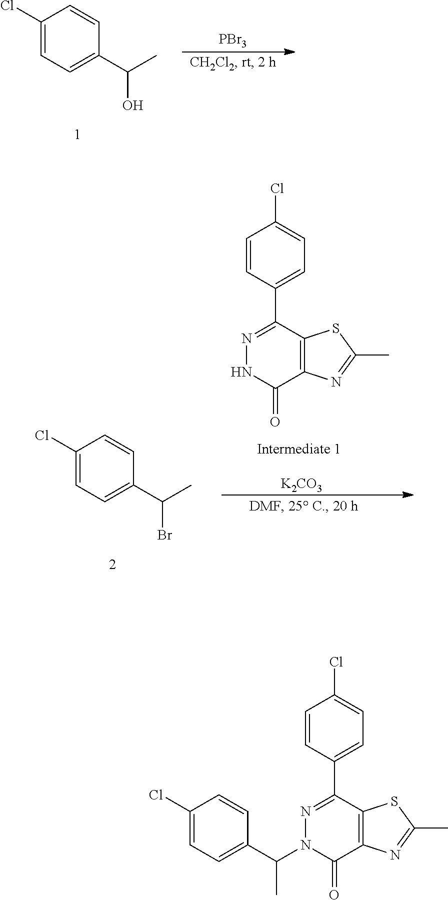

Step 1: Step 1: To a mixture of compound 1 (500 mg, 3.19 mmol, 1.00 eq) in CH₂Cl₂ (5 mL) was added PBr₃ (1.04 g, 3.83 mmol, 1.2 eq) dropwise at 0° C. under N₂. Then the reaction mixture was stirred at room temperature for 4 hours. The pH of reaction mixture was adjusted to 7 with NaHCO₃ (aq) at 0° C. The aqueous layer was extracted with DCM (3×20 mL), dried over Na₂SO₄ and concentrated to afford the crude product compound 2 (420 mg, 56.9%) as a colorless oil.

Step 2: To a mixture of compound 2 (80 mg, 0.29 mmol, 1 eq) and K₂CO₃ (40 mg, 0.29 mmol, 1 eq) in DMF was added intermediate 1 (126 mg, 0.58 mmol, 2 eq) under N₂. The resulting mixture was stirred at 25° C. for 6 hours. The reaction was diluted with H₂O (30 mL) and extracted with EtOAc (30 mL×3). The organic layer was washed with brine, dried over Na₂SO₄, and evaporated. The crude product was purified by SGC (Petroleum ether:Ethyl acetate=2:1) to afford the desired product (81 mg, 52.3%) as a white solid. LCMS: (M+H)+=416.1, Retention time=1.654 min. HPLC: purity=96.108% (254 nm); purity=95.180% (214 nm); Retention time=4.972 min. ¹H NMR (400 MHz, DMSO) δ 7.85 (d, J=8.5 Hz, 2H), 7.66 (d, J=8.5 Hz, 2H), 7.44-7.38 (m, 4H), 6.40 (q, J=7.0 Hz, 1H), 2.88 (s, 3H), 1.79 (d, J=7.0 Hz, 3H).

### Example 47: Synthesis of 2-methyl-5-phenethyl-7-phenylthiazolo[4,5-d]pyridazin-4 (5H)-one I-77

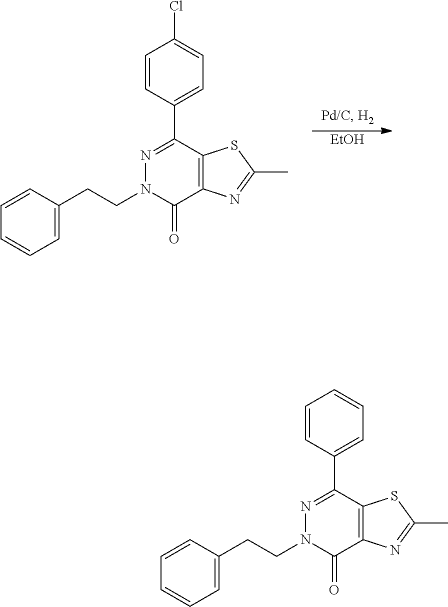 [SMILES: "EtOH" ; Cc1nc2c(=O)n(CCc3ccccc3)nc(-c3ccc(Cl)cc3)c2s1 ; Cc1nc2c(=O)n(CCc3ccccc3)nc(-c3ccccc3)c2s1]

Step 1: To a mixture of 7-(4-chlorophenyl)-2-methyl-5-phenethylthiazolo[4,5-d]pyridazin-4 (5H)-one (200 mg, 0.52 mmol, 1.00 eq) in ethanol (2 mL) were added Pd/C (111 mg, 1.05 mmol, 2 eq) and Et₃N (5 mg, 0.05 mmol, 0.1 eq). The reaction flask was evacuated and refilled with H₂ three times. Then the reaction mixture was stirred at room temperature for 4 hours. The mixture was filtered and evaporated. The residue was purified with LC8AP prep-HPLC to afford the desired product (45 mg, 24.7%) as a white solid. Method A: Instrument: Shimadzu LC8AP PREP-HPLC system; Column: AQ-C18 30*250 mm; Mobile Phase: 0.1% CF₃COOH in H2O/ACN; Gradient: 20% of ACN from 1 min to 10 min, 20% to 60% of ACN from 10 min to 30 min, Column Temperature: 25° C.; Detective Wavelength: 214/254 nm; Flow Rate: 20 mL/min. LCMS: (M+H)⁺=348.1, Retention time=1.461 min HPLC: purity=98.927% (254 nm); purity=99.004% (214 nm); Retention time=4.108 min. ¹H NMR (400 MHz, DMSO) δ 7.73-7.63 (m, 2H), 7.55-7.53 (m, 3H), 7.30-7.20 (m, 5H), 4.51 (t, J=7.3 Hz, 2H), 3.14 (t, J=7.3 Hz, 2H), 2.87 (s, 3H).

### Example 48: 7-(4-chlorophenyl)-5-(1-(4-chlorophenyl)propan-2-yl)-2-methylthiazolo[4,5-d]pyridazin-4 (5H)-one I-79

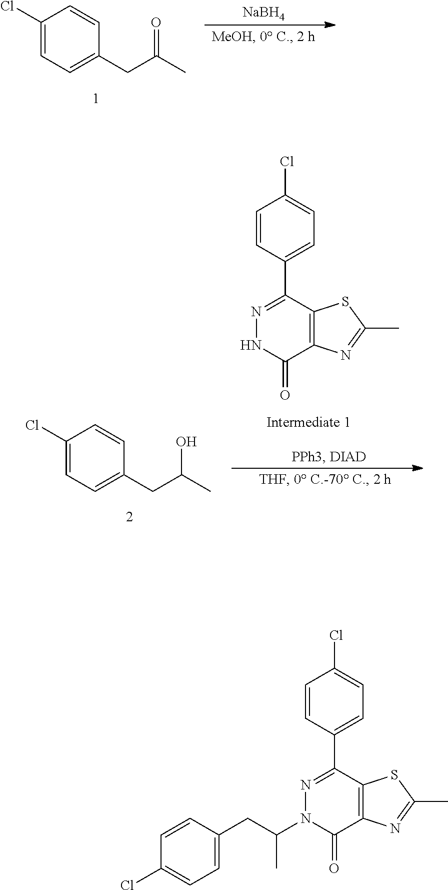

Step 1: To a mixture of Compound 1 (1 g, 5.9 mmol, 1.0 eq) in MeOH (10 mL) was added NaBH₄ (0.33 g, 8.9 mmol, 1.5 eq) portionwise at 0° C. under N₂. The mixture was stirred for 2.0 hours. After the reaction was completed, the mixture was quenched with NaHCO₃ (aq) (20 mL), extracted with DCM (20 mL×3), dried over Na₂SO₄, and concentrated in vacuo to give Compound 2 (1.02 g, 95.8%) as a colorless oil. ¹H NMR (SY-2021-01-013-1A) indicated the desired compound. ¹H NMR (400 MHz, DMSO) δ 7.32-7.30 (m, 2H), 7.22-7.20 (m, 2H), 4.58 (d, J=4.8 Hz, 1H), 3.80 (m, 1H), 2.73-2.52 (m, 2H), 1.02 (d, J=6.20 Hz, 3H).

Step 2: To a mixture of Intermediate 1 (200 mg, 0.72 mmol, 1.0 eq), Compound 2 (135 mg, 0.79 mmol, 1.1 eq), triphenylphosphine (283 mg, 1.08 mmol, 1.5 eq) in THF (5 mL) was added DIAD (218 mg, 1.08 mmol, 1.5 eq) in THF (3 mL) dropwise at 0° C. under N₂. The resulting mixture was stirred at 70° C. for 2 hours. The reaction mixture was diluted with water (20 mL) and extracted with EtOAc (20 mL×2). The organic layer was dried over sodium sulfate and evaporated. The product was purified by prep-HPLC to give I-79 (170 mg, 52.1%) as a white solid. LCMS: (M+H)⁺=431, Retention time=1.478 min. HPLC: purity=98.47% (254 nm); purity=95.28% (214 nm); Retention time=5.094 min. ¹H NMR (400 MHz, DMSO) δ 7.89 (d, J=8.6 Hz, 2H), 7.68 (d, J=8.6 Hz, 2H), 7.25 (d, J=8.4 Hz, 2H), 7.16 (d, J=8.4 Hz, 2H), 5.64-5.48 (m, 1H), 3.21-3.05 (m, 2H), 2.85 (s, 3H), 1.42 (d, J=6.6 Hz, 3H).

### Example 49: 7-(4-chlorophenyl)-5-(2-(4-chlorophenyl)propyl)-2-methylthiazolo[4,5-d]pyridazin-4 (5H)-one I-80

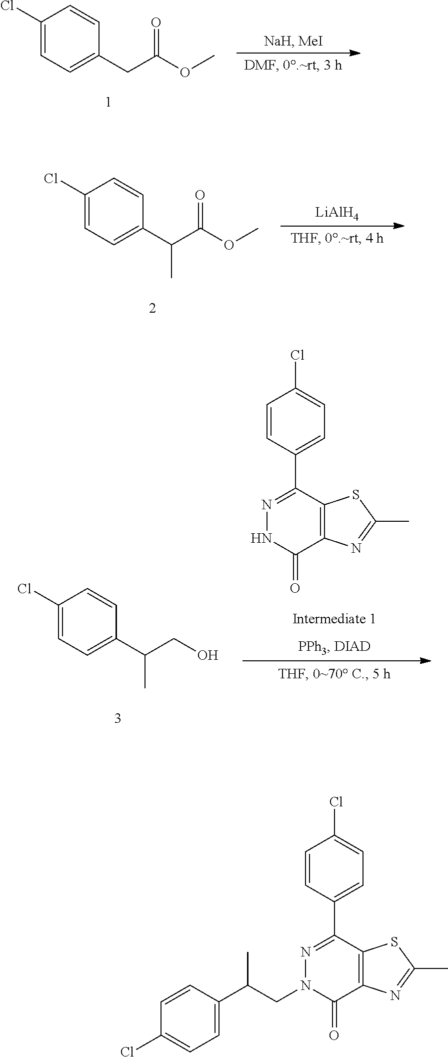 [SMILES: COC(=O)Cc1ccc(Cl)cc1 ; COC(=O)C(C)c1ccc(Cl)cc1 ; "LiAlH4" ; Cc1nc2c(=O)[nH]nc(-c3ccc(Cl)cc3)c2s1 ; CC(CO)c1ccc(Cl)cc1 ; Cc1nc2c(=O)n(CC(C)c3ccc(Cl)cc3)nc(-c3ccc(Cl)cc3)c2s1]

Step 1: To a solution of NaH (0.96 g, 40.08 mmol, 2 eq) in DMF (30 mL) was added methyl 2-(4-chlorophenyl)acetate (3.7 g, 20.04 mmol, 1 eq) dropwise at 0° C. After stirred for 15 minutes, Mel (2.84 g, 20.04 mmol, 1 eq) was added dropwise at the same temperature. Then the reaction mixture was warmed up to 25° C. slowly and stirred for 3 hours. After reaction was completed, the reaction was quenched with an aqueous saturated NH₄Cl solution (100 mL) and extracted with EtOAc (100 mL×3). The combined organic phase was washed with H₂O (100 mL×2) and brine (100 mL×2), dried over Na₂SO₄, filtrated and concentrated under reduced pressure to give methyl 2-(4-chlorophenyl)propanoate (3.5 g, 70.3%) as a yellow oil. ¹H NMR (400 MHz, CDCl₃) δ 7.27-7.27 (m, 2H), 7.23-7.21 (m, 2H), 3.72-3.68 (m, 1H), 3.65 (s, 3H), 1.49-1.47 (d, J=7.2 Hz, 3H).

Step 2: To a solution of methyl 2-(4-chlorophenyl)propanoate (3.5 g, 17.62 mmol, 1 eq) in THF (50 mL) was added LiAlH₄ (802 mg, 21.14 mmol, 1.2 eq) in portions at 0° C. After stirred for 15 minutes, the reaction mixture was warmed up to 25° C. slowly and stirred for 2 hours. After reaction was completed, the reaction mixture was quenched with H₂O (0.8 mL) and 15% NaOH (8 mL). The mixture was then filtrated and the filtrate was dried over anhydrous sodium sulfate, filtrated and concentrated under reduced pressure to give a residue, which was purified by SGC (Petroleum ether/Ethyl acetate=82/18) to afford 2-(4-chlorophenyl)propan-1-ol (2 g, 59.9%) as a colorless oil. ¹H NMR (400 MHz, DMSO) δ 7.33-7.30 (m, 2H), 7.25-7.23 (m, 2H), 4.66-4.63 (t, J=5.2 Hz, 1H), 3.50-3.38 (m, 2H), 2.84-2.75 (m, 1H), 1.17-1.15 (d, J=7.2 Hz, 3H).

Step 3: To a mixture of 7-(4-chlorophenyl)-2-methyl-5H-thiazolo[4,5-d]pyridazin-4-one (300 mg, 1.08 mmol, 1 eq), 2-(4-chlorophenyl)propan-1-ol (203 mg, 1.19 mmol, 1.1 eq) and PPh₃ (425 mg, 1.62 mmol, 1.5 eq) in THF (5 ml) was added a solution of DIAD (284 mg, 1.40 mmol, 1.3 eq) in THF (3 mL) dropwise at 0° C. under N₂. The reaction mixture was stirred at 70° C. for 5 hours. After reaction was completed, the mixture was diluted with water (20 mL) and extracted with EtOAc (20 mL×2). The organic layer was washed with brine (20 mL×2), dried over anhydrous sodium sulfate and concentrated to give a residue, which was purified by SGC (Petroleum ether/Ethyl acetate=60/40) to afford the crude product (200 mg), which was further purified by Prep-HPLC (ACN: 0.1% TFA in H₂O from 70:30 to 95:5 over 10 min) to afford 7-(4-chlorophenyl)-5-(2-(4-chlorophenyl)propyl)-2-methylthiazolo[4,5-d]pyridazin-4 (5H)-one (77.9 mg, 16.6%) as a white solid. LCMS: (M+H)⁺=430.1, Retention time=1.714 min. HPLC: purity=99.216% (214 nm); purity=99.246% (254 nm); Retention time=5.046 min ¹H NMR (400 MHz, DMSO) δ 7.65-7.59 (m, 4H), 7.32-7.27 (m, 4H), 4.47-4.35 (m, 2H), 3.47-3.41 (m, 1H), 2.86 (s, 3H), 1.30-1.28 (d, J=6.8 Hz, 3H).

### Example 50: Synthesis of 7-(4-chlorophenyl)-2-methyl-5-phenethylthiazolo[4,5-d]pyridazin-4 (5H)-one I-81

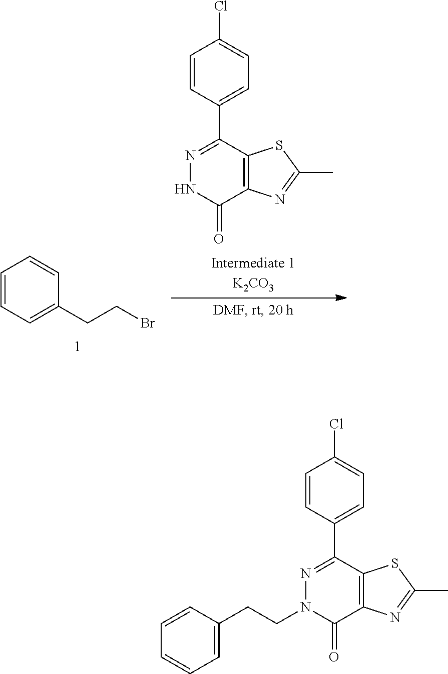 [SMILES: Cc1nc2c(=O)[nH]nc(-c3ccc(Cl)cc3)c2s1 ; BrCCc1ccccc1 ; Cc1nc2c(=O)n(CCc3ccccc3)nc(-c3ccc(Cl)cc3)c2s1]

Step 1: To a solution of Intermediate 1 (80 mg, 0.29 mmol, 1.0 eq) in DMF (3.0 mL) was added K₂CO₃ (119 mg, 0.86 mmol, 3.0 eq) and Compound 1 (266 mg, 1.44 mmol, 5.0 eq). The reaction mixture was stirred at 25° C. for 16 hours. The reaction mixture was diluted with water (20 mL) and extracted with EtOAc (20 mL×2). The organic layer was dried over Na₂SO₄ and evaporated to give the crude product. The residue was purified by SGC (50% ethyl acetate in petroleum ether) to give the desired product (100 mg, 91%) as a white solid. LCMS: (M+H)⁺=382, Retention time=1.582 min. HPLC: purity=99.23% (254 nm); purity=100% (214 nm); Retention time=3.728 min. ¹H NMR (400 MHz, DMSO) δ 7.72-7.70 (m, 2H), 7.63-7.61 (m, 2H), 7.29-7.18 (m, 5H), 4.52-4.49 (t, J=7.2, 2H), 3.15-3.14 (t, J=7.2, 2H), 2.88 (s, 3H).

### Example 51: Synthesis of 7-(3,4-dichlorophenyl)-2-methyl-5-phenethylthiazolo[4,5-d]pyridazin-4 (5H)-one I-82

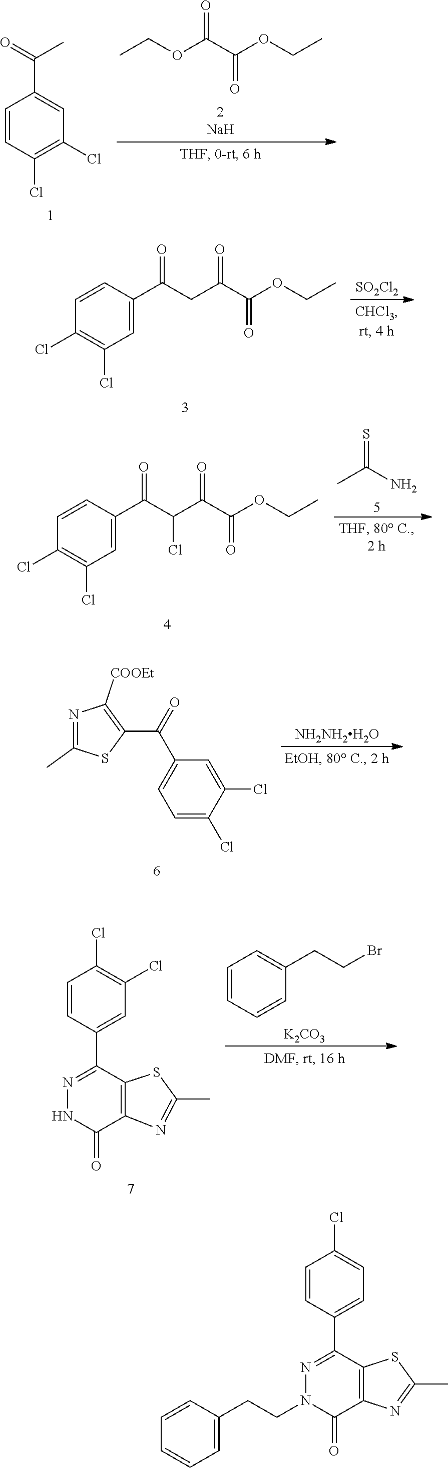

Step 1: To a solution of 1-(3,4-dichlorophenyl)ethanone (5.0 g, 26.5 mmol, 1 eq) in THF (25 mL) was added NaH (1.27 g, 52.9 mmol, 2 eq) in portions at 0° C. After stirred for 30 minutes, diethyl oxalate (5.80 g, 39.7 mmol, 1.5 eq) was added dropwise at the same temperature. Then the reaction mixture was warmed up to 25° C. slowly and stirred for 6 hours. After reaction was completed, the reaction was quenched with HCl (1N, 100 mL) and extracted with EtOAc (100 mL×3). The combined organic phase was washed with brine (100 mL×2), dried over anhydrous sodium sulfate, filtrated and concentrated under reduced pressure to give a residue, which was purified by SGC (Petroleum ether/Ethyl acetate=90/10) to afford ethyl 4-(3,4-dichlorophenyl)-2,4-dioxobutanoate (3.6 g, 42.4%) as a yellow oil. LCMS: (M−H)⁺=286.8, Retention time=1.377 min.

Step 2: To a solution of ethyl 4-(3,4-dichlorophenyl)-2,4-dioxo-butanoate (3.6 g, 12.5 mmol, 1 eq) in chloroform (30 mL) was added SO₂Cl₂ (8.4 g, 62.3 mmol, 5 eq) dropwise at 25° C. under N₂. The reaction mixture was stirred at 25° C. for 4 hours. The mixture was quenched with H₂O (50 mL) and extracted with DCM (50 mL×3). The combined organic phase was washed with brine (50 mL×2), dried over anhydrous sodium sulfate, filtrated and concentrated under reduced pressure to give a residue, which was purified by SGC (Petroleum ether/Ethyl acetate=93/7) to afford ethyl 3-chloro-4-(3,4-dichlorophenyl)-2,4-dioxobutanoate (2.5 g, 59.2%) as a yellow oil. LCMS: (M−H)⁺=321.0, Retention time=1.387 min.

Step 3: To a solution of ethyl 3-chloro-4-(3,4-dichlorophenyl)-2,4-dioxo-butanoate (2.2 g, 6.80 mmol, 1 eq) in THF (30 mL) was added ethanethioamide (613 mg, 8.16 mmol, 1.2 eq). The mixture was stirred at room temperature for 2 hours and then heated to 80° C. for 2 hours. After reaction was completed, the reaction mixture was diluted with H₂O (50 mL) and extracted with EtOAc (50 mL×3). The combined organic phase was washed with brine (50 mL×2), dried over anhydrous sodium sulfate, filtrated and concentrated under reduced pressure to give a residue, which was purified by SGC (Petroleum ether/Ethyl acetate=88/12) to afford ethyl 5-(3,4-dichlorobenzoyl)-2-methylthiazole-4-carboxylate (1.5 g, 60.9%) as a white solid. LCMS: (M+H)⁺=344.0, Retention time=1.471 min.

Step 4: To a solution of ethyl 5-(3,4-dichlorobenzoyl)-2-methyl-thiazole-4-carboxylate (500 mg, 1.45 mmol, 1 eq) in ethanol (5 mL) was added hydrazine hydrate (109 mg, 2.18 mmol, 1.5 eq). The mixture was stirred at 80° C. for 2 hours. After the reaction was completed, the mixture was filtrated and the filter cake was washed with EtOAc. The product was collected and dried under vacuum to afford 7-(3,4-dichlorophenyl)-2-methylthiazolo[4,5-d]pyridazin-4 (5H)-one (350 mg, 73.3%) as a light yellow solid. LCMS: (M+H)⁺=312.0, Retention time=1.233 min.

Step 5: To a solution of 7-(3,4-dichlorophenyl)-2-methyl-5H-thiazolo[4,5-d]pyridazin-4-one (100 mg, 0.32 mmol, 1 eq) in DMF (5 mL) were added (2-bromoethyl)benzene (119 mg, 0.64 mmol, 2 eq) and K₂CO₃ (133 mg, 0.96 mmol, 3 eq) under N₂. The reaction mixture was stirred at 25° C. for 16 hours. After reaction was completed, the reaction mixture was diluted with H₂O and extracted with EtOAc (20 mL×3). The combined organic phase was washed with brine (20 mL×2), dried over anhydrous sodium sulfate, filtrated and concentrated under reduced pressure to give a residue, which was purified by SGC (Petroleum ether/Ethyl acetate=50/50) to afford the crude product (100 mg), which was further purified by prep-HPLC (ACN: 0.1% HCOOH in H₂O from 80:20 to 95:5 over 10 min) to get 7-(3,4-dichlorophenyl)-2-methyl-5-phenethylthiazolo[4,5-d]pyridazin-4 (5H)-one (44.1 mg, 32.3%) as a white solid. LCMS: (M+H)⁺=416.1, Retention time=1.690 min. HPLC: purity=97.061% (214 nm); purity=99.483% (254 nm); Retention time=4.307 min. ¹H NMR (400 MHz, DMSO) δ 7.84-7.82 (d, J=8.4 Hz, 1H), 7.74-7.74 (d, J=2.4 Hz, 1H), 7.68-7.66 (dd, J=2.4 Hz, J=8.4 Hz, 1H), 7.30-7.21 (m, 5H), 4.54-4.50 (t, J=7.2 Hz, 2H), 3.14-3.10 (t, J=7.2 Hz, 2H), 2.88 (s, 3H).

### Example 52: 2-amino-7-(4-chlorophenyl)-5-(2-phenylethyl)thiazolo[4,5-d]pyridazin-4-one I-83

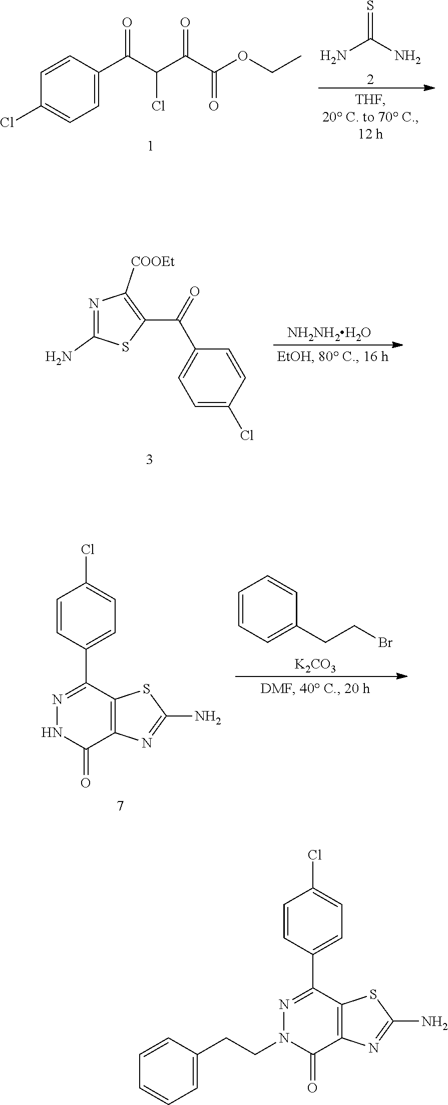

Step 1: To a solution of ethyl 3-chloro-4-(4-chlorophenyl)-2,4-dioxo-butanoate [1.38 g, 4.76 mmol, 1.0 eq] in THF [15 mL] was added thiourea [0.40 g, 5.24 mmol, 1.1 eq]. The resulting mixture was stirred at 20° C. for 12 hours. Then the reaction was stirred at 70° C. for 1 hour. The reaction mixture was diluted water (20 mL) and extracted with ethyl acetate (20 mL×3). The combined organic phase was washed with brine (10 mL×2), dried over anhydrous sodium sulfate, filtrated and concentrated under reduced pressure to give a crude product. The product was purified by flash chromatography (EtOAc/Petroleum ether=35%) to give the desired product as a white solid (1.08 g, 72.2%). LCMS (M+H)⁺=311.09, Retention time=1.025 min. ¹H NMR (400 MHz, DMSO) δ 8.17 (s, 1H), 7.67 (d, J=8.4 Hz, 2H), 7.58 (d, J=8.4 Hz, 2H), 3.79 (q, J=7.2 Hz, 2H), 0.94 (t, J=7.0 Hz, 3H).

Step 2: To a solution of ethyl 2-amino-5-(4-chlorobenzoyl) thiazole-4-carboxylate [1.08 g, 3.48 mmol, 1.0 eq] in EtOH [15 mL] was added hydrazine hydrate [261 mg, 5.21 mmol, 1.5 eq]. The resulting mixture was stirred at 80° C. for 16 hours and then cooled down to room temperature. The product was collected by filtration and washed with ethyl acetate and ethanol to give 2-amino-7-(4-chlorophenyl)-5H-thiazolo[4,5-d]pyridazin-4-one as a yellow solid (670 mg, 68.5%). LCMS (M+H)⁺=278.95, Retention time=0.823 min. ¹H NMR (400 MHz, DMSO) δ 12.93 (s, 1H), 8.21 (s, 2H), 7.75 (d, J=8.5 Hz, 2H), 7.61 (d, J=8.5 Hz, 2H).

Step 3: To a mixture of 2-amino-7-(4-chlorophenyl)-5H-thiazolo[4,5-d]pyridazin-4-one [300 mg, 1.08 mmol, 1.0 eq] and K₂CO₃ [178 mg, 1.29 mmol, 1.2 eq] in DMF [5 mL] was added (2-bromoethyl) benzene [199 mg, 1.08 mmol, 1.0 eq] dropwise. The resulting mixture was stirred at 40° C. for 20 hours. The reaction was diluted with H₂O (10 mL) and extracted with ethyl acetate (3×20 mL). The combined organic phase was washed with brine (5 mL×3), dried over anhydrous sodium sulfate, filtrated and concentrated under reduced pressure to give a residue, which was purified by flash column chromatography (MeOH/DCM=3/97) to afford 2-amino-7-(4-chlorophenyl)-5-(2-phenylethyl)thiazolo[4,5-d]pyridazin-4-one (84 mg, 19.4%) as a yellow solid. LCMS (M+H)⁺=383.10, Retention time=1.428 min. HPLC: purity=93.32% (254 nm); purity=93.61% (214 nm); Retention time=3.522 min. ¹H NMR (400 MHz, DMSO) δ 8.26 (s, 2H), 7.68-7.64 (m, 2H), 7.62-7.57 (m, 2H), 7.32-7.20 (m, 5H), 4.42 (t, J=7.4 Hz, 2H), 3.09 (t, J=7.6 Hz, 2H).

### Example 53: 2-methyl-5-phenethyl-7-(p-tolyl)thiazolo[4,5-d]pyridazin-4 (5H)-one I-84

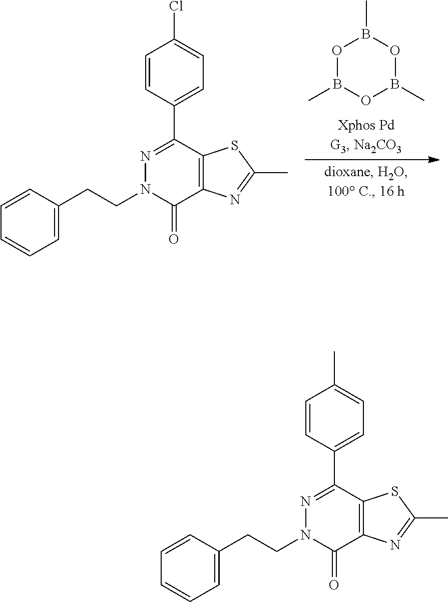

Step 1: To a solution of 7-(4-chlorophenyl)-2-methyl-5-phenethylthiazolo[4,5-d]pyridazin-4 (5H)-one (200 mg, 0.52 mmol, 1.0 eq) in dioxane/H₂O (=10:1) (5.0 mL) were added Na₂CO₃ (166 mg, 1.57 mmol, 3.0 eq), 2,4,6-trimethyl-1,3,5,2,4,6-trioxatriborinane (328 mg, 2.62 mmol, 5.0 eq), and Xphos-Pd-G3 (44 mg, 0.05 mmol, 0.1 eq). The mixture was stirred at 100° C. for 16 hours. The reaction was diluted with water (20 ml) and extracted with EtOAc (20 ml*3). The combined organic layer was washed with brine, dried and concentrated under reduced pressure. The residue was purified with prep-HPLC to the desired product (80 mg, 41.4%) as a yellow solid. LCMS: (M+H)⁺=362, Retention time=1.309 min. HPLC: purity=98.87% (254 nm); purity=98.92% (214 nm); Retention time=3.645 min. ¹H NMR δ 7.59-7.57 (m, 2H), 7.36-7.34 (m, 2H), 7.31-7.16 (m, 5H), 4.52-4.48 (m, 2H), 3.14-3.11 (m, 2H), 2.87 (s, 3H), 2.38 (s, 3H).

### Example 54: N-[7-(4-chlorophenyl)-4-oxo-5-(2-phenylethyl)thiazolo[4,5-d]pyridazin-2-yl]-2-methyl-propanamide 1-85

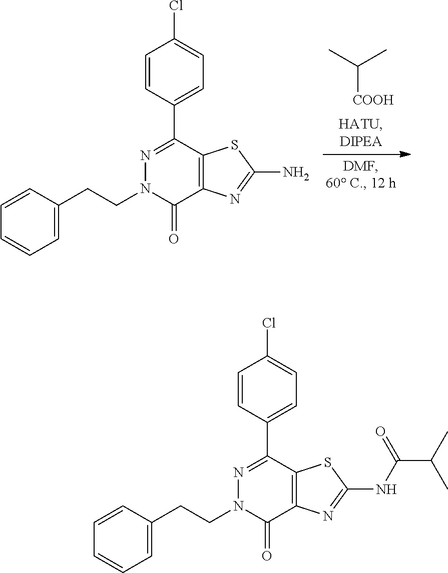

Step 1: To a solution of 2-methylpropanoic acid (17 mg, 0.20 mmol, 1.50 eq) in DMF (2.5 mL) were added HATU (74 mg, 0.20 mmol, 1.50 eq), 2-amino-7-(4-chlorophenyl)-5-(2-phenylethyl)thiazolo[4,5-d]pyridazin-4-one (50 mg, 0.13 mmol, 1.0 eq) and DIEA (0.032 mL, 0.20 mmol, 1.5 eq). The resulting mixture was stirred at 60° C. for 20 hours. After the reaction was cooled to room temperature, the mixture was diluted with water and extracted with ethyl acetate (20 mL×3). The combined organic phase was washed with brine (10 mL×2), dried over anhydrous sodium sulfate, filtrated and concentrated under reduced pressure to give a residue, which was purified by flash chromatography (Petroleum ether/Ethyl acetate=75/25) to give N-[7-(4-chlorophenyl)-4-oxo-5-(2-phenylethyl) thiazolo [4,5-d]pyridazin-2-yl]-2-methyl-propanamide as a white solid (25 mg, 41.4%). LCMS (M+H)⁺=453.00, Retention time=1.290 min. HPLC: purity=98.34% (254 nm); purity=97.29% (214 nm); Retention time=4.965 min

¹H NMR (400 MHz, CDCl₃) δ 11.30 (s, 1H), 7.68 (d, J=8.4 Hz, 2H), 7.47 (d, J=8.4 Hz, 2H), 7.33-7.18 (m, 5H), 4.66 (t, J=7.4 Hz, 2H), 3.24 (t, J=7.4 Hz, 2H), 3.07-2.91 (m, 1H), 1.35 (d, J=5.9 Hz, 6H).

### Example 55: 7-(4-chlorophenyl)-5-(2-phenylethyl)-2-(2-phenylethylamino)thiazolo[4,5-d]pyridazin-4-one 1-87

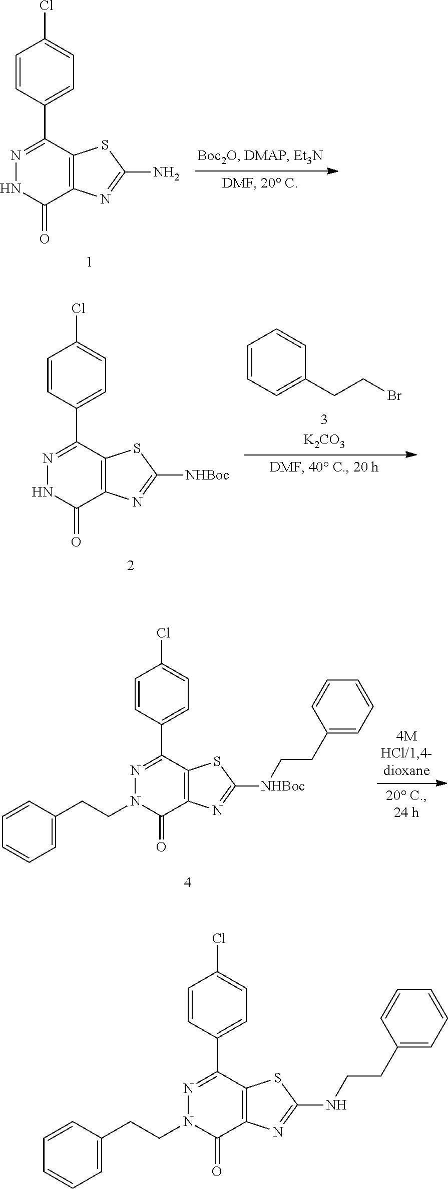

Step 1: To a mixture of 2-amino-7-(4-chlorophenyl)-5H-thiazolo[4,5-d]pyridazin-4-one [115 mg, 0.41 mmol, 1.0 eq] and Boc₂O (108 mg, 0.50 mmol, 1.20 eq) in DMF [5.0 mL] were added DMAP (5.0 mg, 0.041 mmol, 0.1 eq) and Et₃N (63 mg, 0.62 mmol, 1.5 eq). The resulting mixture was stirred at 20° C. for 16 hours. Then the reaction was quenched with saturated NaHCO₃ and extracted with DCM (10 mL×3). The combined organic phase was washed with brine (10 mL×3), dried over anhydrous sodium sulfate, filtrated and concentrated under reduced pressure. The residue was purified by flash chromatography with Petroleum ether/Ethyl acetate (=25/75) to give the tert-butyl N-[7-(4-chlorophenyl)-4-oxo-5H-thiazolo[4,5-d]pyridazin-2-yl]carbamate. (145 mg, 91.8%). LCMS (M+H)⁺=379.13, Retention time=1.199 min

Step 2: To a mixture of tert-butyl N-[7-(4-chlorophenyl)-4-oxo-5H-thiazolo[4,5-d]pyridazin-2-yl]carbamate [145 mg, 0.38 mmol, 1.0 eq] and K₂CO₃ [158 mg, 1.15 mmol, 3.0 eq] in DMF [3 mL] was added (2-bromoethyl) benzene [212 mg, 1.15 mmol, 3.0 eq] dropwise. The resulting mixture was stirred at 40° C. for 12 hours. The reaction mixture was diluted water and extracted with DCM (10 mL×3). The combined organic phase was washed with brine (10 mL×3), dried over anhydrous sodium sulfate, filtrated and concentrated under reduced pressure to give a residue, which was purified by flash chromatography with Petroleum ether/Ethyl acetate (=60/40) to give the tert-butyl N-[7-(4-chlorophenyl)-4-oxo-5-(2-phenylethyl)thiazolo[4,5-d]pyridazin-2-yl]carbamate (78 mg, 34.8%). LCMS [M+H]⁺: 587.31, Retention time=1.977 min

Step 3: To a solution of tert-butyl N-[7-(4-chlorophenyl)-4-oxo-5-(2-phenylethyl) thiazolo [4,5-d]pyridazin-2-yl]-N-(2-phenylethyl) carbamate (23 mg, 0.04 mmol, 1.0 eq) in 1,4-dioxane (0.5 mL) was added 4 M HCl in 1,4-dioxane (2.0 mL) dropwise at 0° C. The reaction mixture was allowed to warm to room temperature and stirred for 24 hours. The mixture was concentrated at 45° C. under reduced pressure. The desired product was obtained by trituration in ethyl acetate (5 mL) as a white solid. (5.23 mg, 23.3%) LCMS (M+H)⁺=487.24, Retention time=1.698 min. HPLC: purity=95.75% (254 nm); purity=84.88% (214 nm); Retention time=4.451 min. ¹H NMR (400 MHz, CDCl₃) δ 7.57-7.48 (m, 2H), 7.46 (t, J=6.8 Hz, 2H), 7.35-7.18 (m, 10H), 4.71-4.54 (m, 2H), 3.80-3.56 (m, 2H), 3.24-3.20 (m, 2H), 3.09-3.05 (m, 2H).

### Example 56: 7-(4-chlorophenyl)-2-(cyclopropylmethylamino)-5-(2-phenylethyl)thiazolo[4,5-d]pyridazin-4-one I-86

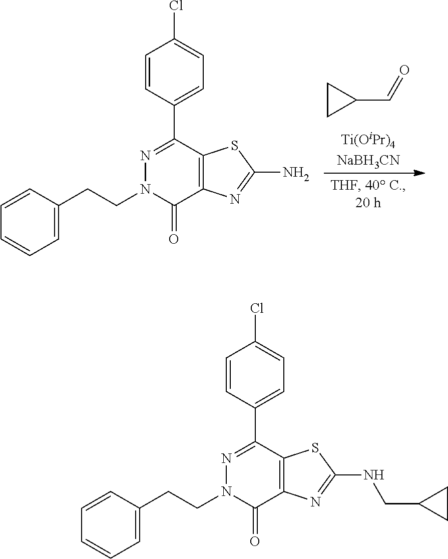 [SMILES: Nc1nc2c(=O)n(CCc3ccccc3)nc(-c3ccc(Cl)cc3)c2s1 ; O=CC1CC1 ; O=c1c2nc(NCC3CC3)sc2c(-c2ccc(Cl)cc2)nn1CCc1ccccc1]

Step 1: To a solution of 2-amino-7-(4-chlorophenyl)-5-(2-phenylethyl) thiazolo [4,5-d]pyridazin-4-one (72 mg, 0.19 mmol, 1.0 eq) in THF (5 mL) were added cyclopropanecarboxaldehyde (132 mg, 1.88 mmol, 10 eq) and Ti(OⁱPr)₄ (160 mg, 0.56 mmol, 3.0 eq). The reaction mixture was stirred at 40° C. for 12 hours. Then NaBH₃CN (47 mg, 0.75 mmol, 4.0 eq) was added and the reaction was continued to stir at 40° C. for 20 hours. The mixture was diluted with water and extracted with DCM (3×20 mL). The combined organic layer was washed with brine, dried over Na₂SO₄, and concentrated under reduced pressure. The crude product was purified by flash chromatography (DCM) and then further purified by recrystallization in DCM (10 mL) at −15° C. The precipitate was filtered and triturated in MeOH/EtOH (3 mL/3 mL) to give the desired product as a white solid. LCMS (M+H)⁺=437.20, Retention time=1.416 min. HPLC: purity=96.46% (254 nm); purity=84.62% (214 nm); Retention time=3.909 min. ¹H NMR (400 MHz, DMSO) δ 8.92 (s, 1H), 7.64 (d, J=8.4 Hz, 2H), 7.59 (d, J=8.4 Hz, 2H), 7.28-7.19 (m, 5H), 4.43 (t, J=7.3 Hz, 2H), 3.28 (t, J=6.0 Hz, 2H), 3.09 (t, J=7.3 Hz, 2H), 1.19-1.02 (m, 1H), 0.63-0.42 (m, 2H), 0.28 (q, J=4.5 Hz, 2H).

### Example 57: 4-[7-(4-chlorophenyl)-2-methyl-4-oxo-thiazolo[4,5-d]pyridazin-5-yl]benzoic acid and 4-(7-(4-chlorophenyl)-2-methyl-4-oxothiazolo[4,5-d]pyridazin-5 (4H)-yl)-N-isopropylbenzamide I-88 and I-92

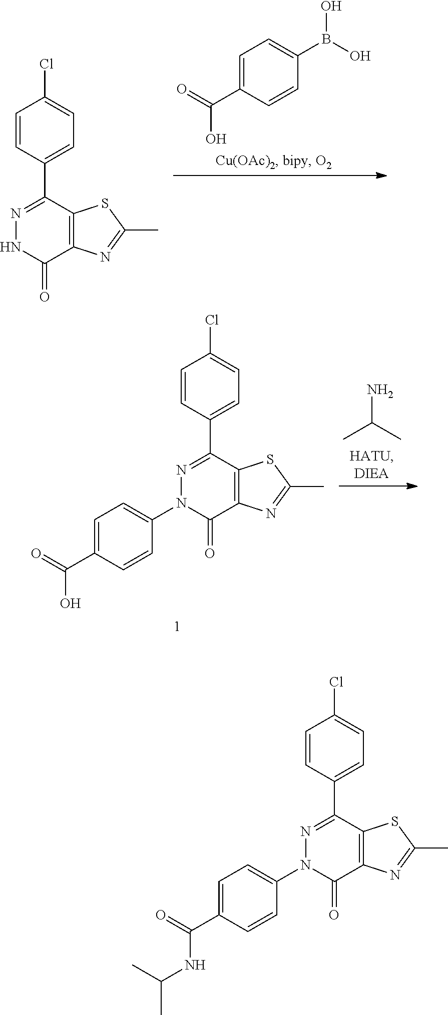

Step 1: A mixture of 7-(4-chlorophenyl)-2-methyl-5H-thiazolo[4,5-d]pyridazin-4-one (300 mg, 1.08 mmol, 1.0 eq), 4-boronobenzoic acid (358 mg, 2.16 mmol, 2.0 eq), Cu(OAc)₂ (196 mg, 1.08 mmol, 1.0 eq), Bi-pyridine (169 mg, 1.08 mmol, 1.0 eq) and Na₂CO₃ (229 mg, 2.17 mmol, 2.0 eq) in DMF (5 mL) was stirred at 70° C. under oxygen atmosphere overnight. The mixture was diluted with water (20 mL) and extracted with CH₂Cl₂ (20 mL*3). The organic layers were combined, washed with brine (50 mL), dried over Na₂SO₄, and concentrated to give compound 1 as a black oil (1 g, >100%, contained DMF, 20% purity). The crude product was used for next step without purification. LCMS: M+H=398.1, Retention time=1.295 min

Step 2: To a mixture of 4-[7-(4-chlorophenyl)-2-methyl-4-oxo-thiazolo[4,5-d]pyridazin-5-yl]benzoic acid (200 mg, 0.5 mmol, 1 g contained DMF, 1.0 eq) in DMF (1 mL) were added HATU (382 mg, 1 mmol, 2.0 eq) and DIPEA (325 mg, 2.51 mmol, 5 eq). The mixture was stirred for 0.5 h at 25° C., and then isopropylamine (89 mg, 1.5 mmol, 3.0 eq) was added. The reaction was stirred at room temperature for 3 hours. The mixture was diluted with water (20 mL) and extracted with CH₂Cl₂ (20 mL*3). The combined organic phases were washed with brine, dried over Na₂SO₄, filtered, and concentrated to give the crude product. The crude product was purified by prep-HPLC to give the desired product (5.77 mg, 2.4%) as a white solid. LCMS: M+H=439.2, Retention time=1.295 min. HPLC: purity=93.45% (254 nm); purity=92.22% (214 nm); Retention time=3.352 min ¹H NMR (400 MHz, DMSO) δ 7.90-7.84 (m, 4H), 7.81-7.80 (d, J=4.4 Hz, 2H), 7.52-7.50 (d, J=4.4 Hz, 2H), 4.34-4.29 (m, 1H), 2.96 (s, 3H), 1.30-1.28 (d, J=6.4 Hz, 6H).

### Example 58: Synthesis of 7-(4-hydroxyphenyl)-2-methyl-5-phenethylthiazolo[4,5-d]pyridazin-4 (5H)-one, I-89, and 7-(4-methoxyphenyl)-2-methyl-5-phenethylthiazolo[4,5-d]pyridazin-4 (5H)-one I-90

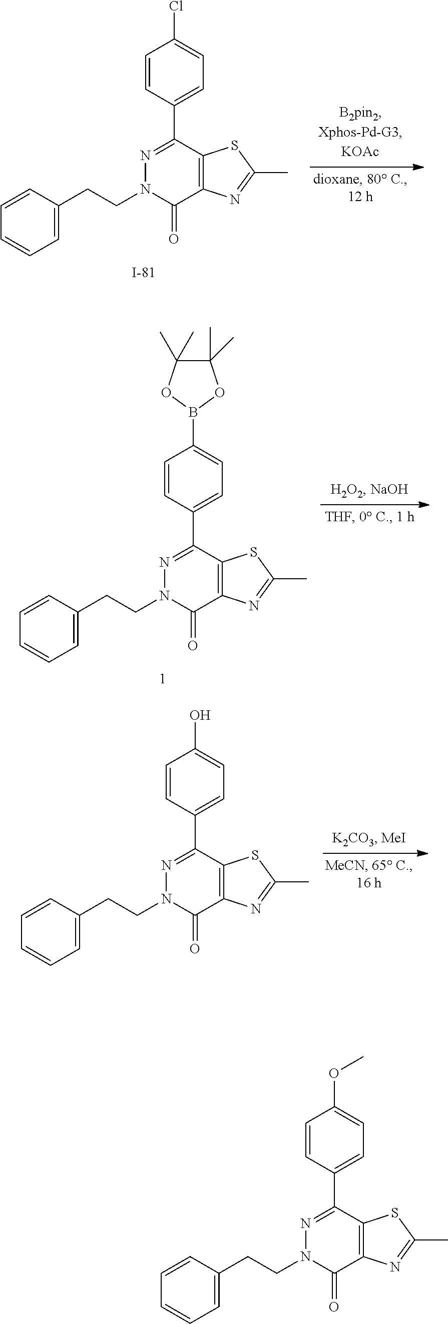

Step 1: To a mixture of I-81 (500 mg, 1.31 mmol, 1.0 eq) in dioxane/H₂O (10:1) (10 mL: 1 mL) were added KOAc (386 mg, 3.93 mmol, 3.0 eq) and B₂(pin)₂ (997 mg, 3.93 mmol, 3.0 eq) at 25° C., Xphos-Pd-G2 (21 mg, 0.026 mmol, 0.02 eq) under N₂. The mixture was stirred at 80° C. for 12 hours. The mixture was diluted with H₂O (20 mL) and extracted with EtOAc (20 mL*3). The organic layers were combined, dried over Na₂SO₄ and evaporated to give the crude product. The crude product was purified by silica gel chromatograph (EtOAc: Petroleum ether=30%) to give Compound 1 (400 mg, 64%) as a white solid. LCMS: (M+H)⁺=474, Retention time=1.723 min.

Step 2: To a solution of Compound 1 [420 mg, 0.89 mmol, 1.0 eq] in THF [5 mL] were added aqueous sodium hydroxide [88 mg, 2.22 mmol, 0.5 mL, 2.5 eq] and hydrogen peroxide (30%) [300 mg] successively at 0° C. The reaction mixture was allowed to warm to room temperature and stirred for 1 hour. The mixture was diluted with H₂O (20 mL) and extracted with DCM (20 mL*3). The organic layers were combined, dried over Na₂SO₄, and evaporated to give the crude product. The crude product was purified by silica gel chromatograph (DCM:MeOH=10:1) to give I-89 (100 mg, 29%) as a white solid. A portion of the product (20 mg) was purified by prep-HPLC to give the desired product (1.62 mg) as a white solid. LCMS: (M+H)⁺=364, Retention time=1.203 min. HPLC: purity=95.50% (254 nm); purity=94.68% (214 nm); Retention time=2.688 min. ¹H NMR (400 MHz, MeOD) δ 7.45-7.37 (m, 2H), 7.30-7.16 (m, 5H), 6.71-6.66 (m, 2H), 4.59 (t, J=7.2 Hz, 2H), 3.32 (s, 3H), 3.21 (t, J=7.2 Hz, 2H).

Step 3: To a solution of I-89 (50 mg, 0.29 mmol, 1.0 eq) in MeCN (5 mL) were added K₂CO₃ (57 mg, 0.41 mmol, 1.4 eq) and methyl iodide (58 mg, 0.41 mmol, 1.4 eq). The reaction mixture was stirred at 65° C. for 16 hours. The reaction mixture was diluted with water (10 mL) and extracted with EtOAc (10 mL*3). The organic layers were combined, dried over sodium sulfate, and evaporated. The product was purified by silica gel chromatograph (Ethyl acetate: Petroleum ether=30%) to give the desired product I-90 (10 mg, 19%) as a white solid. The product (10 mg) was purified by prep-HPLC to give the desired product (5.01 mg) as a white solid. LCMS: (M+H)⁺=378, Retention time=1.212 min. HPLC: purity=96.85% (254 nm); purity=97.29% (214 nm); Retention time=3.357 min. ¹H NMR (400 MHz, DMSO) δ 7.65-7.62 (m, 2H), 7.32-7.16 (m, 5H), 7.11-7.08 (m, 2H), 4.49 (t, J=7.3 Hz, 2H), 3.84 (s, 3H), 3.13 (t, J=7.4 Hz, 2H), 2.87 (s, 3H).

### Example 59: Synthesis of -(4-chlorophenyl)-2-methyl-5-(4-(trifluoromethoxy)phenyl)thiazolo[4,5-d]pyridazin-4 (5H)-one I-91

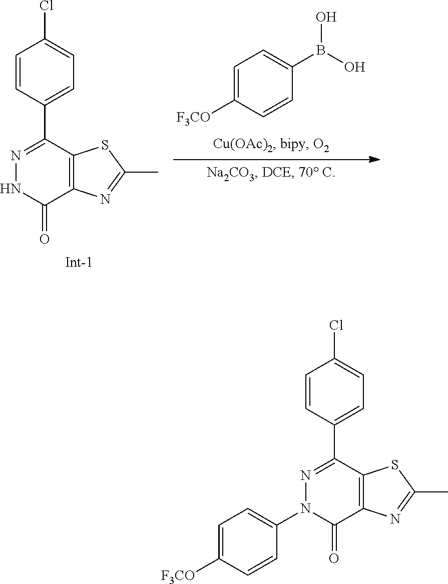 [SMILES: Cc1nc2c(=O)[nH]nc(-c3ccc(Cl)cc3)c2s1 ; OB(O)c1ccc(OC(F)(F)F)cc1 ; Cc1nc2c(=O)n(-c3ccc(OC(F)(F)F)cc3)nc(-c3ccc(Cl)cc3)c2s1]

Step 1: To a mixture of 7-(4-chlorophenyl)-2-methylthiazolo[4,5-d]pyridazin-4 (5H)-one (150 mg, 0.54 mmol, 1 eq) and (4-(trifluoromethoxy)phenyl)boronic acid (122 mg, 0.59 mmol, 1.1 eq) in THF (2 mL) were added Cu(OAc)₂ (98 mg, 0.54 mmol, 1 eq) and Et₃N (164 mg, 1.62 mmol, 3 eq). The mixture was stirred at 25° C. under N₂ atmosphere overnight. After the reaction was completed, the mixture was concentrated under reduced pressure to give the crude product. The crude product was purified by silica gel column (petroleum ether:EtOAc=3:1) to give the desired product as a white solid (80 mg, 33.5%). LCMS: (M+H)+=436.0, Retention time=1.615 min. HPLC: purity=99.88% (254 nm); purity=99.74% (214 nm); Retention time=4.194 min. ¹H NMR (400 MHz, CDCl₃) δ 7.81 (d, J=8.7 Hz, 4H), 7.51 (d, J=8.6 Hz, 2H), 7.36 (d, J=8.5 Hz, 2H), 2.96 (s, 3H).

### Example 60: Synthesis of 5-(4-chloro-2-fluoro-phenyl)-7-[6-(1-cyclopropylpyrazol-4-yl)-3,6-dihydro-2H-pyran-4-yl]-2-methyl-1,7-naphthyridin-8-one I-104

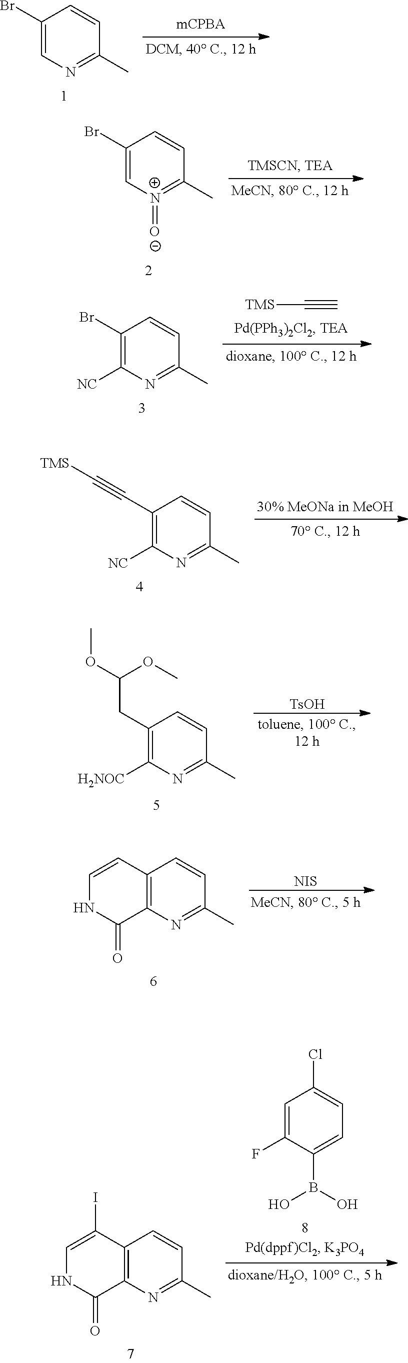

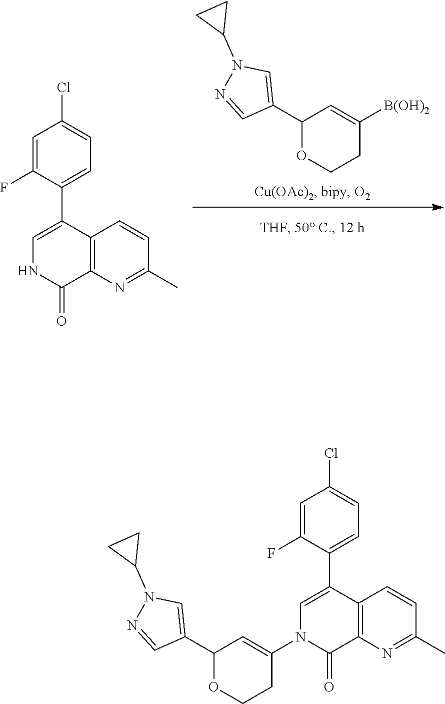 [SMILES: Cc1ccc2c(-c3ccc(Cl)cc3F)c[nH]c(=O)c2n1 ; OB(O)C1=CC(c2cnn(C3CC3)c2)OCC1 ; Cc1ccc2c(-c3ccc(Cl)cc3F)cn(C3=CC(c4cnn(C5CC5)c4)OCC3)c(=O)c2n1]

Step 1: To a solution of 5-bromo-2-methyl-pyridine (5.00 g, 29.1 mmol, 1.0 eq) in DCM (70 mL) was added mCPBA (7.08 g, 34.9 mmol, 1.2 eq). The reaction was stirred at 40° C. for 12 h. The reaction mixture was washed with saturated Na₂S₂O₄ (100 mL) and saturated NaHCO₃ (100 mL). The organics were then separated and dried (Na₂SO₄) before concentration to give 1-blah-5-bromo-2-methyl-pyridine as a yellow solid (5.00 g, 91.5%).

Step 2: To a solution of 1-blah-5-bromo-2-methyl-pyridine (5.00 g, 26.6 mmol, 1.0 eq) in MeCN (100 mL) was added TMSCN (14 mL, 106 mmol, 4.0 eq) and TEA (6.9 mL, 79.8 mmol, 3.0 eq). The reaction was stirred at 80° C. for 12 h. The reaction was concentrated and purified by flash column chromatography eluting with 10% EtOAc in Petroleum ether. The desired fractions were concentrated to dryness in vacuo to give 3-bromo-6-methyl-pyridine-2-carbonitrile as a white solid (4.00 g, 76.3%).

Step 3: To a solution of 3-bromo-6-methyl-pyridine-2-carbonitrile (4.00 g, 20.3 mmol, 1.0 eq) and TRIMETHYLSILYLACETYLENE (3.4 mL, 24.4 mmol, 1.2 eq) in 1,4-dioxane (60 mL) were added Pd(PPh₃)₂Cl₂ (356 mg, 0.51 mmol, 0.025 eq) and CuI (194 mg, 1.02 mmol, 0.05 eq). The reaction was stirred at 100° C. for 12 hours. After cooling to ambient temperature, the mixture was filtered through celite and the filtrate was concentrated under vacuum. The crude was then purified by flash column chromatography eluting with 10% EtOAc in Petroleum ether. The desired fractions were concentrated to dryness in vacuo to afford 6-methyl-3-(2-trimethylsilylethynyl)pyridine-2-carbonitrile as a white solid (3.60 g, 82.7%).

Step 4: A mixture of 6-methyl-3-(2-trimethylsilylethynyl)pyridine-2-carbonitrile (3.6 g, 16.82 mmol, 1.0 eq) in 30% MeONa in MeOH (40 mL) was stirred at 70° C. for 12 hours. The reaction was concentrated to dryness and the residue was taken up in DCM (100 mL) and the organics were washed with water (2×100 mL) and brine (1×100 mL). The organics were then separated and dried (Na₂SO₄) before concentration to dryness. The crude was then purified by flash column chromatography eluting with 50% EtOAc in Petroleum ether. The desired fractions were concentrated to dryness in vacuo to afford 3-(2,2-dimethoxyethyl)-6-methyl-pyridine-2-carboxamide as a brown solid (1.8 g, 47.8%).

Step 5: To a solution of 3-(2,2-dimethoxyethyl)-6-methyl-pyridine-2-carboxamide (1.8 g, 7.93 mmol, 1.0 eq) in toluene (20 mL) was added TsOH (0.41 g, 2.38 mmol, 0.3 eq). The reaction was stirred at 100° C. for 12 hours. The reaction was concentrated and the residue was purified by flash column chromatography eluting with 2% MeOH in DCM. The desired fractions were concentrated to dryness in vacuo to afford 2-methyl-7H-1,7-naphthyridin-8-one as a white solid (1.11 g, 87.5%).

Step 6: To a solution of 2-methyl-7H-1,7-naphthyridin-8-one (1.00 g, 6.24 mmol, 1.0 eq) in MeCN (80 mL) was added NIS (2.81 g, 12.5 mmol, 2.0 eq). The reaction was stirred at 80° C. for 12 hours. The reaction was filtered and solid was dried to afford 5-iodo-2-methyl-7H-1,7-naphthyridin-8-one as a yellow solid (600 mg, 68.5% purity, 23% yield).

Step 7: To a solution of 5-iodo-2-methyl-7H-1,7-naphthyridin-8-one (600 mg, 2.10 mmol, 1.0 eq) and (4-chloro-2-fluoro-phenyl)boronic acid (731 mg, 4.19 mmol, 2.0 eq) in 1,4-dioxane (100 mL) and water (10 mL) were added K₃PO₄ (1.33 g, 6.29 mmol, 3.0 eq) and Pd(dppf)Cl₂ (171 mg, 0.21 mmol, 0.1 eq). The reaction was stirred at 90° C. for 12 hours under N₂. After cooling to ambient temperature, the mixture was filtered through celite and the filtrate was concentrated under vacuum. The residue was purified by flash column chromatography eluting with 2% MeOH in DCM. The desired fractions were concentrated to dryness in vacuo to afford 5-(4-chloro-2-fluoro-phenyl)-2-methyl-7H-1,7-naphthyridin-8-one as a white solid (360 mg, 47.6%). ¹H NMR (400 MHz, DMSO) δ 11.75 (s, 1H), 7.55 (d, J=8.4 Hz, 1H), 7.51 (s, 2H), 7.45 (t, J=8.1 Hz, 1H), 7.39 (d, J=8.2 Hz, 1H), 7.21 (d, J=4.3 Hz, 1H), 2.57 (s, 3H).

Step 8: To a solution of 5-(4-chloro-2-fluoro-phenyl)-2-methyl-7H-1,7-naphthyridin-8-one (200 mg, 0.69 mmol, 1.0 eq) and [6-(1-cyclopropylpyrazol-4-yl)-3,6-dihydro-2H-pyran-4-yl]boronic acid (486 mg, 2.08 mmol, 3.0 eq) in THF (20 mL) were added Cu(OAc)₂ (277 mg, 1.39 mmol, 2.0 eq) and 2,2-bipyridine (216 mg, 1.39 mmol, 2.0 eq). The reaction was stirred at 70° C. for 12 hours under O₂. After cooling to ambient temperature, the mixture was filtered through celite and the filtrate was concentrated under vacuum. The residue was purified by silica gel column chromatography (eluting with MeOH/DCM, 1% to 3%) to afford 5-(4-chloro-2-fluoro-phenyl)-7-[6-(1-cyclopropylpyrazol-4-yl)-3,6-dihydro-2H-pyran-4-yl]-2-methyl-1,7-naphthyridin-8-one as a white solid (100 mg, 26.0%). ¹H NMR (400 MHz, CDCl₃) δ 7.66 (s, 1H), 7.61 (d, J=8.8 Hz, 1H), 7.55 (s, 1H), 7.46 (d, J=8.2 Hz, 1H), 7.30-7.28 (m, 3H), 7.12 (s, 1H), 6.05 (s, 1H), 5.39 (s, 1H), 4.04-3.96 (m, 1H), 3.94-3.91 (m, 1H), 3.60-3.52 (m, 1H), 2.84 (s, 3H), 2.66-2.59 (m, 2H), 1.14-1.13 (m, 2H), 1.04-1.02 (m, 2H).

### Example 61: Synthesis of 4-(3,4-dichlorophenyl)-6,7-dimethyl-2-[2-(oxetan-3-yl)-4-pyridyl]phthalazin-1-one I-107

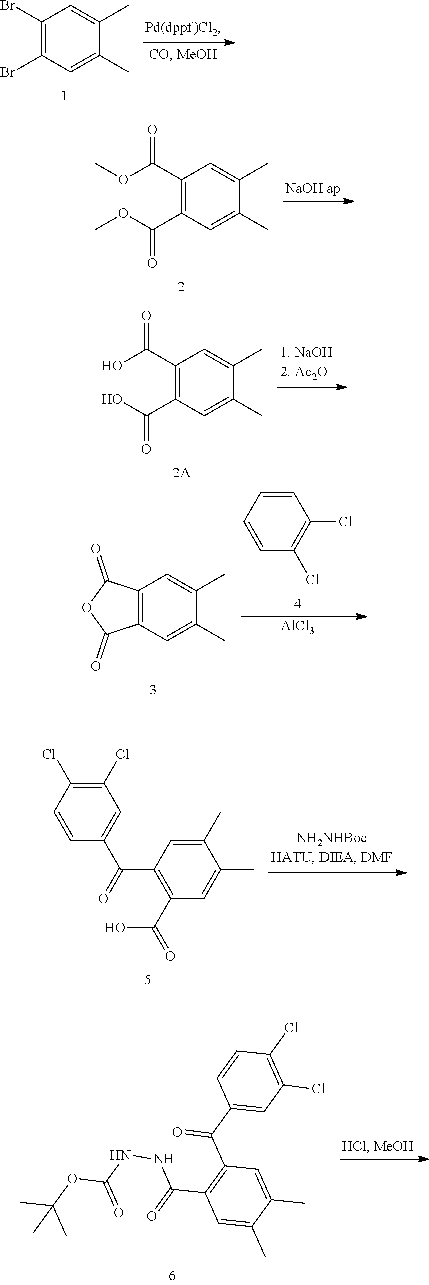

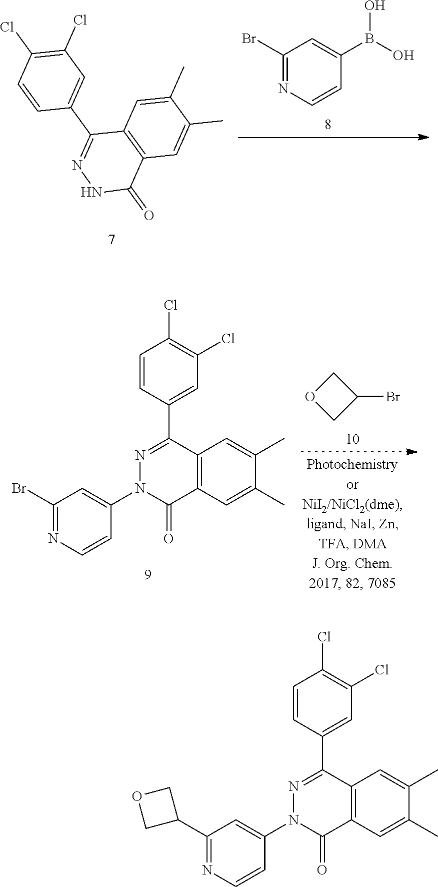 [SMILES: Cc1cc2c(-c3ccc(Cl)c(Cl)c3)n[nH]c(=O)c2cc1C ; OB(O)c1ccnc(Br)c1 ; Cc1cc2c(-c3ccc(Cl)c(Cl)c3)nn(-c3ccnc(Br)c3)c(=O)c2cc1C ; BrC1COC1 ; Cc1cc2c(-c3ccc(Cl)c(Cl)c3)nn(-c3ccnc(C4COC4)c3)c(=O)c2cc1C]

Step 1: To a solution of 1,2-dibromo-4,5-dimethyl-benzene (1.00 eq, 20.00 g, 75.8 mmol) in Methanol (200 mL) was added TEA (3.00 eq, 32 mL, 227 mmol) and Pd(dppf)Cl₂·CH₂Cl₂ (0.100 eq, 6.18 g, 7.58 mmol), the mixture was stirred at 80° C. for 12 h in CO (50 Psi) atmosphere. The reaction mixture was filtered under N₂ atmosphere, the filtrate was concentrated to give a crude product. The crude product was purified by column chromatography on silica gel eluted with petroleum ether/ethyl acetate (5:1). dimethyl 4,5-dimethylbenzene-1,2-dicarboxylate (15.30 g, 68.8 mmol, 90.86% yield) was obtained as white solid, LC-MS: Rt: 0.781 min; 245.1=[M+23]+, ESI+; 100% purity at 220 nm. ¹H NMR (400 MHz, CHLOROFORM-d) δ=7.49 (s, 2H), 3.89 (s, 6H), 2.32 (s, 6H).

Step 2: To a solution of dimethyl 4,5-dimethylbenzene-1,2-dicarboxylate (1.00 eq, 15.30 g, 68.8 mmol) in Methanol (150 mL) and Water (150 mL) was added NaOH (3.00 eq, 8.26 g, 207 mmol), the mixture was stirred at 25° C. for 12 h. The mixture was concentrated under vacuum to removed MeOH and concentrated under vacuum to give a solution. The solution was stirred and added 1N HCl (aq) to PH=2-3, a lot of white solid produced. The mixture was filtered and the filter cake was concentrated under vacuum to give 4,5-dimethylphthalic acid (12.50 g, 64.4 mmol, 93.51% yield) as a white solid. LC-MS: Rt: 0.625 min; 217.1=[M+23]⁺, ESI⁺; 100% purity at 220 nm. ¹H NMR (400 MHz, DMSO-d6) δ=7.44 (s, 2H), 2.28 (s, 6H)

Step 3: A mixture of 4,5-dimethylphthalic acid (1.00 eq, 4.20 g, 21.6 mmol) in ACETIC ANHYDRIDE (7.35 eq, 15 mL, 159 mmol) was stirred at 100° C. for 3 h. The reaction mixture was concentrated in vacuum. The residue was triturated with PE:EA=10:1 (10 mL) to give 5,6-dimethylisobenzofuran-1,3-dione (3440 mg, 19.5 mmol, 90.28% yield) as a gray solid. ¹H NMR (400 MHz, CHLOROFORM-d) δ=7.764 (s, 2H), 2.474 (m, 6H)

Step 4: To a solution of 5,6-dimethylisobenzofuran-1,3-dione (1.00 eq, 1.00 g, 5.68 mmol) in 1,2-DICHLOROBENZENE (31.3 eq, 20 mL, 178 mmol) was added AlCl₃ (6.00 eq, 4541 mg, 34.1 mmol) in N2, the mixture was stirred at 80° C. for 3 h. The reaction mixture was poured into 1N HCl solution (100 mL) at 0° C., the aqueous phase was extracted with EtOAc (50 mL*3). The combined organic phase was washed with brine (50 mL*3), dried with anhydrous Na₂SO₄, filtered and concentrated to give a crude product in vacuum. The crude product was added PE/EA (10/1) (33 mL), the mixture was stirred at 25° C. for 0.5 h, The mixture was filtered and the filter cake was washed with PE, dried in vacuum to give product. 2-(3,4-dichlorobenzoyl)-4,5-dimethyl-benzoic acid (1.49 g, 4.45 mmol, 78.38% yield) was obtained as white solid. LC-MS: Rt: 0.949 min; 345.0=[M+23]⁺, ESI+; 96.5% purity at 220 nm. ¹H NMR (400 MHz, DMSO-d6) δ=13.21-12.98 (m, 1H), 7.82-7.74 (m, 3H), 7.49 (dd, J=2.0, 8.3 Hz, 1H), 7.25 (s, 1H), 2.36 (s, 3H), 2.33 (s, 3H)

Step 5: a solution of 2-(3,4-dichlorobenzoyl)-4,5-dimethyl-benzoic acid (1.00 eq, 1.00 g, 3.09 mmol) in DMF (15 mL) was added tert-butyl N-aminocarbamate (1.00 eq, 409 mg, 3.09 mmol), DIPEA (2.00 eq, 1.1 mL, 6.19 mmol) and HATU (1.20 eq, 1412 mg, 3.71 mmol), the mixture was stirred at 25° C. for 12 h. The reaction mixture was poured into water (60 mL), the mixture was filtered and the filter cake was washed with 20 mL of water, dried in vacuum to give product. The crude product was added PE/EA (10:1) (33 mL), the mixture was stirred at 25° C. for 0.5 h, the mixture was filtered and the filter cake was washed with 20 mL of water, dried in vacuum to give product. tert-butyl N-[[2-(3,4-dichlorobenzoyl)-4,5-dimethyl-benzoyl]amino]carbamate (1.30 g, 2.82 mmol, 91.26% yield) was obtained as white solid. LC-MS: Rt: 1.004 min; 419.1=[M−17]+, ESI+¹H NMR (400 MHz, DMSO-d6) δ=9.14 (s, 1H), 7.69-7.48 (m, 3H), 7.32-7.24 (m, 2H), 7.12 (s, 1H), 2.33 (s, 3H), 2.28 (s, 3H), 1.46-1.14 (m, 9H)

Step 6: A mixture of tert-butyl N-[[2-(3,4-dichlorobenzoyl)-4,5-dimethyl-benzoyl]amino]carbamate (1.00 eq, 1.30 g, 2.97 mmol) in Methanol (15 mL) was added HCl/MeOH (15.0 eq, 11 mL, 44.6 mmol). The mixture was stirred at 25° C. for 12 h. The mixture was concentrated to give a crude product. The crude product used for next step without further purification. 4-(3,4-dichlorophenyl)-6,7-dimethyl-2H-phthalazin-1-one; hydrochloride (1.00 g, 2.81 mmol, 94.59% yield) was obtained as white solid. LC-MS: Rt: 0.878 min; 319.1=[M+H]+, ESI+

Step 7: To a solution of 4-(3,4-dichlorophenyl)-6,7-dimethyl-2H-phthalazin-1-one; hydrochloride (1.00 eq, 500 mg, 1.41 mmol) in DMF (15 mL) was added (2-bromo-4-pyridyl)boronic acid (3.00 eq, 851 mg, 4.22 mmol), PYRIDINE (10.0 eq, 1.1 mL, 14.1 mmol) and Cu(OAc)₂ (1.30 eq, 331 mg, 1.83 mmol), the mixture was stirred at 80° C. for 12 h in O₂. The mixture was added (2-bromo-4-pyridyl)boronic acid (1 eq), the mixture was stirred at 80° C. for 12 h in O₂. The mixture was added (2-bromo-4-pyridyl)boronic acid (1 eq), the mixture was stirred at 80° C. for 12 h in O₂. The mixture was added (2-bromo-4-pyridyl)boronic acid (1 eq), the mixture was stirred at 80° C. for 12 h in O₂. The reaction mixture was poured into water (100 mL), the aqueous phase was extracted with DCM (100 mL*3). The combined organic phase was washed with brine (100 mL*3), dried with anhydrous Na₂SO₄, filtered and concentrated to give a crude product in vacuum. The crude product was purified by column chromatography on silica gel eluted with EA (0˜100%) in PE. 2-(2-bromo-4-pyridyl)-4-(3,4-dichlorophenyl)-6,7-dimethyl-phthalazin-1-one (454 mg, 0.831 mmol, 59.13% yield) was obtained as yellow solid. LC-MS: Rt: 1.112 min; 476.0=[M+H]⁺, ESI+; 87% purity at 220 nm.

Step 8: To a solution of 2-(2-bromo-4-pyridyl)-4-(3,4-dichlorophenyl)-6,7-dimethyl-phthalazin-1-one (1.00 eq, 100 mg, 0.210 mmol) in DMA (7.5 mL) was added 3-bromooxetane (1.20 eq, 35 mg, 0.253 mmol), sodium iodide (0.250 eq, 7.9 mg, 0.0526 mmol), pyridine-2,6-bis(carboximidamide) dihydrochloride (0.1000 eq, 3.4 mg, 0.0210 mmol), TFA (0.1000 eq, 0.0016 mL, 0.0210 mmol), Zinc powder (2.00 eq, 28 mg, 0.421 mmol) and NiCl₂(dme) (0.1000 eq, 4.6 mg, 0.0210 mmol) in N₂, the mixture was stirred at 60° C. for 4 h in N₂. The mixture was filtered and the filtrate was poured into water (40 mL), the aqueous phase was extracted with EA (40 mL*3). The combined organic phase was washed with brine (40 mL*3), dried with anhydrous Na₂SO₄, filtered and concentrated to give a crude product in vacuum. The crude product was purified by prep-HPLC (FA, column: Phenomenex Luna C18 150*25 mm*10 um; mobile phase: [water (FA)-ACN]; B %: 61%-91%, 10 min) and lyophilized to give 4-(3,4-dichlorophenyl)-6,7-dimethyl-2-[2-(oxetan-3-yl)-4-pyridyl]phthalazin-1-one (6.0 mg, 0.0131 mmol, 6.23% yield) was obtained as white solid. LC-MS: Rt: 0.962 min; 452.1=[M+H]+, ESI+; 98.9% purity at 220 nm. ¹H NMR (400 MHz, CHLOROFORM-d) δ=8.67 (d, J=5.4 Hz, 1H), 8.28 (s, 1H), 7.75 (d, J=1.8 Hz, 1H), 7.69-7.63 (m, 2H), 7.57 (d, J=8.3 Hz, 1H), 7.40 (dd, J=2.0, 8.3 Hz, 1H), 7.35 (s, 1H), 5.05-4.97 (m, 2H), 4.97-4.89 (m, 2H), 4.43-4.34 (m, 1H), 2.44 (s, 3H), 2.37 (s, 3H)

### Example 62: Synthesis of 4-(4-methoxyphenyl)-6,7-dimethyl-2-[2-(oxetan-3-yl)-4-pyridyl]phthalazin-1-one I-108

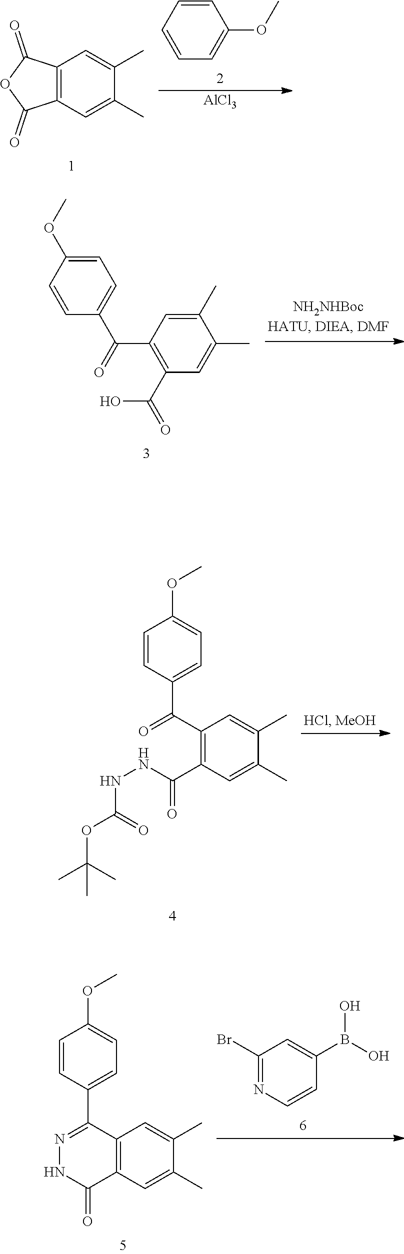

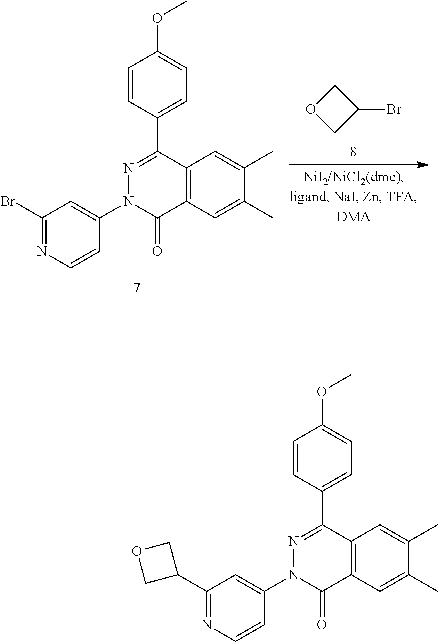 [SMILES: COc1ccc(-c2nn(-c3ccnc(Br)c3)c(=O)c3cc(C)c(C)cc23)cc1 ; BrC1COC1 ; COc1ccc(-c2nn(-c3ccnc(C4COC4)c3)c(=O)c3cc(C)c(C)cc23)cc1]

Step 1: To a solution of 5,6-dimethylisobenzofuran-1,3-dione (1.00 eq, 1.00 g, 5.68 mmol) in ANISOLE (1.80 eq, 1.1 mL, 10.2 mmol) was added AlCl₃ (3.00 eq, 2271 mg, 17.0 mmol) in N₂, the mixture was stirred at 50° C. for 3 h. The reaction mixture was poured into 1N HCl solution (100 mL) at 0° C., the aqueous phase was extracted with DCM (20 mL*3). The combined organic phase was washed with brine (20 mL*3), dried with anhydrous Na₂SO₄, filtered and concentrated to give a crude product in vacuum. The crude product was added PE/EA (10/1) (33 mL), the mixture was stirred at 25° C. for 0.5 h, The mixture was filtered and the filter cake was washed with PE, dried in vacuum to give product. 2-(4-methoxybenzoyl)-4,5-dimethyl-benzoic acid (1.55 g, 5.38 mmol, 94.80% yield) was obtained as a white solid. LC-MS: Rt: 0.874 min; 285.0=[M+H]⁺, ESI+; 98.7% purity at 220 nm. ¹H NMR (400 MHz, DMSO-d6) δ=7.75 (s, 1H), 7.61-7.55 (m, 2H), 7.14 (s, 1H), 7.05-6.97 (m, 2H), 3.83 (s, 3H), 2.34 (s, 3H), 2.31 (s, 3H)

Step 2: To a solution of 2-(4-methoxybenzoyl)-4,5-dimethyl-benzoic acid (1.00 eq, 1.00 g, 3.52 mmol) in DMF (15 mL) was added tert-butyl N-aminocarbamate (1.00 eq, 465 mg, 3.52 mmol), DIPEA (2.00 eq, 1.2 mL, 7.03 mmol) and HATU (1.20 eq, 1605 mg, 4.22 mmol), the mixture was stirred at 25° C. for 12 h. The reaction mixture was poured into water (60 mL), the mixture was filtered and the filter cake was washed with 20 mL of water, dried in vacuum to give product. The crude product was added PE/EA (10:1) (33 mL), the mixture was stirred at 25° C. for 0.5 h, the mixture was filtered and the filter cake was washed with 20 mL of water, dried in vacuum to give product. tert-butyl N-[[2-(4-methoxybenzoyl)-4,5-dimethyl-benzoyl]amino]carbamate (1.50 g, 3.39 mmol, 96.33% yield) was obtained as white solid. LC-MS: Rt: 0.832 min; 381.2=[M−17]+, ESI+1H NMR (400 MHz, DMSO-d6) δ=8.95 (s, 1H), 7.49 (s, 1H), 7.35-7.21 (m, 2H), 7.03 (s, 1H), 6.84 (s, 3H), 3.72 (s, 3H), 2.30 (s, 3H), 2.25 (s, 3H), 1.31-1.24 (m, 9H)

Step 3: A mixture of tert-butyl N-[[2-(4-methoxybenzoyl)-4,5-dimethyl-benzoyl]amino]carbamate (1.00 eq, 1.50 g, 3.76 mmol) in Methanol (15 mL) was added HCl/MeOH (15.0 eq, 14 mL, 56.5 mmol). The mixture was stirred at 25° C. for 12 h. The mixture was concentrated to give a crude product in vacuum. The crude product used for next step without further purification. 4-(4-methoxyphenyl)-6,7-dimethyl-2H-phthalazin-1-one; hydrochloride (1.15 g, 3.63 mmol, 96.43% yield) was obtained as white solid. LC-MS: Rt: 0.868 min; 281.0=[M+H]+, ESI+

Step 4: To a solution of 4-(4-methoxyphenyl)-6,7-dimethyl-2H-phthalazin-1-one; hydrochloride (1.00 eq, 500 mg, 1.58 mmol) in DMF (15 mL) was added (2-bromo-4-pyridyl)boronic acid (3.00 eq, 956 mg, 4.74 mmol), PYRIDINE (10.0 eq, 1.3 mL, 15.8 mmol) and Cu(OAc)₂ (1.30 eq, 371 mg, 2.05 mmol), the mixture was stirred at 80° C. for 12 h in 02. The mixture was added (2-bromo-4-pyridyl)boronic acid (1 eq), the mixture was stirred at 80° C. for 12 h in 02. The mixture was added (2-bromo-4-pyridyl)boronic acid (1 eq), the mixture was stirred at 80° C. for 12 h in 02. The mixture was added (2-bromo-4-pyridyl)boronic acid (1 eq), the mixture was stirred at 80° C. for 12 h in 02. The reaction mixture was poured into water (100 mL), the aqueous phase was extracted with DCM (100 mL*3). The combined organic phase was washed with brine (100 mL*3), dried with anhydrous Na₂SO₄, filtered and concentrated to give a crude product in vacuum. The crude product was purified by column chromatography on silica gel eluted with EA (0˜100%) in PE. 2-(2-bromo-4-pyridyl)-4-(4-methoxyphenyl)-6,7-dimethyl-phthalazin-1-one (219 mg, 0.432 mmol, 27.35% yield) was obtained as yellow solid. LC-MS: Rt: 1.034 min; 436.0=[M+H]+, ESI+; 86% purity at 220 nm. ¹H NMR (400 MHz, DMSO-d6) δ=8.52 (d, J=5.5 Hz, 1H), 8.25 (s, 1H), 8.18 (d, J=1.8 Hz, 1H), 8.00 (dd, J=1.8, 5.6 Hz, 1H), 7.68-7.62 (m, 2H), 7.55 (s, 1H), 7.15 (d, J=8.6 Hz, 2H), 3.87 (s, 3H), 2.49 (s, 3H), 2.41 (s, 3H)

Step 5: To a solution of 2-(2-bromo-4-pyridyl)-4-(4-methoxyphenyl)-6,7-dimethyl-phthalazin-1-one (1.00 eq, 100 mg, 0.229 mmol) in DMA (7.5 mL) was added 3-bromooxetane (1.20 eq, 38 mg, 0.275 mmol), pyridine-2,6-bis(carboximidamide) dihydrochloride (0.1000 eq, 3.7 mg, 0.0229 mmol), SODIUM IODIDE (0.250 eq, 8.6 mg, 0.0573 mmol), TFA (0.1000 eq, 0.0018 mL, 0.0229 mmol), Zinc powder (2.00 eq, 30 mg, 0.458 mmol) and NiCl₂(dme) (0.1000 eq, 5.0 mg, 0.0229 mmol) in N₂, the mixture was stirred at 60° C. for 12 h in N₂. The mixture was filtered and the filtrate was poured into water (40 mL), the aqueous phase was extracted with EA (40 mL*3). The combined organic phase was washed with brine (40 mL*3), dried with anhydrous Na₂SO₄, filtered and concentrated to give a crude product in vacuum. The crude product was purified by prep-HPLC (FA, column: Phenomenex Luna C18 150*25 mm*10 um; mobile phase: [water (FA)-ACN]; B %: 43%-73%, 10 min) and lyophilized to give 4-(4-methoxyphenyl)-6,7-dimethyl-2-[2-(oxetan-3-yl)-4-pyridyl]phthalazin-1-one (12 mg, 0.0272 mmol, 11.89% yield) was obtained as white solid. LC-MS: Rt: 0.891 min; 414.1=[M+H]⁺, ESI+; 97.1% purity at 220 nm. ¹H NMR (400 MHz, CHLOROFORM-d) δ=8.75 (d, J=2.6 Hz, 1H), 8.36 (s, 1H), 7.99-7.77 (m, 2H), 7.68-7.45 (m, 3H), 7.10 (d, J=7.1 Hz, 2H), 5.27-4.88 (m, 4H), 4.48 (s, 1H), 3.94 (s, 3H), 2.57-2.40 (m, 6H)

### Example 63: Synthesis of 2-(2-(3-(benzyloxy)cyclobutyl)pyridin-4-yl)-4-(4-chlorophenyl)-6,7-dimethylphthalazin-1 (2H)-one I-109

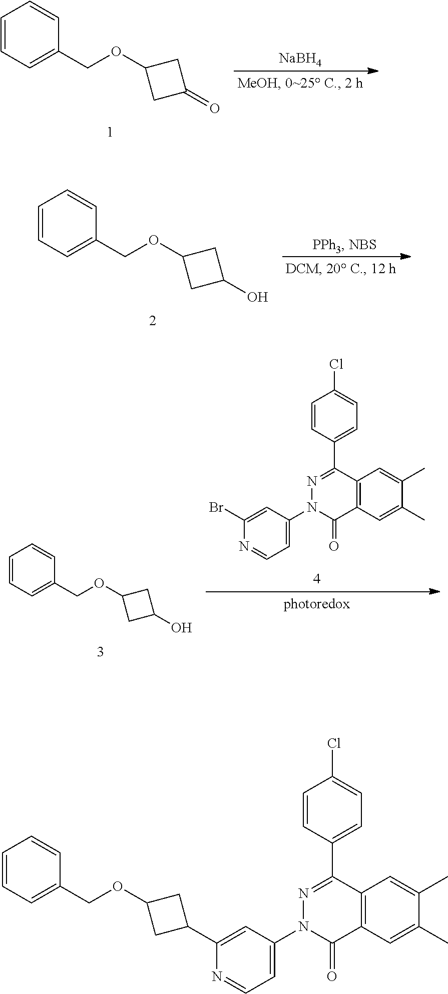

Step 1: To a solution of 3-(benzyloxy)cyclobutan-1-one (1.00 eq, 1000 mg, 5.68 mmol) in Methanol (10 mL) was added NaBH₄ (2.00 eq, 429 mg, 11.4 mmol) at 0° C. under N₂. The mixture was stirred at 25° C. for 2 h. LCMS: HW-2021-01-078-P1A showed one peak without the desired mass (Rt: 0.709 min; [M+H]=N/A at 220 nm). TLC (PE/EtOAc=1/1, the starting material R_{f}=0.3; the desired product R_{f}=0.6) showed that the starting material was consumed completely and a new spot was formed. The reaction mixture was quenched by addition sat. NH₄Cl (40 mL) at 0° C., and then extracted with EtOAc (30 mL×2). The combined organic layers were washed with brine (50 mL), dried over Na₂SO₄, filtered and concentrated under reduced pressure to give a residue. The residue was purified by column chromatography (SiO₂, PE/EtOAc=1/0 to 0/1) to afford 3-(benzyloxy)cyclobutan-1-ol (800 mg, 4.49 mmol, 79.09% yield) as colorless oil, checked by H NMR: HW-2021-01-078-P1A.HW-2021-01-78-1 (P1, achiral): (M+H)⁺=N/A; purity=94% (220 nm); Retention time=0.709 min. ¹H NMR (400 MHz, DMSO-d₆) δ=7.37-7.23 (m, 5H), 5.01 (d, J=6.6 Hz, 1H), 4.33 (s, 2H), 3.72-3.64 (m, 1H), 3.54 (quin, J=7.1 Hz, 1H), 2.51 (br s, 2H), 1.79-1.69 (m, 2H)

Step 2: To a solution of 3-(benzyloxy)cyclobutan-1-ol (1.00 eq, 750 mg, 4.21 mmol) and PPh₃ (2.00 eq, 2207 mg, 8.42 mmol) in DCM (30 mL) was added NBS (2.00 eq, 1498 mg, 8.42 mmol) slowly and stirred at 20° C. for 12 hrs. TLC (PE/EtOAc=5/1, the starting material R_{f}=0.3; the desired product R_{f}=0.6) showed that the starting material was consumed completely and a new spot was formed. The mixture was concentrated under reduced pressure. The residue was purified by column chromatography (SiO₂, PE/EtOAc=1/0 to 5/1) to afford ((3-bromocyclobutoxy)methyl)benzene (900 mg, 3.73 mmol, 88.70% yield) as colorless oil. Confirmed by LCMS: HW-2021-01-085-P1B and H NMR: HW-2021-01-085-P1A. HW-2021-01-85-1 (P1, achiral): (M+H)⁺=N/A; purity=100% (220 nm); Retention time=0.701 min. ¹H NMR (400 MHz, CDCl₃) δ=7.40-7.28 (m, 5H), 4.58-4.47 (m, 2H), 4.43 (s, 2H), 2.80-2.57 (m, 4H).

Step 3: To a 15 mL vial equipped with a stir bar was added ((3-bromocyclobutoxy)methyl)benzene (2.74 eq, 150 mg, 0.622 mmol), 2-(2-bromopyridin-4-yl)-4-(4-chlorophenyl)-6,7-dimethylphthalazin-1 (2H)-one (1.00 eq, 100 mg, 0.227 mmol), Ir[dF(CF3)ppy]₂(dtbpy)(PF₆) (0.0440 eq, 11 mg, 0.00998 mmol), NiCl₂·dtbbpy (0.00500 eq, 2.0 mg, 0.00113 mmol), TTMSS (4.41 eq, 249 mg, 1.00 mmol), Na₂CO₃ (8.81 eq, 212 mg, 2.00 mmol) in DME (1.00 eq, 10 mL, 0.227 mmol). The vial was sealed and placed under nitrogen was added. The reaction was stirred and irradiated with a 34 W blue LED lamp (7 cm away), with cooling fan to keep the reaction temperature at 25° C. for 14 hrs. LCMS: HW-2021-02-004-P1A showed the starting material was consumed completely and 13% desired mass was detected (Rt: 0.979 min; [M+H]⁺=522.4 at 220 nm). The reaction mixture was filtered and concentrated under reduced pressure to give a residue. The residue was purified by prep-HPLC (column: Unisil 3-100 C18 Ultra 150×50 mm×3 um water (FA)-ACN; B %: 50%-80%, 9 min) to afford 10 mg the desired product. LCMS: HW-2021-01-004-P1B1 showed 74% purity (Rt: 0.719 min; [M+H]⁺=522.2 at 220 nm). 10 mg the desired product was purified by prep-TLC (SiO₂, PE/EtOAc=1/1, R_{f}=0.45) to afford 2-(2-(3-(benzyloxy)cyclobutyl)pyridin-4-yl)-4-(4-chlorophenyl)-6,7-dimethylphthalazin-1 (2H)-one (3.1 mg, 0.00593 mmol, 2.61% yield) as off-white solid. Confirmed by LCMS: and H NMR: HW-2021-02-004-P1A and HPLC: (P1, achiral): (M+H)⁺=N/A; purity=100% (220 nm); Retention time=0.701 min. ¹H NMR (400 MHz, CDCl₃) δ=8.65 (d, J=5.4 Hz, 1H), 8.36 (s, 1H), 7.76 (d, J=1.8 Hz, 1H), 7.67 (dd, J=1.9, 5.5 Hz, 1H), 7.61-7.52 (m, 4H), 7.45 (s, 1H), 7.39-7.32 (m, 4H), 7.31-7.28 (m, 1H), 4.49 (s, 2H), 4.16-4.05 (m, 1H), 3.29-3.17 (m, 1H), 2.79-2.68 (m, 2H), 2.51 (s, 3H), 2.43 (s, 3H), 2.40-2.32 (m, 2H).

### Example 64: Synthesis of 4-(4-chlorophenyl)-6,7-dimethyl-2-[2-(oxetan-3-ylamino)-4-pyridyl]phthalazin-1-one I-110

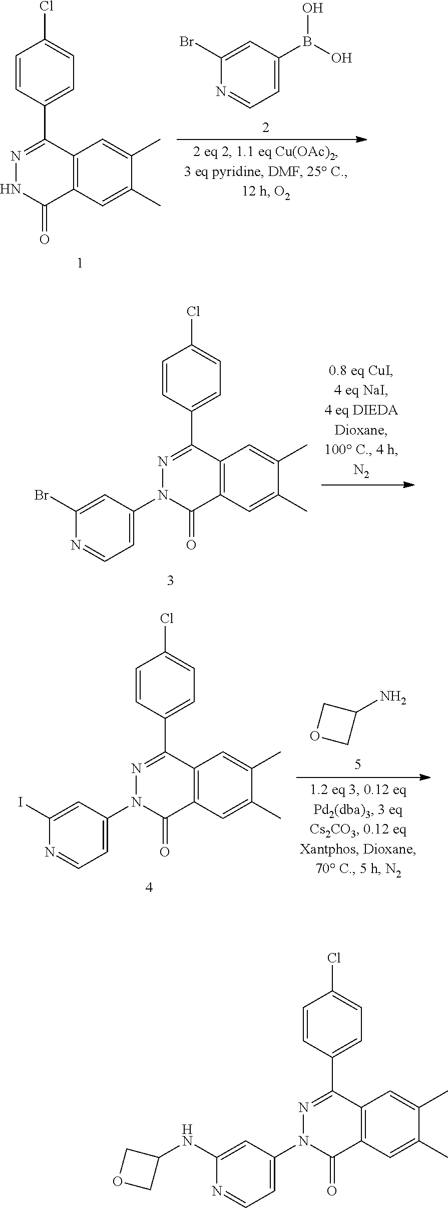

Step 1: A mixture of 4-(4-chlorophenyl)-6,7-dimethyl-2H-phthalazin-1-one (1.00 eq, 600 mg, 2.11 mmol) and (2-bromo-4-pyridyl)boronic acid (2.00 eq, 851 mg, 4.21 mmol) in DMF (20 mL) was added pyridine (3.00 eq, 0.51 mL, 6.32 mmol) and Cu(OAc)₂ (1.70 eq, 648 mg, 3.58 mmol). The mixture was degassed with O₂ for 3 times. The mixture was stirred at 25° C. under O₂ for 12 hours. LCMS (5-95AB/1.5 min): RT=1.129 min, [M+H]⁺ 442.1 showed 69.4% of desired product. The reaction mixture was added to 150 mL water and extracted with EtOAc (150 mL×3). The organic phase was concentrated under vacuum to give a crude. The crude was added MeOH (50 mL) and a lot of solid remained. The mixture was stirred for 0.5 h and filtered. The filter cake was washed with MeOH (50 mL) three times and concentrated under vacuum to give 2-(2-bromo-4-pyridyl)-4-(4-chlorophenyl)-6,7-dimethyl-phthalazin-1-one (977 mg, 1.88 mmol, 89.42% yield) as white solid, LCMS: Rt: 1.107 min; [M+H]⁺=441.7; 85% purity at 220 nm.

Step 2: To a solution of 2-(2-bromo-4-pyridyl)-4-(4-chlorophenyl)-6,7-dimethyl-phthalazin-1-one (1.00 eq, 600 mg, 1.16 mmol) in 1,4-Dioxane (40 mL) was added CuI (0.400 eq, 88 mg, 0.463 mmol), NaI (2.00 eq, 384 mg, 2.31 mmol) and N,N′-dimethylethylenediamine (0.400 eq, 41 mg, 0.463 mmol). Then the reaction mixture was stirred at 100° C. for 3 hours under N₂ atmosphere. LCMS (5-95AB/1.5 min): RT=1.125 min, 487.8=[M+H]⁺, ESI+ showed 79% of desired product. The filtrate was concentrated under reduced pressure to afford a residue. The solvent was concentrated under reduced pressure, then water (50 mL) and DCM (80 mL) was added. A lot of white floccules were appeared. The suspension was filtered through a pad of celite. The filter cake was washed with DCM (40 mL). The filtrate was separated by separating funnel. The organic layers washed with brine and dried over Na₂SO₄, filtered and concentrated under reduced pressure to give a residue. The residue was purified by flash chromatography on silica gel eluting with [DCM]/[ethyl acetate](10:1) to afford the 4-(4-chlorophenyl)-2-(2-iodo-4-pyridyl)-6,7-dimethyl-phthalazin-1-one (500 mg, 0.943 mmol, 81.50% yield) as gray solid, LCMS: Rt: 1.112 min; [M+H]⁺=487.8; 92% purity at 220 nm.

Step 3: To a solution of 4-(4-chlorophenyl)-2-(2-iodo-4-pyridyl)-6,7-dimethyl-phthalazin-1-one (1.00 eq, 200 mg, 0.410 mmol) in 1,4-Dioxane (5 mL) was added oxetan-3-amine (1.10 eq, 33 mg, 0.451 mmol), Cs₂CO₃ (3.00 eq, 401 mg, 1.23 mmol), Pd₂(dba)₃ (0.120 eq, 28 mg, 0.0492 mmol) and XantPhos (0.120 eq, 28 mg, 0.0492 mmol) at 25° C. The reaction mixture was degassed with N₂ for 3 times. The mixture was stirred at 70° C. under N₂ for 4 hours. LCMS (5-95AB/1.5 min): RT=0.885 min, 433.2=[M+H]⁺, ESI+ showed 24% of desired product. The reaction mixture was concentrated under reduced pressure to afford a residue. The residue was diluted with water (30 mL) and then extracted with DCM (20 mL*3). The combined organic layers were dried over Na₂SO₄, filtered and concentrated under reduced pressure to give a residue. The residue was purified by prep-TLC (PE:EtOAc=1:1, R_{f}=0.55) to afford crude product (16 mg) as yellow solid, which confirmed by LCMS (5-95AB/1.5 min): RT=0.596 min, 433.1=[M+H]⁺, ESI+, showed 95% of product and HPLC showed 80% of product. The crude product was further purified by prep-HPLC (Column, [Unisil 3-100 C18 Ultra 150*50 mm*3 um]; mobile phase: [ACN] and [H₂O](conditions: [water (0.225% FA)-ACN], B %: 28%-58%; Detector, UV 254 nm. RT: [10 min]) to afford crude product (8.2 mg) as light yellow solid, which confirmed by LCMS (5-95AB/1.5 min): RT=0.884 min, 433.1=[M+H]⁺, ESI+ showed 73.2% of product. The crude product was further purified by SFC (“Column: REGIS(S,S)WHELK-O1 (250 mm*25 mm, 10 um), Mobile phase: Phase A for CO₂, and Phase B for MeOH+ACN (0.05% DEA); Gradient elution: 60% (0.1% NH₃·H₂O MeOH) in CO₂ Flow rate: 3 mL/min; Detector: PDA Column Temp: 35 C; Back Pressure: 100 Bar). The final product 4-(4-chlorophenyl)-6,7-dimethyl-2-[2-(oxetan-3-ylamino)-4-pyridyl]phthalazin-1-one (2.1 mg, 0.00454 mmol, 1.11% yield) was given as a yellow solid. LCMS: Rt: 0.604 min; [M+H]⁺=433.1; 93.5% purity at 220 nm and ¹H NMR (400 MHz, CHLOROFORM-d) δ ppm 2.42 (s, 3H) 2.51 (s, 3H) 4.60 (s, 2H) 5.02 (br d, J=2.50 Hz, 3H) 5.32-5.45 (m, 1H) 7.06 (s, 1H) 7.20-7.24 (m, 1H) 7.45 (s, 1H) 7.52-7.60 (m, 4H) 8.15 (d, J=5.75 Hz, 1H) 8.35 (s, 1H).

### Example 65: Synthesis of tert-butyl 3-[[4-[5-(4-chlorophenyl)-2,3-dimethyl-8-oxo-pyrido[2,3-d]pyridazin-7-yl]-2-pyridyl]oxy]azetidine-1-carboxylate, I-93, and 7-[2-(azetidin-3-yloxy)-4-pyridyl]-5-(4-chlorophenyl)-2,3-dimethyl-pyrido[2,3-d]pyridazin-8-one; 2,2,2-trifluoroacetic acid I-94

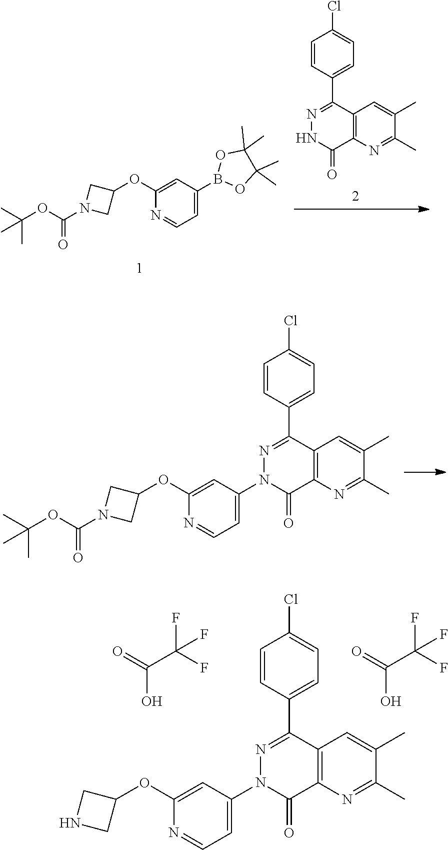

Step 1: To a solution of tert-butyl 3-[[4-(4,4,5,5-tetramethyl-1,3,2-dioxaborolan-2-yl)-2-pyridyl]oxy]azetidine-1-carboxylate (1.00 eq, 20 mg, 0.0532 mmol) and 5-(4-chlorophenyl)-2,3-dimethyl-7H-pyrido[2,3-d]pyridazin-8-one (0.900 eq, 14 mg, 0.0478 mmol), H₃BO₃ (3.00 eq, 9.9 mg, 0.159 mmol) in DMF (1 mL) was added pyridine (5.00 eq, 0.021 mL, 0.266 mmol) and Cu(OAc)₂ (1.10 eq, 11 mg, 0.0585 mmol) under N₂ and then the mixture was stirred for 16 h at 25° C. under 02 (15 psi). LCMS showed the major peak showed desired MS (533.9 [M+H]+; ESI+). The reaction was poured into water (5 mL) and then extracted with EtOAc (3 mL*2) and the organics washed with 10 mL saturated brine solution. The organics were then separated and dried (Na₂SO₄) before concentration to dryness. The crude was then purified by prep-TLC (PE/EA=0/1, R_{f}=0.6) and freeze-drying to give tert-butyl 3-[[4-[5-(4-chlorophenyl)-2,3-dimethyl-8-oxo-pyrido[2,3-d]pyridazin-7-yl]-2-pyridyl]oxy]azetidine-1-carboxylate (2.6 mg, 0.00473 mmol, 8.89% yield) as white solid which was confirmed by QC for LCMS: (M+H)⁺=533.9; purity=97.09% (UV=220 nm); Retention time=1.064 min. 1H NMR (400 MHz, CHLOROFORM-d) δ=8.22-8.17 (m, 1H), 7.77-7.73 (m, 1H), 7.61-7.54 (m, 5H), 7.40-7.36 (m, 1H), 5.40-5.33 (m, 1H), 4.38-4.31 (m, 2H), 4.05-3.97 (m, 2H), 2.84-2.81 (m, 3H), 2.50-2.47 (m, 3H), 1.48-1.44 (m, 9H)

Step 2: To a solution of tert-butyl 3-[[4-[5-(4-chlorophenyl)-2,3-dimethyl-8-oxo-pyrido[2,3-d]pyridazin-7-yl]-2-pyridyl]oxy]azetidine-1-carboxylate (1.00 eq, 20 mg, 0.0375 mmol) in DCM (0.5 mL) was added TFA (69.7 eq, 0.20 mL, 2.61 mmol) and stirred for 1 h at 30° C. LCMS showed the raw material was consumed and two peaks showed desired MS (434.1 [M+H]+; ESI+). The solution was concentration to dryness and purified by prep-HPLC (Phenomenex Synergi Polar-RP 100*25 mm*4 um, water (TFA)-ACN) to give 7-[2-(azetidin-3-yloxy)-4-pyridyl]-5-(4-chlorophenyl)-2,3-dimethyl-pyrido[2,3-d]pyridazin-8-one; 2,2,2-trifluoroacetic acid (3.1 mg, 0.00466 mmol, 12.45% yield) as white solid which was confirmed by LCMS for and H NMR, LCMS: (M+H)⁺=434.1; purity=100% (UV=220 nm); Retention time=0.672 min. 1H NMR (400 MHz, CHLOROFORM-d) δ=11.56-11.23 (m, 1H), 9.81-9.50 (m, 1H), 8.22-8.16 (m, 1H), 7.80-7.76 (m, 1H), 7.74-7.69 (m, 1H), 7.58-7.50 (m, 5H), 5.77-5.52 (m, 1H), 4.86-4.58 (m, 2H), 4.53-4.30 (m, 2H), 2.81-2.76 (m, 3H), 2.51-2.46 (m, 3H).

### Example 66: Synthesis of Compounds [6,7-dimethyl-3-[2-(oxetan-3-yl)-4-pyridyl]-4-oxo-phthalazin-1-yl]trifluoromethanesulfonate I-111

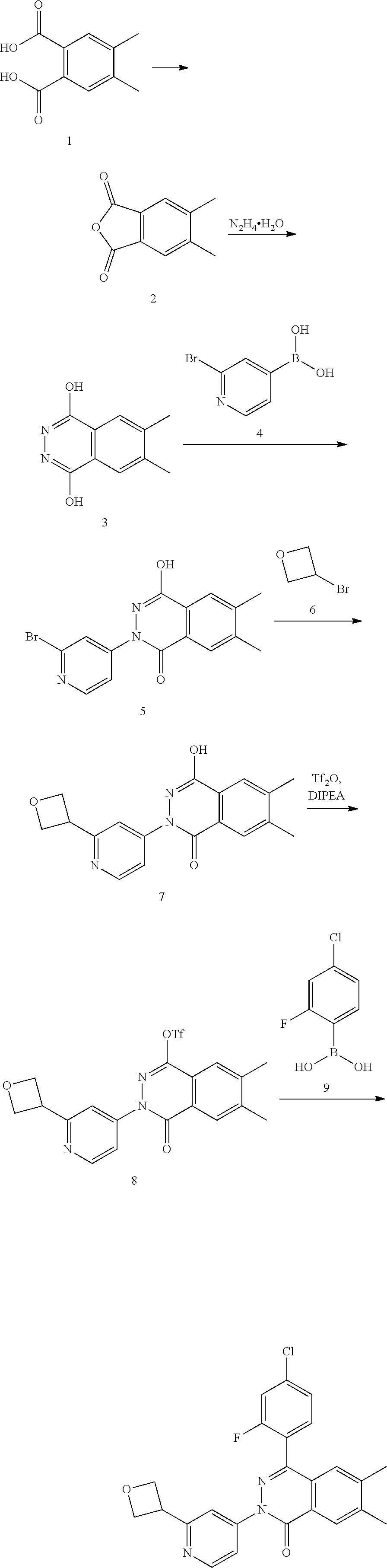

Step 1: A mixture of 4, 5-dimethylphthalic acid (1.00 eq, 4.00 g, 20.6 mmol) in acetic anhydride (7.35 eq, 14 mL, 151 mmol) was stirred at 100° C. for 3 h. LCMS showed the starting material was consumed completely and desired MS was found. The reaction mixture was concentrated in vacuum. The residue was triturated with PE:EA=10:1 (10 mL) to give 5, 6-dimethylisobenzofuran-1, 3-dione 2 (3.00 g, 15.8 mmol, 76.88% yield) as a gray solid. LCMS: (M+H₂O+H)⁺=191.0; purity=45% (UV 254 nm); Retention time=0.58 min.

Step 2: To a solution of 5,6-dimethylisobenzofuran-1,3-dione (1.00 eq, 1.50 g, 8.51 mmol) in Ethanol (50 mL) was added hydrazine monohydrate (1.30 eq, 553 mg, 11.1 mmol) and stirred at 80° C. for 12 h under N₂ atmosphere. LC-MS showed starting material was consumed completely and desired mass was detected. The mixture was added to 200 mL water and extracted with EtOAc (200 mL×3). The organic phase was concentrated under vacuum to give a crude. The crude was added DCM (20 mL) and a lot of solid remained. The mixture was stirred for 0.5 h and filtered. The filter cake was concentrated under vacuum to give 6, 7-dimeth ylphthalazine-1, 4-diol 3 (1560 mg, 8.20 mmol, 96.33% yield) as a white solid. LCMS: (M+H)⁺=191.2; purity=100% (UV 254 nm); Retention time=0.634 min.

Step 3: A mixture of (2-bromo-4-pyridyl)boronic acid (3.00 eq, 3501 mg, 17.4 mmol) and 6,7-dimethylphthalazine-1,4-diol (1.00 eq, 1.10 g, 5.78 mmol) in DMF (30 mL) was added pyridine (3.00 eq, 1.4 mL, 17.4 mmol) and Cu(OAc)₂ (1.30 eq, 1361 mg, 7.52 mmol). The mixture was degassed with O₂ for 3 times. The mixture was stirred at 25° C. under O₂ for 12 h. LC-MS showed that 57% desired MS was detected. The mixture was added to 200 mL water and extracted with EtOAc (200 mL×3). The organic phase was concentrated under vacuum to give a residue. The residue was purified by column chromatography (SiO₂, DCM:MeOH=10/1, Rf=0.7), filtered and filtrate was concentrated to give a white solid 2-(2-bromo-4-pyridyl)-4-hydroxy-6,7-dimethyl-phthalazin-1-one 5 (1100 mg, 2.32 mmol, 40.11% yield) as a white solid. LCMS: (M+H)⁺=346; purity=73% (UV 254 nm); Retention time=0.886 min.

Step 4: To a solution of 2-(2-bromo-4-pyridyl)-4-hydroxy-6,7-dimethyl-phthalazin-1-one (1.00 eq, 200 mg, 0.578 mmol) in DMA (10 mL) was added 3-bromooxetane (1.20 eq, 95 mg, 0.693 mmol), TFA (0.1000 eq, 0.0044 mL, 0.0578 mmol), sodium iodide (0.250 eq, 22 mg, 0.144 mmol), Zinc powder (2.00 eq, 76 mg, 1.16 mmol) and NiCl₂(dme) (0.1000 eq, 13 mg, 0.0578 mmol) in N₂, the mixture was stirred at 60° C. for 4 h in N₂. LCMS showed 70% starting material was still remained and 23% desired mass was detected. The mixture was stirred at 60° C. for 12 h. LC-MS showed that 73% starting material was still remained and 23% desired mass was detected The mixture was filtered and the filtrate was concentrated in vacuo. The crude product was purified by prep-HPLC (FA, Column: 120 g Flash Column Welch Ultimate XB_C18 20-40 m; 120 A, mobile phase: [water (FA)-ACN]; B %: 50%, 10 min). 4-hydroxy-6,7-dimethyl-2-[2-(oxetan-3-yl)-4-pyridyl]phthalazin-1-one 7 (30 mg, 0.0846 mmol, 14.65%% yield) was obtained as gray solid. LCMS: (M+H)⁺=324.1; purity=100% (UV 254 nm); Retention time=0.774 min.

Step 5: To a solution of 4-hydroxy-6,7-dimethyl-2-[2-(oxetan-3-yl)-4-pyridyl]phthalazin-1-one (1.00 eq, 30 mg, 0.0928 mmol) in DCM (1 mL) was added trifluoromethanesulfonic anhydride (1.50 eq, 0.024 mL, 0.139 mmol) and DIPEA (3.00 eq, 0.048 mL, 0.278 mmol) at 0° C. and stirred at 20° C. for 1 h. LCMS showed starting material was consumed completely and desired mass was detected. The reaction mixture was poured into 20 mL of water carefully and extracted with EA (20 mL×3), the combined organic phase was dried over Na₂SO₄ and concentrated to give a residue. The residue was purified by prep-TLC (SiO2, PE: EA=1/1, R_{f}=0.5), filtered and filtrate was concentrated to give [6,7-dimethyl-3-[2-(oxetan-3-yl)-4-pyridyl]-4-oxo-phthalazin-1-yl]trifluoromethanesulfonate (9.0 mg, 0.0198 mmol, 21.30%% yield) 8 as a white solid. LCMS: (M+H)⁺=456.1; purity=100% (UV 254 nm); Retention time=0.838 min.

Step 6: To a solution of (4-chloro-2-fluoro-phenyl)boronic acid (1.50 eq, 5.2 mg, 0.0296 mmol) in 1,4-Dioxane (1 mL) and Water (0.1000 mL) was added [6,7-dimethyl-3-[2-(oxetan-3-yl)-4-pyridyl]-4-oxo-phthalazin-1-yl]trifluoromethanesulfonate (1.00 eq, 9.0 mg, 0.0198 mmol), Na₂CO₃ (2.00 eq, 4.2 mg, 0.0395 mmol) and Pd(dppf)Cl₂DCM (0.0500 eq, 0.81 mg, 0.000988 mmol) under N₂ atmosphere and stirred at 100° C. under N₂ atmosphere for 12 h. LCMS showed starting material was consumed completely and desired mass was detected. The reaction mixture was poured into 20 mL of water carefully and extracted with EA (20 mL×3), the combined organic phase was dried over Na₂SO₄ and concentrated to give a residue. The residue was purified by prep-HPLC (water (FA)-ACN]; B %: 45%-75%, 10 min), the solution was lyophilized to give 4-(4-chloro-2-fluoro-phenyl)-6,7-dimethyl-2-[2-(oxetan-3-yl)-4-pyridyl]phthalazin-1-one (0.93 mg, 0.00213 mmol, 10.80% yield) was obtained as a white solid. LCMS: (M+H)⁺=436.1; purity=100% (UV 220 nm); Retention time=0.917 min. ¹H NMR (400 MHz, CDCl₃) 8.73 (d, J=5.6 Hz, 1H), 8.34 (s, 1H), 7.98 (s, 1H), 7.87-7.85 (m, 1H), 7.52-7.46 (m, 1H), 7.38-7.32 (m, 2H), 7.21 (d, J=2.4 Hz, 1H), 5.119-5.08 (m, 2H), 4.99 (t, J=6.3 Hz, 2H), 4.53-4.44 (m, 1H), 2.51 (s, 3H), 2.43 (s, 3H) LCMS: (M+H)⁺=456.1; purity=100% (UV 254 nm); Retention time=0.838 min.

### Example 67: Synthesis of 5-(4-chlorophenyl)-2,3-dimethyl-7-[2-(oxetan-3-yl)-4-pyridyl]pyrido [2,3-d]pyridazin-8-one I-112

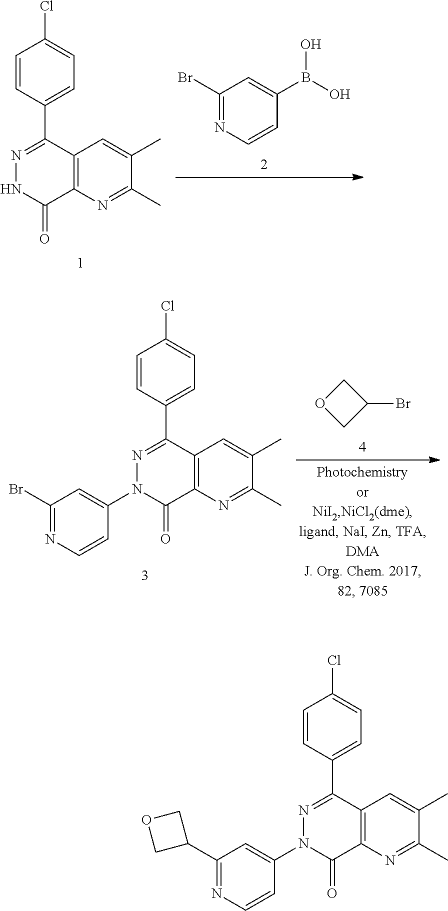 [SMILES: Cc1cc2c(-c3ccc(Cl)cc3)n[nH]c(=O)c2nc1C ; OB(O)c1ccnc(Br)c1 ; Cc1cc2c(-c3ccc(Cl)cc3)nn(-c3ccnc(Br)c3)c(=O)c2nc1C ; BrC1COC1 ; Cc1cc2c(-c3ccc(Cl)cc3)nn(-c3ccnc(C4COC4)c3)c(=O)c2nc1C]

Step 1: To a white suspension of 5-(4-chlorophenyl)-2,3-dimethyl-7H-pyrido[2,3-d]pyridazin-8-one (1.00 eq, 100 mg, 0.350 mmol) in DMF (2 mL) was added (2-bromo-4-pyridyl)boronic acid (2.00 eq, 141 mg, 0.700 mmol), Cu(OAc)₂ (1.10 eq, 70 mg, 0.385 mmol) and PYRIDINE (3.00 eq, 0.085 mL, 1.05 mmol), to give a blue suspension, the mixture was stirred at 25° C. for 12 h in O₂, the mixture was green suspension. The reaction mixture was poured into water (20 mL), the aqueous phase was extracted with EA (20 mL*3). The combined organic phase was washed with brine (20 mL*3), dried with anhydrous Na₂SO₄, filtered and concentrated to give a crude product in vacuum. The crude product was poured into MeOH (10 mL), the mixture was stirred at 20° C. for 0.5 h, the mixture was filtered and the filter cake was washed with 20 mL of MeOH, dried in vacuum to give product. 7-(2-bromo-4-pyridyl)-5-(4-chlorophenyl)-2,3-dimethyl-pyrido[2,3-d]pyridazin-8-one (89 mg, 0.201 mmol, 57.57% yield) was obtained as white solid. LC-MS: Rt: 0.969 min; 443.0=[M+H+2]+, ESI+; 100% purity at 220 nm

Step 2: To a white suspension of 7-(2-bromo-4-pyridyl)-5-(4-chlorophenyl)-2,3-dimethyl-pyrido[2,3-d]pyridazin-8-one (1.00 eq, 60 mg, 0.136 mmol) in DMA (2 mL) was added 3-bromooxetane (1.20 eq, 22 mg, 0.163 mmol), sodium iodide (0.250 eq, 5.1 mg, 0.0340 mmol), pyridine-2,6-bis(carboximidamide) dihydrochloride (0.1000 eq, 2.2 mg, 0.0136 mmol), TFA (0.1000 eq, 0.0010 mL, 0.0136 mmol), to give a white suspension, to the white suspension was added NiCl₂(dme) (0.1000 eq, 3.0 mg, 0.0136 mmol) in N₂, to give a blue suspension, to the blue suspension was added Zinc powder (2.00 eq, 18 mg, 0.272 mmol) in N2, to give a blue suspension, The mixture was stirred at 60° C. for 16 h, the mixture was a red suspension. The mixture was filtered and the filtrate was poured into water (20 mL), the aqueous phase was extracted with EA (20 mL*3). The combined organic phase was washed with brine (20 mL*3), dried with anhydrous Na₂SO₄, filtered and concentrated to give a crude product in vacuum. The crude product was purified by prep-HPLC (FA, column: Phenomenex Luna C18 150*25 mm*10 um; mobile phase: [water (FA)-ACN]; B %: 36%-66%, 10 min) and lyophilized. 5-(4-chlorophenyl)-2,3-dimethyl-7-[2-(oxetan-3-yl)-4-pyridyl]pyrido[2,3-d]pyridazin-8-one (2.8 mg, 0.00648 mmol, 4.77% yield) was obtained as white solid. LC-MS: Rt: 0.841 min; 419.1=[M+H]+, ESI+; 96.9% purity at 220 nm. ¹H NMR (400 MHz, CHLOROFORM-d) δ=8.76 (d, J=5.5 Hz, 1H), 7.89 (d, J=1.8 Hz, 1H), 7.80 (dd, J=2.1, 5.5 Hz, 1H), 7.75 (s, 1H), 7.57 (s, 4H), 5.08 (dd, J=5.9, 8.4 Hz, 2H), 5.04-4.97 (m, 2H), 4.52-4.40 (m, 1H), 2.83 (s, 3H), 2.49 (s, 3H).

### Example 68: Synthesis of 5-(4-chlorophenyl)-2,3-dimethyl-7-(2-(oxetan-3-ylamino)pyridin-4-yl)pyrido[2,3-d]pyridazin-8 (7H)-one I-116

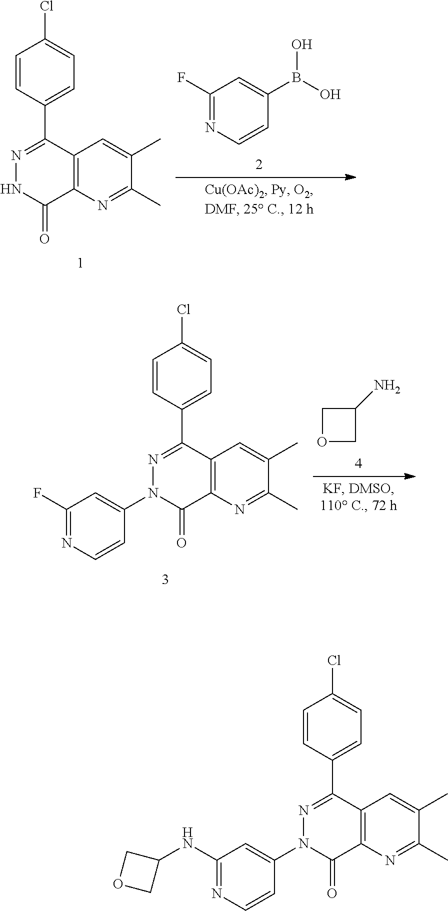

Step 1: A mixture of 5-(4-chlorophenyl)-2,3-dimethylpyrido[2,3-d]pyridazin-8 (7H)-one (1.00 eq, 100 mg, 0.350 mmol) and (2-fluoropyridin-4-yl)boronic acid (2.00 eq, 99 mg, 0.700 mmol) in DMF (5 mL) was added Py (5.00 eq, 0.14 mL, 1.75 mmol) and Cu(OAc)₂ (1.10 eq, 70 mg, 0.385 mmol). The mixture was degassed with 02 for 3 times. The mixture was stirred at 25° C. under 02 for 12 h. LCMS showed 69.6% of desired product (69.6%, Rt: 0.945 min; [M+H]⁺=381.1 at 220 nm). The mixture was diluted with water (40 mL) and extracted with EtOAc (80 mL) twice. The combined organic layers were dried over Na₂SO₄. The solvent was filtered and concentrated under vacuum. The crude product was purified by purified by prep-TLC (DCM/MeOH=10/1, R_{f}=0.6) and column chromatography on silica gel eluted with DCM/MeOH=1/0 to 10/1 (DCM/MeOH=10/1, the desired product R_{f}=0.6) to give 5-(4-chlorophenyl)-7-(2-fluoropyridin-4-yl)-2,3-dimethylpyrido[2,3-d]pyridazin-8 (7H)-one (90 mg, 0.236 mmol, 67.53% yield) as brown solid, checked by LCMS: and H NMR: (P1, achiral): [M+H]=381.1; purity=98.5% (220 nm); Retention time=0.970 min¹H NMR (400 MHz, CDCl₃) δ=8.32 (d, J=5.6 Hz, 1H), 7.96-7.91 (m, 1H), 7.77 (s, 1H), 7.67 (s, 1H), 7.58 (s, 4H), 2.82 (s, 3H), 2.50 (s, 3H).

Step 2: A solution of 5-(4-chlorophenyl)-7-(2-fluoropyridin-4-yl)-2,3-dimethylpyrido[2,3-d]pyridazin-8 (7H)-one (1.00 eq, 100 mg, 0.263 mmol), oxetan-3-amine (20.0 eq, 384 mg, 5.25 mmol) and KF (3.00 eq, 46 mg, 0.788 mmol) in DMSO (5 mL) was stirred at 110° C. for 72 h. LCMS showed the starting material was consumed and a major peak with desired MS (41.4%, Rt: 0.807 min; [M+H]=434.1 at 220 nm). The reaction mixture was purified by prep-HPLC (Waters Xbridge 150×25 mm×5 um; mobile phase: [water (NH₄HCO₃)-ACN]; B %: 37%-67%, 10 min) and lyophilized to give 5-(4-chlorophenyl)-2,3-dimethyl-7-(2-(oxetan-3-ylamino)pyridin-4-yl)pyrido[2,3-d]pyridazin-8 (7H)-one (3.7 mg, 0.00812 mmol, 3.09% yield) as yellow solid, checked by LCMS: H NMR: [M+H]⁺=434.1; purity=95% (220 nm); Retention time=0.810 min. ¹H NMR (400 MHz, CDCl₃) δ=8.19 (d, J=5.6 Hz, 1H), 7.74 (s, 1H), 7.56 (s, 4H), 7.19 (dd, J=1.6, 5.7 Hz, 1H), 7.07 (s, 1H), 5.08-4.98 (m, 4H), 4.61-4.55 (m, 2H), 2.82 (s, 3H), 2.48 (s, 3H)

### Example 69: Synthesis of 5-(4-chlorophenyl)-2,3-dimethyl-7-(2-((tetrahydrofuran-3-yl)amino)pyridin-4-yl)pyrido[2,3-d]pyridazin-8 (7H)-one I-113

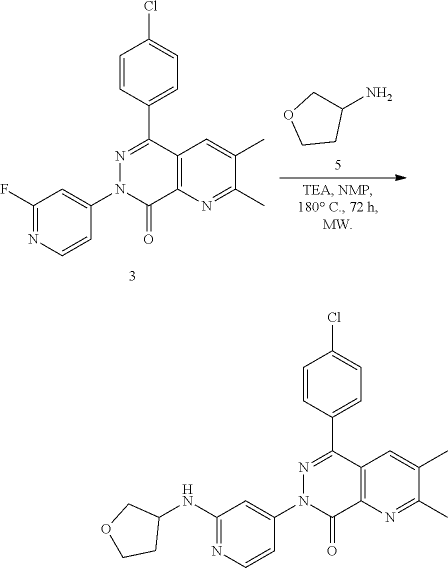

Step 1: A solution of 5-(4-chlorophenyl)-7-(2-fluoropyridin-4-yl)-2,3-dimethylpyrido[2,3-d]pyridazin-8 (7H)-one (1.00 eq, 30 mg, 0.0788 mmol) and TEA (6.00 eq, 0.041 mL, 0.473 mmol) in NMP (2 mL) was added tetrahydrofuran-3-amine (5.00 eq, 34 mg, 0.394 mmol). The reaction vessel was sealed and heated under microwave at 180° C. for 3 h. LCMS showed that 9% the desired mass (9%, Rt: 0.820 min; [M+H]⁺=448.1 at 220 nm). The reaction mixture was added tetrahydrofuran-3-amine (10.0 eq, 69 mg, 0.788 mmol) and sealed and heated in microwave at 180° C. for another 3 h. LCMS showed that 52% the desired mass (52%, Rt: 0.726 min; [M+H]⁺=448.2 at 220 nm). The crude product was purified by prep-HPLC (Phenomenex luna C18 150×25 mm×10 um: [water (0.1% FA)-ACN]; B %: 18%-48%, 12 min) and lyophilized to give 5-(4-chlorophenyl)-2,3-dimethyl-7-(2-((tetrahydrofuran-3-yl)amino)pyridin-4-yl)pyrido[2,3-d]pyridazin-8 (7H)-one (1.9 mg, 0.00418 mmol, 5.30% yield) as light brown solid, which was checked by LCMS and H NMR (P1, racemic): [M+H]⁺=448.3; purity=98.8% (220 nm); Retention time=0.727 min. ¹H NMR (400 MHz, CDCl₃) δ=8.18 (d, J=5.8 Hz, 1H), 7.75 (s, 1H), 7.56 (s, 4H), 7.17-7.08 (m, 2H), 4.94 (br d, J=4.0 Hz, 1H), 4.48-4.38 (m, 1H), 4.06-3.94 (m, 2H), 3.87 (dt, J=5.4, 8.3 Hz, 1H), 3.75 (dd, J=3.0, 9.3 Hz, 1H), 2.82 (s, 3H), 2.48 (s, 3H), 2.39-2.28 (m, 1H), 1.93 (br dd, J=4.4, 8.1 Hz, 1H)

### Example 70: Synthesis of 5-(4-chlorophenyl)-7-(2-((3,3-difluorocyclobutyl)amino)pyridin-4-yl)-2,3-dimethylpyrido[2,3-d]pyridazin-8 (7H)-one I-117

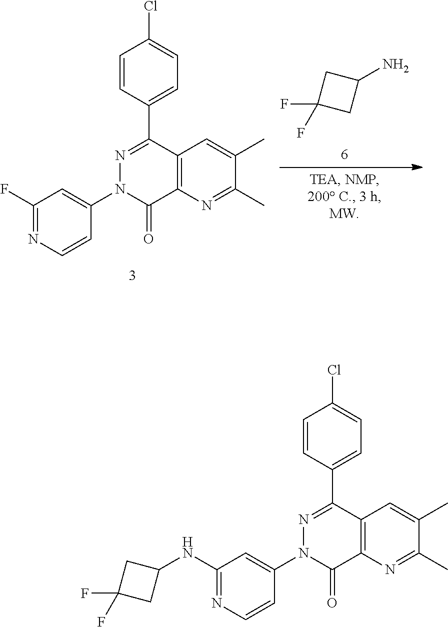

Step 1: A solution of 3,3-difluorocyclobutan-1-amine (10.0 eq, 84 mg, 0.788 mmol) and TEA (6.00 eq, 0.041 mL, 0.473 mmol) in NMP (2 mL) was added 5-(4-chlorophenyl)-7-(2-fluoropyridin-4-yl)-2,3-dimethylpyrido[2,3-d]pyridazin-8 (7H)-one (1.00 eq, 30 mg, 0.0788 mmol). The reaction vessel was sealed and heated under microwave at 200° C. for 3 h. LCMS showed the starting material was consumed completely and a major peak with desired MS (77.9%, Rt: 0.843 min; [M+H]⁺=468.1 at 220 nm). The reaction mixture was purified by prep-HPLC (Phenomenex C18 75×30 mm×3 um; mobile phase: [water (0.1% FA)-ACN]; B %: 25%-55%, 9 min) and lyophilized to give 5-(4-chlorophenyl)-7-(2-((3,3-difluorocyclobutyl)amino)pyridin-4-yl)-2,3-dimethylpyrido[2,3-d]pyridazin-8 (7H)-one (13 mg, 0.0269 mmol, 34.18% yield) as yellow solid, checked by LCMS H NMR, F NMR, HPLC (P1, achiral): [M+H]⁺=468.2; purity=100% (220 nm); Retention time=0.870 min¹H NMR (400 MHz, CDCl₃) δ=8.17 (d, J=5.7 Hz, 1H), 7.75 (s, 1H), 7.56 (s, 4H), 7.21 (dd, J=1.7, 5.7 Hz, 1H), 7.15 (d, J=1.3 Hz, 1H), 5.48-5.28 (m, 1H), 4.15 (br s, 1H), 3.16-3.06 (m, 2H), 2.82 (s, 3H), 2.54-2.45 (m, 5H).

### Example 71: Synthesis of 5-(4-chlorophenyl)-2, 3-dimethyl-7-[6-(oxetan-3-ylamino)-3-pyridyl]pyrido [2, 3-d]pyridazin-8-one; formic acid I-118

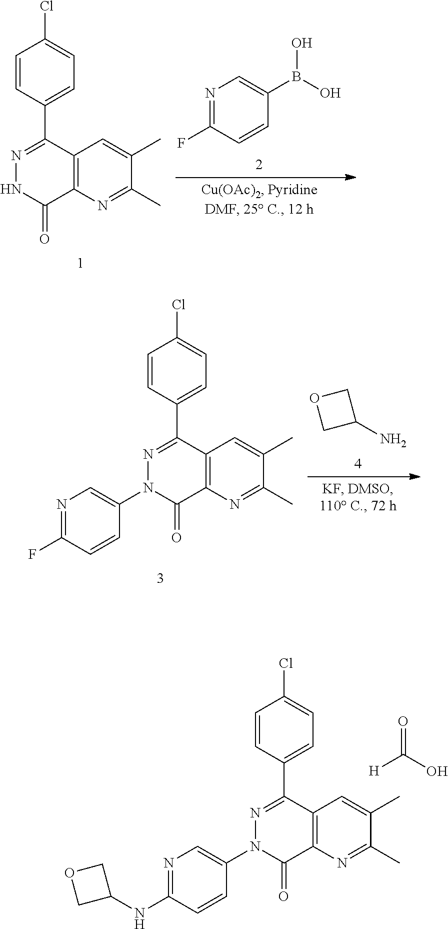

Step 1: A mixture of 5-(4-chlorophenyl)-2,3-dimethyl-7H-pyrido[2,3-d]pyridazin-8-one (1.00 eq, 200 mg, 0.700 mmol) and (6-fluoro-3-pyridyl)boronic acid (2.00 eq, 197 mg, 1.40 mmol) in DMF (5 mL) was added pyridine (5.00 eq, 0.28 mL, 3.50 mmol) and Cu(OAc)₂ (1.10 eq, 139 mg, 0.770 mmol). The mixture was degassed with 02 for 3 times. The mixture was stirred at 25° C. under 02 (15 psi) for 12 h. LCMS showed 87% of desired product. The reaction solution was filtered through celite and evaporated under reduced pressure to give the residue, which was then purified with Flash column (PE:EA=1:1, R_{f}=0.5) and dried in vacuo to give 5-(4-chlorophenyl)-7-(6-fluoro-3-pyridyl)-2,3-dimethyl-pyrido[2, 3-d]pyridazin-8-one (210 mg, 0.489 mmol, 69.80% yield) as white solid. LCMS (M+H)⁺=381.1; Retention time=0.934 min

Step 2: A solution of 5-(4-chlorophenyl)-7-(6-fluoro-3-pyridyl)-2,3-dimethyl-pyrido[2, 3-d]pyridazin-8-one (1.00 eq, 210 mg, 0.551 mmol), oxetan-3-amine (20.0 eq, 806 mg, 11.0 mmol) and KF (6.00 eq, 192 mg, 3.31 mmol) in DMSO (5 mL) was stirred at 110° C. for 12 h. LCMS showed the reactant was consumed and 46% of desired mass was detected, the combined reaction solution (with GL-2021-03-042-22-1) was purified with Prep-HPLC (FA) and lyophilized to give 5-(4-chlorophenyl)-2,3-dimethyl-7-[6-(oxetan-3-ylamino)-3-pyridyl]pyrido [2, 3-d]pyridazin-8-one; formic acid (80 mg, 0.151 mmol, 27.30% yield) as yellow solid. LCMS (M+H)⁺=434.1; purity=96.7% (220 nm); Retention time=0.779 min; ¹H NMR (400 MHz, CDCl₃) δ=8.65-8.58 (m, 1H), 8.56-8.52 (m, 1H), 8.29-8.22 (m, 1H), 7.81-7.76 (m, 1H), 7.66-7.52 (m, 4H), 7.21-7.14 (m, 1H), 4.74-4.60 (m, 2H), 4.53-4.43 (m, 1H), 4.22-4.08 (m, 1H), 3.73-3.57 (m, 1H), 2.85-2.79 (m, 3H), 2.53-2.47 (m, 3H)

### Example 72: Synthesis of 5-(4-chlorophenyl)-2,3-dimethyl-7-[6-[methyl(oxetan-3-yl)amino]-3-pyridyl]pyrido[2,3-d]pyridazin-8-one; formic acid I-119

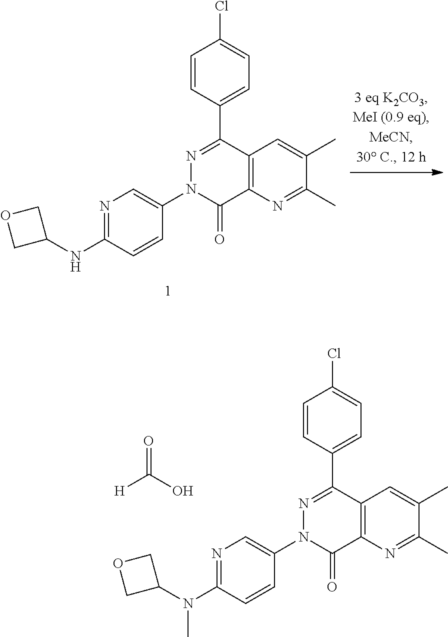 [SMILES: Cc1cc2c(-c3ccc(Cl)cc3)nn(-c3ccc(NC4COC4)nc3)c(=O)c2nc1C ; Cc1cc2c(-c3ccc(Cl)cc3)nn(-c3ccc(N(C)C4COC4)nc3)c(=O)c2nc1C ; O=CO]

Step 1: To a solution suspension of 5-(4-chlorophenyl)-2,3-dimethyl-7-[6-(oxetan-3-ylamino)-3-pyridyl]pyrido[2,3-d]pyridazin-8-one (1.00 eq, 40 mg, 0.0922 mmol) and K₂CO₃ (3.00 eq, 38 mg, 0.277 mmol) in MeCN (1 mL) was added and Mel (0.900 eq, 0.0052 mL, 0.0830 mmol) and the reaction mixture was stirred at 30° C. for 1 hour. LCMS (YG-2021-01-75-P1A1) (5-95AB/1.5 min): RT=0.938 min, 448.2=[M+H]⁺, ESI+ showed 77% of desired product. The reaction mixture was filtered and the filtrate was purified by prep-HPLC (Unisil 3-100 C18 Ultra 150*50 mm*3, um water (FA)-ACN) to afford 5-(4-chlorophenyl)-2,3-dimethyl-7-[6-[methyl(oxetan-3-yl)amino]-3-pyridyl]pyrido[2,3-d]pyridazin-8-one; formic acid (7.8 mg, 0.0159 mmol, 17.20% yield) as light yellow solid, which confirmed by ¹H NMR, LCMS, LCMS: RT=0.791 min, 448.1=[M+H]^{+ 1}H NMR (400 MHz, CDCl₃) δ=8.87-8.76 (m, 1H), 8.67 (s, 1H), 8.42 (br d, J=9.6 Hz, 1H), 7.75 (s, 1H), 7.58 (q, J=8.5 Hz, 4H), 7.00-6.88 (m, 1H), 5.59-5.49 (m, 1H), 4.80-4.68 (m, 1H), 4.30-4.21 (m, 1H), 4.02 (br d, J=13.0 Hz, 1H), 3.88-3.79 (m, 1H), 3.24 (s, 3H), 2.80 (s, 3H), 2.48 (s, 3H)

### Example 73: Synthesis of 5-(4-chlorophenyl)-7-(2-((1-cyclopropyl-1H-pyrazol-4-yl)amino)pyridin-4-yl)-2,3-dimethylpyrido[2,3-d]pyridazin-8 (7H)-one I-120

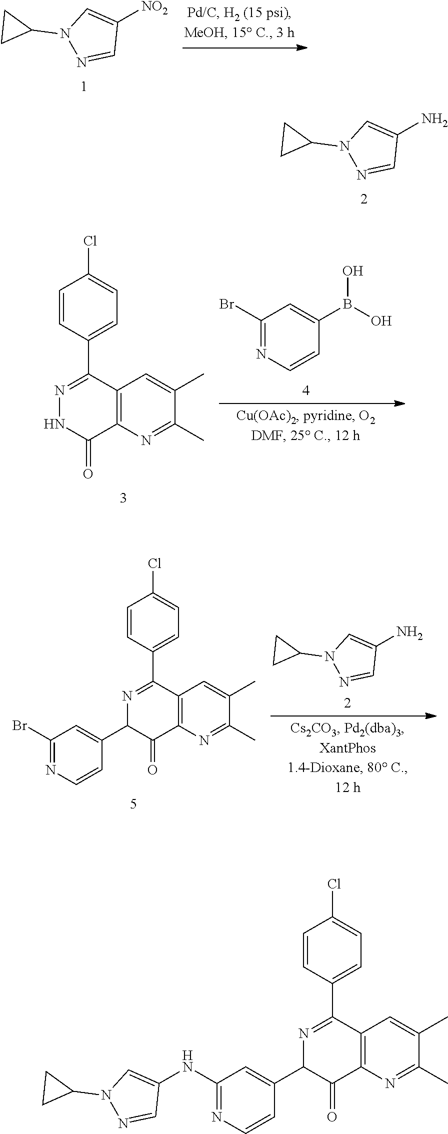

Step 1: To a solution of 1-cyclopropyl-4-nitro-1H-pyrazole (1.00 eq, 900 mg, 5.88 mmol) in MeOH (25 mL) was added Pd/C (0.289 eq, 180 mg, 1.70 mmol) under N₂. The mixture was purged with H₂ (15 psi) 3 times, then the mixture was stirred at 15° C. under H₂ (15 psi) for 3 h. LCMS showed the starting material was consumed completely. The reaction mixture was filtered and concentrated under reduced pressure to give a residue 1-cyclopropyl-1H-pyrazol-4-amine (720 mg, 5.85 mmol, 99.47% yield) as red liquid, checked by H NMR. ¹H NMR (400 MHz, CDCl₃) δ=7.12 (s, 1H), 7.06 (s, 1H), 3.47 (tt, J=3.7, 7.2 Hz, 1H), 1.08-1.03 (m, 2H), 0.97-0.92 (m, 2H)

Step 2: A mixture of 5-(4-chlorophenyl)-2,3-dimethylpyrido[2,3-d]pyridazin-8 (7H)-one (1.00 eq, 200 mg, 0.700 mmol) and (2-bromopyridin-4-yl)boronic acid (2.00 eq, 283 mg, 1.40 mmol) in DMF (5 mL) was added pyridine (5.00 eq, 0.28 mL, 3.50 mmol) and Cu(OAc)₂ (1.10 eq, 139 mg, 0.770 mmol). The mixture was degassed with O₂ for 3 times. The mixture was stirred at 25° C. under O₂ (15 psi) for 12 h. LCMS showed the starting material was consumed and a major peak with desired MS (77%, Rt: 0.975 min; [M+H]⁺=443.0 at 220 nm)). The reaction mixture was partitioned between DCM (100 mL) and water (100 mL). The combined organic layers were washed with brine (50 mL), dried over Na₂SO₄, filtered and concentrated under reduced pressure to give a residue. The crude product was purified by column chromatography on silica gel (eluted with PE/EtOAc=0˜100%, PE/EtOAc=1/1, the desired product R_{f}=0.2) to afford 7-(2-bromopyridin-4-yl)-5-(4-chlorophenyl)-2,3-dimethylpyrido[2,3-d]pyridazin-8 (7H)-one (190 mg, 0.430 mmol, 61.45% yield) as yellow solid, checked by LCMS, H NMR, [M+H]⁺=443.0; purity=95.6% (220 nm); Retention time=0.973 min. ¹H NMR (400 MHz, CDCl₃) δ=8.53-8.43 (m, 1H), 8.16 (br s, 1H), 8.07-7.98 (m, 1H), 7.75 (br s, 1H), 7.61-7.55 (m, 4H), 2.87-2.78 (m, 3H), 2.53-2.43 (m, 3H)

Step 3: To a solution of 1-cyclopropyl-1H-pyrazol-4-amine (0.833 eq, 23 mg, 0.189 mmol) in 1,4-Dioxane (5 mL) was added 7-(2-bromopyridin-4-yl)-5-(4-chlorophenyl)-2,3-dimethylpyrido[2,3-d]pyridazin-8 (7H)-one (1.00 eq, 100 mg, 0.226 mmol), Cs₂CO₃ (2.50 eq, 184 mg, 0.566 mmol), Pd₂(dba)₃ (0.100 eq, 13 mg, 0.0226 mmol) and XantPhos (0.100 eq, 13 mg, 0.0226 mmol) at 25° C. The reaction mixture was degassed with N₂ for 3 times. The mixture was stirred at 80° C. under N₂ for 4 hours. LCMS showed the starting material was consumed and a major peak with desired MS (29%, MS: 484.0 [M+H]+, ESI pos). The reaction mixture was partitioned between EtOAc (30×2 mL) and water (40 mL). The combined organic layers were dried over Na₂SO₄, filtered and concentrated under reduced pressure to give a residue. The crude product was purified by prep-TLC (DCM/MeOH=10/1, Rf=0.2) to give 30 mg (77% yield) and 20 mg (65% yield). 10 mg (77% yield) was purified by prep-HPLC (Phenomenex Luna C18 150×25 mm×10 um; mobile phase: [water (0.1% FA)-ACN]; B %: 24%-54%, 12 min) and lyophilized to give 5-(4-chlorophenyl)-7-(2-((1-cyclopropyl-1H-pyrazol-4-yl)amino)pyridin-4-yl)-2,3-dimethylpyrido[2,3-d]pyridazin-8 (7H)-one (3.0 mg, 0.00620 mmol, 2.74% yield) as yellow solid. 20 mg YT-2021-01-95-1 (65% yield) was purified by prep-HPLC (Phenomenex Luna C18 150×25 mm×10 um; mobile phase: [water (0.1% FA)-ACN]; B %: 27%-57%, 12 min) and lyophilized to give 5-(4-chlorophenyl)-7-(2-((1-cyclopropyl-1H-pyrazol-4-yl)amino)pyridin-4-yl)-2,3-dimethylpyrido[2,3-d]pyridazin-8 (7H)-one (2.0 mg, 0.00413 mmol, 1.83% yield) as yellow solid. [M+H]⁺=484.4; purity=94% (220 nm); Retention time=0.643 min. ¹H NMR (400 MHz, CDCl₃) δ=8.20 (br d, J=5.8 Hz, 1H), 7.72 (d, J=10.0 Hz, 2H), 7.55 (s, 4H), 7.46 (s, 1H), 7.32 (br d, J=7.3 Hz, 1H), 7.24 (dd, J=1.5, 5.8 Hz, 1H), 3.59 (td, J=3.5, 7.2 Hz, 1H), 2.81 (s, 3H), 2.48 (s, 3H), 1.18-1.11 (m, 2H), 1.05-0.98 (m, 2H).

### Example 74: Synthesis of 5-(4-chlorophenyl)-2,3-dimethyl-7-(4-(trifluoromethoxy)phenyl)-1,7-naphthyridin-8 (7H)-one I-122

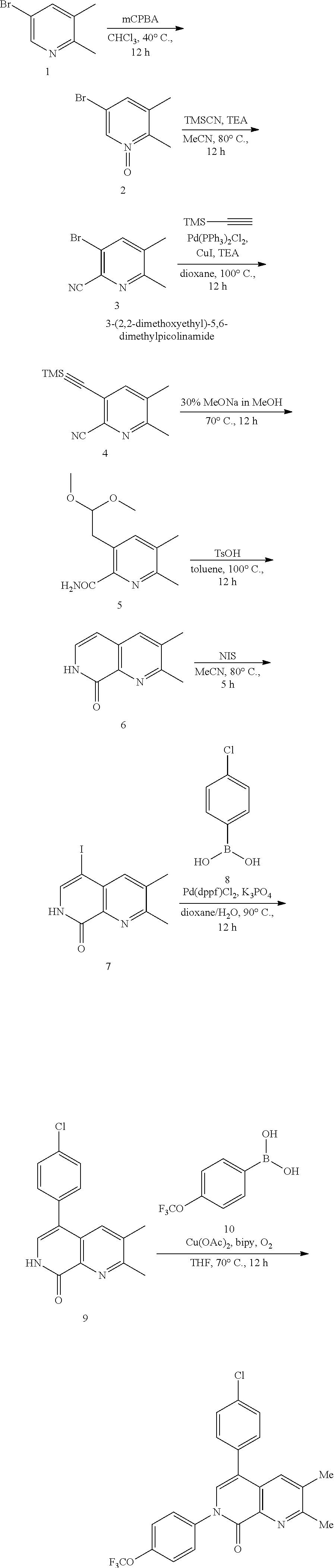

Step 1: To a solution of 5-bromo-2,3-dimethyl-pyridine (10 g, 53.7 mmol, 1.0 eq) in chloroform (100 mL) was added 3-chloroperbenzoic acid (16.37 g, 80.6 mmol, 1.50 eq). The mixture was then stirred at 60° C. for 12 hours. The reaction mixture was quenched with Na₂SO_{3(aq)}, washed with NaHCO₃ (aq), and then extracted with DCM (60 mL×3). The organic phase was concentrated under reduced pressure. The crude was purified by column chromatography (SiO₂, Petroleum ether: ethyl acetate=0% to 100%) to give the product 5-bromo-2,3-dimethyl-pyridine 1-oxide (10.20 g, 50.5 mmol, 93.9% yield) as white solid.

Step 2: To a solution of 5-bromo-2,3-dimethyl-pyridine 1-oxide (10.2 g, 50.5 mmol, 1.0 eq) in MeCN (120 mL) was added TEA (21 mL, 151 mmol, 3.0 eq) and TMSCN (25 mL, 202 mmol, 4.0 eq). The reaction was stirred at 100° C. for 12 hours. The reaction was concentrated to dryness and the residue was purified by flash column chromatography eluting with 10% ethyl acetate in petroleum ether. The desired fractions were concentrated to give 3-bromo-5,6-dimethyl-pyridine-2-carbonitrile (8.20 g, 38.9 mmol, 77.0% yield).

Step 3: To a solution of 3-bromo-5,6-dimethyl-pyridine-2-carbonitrile (8.2 g, 38.9 mmol, 1.0 eq) and trimethylsilylacetylene (11 mL, 77.7 mmol, 2.0 eq) in 1,4-dioxane (200 mL) were added Pd(dppf)Cl₂ (1.59 g, 1.94 mmol, 0.05 eq), CuI (740 mg, 3.89 mmol, 0.1 eq) and TEA (19 mL, 136 mmol, 3.5 eq). The reaction was stirred at 100° C. for 12 hours. After cooling to ambient temperature, the mixture was filtered through celite, and the filtrate was concentrated under vacuum. The residue was purified by silica gel column chromatography (eluting with ethyl acetate/petroleum ether, 5% to 20%) to give 5,6-dimethyl-3-(2-trimethylsilylethynyl)pyridine-2-carbonitrile (7.00 g, 30.7 mmol, 78.9% yield).

Step 4: A mixture of 5,6-dimethyl-3-(2-trimethylsilylethynyl)pyridine-2-carbonitrile (7.0 g, 30.7 mmol, 1.0 eq) in 30% MeONa in MeOH (150 mL, 30.7 mmol, 1.0 eq) was stirred at 70° C. for 12 hours. After cooling to ambient temperature, the mixture was concentrated under vacuum. The residue was diluted with water and extracted with DCM. The combined organic phases were washed with water and brine, dried over sodium sulfate, concentrated under vacuum, and purified by silica gel column chromatography (eluting with ethyl acetate/petroleum ether, 30% to 60%) to give 3-(2,2-dimethoxyethyl)-5,6-dimethyl-pyridine-2-carboxamide (2.00 g, 8.39 mmol, 27.4% yield).

Step 5: To a solution of 3-(2,2-dimethoxyethyl)-5,6-dimethyl-pyridine-2-carboxamide (2.0 g, 8.39 mmol, 1.0 eq) in toluene (60 mL) was added TsOH (289 mg, 1.68 mmol, 0.2 eq). The reaction was stirred at 100° C. for 12 hours. After cooling to ambient temperature, the mixture was concentrated under vacuum. The residue was purified by silica gel column chromatography (eluting with MeOH/DCM, 0% to 10%) to give 2,3-dimethyl-7H-1,7-naphthyridin-8-one (700 mg, 4.02 mmol, 47.9% yield).

Step 6: To a solution of 2,3-dimethyl-7H-1,7-naphthyridin-8-one (50 mg, 0.29 mmol, 1.0 eq) in DMF (2 mL) was added NIS (71 mg, 0.32 mmol, 1.1 eq). The reaction was stirred at 50° C. for 12 hours. The reaction was concentrated and then purified by flash column chromatography eluting with 2% MeOH in DCM. The desired fractions were concentrated to dryness in vacuo to give 5-iodo-2,3-dimethyl-7H-1,7-naphthyridin-8-one (50 mg, 0.167 mmol, 58.1% yield).

Step 7: To a solution of 5-iodo-2,3-dimethyl-7H-1,7-naphthyridin-8-one (50 mg, 0.167 mmol, 1.0 eq) and (4-chlorophenyl)boronic acid (78 mg, 0.5 mmol, 3.0 eq) in 1,4-dioxane (8 mL) and water (1 mL) were added Pd(dppf)Cl₂ (14 mg, 0.0167 mmol, 0.1 eq) and K₃PO₄ (106 mg, 0.5 mmol, 3.0 eq). The reaction was stirred at 90° C. for 6 hours under N₂. After cooling to ambient temperature, the mixture was filtered through celite and the filtrate was concentrated under vacuum. The residue was purified by silica gel column chromatography (eluting with MeOH/DCM, 1% to 5%) to give 5-(4-chlorophenyl)-2,3-dimethyl-7H-1,7-naphthyridin-8-one (50 mg, 0.149 mmol, 89.6% yield).

Step 8: To a solution of 5-(4-chlorophenyl)-2,3-dimethyl-7H-1,7-naphthyridin-8-one (50 mg, 0.176 mmol, 1.0 eq) and [4-(trifluoromethoxy)phenyl]boronic acid (108 mg, 0.527 mmol, 3.0 eq) in THF (8 mL) and DMF (2 mL) were added Cu(OAc)₂ (105 mg, 0.527 mmol, 3.0 eq) and 2,2-bipyridine (82.2 mg, 0.527 mmol, 3.0 eq). The mixture was filtered through celite and the filtrate was concentrated under vacuum. The residue was purified by prep-HPLC(FA) to give 5-(4-chlorophenyl)-2,3-dimethyl-7-[4-(trifluoromethoxy)phenyl]-1,7-naphthyridin-8-one (8.1 mg, 0.0182 mmol, 5.8% yield). ¹H NMR (400 MHz, CDCl₃) δ 7.72-7.68 (m, 3H), 7.58-7.52 (m, 6H), 7.50 (s, 1H), 2.61 (s, 3H), 2.38 (s, 3H).

### Example 75: Synthesis of 6-[(2R,4S)-2-(1-cyclopropyl-1H-pyrazol-4-yl)tetrahydro-2H-pyran-4-yl]-8-[2-fluoro-4-(trifluoromethyl)phenyl]-2,3-dimethylpyrido[3,4-b]pyrazin-5 (6H)-one I-123

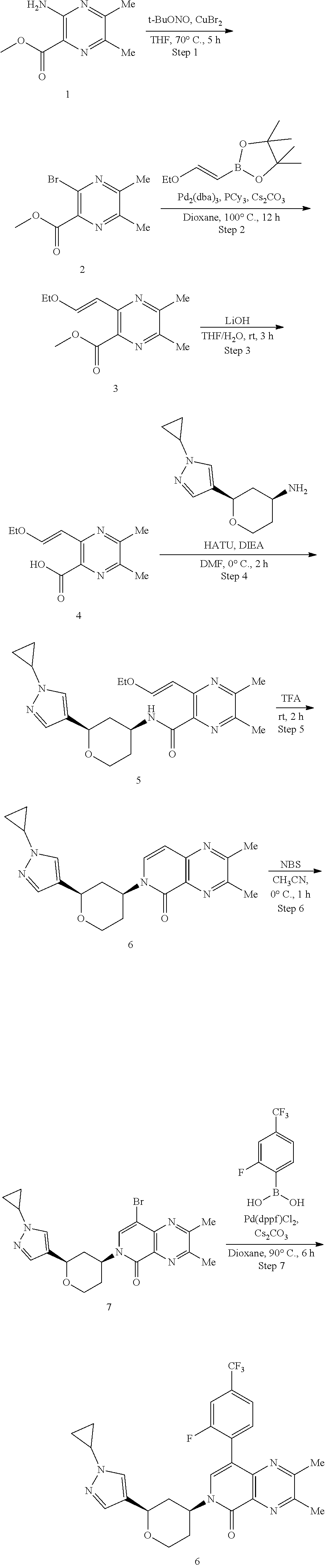

Step 1: To a solution of CuBr₂ (2.95 g, 13.25 mmol, 1.5 equiv) in THF (30 mL) was added t-BuONO (1.37 g, 13.25 mmol, 1.5 equiv) at room temperature under nitrogen. After stirring at 70° C. for 10 minutes, the solution was cooled to room temperature and a solution of methyl 3-amino-5,6-dimethylpyrazine-2-carboxylate (1.60 g, 8.83 mmol, 1.0 equiv) in THF (10 mL) was added dropwise. The mixture was then stirred at 70° C. for 2 hours. LCMS (CZ-2022-01-019) indicated the starting material was consumed completely and 60% desired compound was detected. The reaction mixture was cooled to room temperature, quenched with water, and extracted with ethyl acetate (30 mL×3). The organic phase was dried over Na₂SO₄ and concentrated under reduced pressure. The crude product was purified by column chromatography (SiO₂, Petroleum ether: ethyl acetate=0% to 20%) to give the product methyl 3-bromo-5,6-dimethylpyrazine-2-carboxylate (879 mg, 3.59 mmol, 40% yield) as a yellow solid. ¹H NMR (400 MHz, DMSO) δ 3.91 (s, 3H), 2.54 (s, 3H), 2.49 (s, 3H).

Step 2: A mixture of Pd₂(dba)₃ (176 mg, 0.3 mmol, 0.1 equiv), PCy₃ (172 mg, 0.62 mmol, 0.2 equiv), Cs₂CO₃ (3.98 g, 12.2 mmol, 3.0 equiv) and methyl 3-bromo-5,6-dimethyl-pyrazine-2-carboxylate (750 mg, 3.06 mmol, 1.0 equiv) was prepared in a flask under nitrogen. Then 1,4-dioxane (20 mL) and 2-[(E)-2 ethoxyvinyl]-4,4,5,5-tetramethyl-1,3,2-dioxaborolane (909 mg, 4.59 mmol, 1.5 equiv) were added and the mixture was stirred at 100° C. for 5 h. LCMS indicated the starting material was consumed completely and 80% desired compound was detected. Then the suspension was cooled to room temperature, filtered through a plug of celite, washed with water and extracted with EtOAc. The organic phase was dried over Na₂SO₄ and concentrated under reduced pressure. The crude product was purified by column chromatography (silica gel, EtOAc/PE=1:1) to give the product methyl (E)-3-(2-ethoxyvinyl)-5,6-dimethylpyrazine-2-carboxylate (520 mg, 2.20 mmol, 72% yield) as a yellow solid. ¹H NMR (400 MHz, DMSO) δ 7.74 (d, J=6.4 Hz, 1H), 6.52 (d, J=8.0 Hz, 1H), 4.00 (q, J=8.13 Hz, 2H), 3.85 (s, 3H), 2.49 (s, 3H), 2.44 (s, 3H), 1.28 (t, J=7 Hz, 3H).

Step 3: To a solution of methyl (E)-3-(2-ethoxyvinyl)-5,6-dimethylpyrazine-2-carboxylate (520 mg, 2.20 mmol, 1.0 equiv) in THF (15 mL) and water (5 mL) was added LiOH (102 mg, 4.40 mmol, 2.0 equiv). The mixture was stirred at room temperature for 3 hours. LCMS indicated that the starting material was consumed completely, and 80% desired compound was detected. The resulting solution was treated with HCl to pH 5 and dried by a freeze dryer to give the product (E)-3-(2-ethoxyvinyl)-5,6-dimethylpyrazine-2-carboxylic acid. The crude product was used for the next step without further purification. LC-MS: Rt: 1.335 min, m/z: [M+H]⁺=223.1. 80% purity at 254 nm

Step 4: A solution of 3-[(E)-2-ethoxyvinyl]-5,6-dimethyl-pyrazine-2-carboxylic acid (333 mg, 1.5 mmol, 1.0 equiv), 2-(1-cyclopropylpyrazol-4-yl)tetrahydropyran-4-amine (310 mg, 1.5 mmol, 1.0 equiv) and HATU (856 mg, 2.25 mmol, 1.5 equiv) in DMF (20 mL) was prepared in a flask under nitrogen. Then DIEA (582 mg, 4.5 mmol, 3.0 equiv) was added and the solution was stirred at 0° C. for 1 h. LCMS indicated that the starting material was consumed, and desired compound was detected. The reaction was quenched with water and extracted with EtOAc. The organic phase was washed with brine, dried over Na₂SO₄ and concentrated under reduced pressure. The crude product was purified by column chromatography (silica, Petroleum ether/Ethyl acetate=1:2) to give the desired product N-[(2R,4S)-2-(1-cyclopropyl-1H-pyrazol-4-yl)tetrahydro-2H-pyran-4-yl]-3-[(E)-2-ethoxyvinyl]-5,6-dimethylpyrazine-2-carboxamide (480 mg, 1.17 mmol, 78% yield) as a yellow oil. ¹H NMR (400 MHz, DMSO) δ 8.50 (d, J=8.0 Hz, 1H), 7.72-7.66 (m, 2H), 7.34 (s, 1H), 6.88 (d, J=12.4 Hz, 1H), 4.38 (d, J=10.4 Hz, 1H), 4.15-4.04 (m, 1H), 3.99-3.93 (m, 2H), 3.68-3.61 (m, 1H), 3.56 (t, J=11.4 Hz, 1H), 2.56 (d, J=12.4 Hz, 1H), 2.47 (s, 3H), 2.47 (s, 3H), 2.05 (d, J=15.2 Hz, 1H), 1.78 (d, J=11.6 Hz, 1H), 1.67-1.58 (m, 2H), 1.29-1.24 (m, 3H), 0.99-0.88 (m, 4H).

Step 5: A solution of N-[(2R,4S)-2-(1-cyclopropylpyrazol-4-yl)tetrahydropyran-4-yl]-3-[(E)-2-ethoxyvinyl]-5,6-dimethyl-pyrazine-2-carboxamide (480 mg, 1.17 mmol, 1.0 equiv) in TFA (2.0 mL) was prepared under nitrogen. Then the reaction was stirred at room temperature for 3 hours. LCMS indicated that the starting material was consumed completely, and desired compound was detected. The reaction was quenched with water and extracted with EtOAc. The organic phase was dried over Na₂SO₄ and concentrated under reduced pressure. The crude product was purified by column chromatography (silica gel, DCM/MeOH=10:1) to give the product 6-[(2R,4S)-2-(1-cyclopropyl-1H-pyrazol-4-yl)tetrahydro-2H-pyran-4-yl]-2,3-dimethylpyrido[3,4-b]pyrazin-5 (6H)-one as a brown solid (262 mg, 0.72 mmol, 61% yield). ¹H NMR (400 MHz, CDCl₃) δ 7.86 (d, J=7.6 Hz, 1H), 7.74 (s, 1H), 7.40 (s, 1H), 6.66 (d, J=8.0 Hz, 1H), 5.22-5.12 (m, 1H), 4.54-4.51 (m, 1H), 4.09-4.06 (m, 1H), 3.74-3.65 (m, 2H), 2.63 (s, 6H), 2.06-2.01 (m, 3H), 1.77 (d, J=9.6 Hz, 1H), 0.98-0.90 (m, 4H).

Step 6: To a solution of 6-[(2R,4S)-2-(1-cyclopropyl-1H-pyrazol-4-yl)tetrahydro-2H-pyran-4-yl]-2,3-dimethylpyrido[3,4-b]pyrazin-5 (6H)-one (240 mg, 0.66 mmol, 1.0 equiv) in MeCN (10 mL) was added NBS (129 mg, 0.72 mmol, 1.1 equiv) under nitrogen. The solution was stirred at 0° C. for 1 h. LCMS indicated that the starting material was consumed and ˜50% desired product was detected. The reaction was quenched with water and extracted with EtOAc. The organic phase was dried over Na₂SO₄ and concentrated under reduced pressure. The crude product was purified by column chromatography (silica gel, EtOAc/Et₃N=20:1) to give 8-bromo-6-[(2R,4S)-2-(1-cyclopropyl-1H-pyrazol-4-yl)tetrahydro-2H-pyran-4-yl]-2,3-dimethylpyrido[3,4-b]pyrazin-5 (6H)-one (57 mg, 0.13 mmol, 20% yield) as a white solid. ¹H NMR (400 MHz, DMSO) δ 8.22 (s, 1H), 7.75 (s, 1H), 7.41 (s, 1H), 5.20-5.12 (m, 1H), 4.52 (dd, J=1.6, 10.8 Hz, 1H), 4.08-4.05 (m, 1H), 3.71-3.63 (m, 2H), 2.69 (s, 3H), 2.67 (s, 3H), 2.17 (q, J=11.9 Hz, 1H), 2.03-2.01 (m, 1H), 1.78 (d, J=12.4 Hz, 1H), 1.18 (t, J=7.0 Hz, 1H), 1.01-0.91 (m, 4H).

Step 7: A mixture of 8-bromo-6-[(2R,4S)-2-(1-cyclopropyl-1H-pyrazol-4-yl)tetrahydro-2H-pyran-4-yl]-2,3-dimethylpyrido[3,4-b]pyrazin-5 (6H)-one (50 mg, 0.11 mmol, 1.0 equiv), [2-fluoro-4-(trifluoromethyl)phenyl]boronic acid (47 mg, 0.23 mmol, 2.0 equiv), Pd(dppf)Cl₂ (8 mg, 0.01 mmol, 0.1 equiv) and Cs₂CO₃ (110 mg, 0.34 mmol, 3.0 equiv) was prepared in a flask under nitrogen, then 1,4-dioxane was added. The solution was stirred at 90° C. for 3 hours. LCMS indicated that the starting material was consumed, and desired product was detected. The suspension was filtered through a plug of silica gel and concentrated under reduced pressure. The crude product was purified by column chromatography (silica gel, EtOAc/Et₃N=20:1) to give the product 6-[(2R,4S)-2-(1-cyclopropyl-1H-pyrazol-4-yl)tetrahydro-2H-pyran-4-yl]-8-[2-fluoro-4-(trifluoromethyl)phenyl]-2,3-dimethylpyrido[3,4-b]pyrazin-5 (6H)-one (22 mg, 0.04 mmol, 37% yield) was brown solid. LC-MS: Rt: 1.37 min, m/z: 528.2 [M+H]+. 99% purity at 254 nm. ¹H NMR (400 MHz, DMSO) δ 8.03 (s, 1H), 7.79-7.75 (m, 2H), 7.73 (s, 1H), 7.69 (d, J=8.0 Hz, 1H), 7.40 (s, 1H), 5.29-5.22 (m, 1H), 4.55 (dd, J=1.2, 10.8 Hz, 1H), 3.08 (dd, J=3.2, 11.2 Hz, 1H), 3.72 (t, J=11.0 Hz, 1H), 3.67-3.61 (m, 1H), 2.65 (s, 3H), 2.55 (s, 3H), 2.18 (q, 11.7 Hz, 1H), 2.07-2.01 (m, 2H), 1.83 (d, J=12.0 Hz, 1H), 0.98-0.86 (m, 4H). HPLC: Rt: 3.85 min, 98% purity at 214 nm

### Example 75: Synthesis of 5-(4-fluorophenyl)-2,3-dimethyl-7-(4-(trifluoromethoxy)phenyl)pyrido[2,3-d]pyridazin-8 (7H)-one I-121

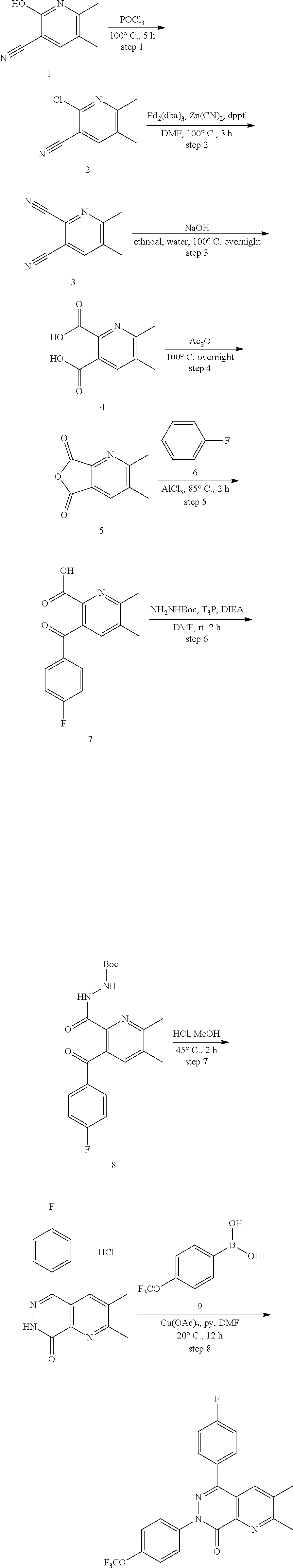

### Step 1: 2-chloro-5,6-dimethylnicotinonitrile

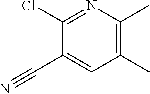

A mixture of 5,6-dimethyl-2-oxo-1H-pyridine-3-carbonitrile (1.0 g, 6.75 mmol, 1.00 eq) in POCl₃ (6.0 mL, 64.2 mmol, 9.51 eq) was stirred at 20° C. for 1 h, then the reaction mixture was stirred at 100° C. for 5 h. The reaction mixture was concentrated, and the residue was poured into saturated aqueous NaHCO₃ and adjusted pH>7. The mixture was extracted with ethyl acetate. The organic layer was washed with brine, and dried by Na₂SO₄. The solution was concentrated to give 2-chloro-5,6-dimethyl-pyridine-3-carbonitrile (1.1 g, 97.8%). LC-MS [M+H]⁺=167.0 R.T=0.930 min Step 2: 5,6-dimethylpyridine-2,3-dicarbonitrile

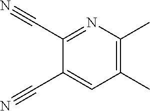

To a stirred solution of 2-chloro-5,6-dimethyl-pyridine-3-carbonitrile (1.10 g, 6.60 mmol, 1.0 eq) in DMF (12 mL) were added dppf (366 mg, 0.66 mmol, 0.1 eq), Zn(CN)₂ (814 mg, 6.93 mmol, 1.05 eq), and Pd₂(dba)₃ (380 mg, 0.66 mmol, 0.1 eq) at 25° C. under N₂. The mixture was heated to 100° C. under N₂ for 3 h. The reaction mixture was filtered and quenched by addition of 1N HCl (100 mL) at 0° C., and then extracted with ethyl acetate (80 mL×2). The combined organic layers were washed with brine (100 mL), dried over Na₂SO₄, filtered and concentrated under reduced pressure to give a residue. The residue was purified by column chromatography to obtain 5,6-dimethylpyridine-2,3-dicarbonitrile (0.85 g, 81.9%). LC-MS [M+H]⁺=158 R.T=0.852 min Step 3: 5,6-dimethylpyridine-2,3-dicarboxylic acid

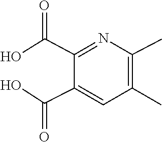

To a solution of 5,6-dimethylpyridine-2,3-dicarbonitrile (1.90 g, 12.1 mmol, 1.00 eq) in ethanol (25 mL) and water (25 mL) was added NaOH (7.25 g, 181 mmol, 15.0 eq). The mixture was stirred at 100° C. for 16 h. The reaction mixture was diluted by addition of H₂O (80 mL) at 0° C. and extracted with ethyl acetate (100 mL×2). The combined aqueous layers were adjusted to pH<7 with 1N HCl. The combined aqueous phase was concentrated under vacuum to give a yellow solid. The solid was dissolved in MeOH (50 mL) and filtered. The filtrated was concentrated under vacuum to give crude 5,6-dimethylpyridine-2,3-dicarboxylic acid (1.30 g, 55.1%). LC-MS [M+H]⁺=196.0 R.T=0.182 min Step 4: 2,3-dimethylfuro[3,4-b]pyridine-5,7-dione

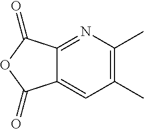

A mixture of 5,6-dimethylpyridine-2,3-dicarboxylic acid (2.0 g, 10.2 mmol, 1.0 eq) in acetic anhydride (16 mL, 170 mmol, 16.5 eq) was stirred at 100° C. for 16 h. LCMS (A drop of reaction mixture quenched by MeOH) showed that the starting material was consumed completely and the desired ester mass was detected (MS: 210.1=[M+MeOH]⁺, ESI+). The mixture was concentrated under reduced pressure to give 2,3-dimethylfuro[3,4-b]pyridine-5,7-dione (1.90 g, 104.7%). LC-MS [M+MeOH+H]+=210.1 R.T=0.492, 0.733 min Step 5: 3-(4-fluorobenzoyl)-5,6-dimethylpicolinic acid

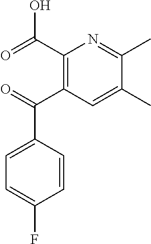 [SMILES: Cc1cc(C(=O)c2ccc(F)cc2)c(C(=O)O)nc1C]

To a stirred solution of 2,3-dimethylfuro[3,4-b]pyridine-5,7-dione (85 mg, 0.48 mmol, 1.0 eq) in DCE (2 mL) were added fluorobenzene (138 mg, 1.44 mmol, 3.0 eq) and AiCl₃ (192 mg, 1.44 mmol, 3.0 eq). The mixture was stirred at 85° C. for 2 h. The resulting mixture was diluted with water (20 mL) and extracted with DCM (50 mL×3). The combined organic phases were washed with brine, dried over sodium sulfate, concentrated to afford 3-(4-fluorobenzoyl)-5,6-dimethyl-pyridine-2-carboxylic acid (130 mg, 99.12%). LC-MS [M+H]⁺=274.0 R.T=0.838 min

### Step 6: tert-butyl 2-(3-(4-fluorobenzoyl)-5,6-dimethylpicolinoyl)hydrazine-1-carboxylate

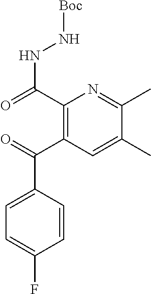 [SMILES: Cc1cc(C(=O)c2ccc(F)cc2)c(C(=O)NNC(=O)OC(C)(C)C)nc1C]

To a mixture of 3-(4-chlorobenzoyl)-5,6-dimethyl-pyridine-2-carboxylic acid (130 mg, 0.45 mmol, 1.0 eq), tert-butyl N-aminocarbamate (119 mg, 0.90 mmol, 2.0 eq) and DIPEA (0.39 mL, 2.24 mmol, 5.0 eq) in DMF (2 mL) was added T₃P (428 mg, 1.35 mmol, 3.0 eq). The mixture was stirred at 25° C. for 2 h. The mixture was diluted with water (20 mL) and extracted with ethyl acetate (50 mL×2). The combined organic layers were washed with brine (50 mL) and dried over Na₂SO₄. The solvent was filtered and the filtrate was concentrated under reduced pressure to give tert-butyl N-[[3-(4-chlorobenzoyl)-5,6-dimethyl-pyridine-2-carbonyl]amino]carbamate (185 mg, 102.1%). LC-MS [M+H]⁺=388.0 R.T=1.028 min

### Step 7: 5-(4-fluorophenyl)-2,3-dimethylpyrido[2,3-d]pyridazin-8 (7H)-one

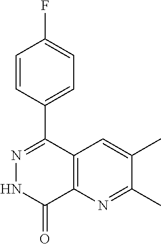 [SMILES: Cc1cc2c(-c3ccc(F)cc3)n[nH]c(=O)c2nc1C]

A mixture of tert-butyl N-[[3-(4-fluorobenzoyl)-5,6-dimethyl-pyridine-2-carbonyl]amino]carbamate (185 mg, 0.48 mmol, 1.0 eq) in HCl in methanol (4 M, 2.5 mL, 20 eq) was stirred at 45° C. for 2 h. The reaction mixture was concentrated under reduced pressure to give 5-(4-fluorophenyl)-2,3-dimethyl-7H-pyrido[2,3-d]pyridazin-8-one (160 mg, 109.6%). LC-MS [M+H]⁺=270.2 R.T=1.020 min

### Step 8: 5-(4-fluorophenyl)-2,3-dimethyl-7-(4-(trifluoromethoxy)phenyl)pyrido[2,3-d]pyridazin-8 (7H)-one

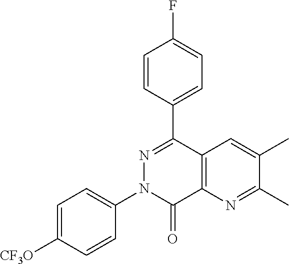

To a mixture of 5-(4-fluorophenyl)-2,3-dimethyl-7H-pyrido[2,3-d]pyridazin-8-one (130 mg, 0.48 mmol, 1.0 eq) and [4-([4-(trifluoromethoxy)phenyl]boronic acid (199 mg, 0.97 mmol, 2.0 eq) in DMF (2 mL) were added pyridine (115 mg, 1.45 mmol, 3.0 eq) and Cu(OAc)₂ (96 mg, 0.53 mmol, 1.1 eq). The mixture was degassed with O₂ for 3 times. The mixture was stirred at 25° C. under O₂ for 12 h. The mixture was filtered and the filtrate was purified by prep-HPLC to get 5-(4-fluorophenyl)-2,3-dimethyl-7-[4-(trifluoromethoxy)phenyl]pyrido[2,3-d]pyridazin-8-one (37 mg, 7.85%). LC-MS [M+H]⁺=430.0 R.T=1.405 min 1H NMR (400 MHz, DMSO) δ 7.85-7.83 (m, 3H), 7.73-7.71 (m, 2H), 7.54-7.52 (m, 2H), 7.43-7.39 (m, 2H), 2.70 (s, 3H), 2.45 (s, 3H).

### Table B. Exemplary Compounds

The compounds disclosed below in Table 1 were made by a method of the present disclosure or a similar method. The appropriate reagents, starting materials and conditions necessary for synthesizing the compounds of Table 1 would be apparent to a person of ordinary skill in the art. Compounds designated with “(+/−)” were isolated as a mixture of diastereomers sharing the same relative stereochemistry (ie. cis or trans). Compounds designated with “(rac)” were isolated as a mixture of all possible stereoisomers of the shown compound. Compounds lacking either designation were isolated with the specific stereochemistry shown, such that the specific stereoisomer shown made up at least 9000 of the isolated product.

| | | | Method used to |
|---|---|---|---|
| Ex # | Structure | Name | synthesize |
| 1 | 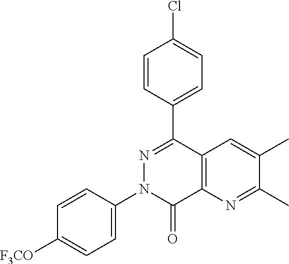 [SMILES: Cc1cc2c(-c3ccc(Cl)cc3)nn(-c3ccc(OC(F)(F)F)cc3)c(=O)c2nc1C] | 5-(4-chlorophenyl)-2,3-dimethyl-7-(4- (trifluoromethoxy)phenyl)pyrido[2,3- d]pyridazin-8(7H)-one | |
| 2 | 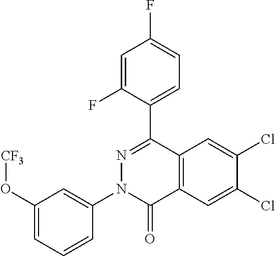 [SMILES: O=c1c2cc(Cl)c(Cl)cc2c(-c2ccc(F)cc2F)nn1-c1cccc(OC(F)(F)F)c1] | 6,7-dichloro-4-(2-fluoro-4- (trifluoromethyl)phenyl)-2-(3- (trifluoromethoxy)phenyl)phthalazin- 1(2H)-one | |
| 3 | 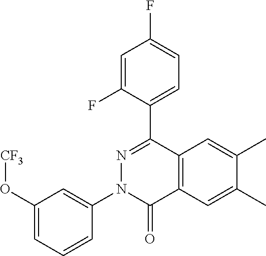 [SMILES: Cc1cc2c(-c3ccc(F)cc3F)nn(-c3cccc(OC(F)(F)F)c3)c(=O)c2cc1C] | 4-(2-fluoro-4-(trifluoromethyl)phenyl)- 6,7-dimethyl-2-(3- (trifluoromethoxy)phenyl)phthalazin- 1(2H)-one | |
| 4 | 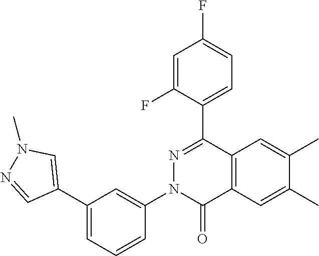 [SMILES: Cc1cc2c(-c3ccc(F)cc3F)nn(-c3cccc(-c4cnn(C)c4)c3)c(=O)c2cc1C] | 4-(2-fluoro-4-(trifluoromethyl)phenyl)- 6,7-dimethyl-2-(3-(1-methyl-1H- pyrazol-4-yl)phenyl)phthalazin-1(2H)- one | |
| 5 | 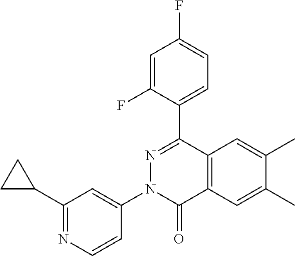 [SMILES: Cc1cc2c(-c3ccc(F)cc3F)nn(-c3ccnc(C4CC4)c3)c(=O)c2cc1C] | 2-(2-cyclopropylpyridin-4-yl)-4-(2- fluoro-4-(trifluoromethyl)phenyl)-6,7- dimethylphthalazin-1(2H)-one | |
| 6 | 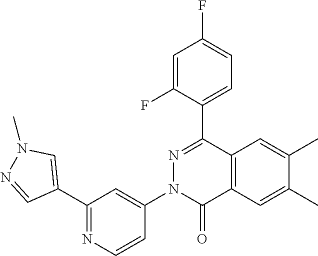 [SMILES: Cc1cc2c(-c3ccc(F)cc3F)nn(-c3ccnc(-c4cnn(C)c4)c3)c(=O)c2cc1C] | 4-(2-fluoro-4-(trifluoromethyl)phenyl)- 6,7-dimethyl-2-(2-(1-methyl-1H- pyrazol-4-yl)pyridin-4-yl)phthalazin- 1(2H)-one | |
| 7 | 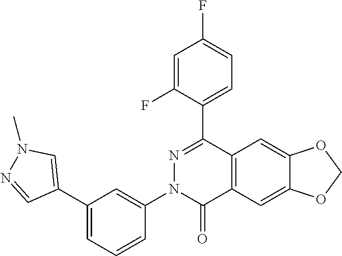 [SMILES: Cn1cc(-c2cccc(-n3nc(-c4ccc(F)cc4F)c4cc5c(cc4c3=O)OCO5)c2)cn1] | 8-(2-fluoro-4-(trifluoromethyl)phenyl)- 6-(3-(1-methyl-1H-pyrazol-4- yl)phenyl)-[1,3]dioxolo[4,5- g]phthalazin-5(6H)-one | |
| 8 | 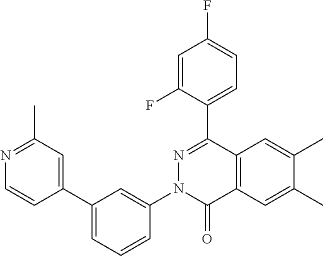 [SMILES: Cc1cc(-c2cccc(-n3nc(-c4ccc(F)cc4F)c4cc(C)c(C)cc4c3=O)c2)ccn1] | 4-(2-fluoro-4-(trifluoromethyl)phenyl)- 6,7-dimethyl-2-(3-(2-methylpyridin-4- yl)phenyl)phthalazin-1(2H)-one | |
| 9 | 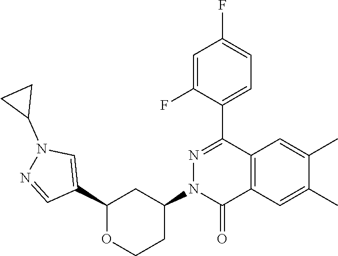 [SMILES: Cc1cc2c(-c3ccc(F)cc3F)nn([C@H]3CCO[C@@H](c4cnn(C5CC5)c4)C3)c(=O)c2cc1C] | 2-((2R,4S)-2-(1-cyclopropyl-1H- pyrazol-4-yl)tetrahydro-2H-pyran-4-yl)- 4-(2-fluoro-4-(trifluoromethyl)phenyl)- 6,7-dimethylphthalazin-1(2H)-one | |
| 10 | 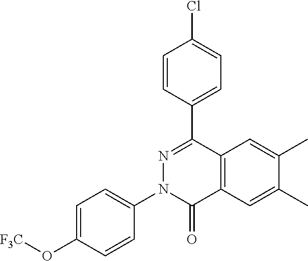 [SMILES: Cc1cc2c(-c3ccc(Cl)cc3)nn(-c3ccc(OC(F)(F)F)cc3)c(=O)c2cc1C] | 4-(4-chlorophenyl)-6,7-dimethyl-2-(4- (trifluoromethoxy)phenyl)phthalazin- 1(2H)-one | |
| 11 | 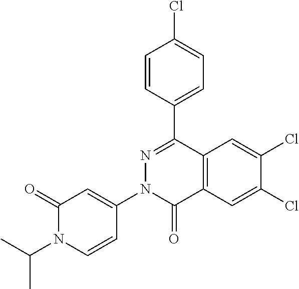 [SMILES: CC(C)n1ccc(-n2nc(-c3ccc(Cl)cc3)c3cc(Cl)c(Cl)cc3c2=O)cc1=O] | 6,7-dichloro-4-(4-chlorophenyl)-2-(1- isopropyl-2-oxo-1,2-dihydropyridin-4- yl)phthalazin-1(2H)-one | |
| 12 | 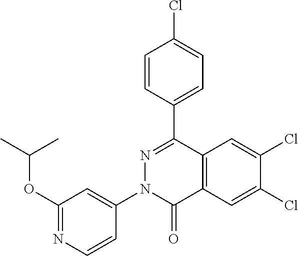 [SMILES: CC(C)Oc1cc(-n2nc(-c3ccc(Cl)cc3)c3cc(Cl)c(Cl)cc3c2=O)ccn1] | 6,7-dichloro-4-(4-chlorophenyl)-2-(2- isopropoxypyridin-4-yl)phthalazin- 1(2H)-one | |
| 13 | 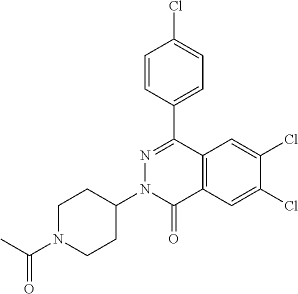 [SMILES: CC(=O)N1CCC(n2nc(-c3ccc(Cl)cc3)c3cc(Cl)c(Cl)cc3c2=O)CC1] | 2-(1-acetylpiperidin-4-yl)-6,7-dichloro- 4-(4-chlorophenyl)phthalazin-1(2H)- one | |
| 14 | 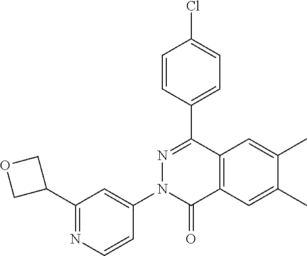 [SMILES: Cc1cc2c(-c3ccc(Cl)cc3)nn(-c3ccnc(C4COC4)c3)c(=O)c2cc1C] | 4-(4-chlorophenyl)-6,7-dimethyl-2-(2- (oxetan-3-yl)pyridin-4-yl)phthalazin- 1(2H)-one | |
| 15 | 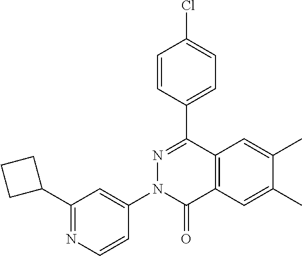 [SMILES: Cc1cc2c(-c3ccc(Cl)cc3)nn(-c3ccnc(C4CCC4)c3)c(=O)c2cc1C] | 4-(4-chlorophenyl)-2-(2- cyclobutylpyridin-4-yl)-6,7- dimethylphthalazin-1(2H)-one | |
| 16 | 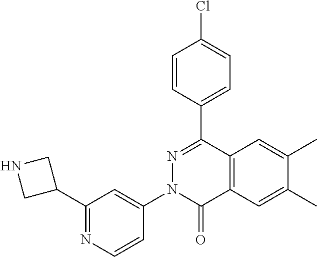 [SMILES: Cc1cc2c(-c3ccc(Cl)cc3)nn(-c3ccnc(C4CNC4)c3)c(=O)c2cc1C] | 2-(2-(azetidin-3-yl)pyridin-4-yl)-4-(4- chlorophenyl)-6,7-dimethylphthalazin- 1(2H)-one | |
| 17 | 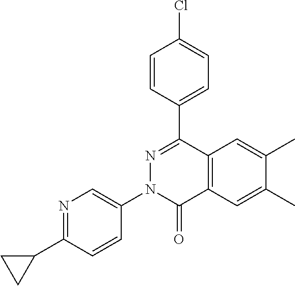 [SMILES: Cc1cc2c(-c3ccc(Cl)cc3)nn(-c3ccc(C4CC4)nc3)c(=O)c2cc1C] | 4-(4-chlorophenyl)-2-(6- cyclopropylpyridin-3-yl)-6,7- dimethylphthalazin-1(2H)-one | |
| 18 | 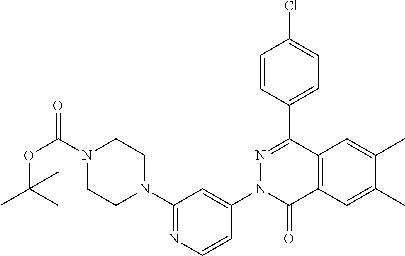 [SMILES: Cc1cc2c(-c3ccc(Cl)cc3)nn(-c3ccnc(N4CCN(C(=O)OC(C)(C)C)CC4)c3)c(=O)c2cc1C] | tert-butyl 4-(4-(4-(4-chlorophenyl)-6,7- dimethyl-1-oxophthalazin-2(1H)- yl)pyridin-2-yl)piperazine-1- carboxylate | |
| 19 | 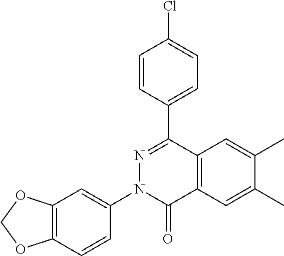 [SMILES: Cc1cc2c(-c3ccc(Cl)cc3)nn(-c3ccc4c(c3)OCO4)c(=O)c2cc1C] | 2-(benzo[d][1,3]dioxol-5-yl)-4-(4- chlorophenyl)-6,7-dimethylphthalazin- 1(2H)-one | |
| 20 | 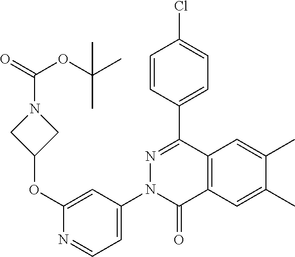 [SMILES: Cc1cc2c(-c3ccc(Cl)cc3)nn(-c3ccnc(OC4CN(C(=O)OC(C)(C)C)C4)c3)c(=O)c2cc1C] | tert-butyl 3-((4-(4-(4-chlorophenyl)-6,7- dimethyl-1-oxophthalazin-2(1H)- yl)pyridin-2-yl)oxy)azetidine-1- carboxylate | |
| 21 | 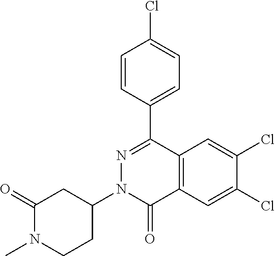 [SMILES: CN1CCC(n2nc(-c3ccc(Cl)cc3)c3cc(Cl)c(Cl)cc3c2=O)CC1=O] | 6,7-dichloro-4-(4-chlorophenyl)-2-(1- methyl-2-oxopiperidin-4-yl)phthalazin- 1(2H)-one | |
| 22 | 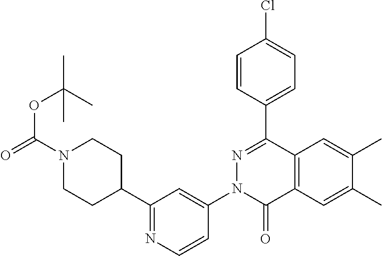 [SMILES: Cc1cc2c(-c3ccc(Cl)cc3)nn(-c3ccnc(C4CCN(C(=O)OC(C)(C)C)CC4)c3)c(=O)c2cc1C] | tert-butyl 4-(4-(4-(4-chlorophenyl)-6,7- dimethyl-1-oxophthalazin-2(1H)- yl)pyridin-2-yl)piperidine-1-carboxylate | |
| 23 | 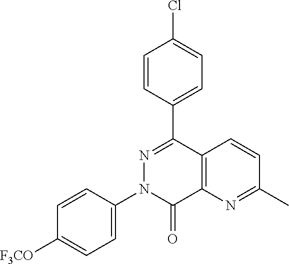 [SMILES: Cc1ccc2c(-c3ccc(Cl)cc3)nn(-c3ccc(OC(F)(F)F)cc3)c(=O)c2n1] | 5-(4-chlorophenyl)-2-methyl-7-(4- (trifluoromethoxy)phenyl)pyrido[2,3- d]pyridazin-8(7H)-one | |
| 24 | 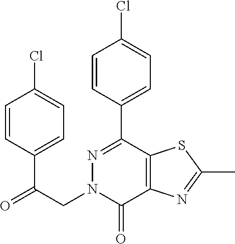 [SMILES: Cc1nc2c(=O)n(CC(=O)c3ccc(Cl)cc3)nc(-c3ccc(Cl)cc3)c2s1] | 7-(4-chlorophenyl)-5-(2-(4- chlorophenyl)-2-oxoethyl)-2- methylthiazolo[4,5-d]pyridazin-4(5H)- one | Method 2A |
| 25 | 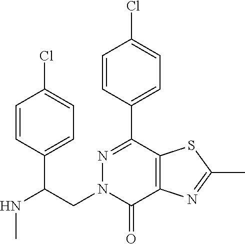 [SMILES: CNC(Cn1nc(-c2ccc(Cl)cc2)c2sc(C)nc2c1=O)c1ccc(Cl)cc1] | 7-(4-chlorophenyl)-5-(2-(4- chlorophenyl)-2-(methylamino)ethyl)- 2-methylthiazolo[4,5-d]pyridazin- 4(5H)-one | Method 2B |
| 26 | 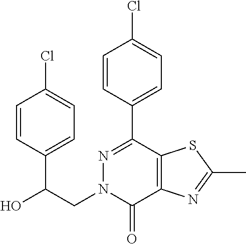 [SMILES: Cc1nc2c(=O)n(CC(O)c3ccc(Cl)cc3)nc(-c3ccc(Cl)cc3)c2s1] | 7-(4-chlorophenyl)-5-(2-(4- chlorophenyl)-2-hydroxyethyl)-2- methylthiazolo[4,5-d]pyridazin-4(5H)- one | Method 2C |
| 27 | 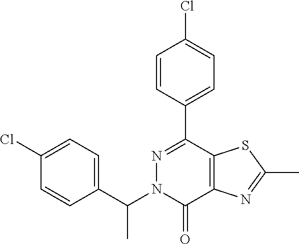 [SMILES: Cc1nc2c(=O)n(C(C)c3ccc(Cl)cc3)nc(-c3ccc(Cl)cc3)c2s1] | 7-(4-chlorophenyl)-5-(1-(4- chlorophenyl)ethyl)-2- methylthiazolo[4,5-d]pyridazin-4(5H)- one | Method 2A, starting from 1-(1- bromoethyl)-4- chlorobenzene |
| 28 | 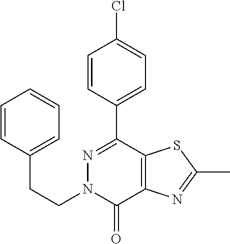 [SMILES: Cc1nc2c(=O)n(CCc3ccccc3)nc(-c3ccc(Cl)cc3)c2s1] | 7-(4-chlorophenyl)-2-methyl-5- phenethylthiazolo[4,5-d]pyridazin- 4(5H)-one | Method 3A |
| 29 | 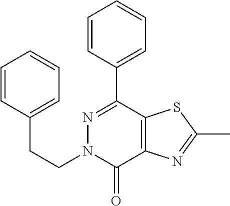 [SMILES: Cc1nc2c(=O)n(CCc3ccccc3)nc(-c3ccccc3)c2s1] | 2-methyl-5-phenethyl-7- phenylthiazolo[4,5-d]pyridazin-4(5H)- one | Method 3B |
| 30 | 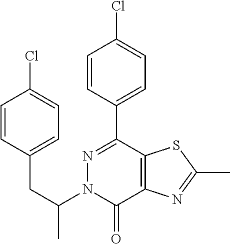 [SMILES: Cc1nc2c(=O)n(C(C)Cc3ccc(Cl)cc3)nc(-c3ccc(Cl)cc3)c2s1] | 7-(4-chlorophenyl)-5-(1-(4- chlorophenyl)propan-2-yl)-2- methylthiazolo[4,5-d]pyridazin-4(5H)- one | Method 4 |
| 31 | 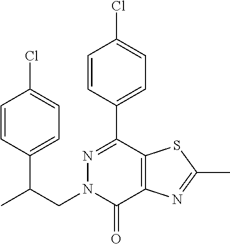 [SMILES: Cc1nc2c(=O)n(CC(C)c3ccc(Cl)cc3)nc(-c3ccc(Cl)cc3)c2s1] | 7-(4-chlorophenyl)-5-(2-(4- chlorophenyl)propyl)-2- methylthiazolo[4,5-d]pyridazin-4(5H)- one | Method 4, starting with 2-(4- chlorophenyl)propan- 1-ol |
| 32 | 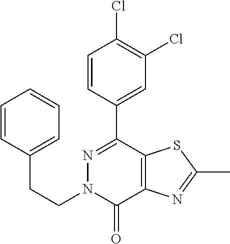 [SMILES: Cc1nc2c(=O)n(CCc3ccccc3)nc(-c3ccc(Cl)c(Cl)c3)c2s1] | 7-(3,4-dichlorophenyl)-2-methyl-5- phenethylthiazolo[4,5-d]pyridazin- 4(5H)-one | Method 2A, starting with (2- bromoethyl)benzene and Int-2 |
| 33 | 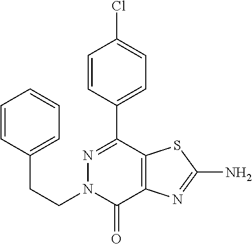 [SMILES: Nc1nc2c(=O)n(CCc3ccccc3)nc(-c3ccc(Cl)cc3)c2s1] | 2-amino-7-(4-chlorophenyl)-5- phenethylthiazolo[4,5-d]pyridazin- 4(5H)-one | Method 2A, starting with (2- bromoethyl)benzene and Int-4 |
| 34 | 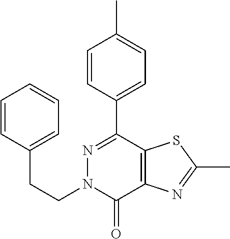 [SMILES: Cc1ccc(-c2nn(CCc3ccccc3)c(=O)c3nc(C)sc23)cc1] | 2-methyl-5-phenethyl-7-(p- tolyl)thiazolo[4,5-d]pyridazin-4(5H)- one | Method 3C |
| 35 | 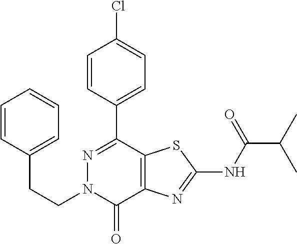 [SMILES: CC(C)C(=O)Nc1nc2c(=O)n(CCc3ccccc3)nc(-c3ccc(Cl)cc3)c2s1] | N-[7-(4-chlorophenyl)-4-oxo-5-(2- phenylethyl)thiazolo[4,5-d]pyridazin-2- yl]-2-methyl-propanamide | Method 5A |
| 36 | 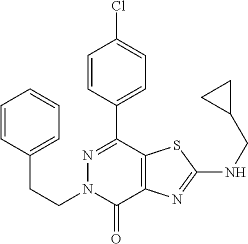 [SMILES: O=c1c2nc(NCC3CC3)sc2c(-c2ccc(Cl)cc2)nn1CCc1ccccc1] | 7-(4-chlorophenyl)-2- (cyclopropylmethylamino)-5-(2- phenylethyl)thiazolo[4,5-d]pyridazin-4- one | Method 5B |
| 37 | 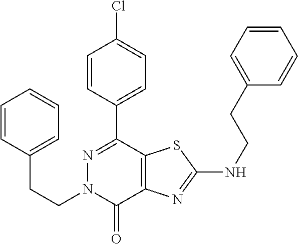 [SMILES: O=c1c2nc(NCCc3ccccc3)sc2c(-c2ccc(Cl)cc2)nn1CCc1ccccc1] | 7-(4-chlorophenyl)-5-(2-phenylethyl)-2- (2-phenylethylamino)thiazolo[4,5- d]pyridazin-4-one | Method 6 |
| 38 | 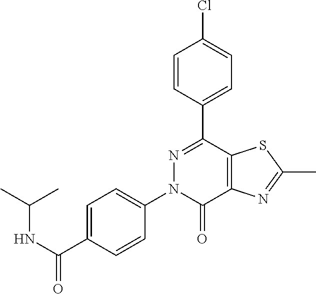 [SMILES: Cc1nc2c(=O)n(-c3ccc(C(=O)NC(C)C)cc3)nc(-c3ccc(Cl)cc3)c2s1] | 4-(7-(4-chlorophenyl)-2-methyl-4- oxothiazolo[4,5-d]pyridazin-5(4H)-yl)- N-isopropylbenzamide | |
| 39 | 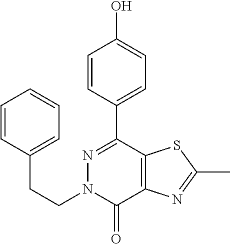 [SMILES: Cc1nc2c(=O)n(CCc3ccccc3)nc(-c3ccc(O)cc3)c2s1] | 7-(4-hydroxyphenyl)-2-methyl-5- phenethylthiazolo[4,5-d]pyridazin- 4(5H)-one | |
| 40 | 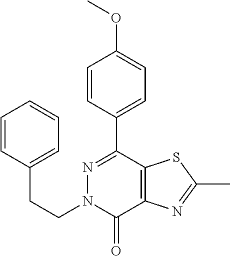 [SMILES: COc1ccc(-c2nn(CCc3ccccc3)c(=O)c3nc(C)sc23)cc1] | 7-(4-methoxyphenyl)-2-methyl-5- phenethylthiazolo[4,5-d]pyridazin- 4(5H)-one | |
| 41 | 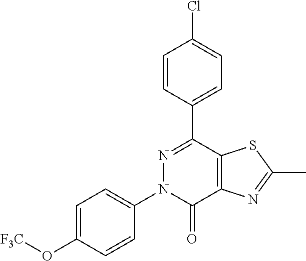 [SMILES: Cc1nc2c(=O)n(-c3ccc(OC(F)(F)F)cc3)nc(-c3ccc(Cl)cc3)c2s1] | 7-(4-chlorophenyl)-2-methyl-5-(4- (trifluoromethoxy)phenyl)thiazolo[4,5- d]pyridazin-4(5H)-one | |
| 42 | 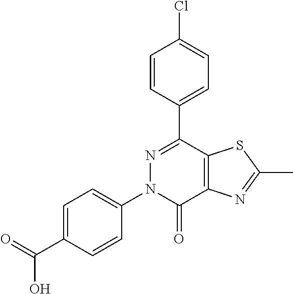 [SMILES: Cc1nc2c(=O)n(-c3ccc(C(=O)O)cc3)nc(-c3ccc(Cl)cc3)c2s1] | 4-(7-(4-chlorophenyl)-2-methyl-4- oxothiazolo[4,5-d]pyridazin-5(4H)- yl)benzoic acid | |

TABLE C
Analytical data for compounds of Table B

| Ex # | NMR | M + H |
|---|---|---|
| 1 | ¹HNMR (400 MHz, CDCl₃) δ = 7.87-7.81 (m, 2H), 7.75 (s, 1H), 7.55 (d, J = 1.2 | 445.8 |
| | Hz, 3H), 7.33 (d, J = 8.8 Hz, 2H), 2.82 (s, 3H), 2.48 (s, 3H) | |
| 24 | ¹H NMR (400 MHz, CDCl3) δ 7.98-7.96 (m, 2H), 7.75-7.72 (m, 2H), 7.51-7.45 (m, | 430.1 |
| | 4H), 5.82 (s, 2H), 2.96 (s, 3H) | |
| 25 | ¹H NMR (400 MHz, DMSO) δ 7.66-7.60 (m, 4H), 7.39-7.33 (m, 4H), 4.57-4.52 (m, | 445.1 |
| | 1H), 4.29-4.24 (m, 1H), 4.11-4.07 (t, J = 6.4 Hz, 1H), 2.87 (s, 3H), 2.12 (s, 3H) | |
| 26 | ¹H NMR (400 MHz, DMSO) δ 7.72-7.70 (m, 2H), 7.64-7.62 (m, 2H), 7.39 (s, 4H), | 432.1 |
| | 5.72 (s, 1H), 5.14-5.10 (m, 1H), 4.52-4.46 (m, 1H), 4.32-4.27 (m, 1H), 2.88 (s, | |
| | 3H) | |
| 27 | ¹H NMR (400 MHz, DMSO) δ 7.85 (d, J = 8.5 Hz, 2H), 7.66 (d, J = 8.5 Hz, 2H), | 416.1 |
| | 7.44-7.38 (m, 4H), 6.40 (q, J = 7.0 Hz, 1H), 2.88 (s, 3H), 1.79 (d, J = 7.0 Hz, 3H). | |
| 28 | ¹H NMR (400 MHz, DMSO) δ 7.72-7.70 (m, 2H), 7.63-7.61 (m, 2H), 7.29-7.18 (m, | 382 |
| | 5H), 4.52-4.49 (t, J = 7.2, 2H), 3.15-3.14 (t, J = 7.2, 2H), 2.88 (s, 3H). | |
| 29 | ¹H NMR (400 MHz, DMSO) δ 7.73-7.63 (m, 2H), 7.55-7.53 (m, 3H), 7.30-7.20 | 348.1 |
| | (m, 5H), 4.51 (t, J = 7.3 Hz, 2H), 3.14 (t, J = 7.3 Hz, 2H), 2.87 (s, 3H) | |
| 30 | ¹H NMR (400 MHz, DMSO) δ 7.89 (d, J = 8.6 Hz, 2H), 7.68 (d, J = 8.6 Hz, 2H), | 431 |
| | 7.25 (d, J = 8.4 Hz, 2H), 7.16 (d, J = 8.4 Hz, 2H), 5.64-5.48 (m, 1H), 3.21-3.05 | |
| | (m, 2H), 2.85 (s, 3H), 1.42 (d, J = 6.6 Hz, 3H). | |
| 31 | ¹H NMR (400 MHz, DMSO) δ 7.65-7.59 (m, 4H), 7.32-7.27 (m, 4H), 4.47-4.35 (m, | 430.1 |
| | 2H), 3.47-3.41 (m, 1H), 2.86 (s, 3H), 1.30-1.28 (d, J = 6.8 Hz, 3H). | |
| 32 | ¹H NMR (400 MHz, DMSO) δ 7.84-7.82 (d, J = 8.4 Hz, 1H), 7.74-7.74 (d, J = 2.4 | 416.1 |
| | Hz, 1H), 7.68-7.66 (dd, J = 2.4 Hz, J = 8.4 Hz, 1H), 7.30-7.21 (m, 5H), 4.54-4.50 (t, | |
| | J = 7.2 Hz, 2H), 3.14-3.10 (t, J = 7.2 Hz, 2H), 2.88 (s, 3H). | |
| 33 | ¹H NMR (400 MHz, DMSO) δ 8.26 (s, 2H), 7.68-7.64 (m, 2H), 7.62-7.57 (m, | 383.10 |
| | 2H), 7.32-7.20 (m, 5H), 4.42 (t, J = 7.4 Hz, 2H), 3.09 (t, J = 7.6 Hz, 2H). | |
| 34 | ¹H NMR δ 7.59-7.57 (m, 2H), 7.36-7.34 (m, 2H), 7.31-7.16 (m, 5H), 4.52-4.48 (m, | 362 |
| | 2H), 3.14-3.11 (m, 2H), 2.87 (s, 3H), 2.38 (s, 3H). | |
| 35 | ¹H NMR (400 MHz, CDCl3) δ 11.30 (s, 1H), 7.68 (d, J = 8.4 Hz, 2H), 7.47 (d, J = | 453.00 |
| | 8.4 Hz, 2H), 7.33-7.18 (m, 5H), 4.66 (t, J = 7.4 Hz, 2H), 3.24 (t, J = 7.4 Hz, 2H), | |
| | 3.07-2.91 (m, 1H), 1.35 (d, J = 5.9 Hz, 6H). | |
| 36 | ¹H NMR (400 MHz, DMSO) δ 8.92 (s, 1H), 7.64 (d, J = 8.4 Hz, 2H), 7.59 (d, J = | 437.20 |
| | 8.4 Hz, 2H), 7.28-7.19 (m, 5H), 4.43 (t, J = 7.3 Hz, 2H), 3.28 (t, J = 6.0 Hz, 2H), | |
| | 3.09 (t, J = 7.3 Hz, 2H), 1.19-1.02 (m, 1H), 0.63-0.42 (m, 2H), 0.28 (q, J = 4.5 | |
| | Hz, 2H). | |
| 37 | ¹H NMR (400 MHz, CDCl3) δ 7.57-7.48 (m, 2H), 7.46 (t, J = 6.8 Hz, 2H), 7.35- | 487.24 |
| | 7.18 (m, 10H), 4.71-4.54 (m, 2H), 3.80-3.56 (m, 2H), 3.24-3.20 (m, 2H), 3.09- | |
| | 3.05 (m, 2H). | |

### Example A3: In Vitro Assay Data

### In Vitro Measurement of Triggering Receptor Expressed on Myeloid Cells 2 Activity Using Cellular Phosphorylation of Spleen Tyrosine Kinase (“Syk”) Assays

Measurement of TREM2 agonist potency was done using a HEK cell line expressing human TREM2 and DAP12 (HEK293T-hTREM2 cells). Binding of small molecules to, and activation of, TREM2 increases the phosphorylation of Syk. The resultant levels of Syk phosphorylation are measured using a commercial AlphaLisa reagent kit. To perform the assay, HEK-hTREM2 cells were plated at 14,000 cells per well in a 384 well plate, in 25 μL of complete growth media and incubated at 37° C., 5% CO₂ for 20-24 hours.

Prior to the assay, test compounds were diluted in the 384 well plates in assay buffer and allowed to equilibrate for 30 minutes. Growth media was removed from cell plates by inversion on blotting paper, and 25 μL of test articles in assay buffer was added to cells. Cells were incubated for 45 minutes at room temperature. After 45 minutes, assay buffer was removed and 10 μL of lysis buffer was added. Plates were shaken for 20 minutes at 350 RPM at room temperature. After complete lysis, AlphaLisa reagents were added to the lysate, and fluourescence intensity was measured using a Perkin Elmer Envision plate reader. Intensities were used to generate a standard curve, and % activation was calculated. Curve fitting was performed using Prism v9 software, log(agonist) vs response—variable slope (four parameters), and EC50s were calculated from the curve fit.

The results presented in Table D have been generated with the in vitro assay described above. This assay may be used to test any of the compounds described herein to assess and characterize a compound's ability to act as an agonist of TREM2.

Compounds designated as “A” demonstrated an EC50 of ≤0.05 μM. Compounds designated as “B” demonstrated an EC50>0.05 μM and ≤0.5 μM. Compounds designated as “C” demonstrated an EC50>0.5 μM and ≤3.0 μM. Compounds designated as “D” demonstrated an EC50>3.0 μM and ≤100 μM. Compounds designated as “-” had not been tested as of the filing of the present application, but can be tested using the methods described herein.

TABLE D
hTREM2 EC50 Data (HEK293 Cells)

| Ex # | hTREM2 EC50 μM |
|---|---|
| 1 | C |
| 2 | D |
| 3 | D |
| 4 | D |
| 5 | C |
| 6 | C |
| 7 | D |
| 8 | D |
| 9 | C |
| 10 | C |
| 11 | C |
| 12 | C |
| 13 | D |
| 14 | C |
| 15 | C |
| 16 | C |
| 17 | D |
| 18 | C |
| 19 | D |
| 20 | C |
| 21 | D |
| 22 | C |
| 23 | C |
| 24 | D |
| 25 | D |
| 26 | D |
| 27 | D |
| 28 | D |
| 29 | D |
| 30 | D |
| 31 | D |
| 32 | D |
| 33 | D |
| 34 | D |
| 35 | D |
| 36 | D |
| 37 | D |
| 38 | D |
| 39 | D |
| 40 | D |
| 41 | D |
| 42 | B |

TABLE D-2
hTREM2 EC₅₀ Data (HEK293 Cells)

| I-# | hTREM2 EC₅₀ |
|---|---|
| I-104 | — |
| I-107 | C |
| I-108 | C |
| I-109 | C |
| I-110 | — |
| I-93 | C |
| I-111 | C |
| I-112 | D |
| I-113 | C |
| I-116 | C |
| I-117 | C |
| I-118 | C |
| I-94 | C |
| I-119 | C |
| I-120 | B |
| I-121 | D |
| I-122 | C |
| I-123 | C |

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of Formula I 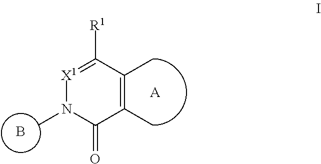
or a tautomer thereof, or a pharmaceutically acceptable salt of said compound or said tautomer, wherein
R¹ is an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, optionally substituted OCH₂—(C₃₋₆cycloalkyl), or a cyclic group selected from a 3-8 membered saturated or partially unsaturated monocyclic carbocyclic ring, a 5-12 membered saturated or partially unsaturated bridged carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 6-12 membered saturated or partially unsaturated bridged heterocyclic ring (having 1-4 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X¹ is CH, CR¹⁶, or N;
Ring A together with the 6-membered ring system to which it is fused forms a bicyclic ring system of formula 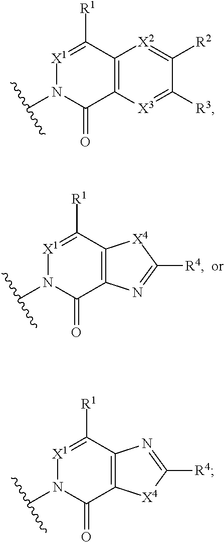
R² and R³ are each independently selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
provided that at least one of R² and R³ is not hydrogen;
or R² and R³ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X² is CH, CR¹⁴, or N;
X³ is CH, CR¹⁵, or N;
R⁴ is selected from hydrogen, an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁴ is NR, O or S;
Ring B is 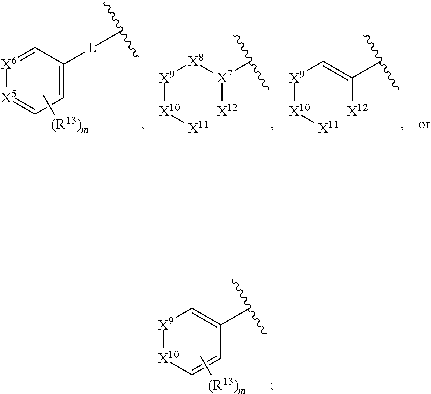
L is a bond or an optionally substituted straight chain or branched C₁₋₆ alkylene;
X⁵ is CH, N or CR⁵;
X⁶ is CH, N or CR⁶;
provided that when one of X⁵ or X⁶ is N, the other is not N;
R⁵ and R⁶ are each independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, halogen, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or R⁵ and R⁶ are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
X⁷ is N, CH, or CR⁷;
X⁸ is O, NR⁸, C(R⁸)₂, CHR⁸, SO₂, or C═O;
X⁹ is O, NR⁹, C(R⁹)₂, CHR⁹, SO₂, or C═O;
X¹⁰ is O, NR¹⁰, C(R¹⁰)₂, CHR¹⁰, SO₂, or C═O;
X¹¹ is O, NR¹¹, C(R¹¹)₂, CHR¹¹, SO₂, or C═O;
X¹² is a direct bond, O, NR¹², C(R¹²)₂, CHR¹², —CH₂CH₂—, —OCH₂—, SO₂, or C═O;
R⁷ is an optionally substituted aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
each of R⁸, R⁹, R¹⁰, R¹¹, and R¹² is independently selected from hydrogen, an optionally substituted C₁₋₆ aliphatic group, —OR, —CN, —NR₂, —C(═O)R, —C(═O)OR, —C(═O)NR₂, —SO₂R, —SO₂NR₂, C₁₋₆haloalkyl, C₁₋₆haloalkoxy, or a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
or any two of R⁷, R⁸, R⁹, R¹⁰, R¹¹, and R¹² are taken together with their intervening atoms to form a cyclic group selected from a 3-8 membered saturated or partially unsaturated monocyclic carbocyclic ring, a 7-12 membered saturated or partially unsaturated bicyclic carbocyclic ring, phenyl, an 8-10 membered bicyclic aromatic carbocyclic ring, a 3-8 membered saturated or partially unsaturated monocyclic heterocyclic ring (having 1-2 heteroatoms independently selected from nitrogen, oxygen, and sulfur), a 7-12 membered saturated or partially unsaturated bicyclic heterocyclic ring (having 1-4 heteroatoms independently selected from nitrogen, oxygen, and sulfur), a 5-6 membered monocyclic heteroaromatic ring (having 1-4 heteroatoms independently selected from nitrogen, oxygen, and sulfur), and an 8-10 membered bicyclic heteroaromatic ring (having 1-5 heteroatoms independently selected from nitrogen, oxygen, and sulfur), wherein the cyclic group is optionally substituted;
R¹³ is an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁴ and R¹⁵ are each independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
m is 0, 1 or 2;
R¹⁴ and R¹⁵ are each independently an optionally substituted C₁₋₆ aliphatic group, halogen, —OR, —CN, —NR₂, —C(═O)R, —C(═O)OR, —C(═O)NR₂, —SO₂R, —SO₂NR₂, C₁₋₆haloalkyl, or C₁₋₆haloalkoxy;
R¹⁶ is an optionally substituted C₁₋₆ aliphatic group;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same nitrogen are taken together with their intervening atoms to form an optionally substituted 4-7 membered saturated, partially unsaturated, or heteroaryl ring (having 0-3 heteroatoms, in addition to the nitrogen, independently selected from nitrogen, oxygen, and sulfur).

2. The compound of claim 1, wherein R¹ is optionally substituted C₃₋₆cycloalkyl, optionally substituted spiro[3.3]heptanyl, optionally substituted spiro[5.2]octanyl, optionally substituted 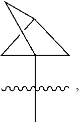 [SMILES: *C12CC(C1)C2]
optionally substituted cyclopent-1-en-1-yl, optionally substituted cyclohex-1-en-1-yl, optionally substituted phenyl, optionally substituted pyridinyl, optionally substituted aziridine-1-yl, optionally substituted pyrrolidine-1-yl, optionally substituted azabicyclo[3.1.0]hexan-3-yl, optionally substituted piperidine-1-yl, or optionally substituted —OCH₂—(C₃₋₄cycloalkyl).

3. The compound of claim 1, wherein R¹ is optionally substituted phenyl.

4. The compound of claim 1, wherein R¹ is:
(A) —CH₂CH₂CF₃, 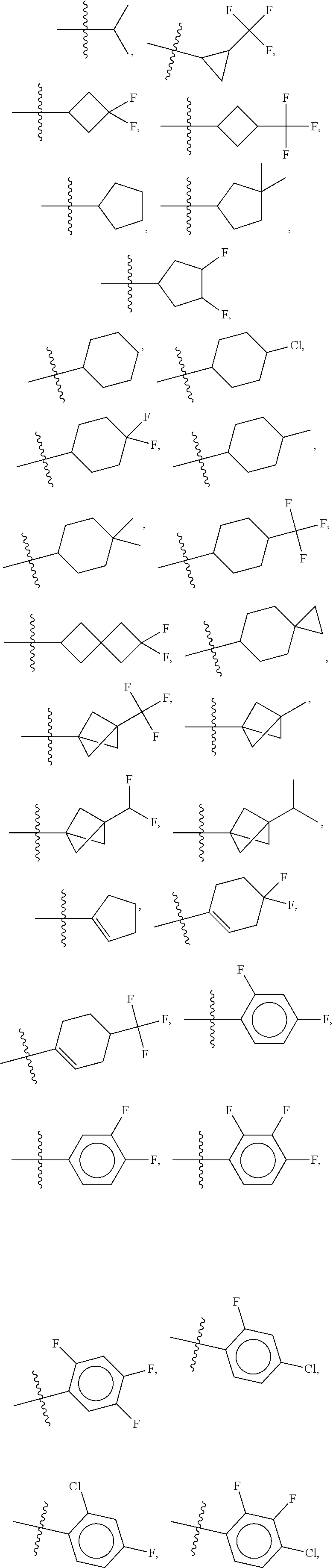 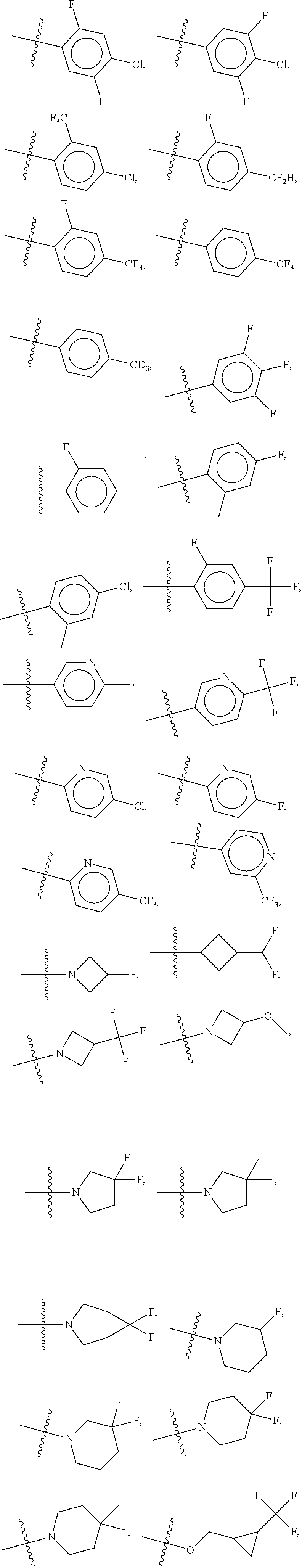 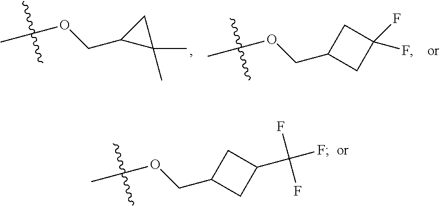
(B) a substituent selected from:

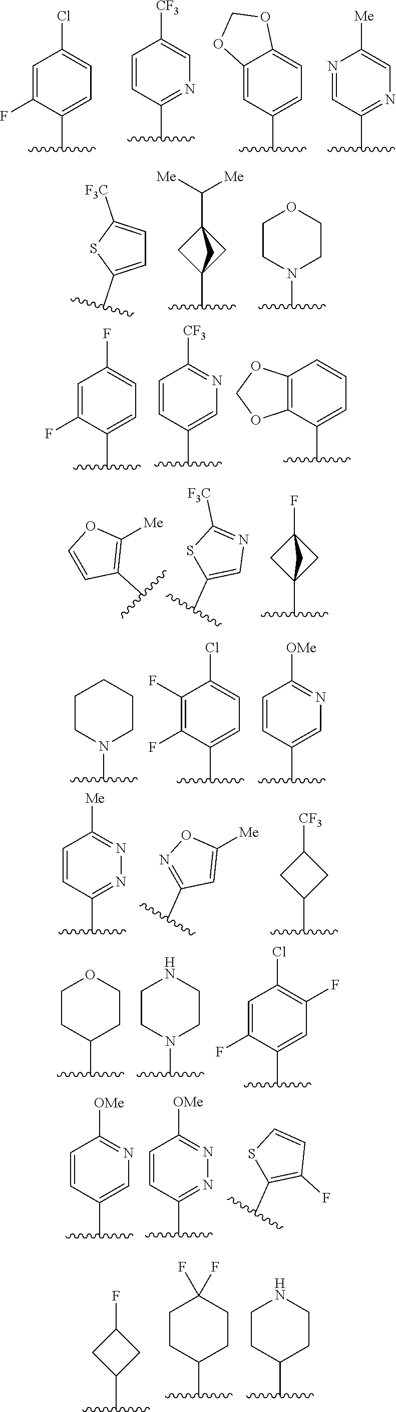

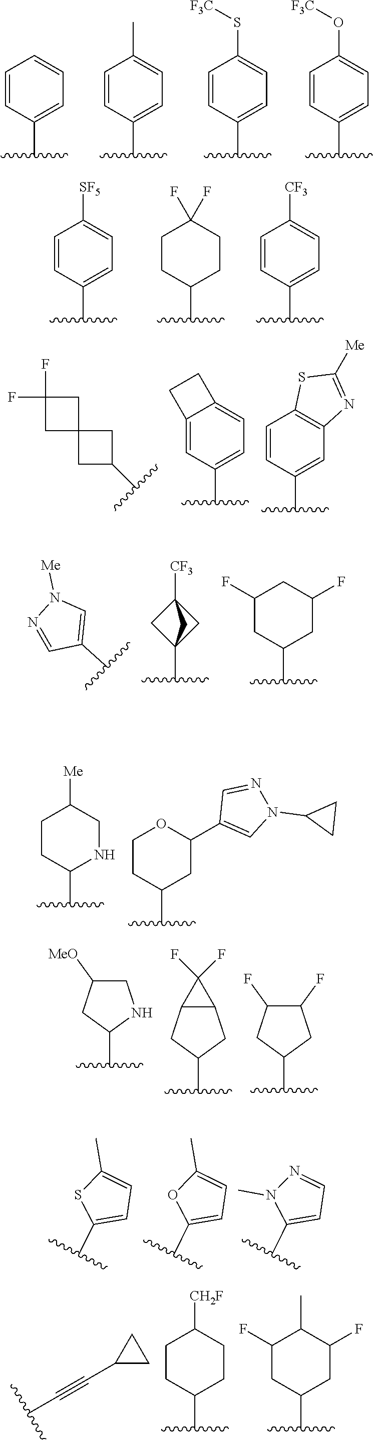

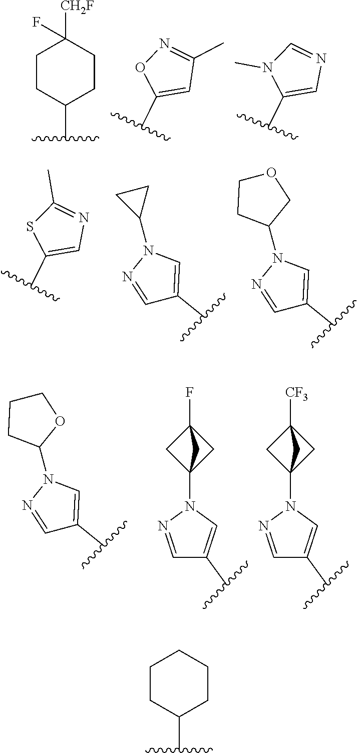

5. The compound of any one of claims 1-4, wherein X¹ is CH or N.

6. The compound of any one of claims 1-5, wherein X² is CH or N.

7. The compound of any one of claims 1-5, wherein X³ is S.

8. The compound of any one of claims 1-5, wherein X⁴ is S.

9. The compound of any one of claims 1-4, wherein Ring A together with the 6-membered ring system to which it is fused forms a bicyclic ring system of formula

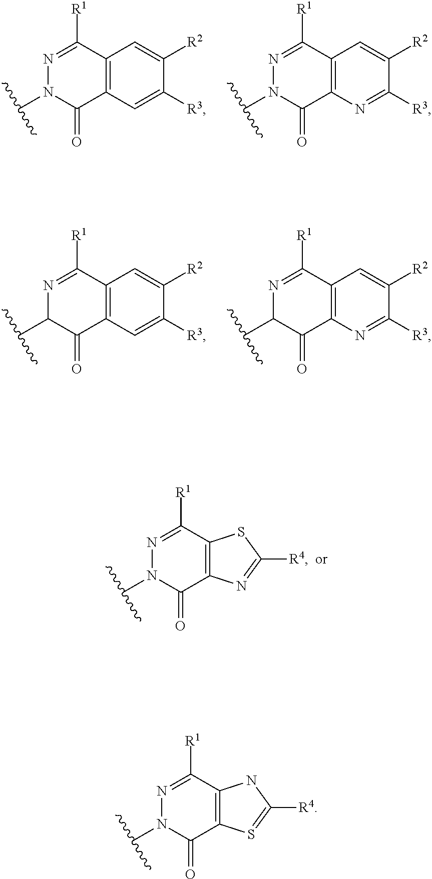

10. The compound of any one of claims 1-9, wherein Ring B is

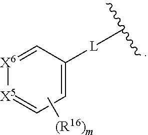

11. The compound of any one of claims 1-10, wherein Ring B is

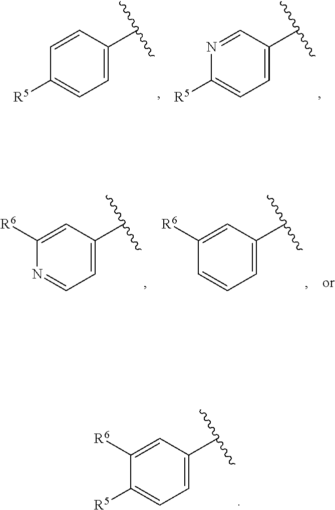

12. The compound of any one of claims 1-10, wherein R⁵ and R⁶ are each independently selected from hydrogen or one of the following:

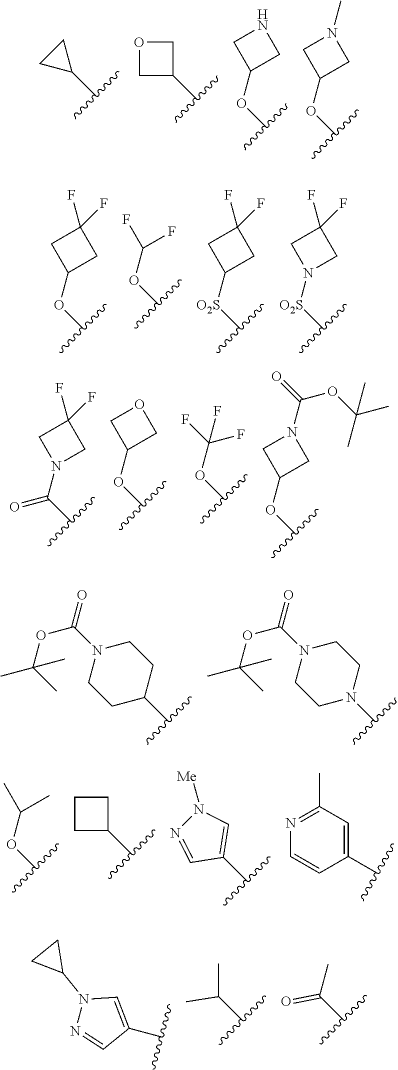

13. The compound of any one of claims 1-9, wherein Ring B is

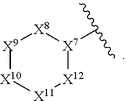

14. The compound of any one of claims 1-9, wherein Ring B is selected from:

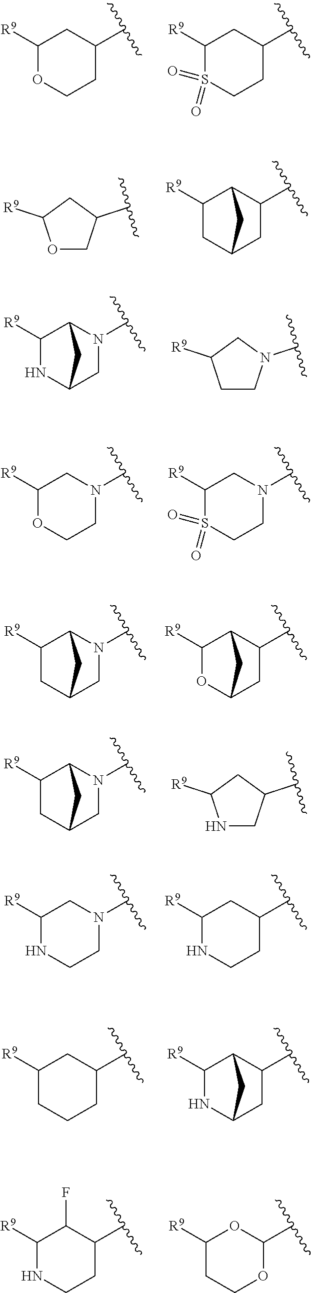

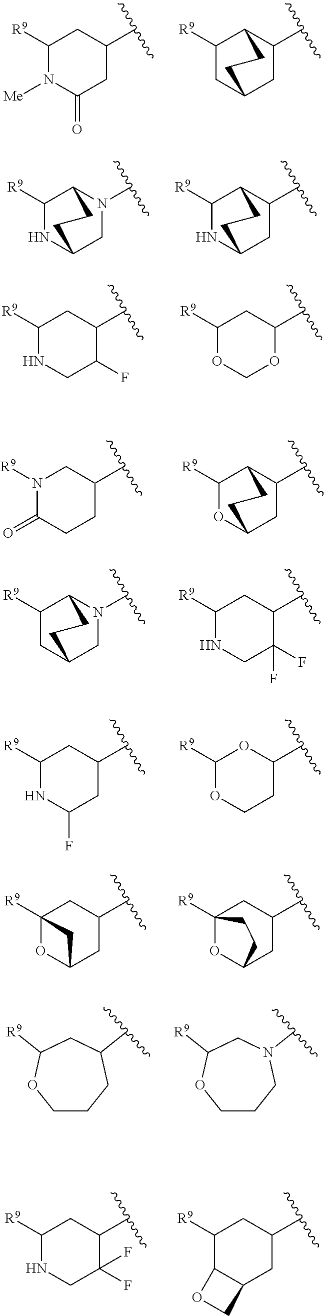

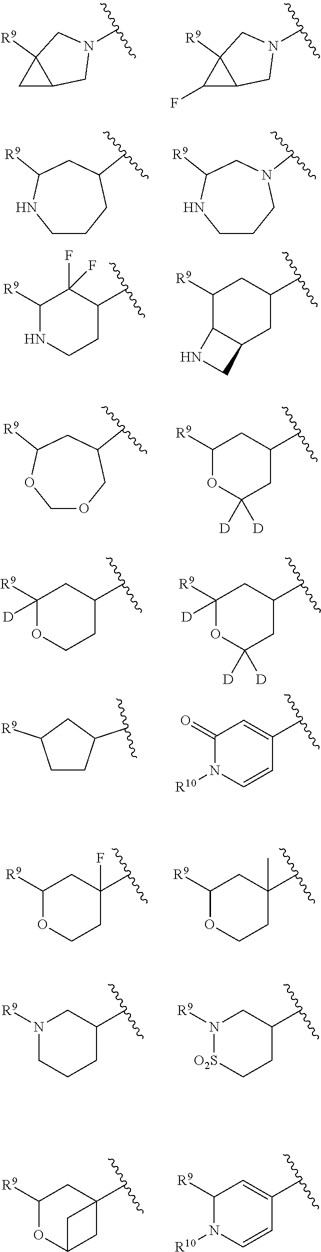

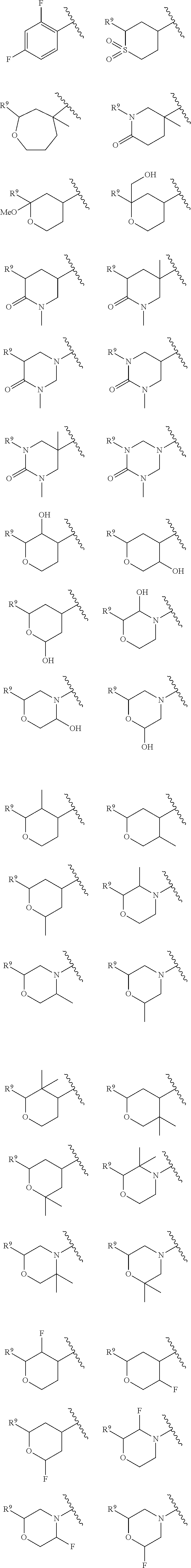

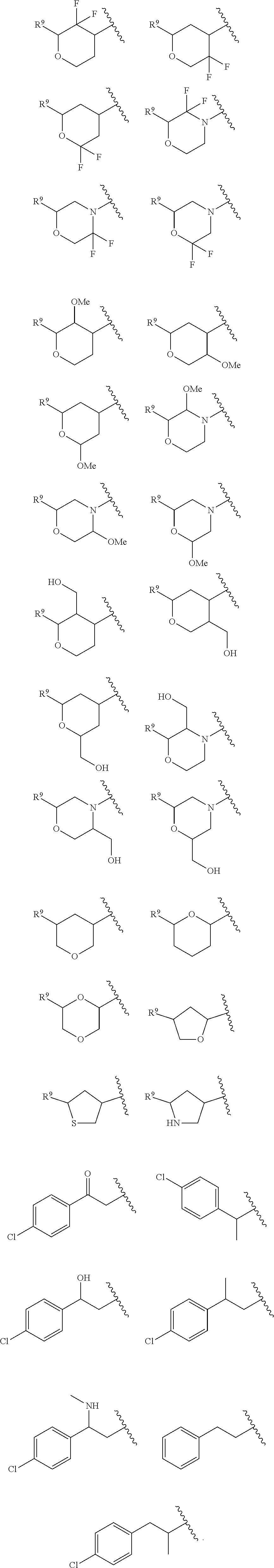

15. The compound of any one of claims 1-9, wherein Ring B is:

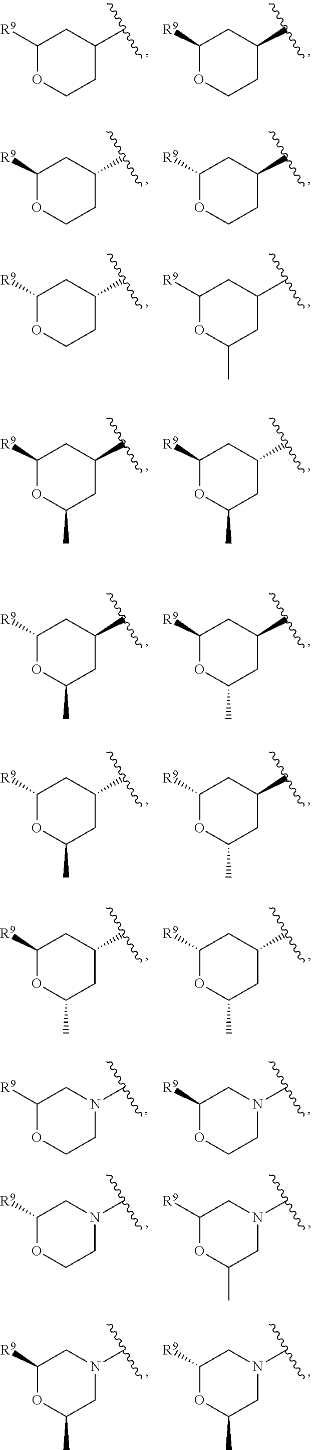

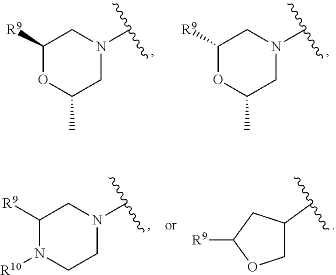

16. The compound of any one of claims 1-9, 13 and 14, wherein R⁹ is selected from:

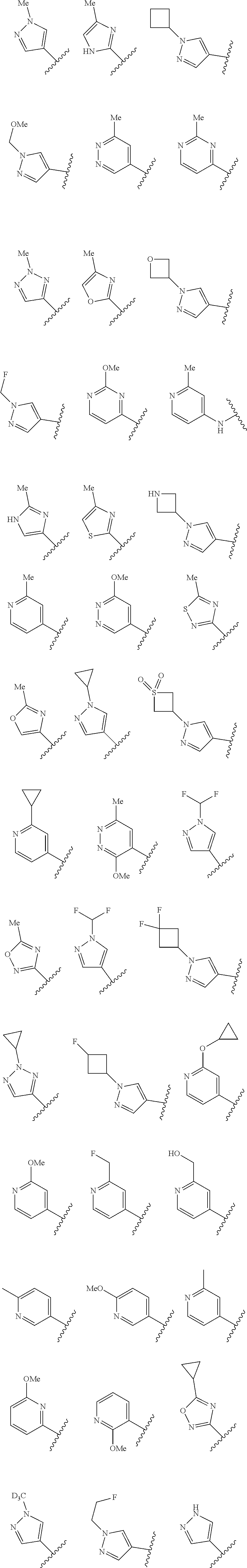

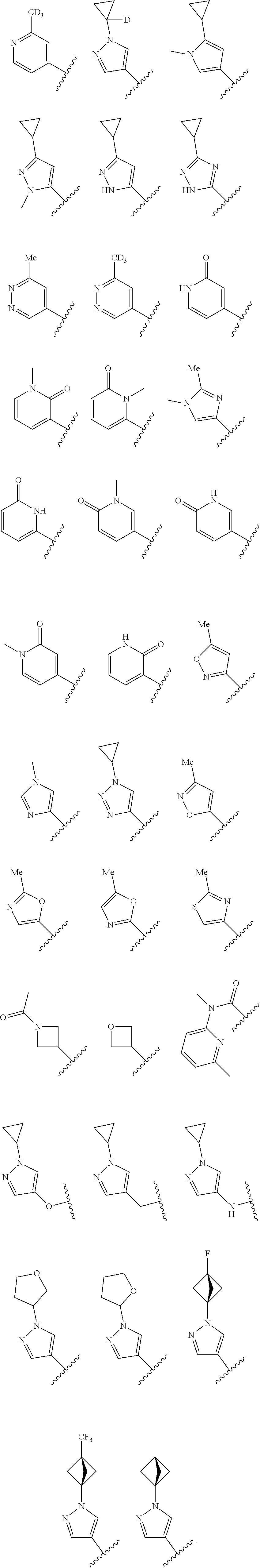

17. The compound of any one of claims 1-9, 13 and 14, wherein R⁹ is

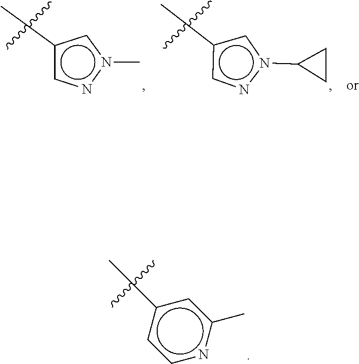

18. The compound of any one of claims 1-17, wherein the compound is a compound of Formula II, IIa, IIa*, IIa**, IIb, IIb*, IIb**, IIb***, IIb′, IIb″, IIb′″, IIb″″, IIb′″″, III, IIIa, IIIb, IIIc, IV, IVa, IVb, or IVc.

19. A compound of Table A, or a pharmaceutically acceptable salt thereof.

20. A pharmaceutical composition comprising the compound according to any one of claims 1-19, or a tautomer thereof, or a pharmaceutically acceptable salt of said compound or said tautomer, and a pharmaceutically acceptable excipient.

21. A compound according to any one of claims 1-19, or a tautomer thereof, or a pharmaceutically acceptable salt of said compound or said tautomer, or the pharmaceutical composition according to claim 20 for use as a medicament.

22. A compound according to any one of claims 1-19, or a tautomer thereof, or a pharmaceutically acceptable salt of said compound or said tautomer, or the pharmaceutical composition according to claim 20 for use in treating or preventing a condition associated with a loss of function of human TREM2.

23. A compound according to any one of claims 1-19, or a tautomer thereof, or a pharmaceutically acceptable salt of said compound or said tautomer, or the pharmaceutical composition according to claim 20 for use in treating or preventing Parkinson's disease, rheumatoid arthritis, Alzheimer's disease, Nasu-Hakola disease, frontotemporal dementia, multiple sclerosis, prion disease, or stroke.

24. Use of the compound according to any one of claims 1-19, or a tautomer thereof, or a pharmaceutically acceptable salt of said compound or said tautomer, or the pharmaceutical composition according to claim 20 in the preparation of a medicament for treating or preventing a condition associated with a loss of function of human TREM2.

25. Use of the compound according to any one of claims 1-19, or a tautomer thereof, or a pharmaceutically acceptable salt of said compound or said tautomer, or the pharmaceutical composition according to claim 20 in the preparation of a medicament for treating or preventing Parkinson's disease, rheumatoid arthritis, Alzheimer's disease, Nasu-Hakola disease, frontotemporal dementia, multiple sclerosis, prion disease, or stroke.

26. A method of treating or preventing a condition associated with a loss of function of human TREM2 in a subject in need thereof, the method comprising administering to the subject a therapeutically effective amount of the compound according to any one of claims 1-19, or a tautomer thereof, or a pharmaceutically acceptable salt of said compound or said tautomer.

27. A method of treating or preventing Parkinson's disease, rheumatoid arthritis, Alzheimer's disease, Nasu-Hakola disease, frontotemporal dementia, multiple sclerosis, prion disease, or stroke in a subject in need thereof, the method comprising administering to the subject a therapeutically effective amount of the compound according to any one of claims 1-19, or a tautomer thereof, or a pharmaceutically acceptable salt of said compound or said tautomer.
